(19) United States
(12) Patent Application Publication
(10) Pub. No.: US 2018/0201592 A1
(43) Pub. Date: Jul. 19, 2018

(54) 2-AMINO-1,3,4-THIADIAZINE AND 2-AMINO-1,3,4-OXADIAZINE BASED ANTIFUNGAL AGENTS

(71) Applicant: F2G Limited, Manchester (GB)

(72) Inventors: Graham Edward Morris SIBLEY, Stockport (GB); Lars Jonas MALMSTRÖM, Uppsala (SE); Johanna Maria LARSSON, Uppsala (SE)

(21) Appl. No.: 15/744,510

(22) PCT Filed: Jul. 14, 2016
(86) PCT No.: PCT/GB2016/052132

§ 371 Date: Jan. 12, 2018

(30) Foreign Application Priority Data
Jul. 14, 2015 (GB) 1512286.4

(51) Int. Cl. C07D 285/16; C07D 417/06; C07D 417/12; A61K 31/549; A61K 45/06; A61P 31/10

(52) U.S. Cl. CPC C07D 285/16; C07D 417/06; A61P 31/10; A61K 31/549; A61K 45/06; C07D 417/12

(57) ABSTRACT

The invention provides a compound which is a diazine of formula (I) or a tautomer thereof, or a pharmaceutically acceptable salt thereof, for use as an antifungal agent: (I) wherein X, N′, C′, A and E are as defined herein. The invention also provides a compound of Formula (I) as defined herein.

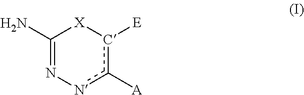

### FIELD OF THE INVENTION

The present invention relates to compounds which are diazines of formula (I). The compounds may be used in the treatment of an animal or human body. The compound may, for example, be used for treating or preventing fungal infection.

### BACKGROUND

Invasive fungal infections are well recognised as diseases of the immunocompromised host. Over the last twenty years there have been significant rises in the number of recorded instances of fungal infection (Groll et al., 1996. J Infect 33, 23-32). In part this is due to increased awareness and improved diagnosis of fungal infection. However, the primary cause of this increased incidence is the vast rise in the number of susceptible individuals. This is due to a number of factors including new and aggressive immunosuppressive therapies, increased survival in intensive care, increased numbers of transplant procedures and the greater use of antibiotics worldwide.

In certain patient groups, fungal infection occurs at high frequency; lung transplant recipients have a frequency of up to 20% colonisation and infection with a fungal organism and fungal infection in allogenic haemopoetic stem cell transplant recipients is as high as 15% (Ribaud et al., 1999, Clin Infect Dis. 28:322-30).

Conventional treatment of fungal infections rely on four classes of antifungal drug: the polyenes (e.g., amphotericin B), the azoles (e.g., ketoconazole or itraconazole), the echinocandins (e.g., caspofungin) and flucytosine. The polyenes are the oldest class of antifungal agent being first introduced in the 1950's. The exact mode of action remains unclear but polyenes are only effective against organisms that contain sterols in their outer membranes. It has been proposed that amphotericin B interacts with membrane sterols to produce pores allowing leakage of cytoplasmic components and subsequent cell death. Azoles work by inhibition of the 14α-demethylase via a cytochrome P450-dependent mechanism. This leads to a depletion of the membrane sterol ergosterol and the accumulation of sterol precursors resulting in a plasma membrane with altered fluidity and structure. Echinocandins work by the inhibition of the cell wall synthetic enzyme (β-glucan synthase. This leads to abnormal cell wall formation, osmotic sensitivity and cell lysis. Flucytosine is a pyrimidine analogue interfering with cellular pyrimidine metabolism as well DNA, RNA and protein synthesis. However widespread resistance to flucyotosine limits its therapeutic use.

Conventional antifungal agents act primarily against only two cellular targets; membrane sterols (polyenes and azoles) and (β-glucan synthase (echinocandins). However, resistance to both azoles and polyenes has been widely reported leaving only the recently introduced echinocandins to combat invasive fungal infections. As the use of echinocandins increases, resistance by fungi will inevitably occur. There is therefore a pressing need for new classes of antifungal agent.

### SUMMARY OF THE INVENTION

The present inventors have found that compounds which are diazines of formula (I) are active as anti-fungal agents. In particular, the compounds inhibit the growth of human pathogenic fungi such as Aspergillus and therefore may be used to treat fungal infection and disease. The compounds exhibit broad spectrum activity across a range of moulds and yeasts including those associated with ‘difficult to treat’ infections.

Accordingly, the present invention provides a compound which is a diazine of formula (I) or a tautomer thereof, or a pharmaceutically acceptable salt thereof, for use as an antifungal agent:

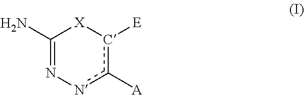

wherein:
X represents O or S;
the moiety

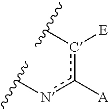

represents —N(D)-C(A)═C(E)- or —N═C(A)-C(R¹)(E)-;
D represents H or C₁-C₆ alkyl, wherein the alkyl group of D is unsubstituted or is substituted with 1, 2 or 3 substituents selected from halogen, OH, and C₁-C₂ alkoxy; and wherein the alkyl group of D is uninterrupted or is interrupted by —O—, —C(O)—, —OC(O)— or —C(O)O—;
R¹ is H or C₁-C₂ alkyl;
One group selected from A and E represents a group Q1, and the other group selected from A and E represents a group Q2;
Q1 represents
   H or C₁-C₈ alkyl, wherein the alkyl group of Q1 is unsubstituted or is substituted with 1, 2 or 3 substituents selected from halogen, OH, and C₁-C₂ alkoxy; and wherein the alkyl group of Q1 is uninterrupted or is interrupted by O, —C(O)—, —OC(O)— or —C(O)O—;
or
   (ii) an alkylene group which is bonded to an atom of group Q2 to form a C₅-C₆ carbocyclyl or 5- to 6-membered heterocyclyl moiety, wherein the carbocylyl or heterocyclyl moiety is saturated or partially unsaturated; and wherein the carbocyclyl or heterocyclyl moiety is unsubstituted or is substituted with 1, 2 or 3 substituents selected from halogen, C₁-C₄ alkoxy, unsubstituted C₁-C₄ alkyl and C₁-C₄ alkyl substituted with 1, 2 or 3 substituents independently selected from halogen and —OH;
Q2 represents a group -L-T or -T, wherein
   L is selected from C₁-C₁₂ alkylene and C₂-C₁₂ alkenylene, wherein the alkylene or alkenylene group of L is unsubstituted or is substituted with 1, 2 or 3 groups selected from halogen, C₁-C₄ alkoxy and —OH; and wherein the alkylene or alkenylene group of L may optionally terminate in and/or be interrupted by a heteromoiety selected from —O—, —S—, —C(O)—, —OC(O)—, —C(O)O—, —NR²—, —NR²C(O)—, and —C(O)NR²—; and
   when Q2 is -L-T, then T is H, aryl, heteroaryl, cycloalkyl or heterocyclyl, and when Q2 is -T, then T is aryl, heteroaryl, cycloalkyl or heterocyclyl, wherein the aryl, heteroaryl, cycloalkyl or heterocyclyl group of T is unsubstituted or is substituted by 1, 2 or 3 groups V;
each group V is independently selected from C₁-C₆ alkoxy, unsubstituted C₁-C₁₀ alkyl, C₁-C₁₀ alkyl which is substituted with 1, 2 or 3 groups selected from halogen and C₁-C₃ alkoxy, C₂-C₆ alkenyl, C₂-C₆ alkynyl, C₃-C₆ cycloalkyl, halogen, aryl, (C₁-C₆ alkyl)-aryl, aryloxy, aryloxy-(C₁-C₆ alkyl), —CN, NO₂, —(C₁-C₆ alkyl)-C(O)O(C₁-C₆ alkyl) and —C(O)O(C₁-C₆ alkyl); and
R² is H or C₁-C₂ alkyl.

The invention also provides a compound of Formula (I) wherein X, N′, C′, A and E are as defined herein.

### DETAILED DESCRIPTION OF THE INVENTION

As used herein, a C₁-C₁₂ alkyl group is a linear or branched alkyl group containing from 1 to 12 carbon atoms. Sometimes, a C₁-C₁₂ alkyl group is a C₄-C₁₂ alkyl group or a C₅-C₁₂ alkyl group. Often, a C₁-C₁₂ alkyl group is a C₁-C₁₀ alkyl group. A C₁-C₁₀ alkyl group is often a C₁-C₈ alkyl group or a C₁-C₆ alkyl group. Examples of C₁-C₆ alkyl groups include methyl, ethyl, propyl, butyl, pentyl and hexyl. A C₁-C₆ alkyl group is often a C₁-C₄ alkyl group. Examples of C₁-C₄ alkyl groups include methyl, ethyl, n-propyl, iso-propyl, n-butyl, sec-butyl and tert-butyl. A C₁-C₄ alkyl group is often a C₁-C₃ alkyl group such as a C₁-C₂ alkyl group. A C₁-C₂ alkyl group is methyl or ethyl, typically methyl. For the avoidance of doubt, where two alkyl groups are present, the alkyl groups may be the same or different.

As used herein, a C₁-C₁₂ alkylene group is an unsubstituted or substituted bidentate moiety obtained by removing two hydrogen atoms from a C₁-C₁₂ alkyl group defined herein. The two hydrogen atoms may be removed from the same carbon atom or from different carbon atoms. Sometimes, a C₁-C₁₂ alkylene group is a C₄-C₁₂ alkylene group or a C₅-C₁₂ alkylene group. Examples of C₁-C₁₂ alkylene groups include C₁-C₁₀ alkylene groups, such as C₃-C₇ and C₄-C₆ alkylene groups. Examples of C₁-C₁₀ alkylene groups also include C₁-C₆ alkylene groups such as methylene, ethylene, propylene, butylene, pentylene and hexylene. A C₁-C₆ alkylene group is often a C₁-C₄ alkylene group. Examples of C₁-C₄ alkylene groups include methylene, ethylene, n-propylene, iso-propylene, n-butylene, sec-butylene and tert-butylene. A C₁-C₄ alkylene group is often a C₁-C₃ alkylene group such as a C₁-C₂ alkylene group. A C₁-C₂ alkyl group is methylene or ethylene, typically methylene. For the avoidance of doubt, where two alkylene groups are present, the alkylene groups may be the same or different.

As used herein, a C₂-C₁₂ alkenyl group is a linear or branched alkenyl group containing from 2 to 12 carbon atoms and having one or more, e.g. one or two, double bonds. Sometimes, a C₂-C₁₂ alkenyl group is a C₄-C₁₂ alkenyl group or a C₅-C₁₂ alkenyl group. Often, a C₂-C₁₂ alkenyl group is a C₂-C₁₀ alkenyl group. A C₂-C₁₀ alkenyl group is often a C₂-C₈ alkenyl group or a C₂-C₆ alkenyl group. Examples of C₂-C₆ alkenyl groups include ethenyl, propenyl, butenyl, pentenyl and hexenyl. A C₂-C₆ alkenyl group is often a C₂-C₄ alkenyl group. Examples of C₂-C₄ alkenyl groups include ethenyl, n-propenyl, iso-propenyl, n-butenyl, sec-butenyl and tert-butenyl. A C₂-C₄ alkenyl group is often a C₂-C₃ alkenyl group such as ethenyl. For the avoidance of doubt, where two alkenyl groups are present, the alkenyl groups may be the same or different.

As used herein, a C₂-C₆ alkynyl group or moiety can be linear or branched but is preferably linear. It contains one or more carbon-carbon triple bonds. It is preferably a C₂-C₄ alkynyl group, more preferably a C₂-C₃ alkynyl group. Suitable such alkynyl groups and moieties include ethynyl, propynyl, butynyl, pentynyl, and hexynyl and isomers thereof.

As used herein, a C₂-C₁₂ alkenylene group is an unsubstituted or substituted bidentate moiety obtained by removing two hydrogen atoms from a C₂-C₁₂ alkenyl group as defined herein. The two hydrogen atoms may be removed from the same carbon atom or from different carbon atoms. Sometimes, a C₂-C₁₂ alkenylene group is a C₄-C₁₂ alkenylene group or a C₅-C₁₂ alkenylene group. Examples of C₂-C₁₂ alkenylene groups include C₂-C₁₀ alkenylene groups, such as C₃-C₇ and C₄-C₆ alkenylene groups. Examples of C₂-C₁₀ alkenylene groups also include C₂-C₆ alkylene groups such as ethenylene, propenylene, butenylene, pentenylene and hexenylene. A C₂-C₆ alkenylene group is often a C₂-C₄ alkenylene group. Examples of C₂-C₄ alkenylene groups include ethenylene, n-propenylene, iso-propenylene, n-butenylene, sec-butenylene and tert-butenylene. A C₂-C₄ alkenylene group is often a C₂-C₃ alkenylene group such as ethenylene. For the avoidance of doubt, where two alkenylene groups are present, the alkenylene groups may be the same or different.

As used herein, a C₁-C₆ alkoxy group is typically a said C₁-C₆ alkyl group attached to an oxygen atom. Typically, a C₁-C₆ alkoxy group is a C₁-C₄ alkoxy group. Often, a C₁-C₄ alkoxy group is a C₁-C₃ alkoxy group. Examples of C₁-C₄ alkoxy groups include methoxy, ethoxy, propoxy and butoxy. Typically, a C₁-C₃ alkoxy group is a C₁-C₂ alkoxy group such as a methoxy or ethoxy group. For the avoidance of doubt, where two alkoxy groups are present, the alkoxy groups may be the same or different.

An alkyl, alkylene, alkenyl, alkynyl, alkenylene or alkoxy group as used herein may be unsubstituted or substituted. Substituted alkyl, alkylene, alkenyl, alkenylene or alkoxy groups typically carry one or more, e.g. one, two or three e.g. one, or two, e.g. one substituent selected from halogen, OH, and C₁-C₄ alkoxy. The substituents on a substituted alkyl, alkylene, alkenyl, alkenylene or alkoxy group are typically themselves unsubstituted.

As used herein, a halogen is typically chlorine, fluorine, bromine or iodine and is preferably chlorine, bromine or fluorine, for example chlorine or fluorine. Fluorine is preferred. For the avoidance of doubt, where two halogen atoms are present, the halogen atoms may be the same or different.

As used herein, a C₃-C₆ cycloalkyl group is a cyclic hydrocarbon containing from 3 to 6, e.g. 3, 4 or 5 carbon atoms. Unless otherwise stated, a cycloalkyl group is typically a C₃-C₆ cycloalkyl group. Examples of C₃-C₆ cycloalkyl group include cyclopropyl, cyclobutyl, cyclopentyl and cyclohexyl groups. In one aspect of the invention, a C₃-C₆ cycloalkyl group is a C₃-C₄ cycloalkyl group, i.e. cyclopropyl or cyclobutyl, in particular cyclopropyl. For the avoidance of doubt, where two cycloalkyl groups are present, the cycloalkyl groups may be the same or different. A preferred example of a cycloalkyl group is cyclohexyl. Another preferred example is cyclopropyl.

As used herein, a carbocyclyl moiety is a monocyclic or fused cyclic hydrocarbon. A carbocylyl moiety may be saturated and thus contain 0 double bonds, or may be partially unsaturated. Typically, a carbocylyl moiety is saturated. In one aspect, a carbocyclyl moiety is a 5- or 6-membered carbocylyl moiety. Often, a carbocyclyl moiety is fused to another ring such as a diazine ring and/or a group T (when T is cyclic) as defined herein. Examples of carbocyclyl moieties include cyclohexyl and cyclohexeneyl moieties.

As used herein, a heterocyclyl group is a monocyclic or fused cyclic group containing at least one heteroatom, and typically one or two heteroatoms. The heteroatom or heteroatoms are typically selected from —O—, N, and S. In one aspect, a heterocyclyl group is typically a 5- or 6-membered heterocyclyl group. Alternatively, the heterocyclyl group may be an 8- to 10-membered heterocyclyl group, for example a fused ring structure having a 5-6-membered heterocyclyl moiety fused to a phenyl ring. A heterocyclyl group as used herein includes groups having one or more, e.g. 1 or 2, ═O groups bound to the ring, e.g. isoindoline-1,3-dione. A heterocyclyl group may be saturated and thus contain 0 double bonds, or may be partially unsaturated. Typically, a heterocyclyl group is saturated. For the avoidance of doubt, where two heterocyclyl groups are present, the heterocyclyl groups may be the same or different. A preferred example of an 8- to 10-membered heterocyclyl group is isoindoline-1,3-dione. In one aspect, a heterocyclyl group is bonded to the remainder of the molecule by one bond. In another aspect a heterocyclyl group is a heterocyclyl moiety which is bonded to the remainder of the molecule by two or more, e.g. 2 bonds, or is fused to the remainder of the molecule.

Examples of 5- and 6-membered saturated heterocyclyl groups include thiolanyl, tetrahydrofuranyl, pyrrolidinyl, imidazolidinyl, pyrazolidinyl, oxazolidinyl, isoxazolidinyl, thiazolidinyl, isothiazolidinyl, 1,2-dioxolanyl, 1,3-dioxolanyl, 1,2-dithiolanyl, 1,3-dithiolanyl, piperidinyl, oxanyl, thianyl, piperazinyl, morpholinyl, thiomorpholinyl, dioxanyl, and dithianyl, for example, piperidinyl, piperazinyl, morpholinyl and thiomorpholinyl. A preferred example is piperidinyl.

Examples of 5- and 6-membered partially unsaturated heterocyclyl groups include dihydropyrrolyl, dihydrofuranyl, dihydrothiophenyl, dihydroimidazolyl, dihydropyrazolyl, dihydrooxazolyl, dihydroisoxazolyl, dihydrothiazolyl, dihydropyridinyl, dihydropyranyl, dihydrothiopyranyl, dihydrodiazinyl, dihydrooxazinyl, dihydrothiazinyl, dihydrodioxinyl, and dihydrodithiinyl, for example, dihydropyrrolyl, dihydrofuranyl and dihydrothiophenyl.

A cycloalkyl, carbocyclyl or heterocyclyl group may be unsubstituted or may be optionally substituted by 1, 2 or 3, typically 1 or 2, e.g. by 1 substituent selected from selected from halogen, OH, C₁-C₄ alkyl, C₁-C₄ alkoxy. Alkyl substituents on a cycloalkyl, carbocyclyl or heterocyclyl group may themselves be substituted, for example with 1, 2 or 3 substituents independently selected from halogen and OH. Substituents on a cycloalkyl, carbocyclyl or heterocyclyl group are typically themselves unsubstituted. Where stated, a cycloalkyl or heterocyclyl group is unsubstituted or substituted by 1, 2 or 3 groups V, wherein a group V is as defined herein.

As used herein, an aryl group is a substituted or unsubstituted, monocyclic or fused polycyclic aromatic group. Examples of aryl groups include C₆-C₁₀ aryl groups which contain from 6 to 10 carbon atoms in the ring portion. Examples include phenyl (i.e. monocyclic), naphthyl, indenyl and indanyl (i.e. fused bicyclic) groups. Phenyl and naphthyl, in particular phenyl, are preferred. For the avoidance of doubt, where two aryl groups are present, the aryl groups may be the same or different.

As used herein, a heteroaryl group is a substituted or unsubstituted aromatic group. A heteroaryl group includes at least one heteroatom, for example 1, 2 or 3 heteroatoms, typically selected from —O—, S and N. Examples include 5- to 6-membered heteroaryl groups which contain from 5 to 6 atoms in the ring portion. Alternatively, the heteroaryl group may be an 8- to 10-membered heteroaryl group, for example a fused ring structure having a 5-6-membered heteroaryl moiety fused to a phenyl ring. For the avoidance of doubt, where two heteroaryl moieties are present, the heteroaryl moieties may be the same or different.

Examples of 5- and 6-membered heteroaryl groups include pyrrolyl, furanyl, thienyl, thiophenyl, imidazolyl, pyrazolyl, oxazolyl, thiazolyl, isoxazolyl, pyridinyl, pyrazinyl, pyrimidinyl, pyranyl, thiopyranyl, diazinyl, oxazinyl, thiazinyl, dioxinyl, and dithiinyl. Preferred examples include pyridyl, pyrimidinyl and pyrazinyl. Benzofuranyl is an example of an 8- to 10-membered heteroaryl group. Benzothiazole is a further example of an 8- to 10-membered heteroaryl group.

An aryl or heteroaryl group may be unsubstituted or substituted with 1, 2 or 3, typically 1 or 2 such as e.g. 1 substituent. Suitable substituents include, for example, halogen, OH, and C₁-C₄ alkoxy. Where stated, an aryl or heteroaryl group is unsubstituted or substituted by 1, 2 or 3 groups V, wherein a group V is as defined herein.

As used herein, an aryloxy group is typically a said aryl group attached to an oxygen atom. Typically, an aryloxy group is a C₆-C₁₀ aryloxy group such as phenoxy (—O-Ph) or napthyloxy (—O-napthyl). Phenoxy is preferred. For the avoidance of doubt, when two aryloxy moieties are present, the aryloxy moieties may be the same or different.

As used herein, a salt is typically a pharmaceutically acceptable salt. As used herein a pharmaceutically acceptable salt is a salt with a pharmaceutically acceptable acid or base. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulphate, bisulphate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulphonate, ethanesulphonate, benzenesulphonate, p-toluenesulphonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts, for example, sodium salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

In Formula (I), the stereochemistry is not limited. In particular, compounds of formula (I) containing one or more chiral centre may be used in enantiomerically or diastereoisomerically pure form, or in the form of a mixture of isomers. Further, for the avoidance of doubt, the compounds as described herein may be used in any tautomeric form. Typically, the agent or substance described herein contains at least 50%, preferably at least 60, 75%, 90% or 95% of a compound according to Formula (I) which is enantiomerically or diasteriomerically pure. Thus, the compound is preferably substantially optically pure.

In the compound of formula (I), X typically represents S.

In the compounds of Formula (I), the moiety

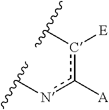

represents —N(D)-C(A)═C(E)- or —N═C(A)-C(R¹)(E)-. (For the avoidance of doubt, C is carbon, and N is nitrogen). Thus, the compound of Formula (I) is of Formula (Ia) [when the moiety is —N═C(A)-C(R¹)(E)-] or Formula (Ib) [when the moiety is —N(D)-C(A)═C(E)-].

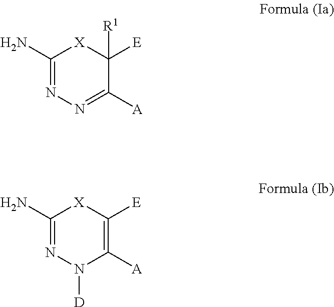

Typically, the compound is of Formula (Ia). R¹ typically represents H or C₁-C₂ alkyl; more typically R¹ represents H or methyl, most often R¹ is H. Thus, most often, the compound is of Formula (Ic).

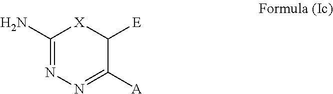

D, when present, is typically H or C₁-C₆ alkyl, wherein the alkyl group is unsubstituted or is substituted with 1 or 2 substituents selected from halogen, OH and C₁-C₂ alkoxy. Often, the alkyl group of D is a C₁-C₄ alkyl group, such as C₁-C₂ alkyl group e.g. a methyl group. Often, the alkyl group of D is unsubstituted or is substituted with 1 substitutent selected from C₁-C₂ alkoxy, and most typically the alkyl group of D is unsubstituted. Thus, usually D is H or a C₁-C₄ alkyl group which is unsubstituted or is substituted with 1 substitutent selected from C₁-C₂ alkoxy, and more usually D is H or unsubstituted methyl. Most often, D is H.

In Formula (I), one group selected from A and E represents a group Q1, and the other group selected from A and E represents a group Q2.

Q1 Represents

(i) H or C₁-C₈ alkyl, wherein the alkyl group of Q1 is unsubstituted or is substituted with 1, 2 or 3 substituents selected from halogen, OH, and C₁-C₂ alkoxy; and wherein the alkyl group of Q1 is uninterrupted or is interrupted by —O—, —C(O)—, —OC(O)— or —C(O)O—;

or

(ii) an alkylene group which is bonded to an atom of group Q2 to form a C₅-C₆ carbocyclyl or 5- to 6-membered heterocyclyl moiety, wherein the carbocylyl or heterocyclyl moiety is saturated or partially unsaturated; and wherein the carbocyclyel or heterocyclyl moiety is unsubstituted or is substituted with 1, 2 or 3 substituents selected from halogen, C₁-C₄ alkoxy, unsubstituted C₁-C₄ alkyl and C₁-C₄ alkyl substituted with 1, 2 or 3 substituents independently selected from halogen and —OH.

When Q1 is according to option (i), Q1 is often H or C₁-C₆ alkyl which is unsubstituted or is substituted with 1 substituent selected from halogen and C₁-C₂ alkoxy; and wherein the alkyl group of Q1 is uninterrupted or is interrupted by —O—. More often, when Q1 is according to option (i), Q1 is H or C₂-C₆ alkyl which is unsubstituted and which is uninterrupted or is interrupted by —O—; still more often, Q1 is H or C₄-C₆ alkyl which is unsubstituted and which is uninterrupted or is interrupted by —O—; for example, Q1 is most often H or unsubstituted C₆ alkyl which is interrupted by —O—. Most often, when Q1 is according to option (i), Q1 is H.

When Q1 is according to option (ii), Q1 is usually a C₁-C₃ alkylene group which is bonded to an atom of group A to form, together with the diazine ring atoms to which they are attached, a C₅-C₆ carbocyclyl moiety. More usually, when Q1 is according to option (ii), Q1 is a C₁-C₂ alkylene group, e.g. methylene or ethylene. Typically, Q1 is bonded to A at the terminal atom on the alkylene chain of Q1. Q1 may be bonded to any available atom of A, which may be the terminal atom of A, or an atom within the group A.

The alkylene group of Q1 may be bonded to an atom on a cyclic group of Q2, such that the carbocyclyl or heterocyclyl, preferably carbocyclyl, moiety which is formed is fused to the cyclic group of Q2. Often, the carbocyclyl moiety which is formed is unsubstituted or is substituted by 1 or 2 substituents selected from halogen, C₁-C₄ alkoxy and unsubstituted C₁-C₄ alkyl, such as by 1 substitutent selected from C₁-C₄ alkoxy, e.g. C₁-C₂ or C₃-C₄ alkoxy, e.g. C₄ alkoxy.

Examples of the carbocyclyl moiety formed by Q1 and Q2 together with the diazine ring atoms to which they are attached include cyclohexyl and cyclohexenyl according to Formula (II).

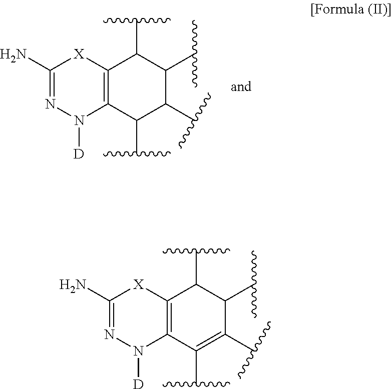

Thus, examples of the moieties formed according to option (ii) of Q1 include the moieties shown in Formula (IIa)

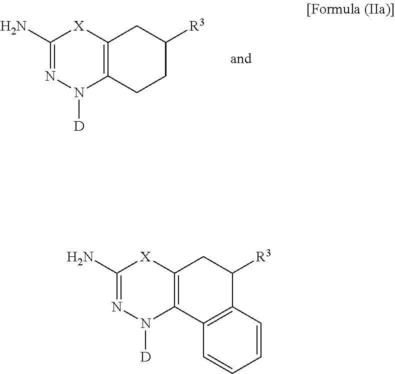

In Formula (IIa), R³ is selected from, for example, H and C₁-C₄ alkoxy.

Most often, Q1 is according to option (i).

In Formula (I), Q2 represents the group -L-T or -T. In one embodiment, Q2 represents -L-T. In another embodiment, Q2 represents T.

L is typically selected from C₁-C₁₀ alkylene and C₂-C₁₀ alkenylene. L is often C₃-C₇ alkylene such as C₄-C₆ alkylene e.g. C₅ alkylene. L may be C₆-C₈ alkylene such as C₇ alkylene. L is sometimes C₃-C₇ alkenylene such as C₄-C₆ alkylene e.g. C₅ alkylene. Othertimes, L is C₆-C₈ alkenylene such as C₇ alkenylene. Thus, often, L is selected from C₃-C₇ alkylene and C₃-C₇ alkenylene; still more typically L is selected from C₄-C₆ alkylene and C₄-C₆ alkenylene or L is selected from C₆-C₈ alkylene and C₆-C₈ alkenylene. L is typically alkylene. Thus, preferably, L is C₁-C₁₀ alkylene such as C-C₆ alkylene e.g. C₅ alkylene. L is typically unsubstituted or is substituted by 1 or 2 groups selected from halogen and C₁-C₄ alkoxy, e.g. chlorine, bromine or fluorine, or methoxy or ethoxy. Most typically, L is unsubstituted.

L is typically uninterrupted or is interrupted by a heteromoiety selected from —O—, —S—, —C(O)—, —OC(O)—, —C(O)O—, —NR²—, —NR²C(O)—, and —C(O)NR²—; more often by a heteromoiety selected from —O—, —S—, —C(O)O—, —NR²—, and —C(O)NR²—, still more often by a heteromoiety selected from —O—, —S—, and —C(O)O—, such as by —O— or by —C(O)O—, e.g. by —O For the avoidance of doubt, the left hand end of the heteromoiety as depicted is the end closest to the diazine ring of Formula (I).

L may terminate in a heteromoiety selected from —O—, —S—, —C(O)—, —OC(O)—, —C(O)O—, —NR²—, —NR²C(O)—, and —C(O)NR²—; more often by a heteromoiety selected from —O—, —S—, —C(O)O—, —NR²—, and —C(O)NR²—, still more often by a heteromoiety selected from —O—, —S—, and —C(O)O—, such as by —O— or by —C(O)O—, e.g. by —O—. R² is typically H or methyl, most typically R² is H. Typically, when T is H, L does not terminate in a heteromoiety. For the avoidance of doubt, the left hand end of the heteromoiety as depicted is the end closest to the diazine ring of formula (I).

L may be interrupted by a heteromoiety and/or terminate in a heteromoiety as described herein. More often, L is interrupted by a heteromoiety as described herein.

Typically, when Q2 is -L-T, T is H or represents aryl or heteroaryl. More often, T is H or represents aryl. When Q2 is -T, T is often aryl or heteroaryl, more often aryl. When T is heteroaryl, T may often be a 5- to 10-membered heteroaryl group. For example, T may be a 6-membered heteroaryl group, or a 9-membered heteroaryl group. For example, T may be selected from the group consisting of benzofuranyl and pyridyl. When T is aryl, T may often be a 6- to 10-membered aryl group. For example, T may be a 6-membered aryl group such as phenyl or a 10-membered aryl group such as napthyl. Most often, T is phenyl. When T is heterocyclyl, it may be isoindoline-1,3-dione. When T is cycloalkyl, it may be C₅- or C₆-cycloalkyl such as cyclohexyl.

In one emobodiment, T cannot be benzofuran, napthofuran or 2H-chromen-2-one. For example, when Q2 is -T, then T is aryl or heteroaryl, on the proviso that T is not benzofuran, napthofuran or 2H-chromen-2-one. As a further example, when T is heteroaryl, T may often be a 5- to 10-membered heteroaryl group other than benzofuran or 2H-chromen-2-one. For example, T may be a 5-membered heteroaryl group, 6-membered heteroaryl group, or a 9-membered heteroaryl group other than benzofuran. For example, T may be pyridyl, thienyl, tetrahydrofuran or benzothiazole. Preferably, T is a 5-membered heteroaryl group or 6-membered heteroaryl group. For example, T may be pyridyl, thienyl or tetrahydrofuran.

T is often unsubstituted or is substituted by 1 or 2 groups V. More often, T is unsubstituted or is substituted by 1 group V. In another aspect, T is substituted by 1 or 2 groups V. Most often T is substituted by 1 group V.

In one embodiment, each group V is independently selected from C₁-C₆ alkoxy, unsubstituted C₁-C₁₀ alkyl, C₁-C₁₀ alkyl which is substituted with 1, 2 or 3 groups selected from halogen and C₁-C₃ alkoxy, C₃-C₆ cycloalkyl, halogen, aryl, (C₁-C₆ alkyl)-aryl, aryloxy, aryloxy-(C₁-C₆ alkyl), —CN, and —C(O)O(C₁-C₆ alkyl).

Usually, each group V is independently selected from C₁-C₄ alkoxy, unsubstituted C₁-C₄ alkyl, C₁-C₄ alkyl which is substituted with 1, 2 or 3 groups selected from halogen, C₂-C₄ alkenyl, C₂-C₄ alkynyl, C₃-C₆ cycloalkyl, halogen, aryl, (C₁-C₆ alkyl)-aryl, aryloxy, aryloxy-(C₁-C₆ alkyl), —CN, NO₂, —(C₁-C₄ alkyl)—C(O)O(C₁-C₄ alkyl) and —C(O)O(C₁-C₄ alkyl).

Typically, each group V is independently selected from C₁-C₄ alkoxy, unsubstituted C₁-C₄ alkyl, C₁-C₄ alkyl which is substituted with 1, 2 or 3 halogen atoms, C₃-C₆ cycloalkyl, halogen, aryl, (C₁-C₄ alkyl)-aryl, aryloxy, aryloxy-(C₁-C₄ alkyl), —CN, and —C(O)O(C₁-C₄ alkyl). Most often each V is selected from unsubstituted C₁-C₃ alkyl, halogen, —C(O)O(C₁-C₃ alkyl), (C₁-C₃ alkyl)-aryl and aryloxy. Usually, when V comprises an aryl moiety, the aryl moiety is phenyl.

Usually, A represents Q2 and E represents Q1. Thus, often, the compound for use as an antifungal agent is a compound of Formula (I) wherein:
X represents O or S;
the moiety

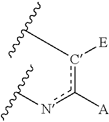

represents —N(D)-C(A)═C(E)- or —N═C(A)-C(R¹)(E)-;
D represents H or C₁-C₆ alkyl, wherein the alkyl group of D is unsubstituted or is substituted with 1, 2 or 3 substituents selected from halogen, OH, and C₁-C₂ alkoxy; and wherein the alkyl group of D is uninterrupted or is interrupted by —O—, —C(O)—, —OC(O)— or —C(O)O—;
R¹ is H or C₁-C₂ alkyl;
E represents
   (i) H or C₁-C₈ alkyl, wherein the alkyl group of E is as defined for Q1 herein; or
   (ii) an alkylene group which is bonded to an atom of group A to form a C₅-C₆ carbocyclyl or 5- to 6-membered heterocyclyl moiety as defined for Q1 herein;
and
A represents a group -L-T or -T, wherein L and T are as defined for Q2 herein.

Therefore, for example, in one embodiment, the compound for use as an antifungal agent is a compound of formula (Ia)

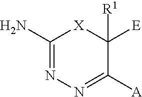

wherein:
X represents S;
R¹ represents H or C₁-C₂ alkyl;
E represents either:
(i) H or C₁-C₆ alkyl which is unsubstituted or is substituted with 1 substituent selected from halogen, and C₁-C₂ alkoxy; and wherein the alkyl group of E is uninterrupted or is interrupted by —O—; or
(ii) a C₁-C₆ alkylene group which is bonded to an atom of group A to form a C₅-C₆ carbocyclyl moiety which is unsubstituted or is substituted by 1 or 2 substituents selected from halogen, C₁-C₄ alkoxy and unsubstituted C₁-C₄ alkyl;
A represents the group -L-T or -T;
L is selected from C₁-C₁₀ alkylene and C₂-C₁₀ alkenylene, wherein the alkylene or alkenylene group is unsubstituted or is substituted by 1 or 2 groups selected from halogen, and C₁-C₄ alkoxy, and wherein the alkylene or alkenylene group is uninterrupted or is interrupted by a heteromoiety selected from —O—, —S—, —C(O)O—, —NR²—, and —C(O)NR²— and wherein the alkylene or alkenylene group optionally terminates in a heteromoiety selected from —O—, —S—, —C(O)—, —OC(O)—, —C(O)O—, —NR²—, —NR²C(O)—, and —C(O)NR²—, wherein R² represents H or methyl;
when A is -L-T, then T is H or represents a 5- to 10-membered heteroaryl group or a 6- to 10-membered aryl group, and when A is -T, then T represents a 5- to 10-membered heteroaryl group or a 6- to 10-membered aryl group; wherein the aryl or heteroaryl group of T is unsubstituted or is substituted by 1 or 2 groups V;
Each group V is independently selected from C₁-C₄ alkoxy, unsubstituted C₁-C₄ alkyl, C₁-C₄ alkyl which is substituted with 1, 2 or 3 halogen atoms, C₃-C₆ cycloalkyl, halogen, aryl, (C₁-C₄ alkyl)-aryl, aryloxy, aryloxy-(C₁-C₄ alkyl), —CN, and —C(O)O(C₁-C₄ alkyl).

In a further example, the compound for use as an antifungal agent is a compound of Formula (Ia):

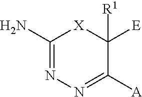

wherein:
X represents S;
R¹ represents H or C₁-C₂ alkyl;
E represents either:
(i) H or C₁-C₆ alkyl which is unsubstituted or is substituted with 1 substituent selected from halogen, and C₁-C₂ alkoxy; and wherein the alkyl group of E is uninterrupted or is interrupted by −O—; or
(ii) a C₁-C₆ alkylene group which is bonded to an atom of group A to form a C₅-C₆ carbocyclyl moiety which is unsubstituted or is substituted by 1 or 2 substituents selected from halogen, C₁-C₄ alkoxy and unsubstituted C₁-C₄ alkyl;
A represents the group -L-T or -T;
L is selected from C₁-C₁₀ alkylene and C₂-C₁₀ alkenylene, wherein the alkylene or alkenylene group is unsubstituted or is substituted by 1 or 2 groups selected from halogen, and C₁-C₄ alkoxy, and wherein the alkylene or alkenylene group is uninterrupted or is interrupted by a heteromoiety selected from —O—, —S—, —C(O)O—, —NR²—, and —C(O)NR²— and wherein the alkylene or alkenylene group optionally terminates in a heteromoiety selected from —O—, —S—, —C(O)—, —OC(O)—, —C(O)O—, —NR²—, —NR²C(O)—, and —C(O)NR²—, wherein R² represents H or methyl;
when A is -L-T, then T is H or represents a 5- to 10-membered heteroaryl group or a 6- to 10-membered aryl group, and when A is -T, then T represents a 5- to 10-membered heteroaryl group or a 6- to 10-membered aryl group; wherein the aryl or heteroaryl group of T is unsubstituted or is substituted by 1 or 2 groups V;
each group V is independently selected from C₁-C₄ alkoxy, unsubstituted C₁-C₄ alkyl, C₁-C₄ alkyl which is substituted with 1, 2 or 3 halogen atoms, C₂-C₄ alkenyl, C₂-C₄ alkynyl, C₃-C₆ cycloalkyl, halogen, aryl, (C₁-C₄ alkyl)-aryl, aryloxy, aryloxy-(C₁-C₄ alkyl), —CN, NO₂, —(C₁-C₄ alkyl)-C(O)O(C₁-C₄ alkyl), and —C(O)O(C₁-C₄ alkyl).

In a further emobodiment, the compound for use as an antifungal agent is a compound of Formula (Ia):

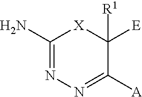

wherein
X represents S;
R¹ represents H or methyl
E represents either:
(i) H or C₄-C₆ alkyl which is unsubstituted and which is uninterrupted or is interrupted by —O—; or
(ii) E is a C₁-C₄ alkylene group which is bonded to an atom of group A to form a C₆ carbocyclylene group which is unsubstituted or is substituted by 1 substitutent selected from C₁-C₄ alkoxy
A represents the group -L-T or -T;
L is selected from C₁-C₁₀ alkylene and C₂-C₁₀ alkenylene, wherein the alkylene or alkenylene group is unsubstituted or is substituted by 1 group selected from halogen and C₁-C₄ alkoxy, and wherein the alkylene or alkenylene group is uninterrupted or is interrupted by a heteromoiety selected from —O—, —S—, and —C(O)O—, and wherein the alkylene or alkenylene group optionally terminates in a heteromoiety selected from —O—, and —C(O)O—;
when A is -L-T, then T is H or represents phenyl, napthyl, benzofuranyl, pyridyl, isoindoline-1,3-dione, benzothiazole, tetrahydrofuran, thienyl or cyclohexyl, and when A is -T, then T represents phenyl, napthyl, benzofuranyl, pyridyl, isoindoline-1,3-dione, benzothiazole, tetrahydrofuran, thienyl or cyclohexyl; wherein T is unsubstituted or is substituted by 1 V group;
each V group is independently selected from C₁-C₄ alkoxy, unsubstituted C₁-C₄ alkyl, C₁-C₄ alkyl which is substituted with 1, 2 or 3 halogen atoms, C₂-C₄ alkenyl, C₂-C₄ alkynyl, C₃-C₆ cycloalkyl, halogen, aryl, (C₁-C₄ alkyl)-aryl, aryloxy, aryloxy-(C₁-C₄ alkyl), —CN, NO₂, —(C₁-C₄ alkyl)-C(O)O(C₁-C₄ alkyl), and —C(O)O(C₁-C₄ alkyl).

In a preferred embodiment, the compound for use as an antifungal agent is a compound of formula (Ia) wherein:
X represents S;
R¹ represents H or methyl
E represents either:
(i) H or C₄-C₆ alkyl which is unsubstituted and which is uninterrupted or is interrupted by —O—; or
(ii) E is a C₁-C₄ alkylene group which is bonded to an atom of group A to form a C₆ carbocyclylene group which is unsubstituted or is substituted by 1 substitutent selected from C₁-C₄ alkoxy
A represents the group -L-T or -T;
L is selected from C₁-C₁₀ alkylene and C₂-C₁₀ alkenylene, wherein the alkylene or alkenylene group is unsubstituted or is substituted by 1 group selected from halogen and C₁-C₄ alkoxy, and wherein the alkylene or alkenylene group is uninterrupted or is interrupted by a heteromoiety selected from —O—, —S—, and —C(O)O—, and wherein the alkylene or alkenylene group optionally terminates in a heteromoiety selected from —O—, and —C(O)O—;
when A is L-T, then T is H or represents phenyl, napthyl, benzofuranyl, pyridyl, isoindoline-1,3-dione or cyclohexyl, and when A is T, then T represents phenyl, napthyl, benzofuranyl, pyridyl, isoindoline-1,3-dione or cyclohexyl; wherein T is unsubstituted or is substituted by 1 V group;
each V group is independently selected from C₁-C₄ alkoxy, unsubstituted C₁-C₄ alkyl, C₁-C₄ alkyl which is substituted with 1, 2 or 3 halogen atoms, C₃-C₆ cycloalkyl, halogen, aryl, (C₁-C₄ alkyl)-aryl, aryloxy, aryloxy-(C₁-C₄ alkyl), —CN, and —C(O)O(C₁-C₄ alkyl).

In a more preferred embodiment, the compound for use as an antifungal agent is a compound of formula (Ia) wherein:
X represents S;
R¹ represents H
E represents H or C₄-C₆ alkyl which is unsubstituted and which is uninterrupted or is interrupted by —O—;
A represents a group -L-T or -T;
L is selected from C₁-C₁₀ alkylene and C₂-C₁₀ alkenylene, wherein the alkylene or alkenylene group is unsubstituted, and wherein the alkylene or alkenylene group is uninterrupted or is interrupted by a heteromoiety selected from —O— and —C(O)O—, and wherein the alkylene or alkenylene group optionally terminates in —O—;
when A is L-T, then T is H or represents phenyl, and when A is T, then T is phenyl; wherein the phenyl group is unsubstituted or is substituted by 1 group V;
each V is independently unsubstituted C₁-C₃ alkyl, halogen, —C(O)O(C₁-C₃ alkyl), (C₁-C₃ alkyl)-aryl and aryloxy.

The compound for use as an anti-fungal agent may be selected from: 5-(5-pentoxypentyl)-6˜{H}-1,3,4-thiadiazin-2-amine; 5-[3-(2-phenylethoxy)propyl]-6˜{H}-1,3,4-thiadiazin-2-amine; 5-[4-(4-ethylphenoxy)butyl]-6˜{H}-1,3,4-thiadiazin-2-amine; 5-[5-(2-pyridyloxy)pentyl]-6˜{H}-1,3,4-thiadiazin-2-amine; 5-[2-(2-phenoxyethoxy)ethyl]-6˜{H}-1,3,4-thiadiazin-2-amine; 5-[4-[[2-(2-phenylethyl)phenyl]methoxy]butyl]-6˜{H}-1,3,4-thiadiazin-2-amine; 5-[4-[(3,5-dichlorophenyl)methoxy]butyl]-6˜{H}-1,3,4-thiadiazin-2-amine; 5-[4-[(4-chloro-2-fluoro-phenyl)methoxy]butyl]-6˜{H}-1,3,4-thiadiazin-2-amine; 5-[4-[(2-cyclopropylphenyl)methoxy]butyl]-6˜{H}-1,3,4-thiadiazin-2-amine; 5-[4-[[2-(trifluoromethyl)phenyl]methoxy]butyl]-6˜{H}-1,3,4-thiadiazin-2-amine; 5-[4-[(2,5-difluorophenyl)methoxy]butyl]-6˜{H}-1,3,4-thiadiazin-2-amine; 5-[4-[(2-chloro-6-fluoro-phenyl)methoxy]butyl]-6˜{H}-1,3,4-thiadiazin-2-amine; 5-[4-[(3-butylphenyl)methoxy]butyl]-6˜{H}-1,3,4-thiadiazin-2-amine; 5-[4-[(4-bromophenyl)methoxy]butyl]-6˜{H}-1,3,4-thiadiazin-2-amine; 5-[4-[(4-chlorophenyl)methylsulfanyl]butyl]-6˜{H}-1,3,4-thiadiazin-2-amine; 5-[4-(2-phenylethoxy)butyl]-6˜{H}-1,3,4-thiadiazin-2-amine; 4-(2-amino-6˜{H}-1,3,4-thiadiazin-5-yl)butyl acetate; 5-[4-[(3-ethylphenyl)methoxy]butyl]-6˜{H}-1,3,4-thiadiazin-2-amine; 5-[4-[[3-(2-phenylethyl)phenyl]methoxy]butyl]-6˜{H}-1,3,4-thiadiazin-2-amine; 5-[4-[3-(2,4-difluorophenyl)propoxy]butyl]-6˜{H}-1,3,4-thiadiazin-2-amine; 5-[4-[(3-phenylphenyl)methoxy]butyl]-6˜{H}-1,3,4-thiadiazin-2-amine; 5-[4-[[3-(trifluoromethyl)phenyl]methoxy]butyl]-6˜{H}-1,3,4-thiadiazin-2-amine; 5-[4-[(4-chloro-3-fluoro-phenyl)methoxy]butyl]-6˜{H}-1,3,4-thiadiazin-2-amine; 5-[4-[(3,5-difluorophenyl)methoxy]butyl]-6˜{H}-1,3,4-thiadiazin-2-amine; 5-[4-[(3-phenoxyphenyl)methoxy]butyl]-6˜{H}-1,3,4-thiadiazin-2-amine; 5-[4-[(2,4-dichlorophenyl)methoxy]butyl]-6˜{H}-1,3,4-thiadiazin-2-amine; 5-[4-[(3-bromophenyl)methoxy]butyl]-6˜{H}-1,3,4-thiadiazin-2-amine; 5-[4-[(3-cyclopropylphenyl)methoxy]butyl]-6˜{H}-1,3,4-thiadiazin-2-amine; 5-[4-[(4-fluoro-3-methyl-phenyl)methoxy]butyl]-6˜{H}-1,3,4-thiadiazin-2-amine; 5-[4-[(4-fluorophenyl)methoxy]butyl]-6˜{H}-1,3,4-thiadiazin-2-amine; 5-[4-[(4-methoxyphenyl)methoxy]butyl]-6˜{H}-1,3,4-thiadiazin-2-amine; 5-[4-[(2,4-difluorophenyl)methoxy]butyl]-6˜{H}-1,3,4-thiadiazin-2-amine; 5-[4-[(3-chlorophenyl)methoxy]butyl]-6˜{H}-1,3,4-thiadiazin-2-amine; 5-[4-(m-tolylmethoxy)butyl]-6˜{H}-1,3,4-thiadiazin-2-amine; 5-[4-[(4-butylphenyl)methoxy]butyl]-6˜{H}-1,3,4-thiadiazin-2-amine; 5-[4-[[4-(trifluoromethyl)phenyl]methoxy]butyl]-6˜{H}-1,3,4-thiadiazin-2-amine; 5-[4-[(4-chlorophenyl)methoxy]butyl]-6˜{H}-1,3,4-thiadiazin-2-amine; 5-[5-(4-butylphenoxy)pentyl]-6˜{H}-1,3,4-thiadiazin-2-amine; 5-[2-[(4-butylphenyl)methoxy]ethyl]-6˜{H}-1,3,4-thiadiazin-2-amine; 5-[2-[3-(4-methoxyphenyl)propoxy]ethyl]-6˜{H}-1,3,4-thiadiazin-2-amine; 5-[5-(4-ethylphenoxy)pentyl]-6˜{H}-1,3,4-thiadiazin-2-amine; 5-[2-(4-methoxybutoxy)ethyl]-6˜{H}-1,3,4-thiadiazin-2-amine; 5-[2-(3-phenylpropoxy)ethyl]-6˜{H}-1,3,4-thiadiazin-2-amine; butyl 4-(2-amino-6˜{H}-1,3,4-thiadiazin-5-yl)benzoate; 5-[5-(3-ethylphenoxy)pentyl]-6˜{H}-1,3,4-thiadiazin-2-amine; 5-(4-butoxyphenyl)-4-methyl-1,3,4-thiadiazin-2-amine; 5-(4-benzyloxybutyl)-6˜{H}-1,3,4-thiadiazin-2-amine; 5-(4-methoxyphenyl)-4-methyl-1,3,4-thiadiazin-2-amine; 5-(benzofuran-2-yl)-4-methyl-1,3,4-thiadiazin-2-amine; 4-(2-amino-4-methyl-1,3,4-thiadiazin-5-yl)benzonitrile; ethyl 4-(2-amino-4-methyl-1,3,4-thiadiazin-5-yl)benzoate; 5-(3,4-dichlorophenyl)-4-methyl-1,3,4-thiadiazin-2-amine; 4-methyl-5-(p-tolyl)-1,3,4-thiadiazin-2-amine; 5-[2-(4-pentoxyphenyl)ethyl]-6˜{H}-1,3,4-thiadiazin-2-amine; 5-(5-benzyloxypentyl)-6˜{H}-1,3,4-thiadiazin-2-amine; 5-[4-(benzofuran-2-yl)butyl]-6˜{H}-1,3,4-thiadiazin-2-amine; 5-(5-methoxypentyl)-4-methyl-3˜{H}-1,3,4-thiadiazin-2-imine; 5-(4-chlorophenyl)-4-methyl-1,3,4-thiadiazin-2-amine; 2-[5-(2-amino-6˜{H}-1,3,4-thiadiazin-5-yl)pentyl]isoindoline-1,3-dione; 5-[5-(4-fluorophenoxy)pentyl]-6˜{H}-1,3,4-thiadiazin-2-amine; 5-[(˜{E})-hept-1-enyl]-6˜{H}-1,3,4-thiadiazin-2-amine; 4-(5-methoxypentyl)-5-methyl-1,3,4-thiadiazin-2-amine; 5-[5-(2-pyridyl)pentyl]-6˜{H}-1,3,4-thiadiazin-2-amine; 5-[5-(˜{N}-methylanilino)pentyl]-6˜{H}-1,3,4-thiadiazin-2-amine; 5-(6-methoxyhexyl)-6˜{H}-1,3,4-thiadiazin-2-amine; 5-(7-phenoxyheptyl)-6˜{H}-1,3,4-thiadiazin-2-amine; 5-(7-methoxyheptyl)-6˜{H}-1,3,4-thiadiazin-2-amine; 5-(4-methoxybutyl)-6˜{H}-1,3,4-thiadiazin-2-amine; 6-(2-amino-6˜{H}-1,3,4-thiadiazin-5-yl)hexanoic acid; ethyl 6-(2-amino-6˜{H}-1,3,4-thiadiazin-5-yl)hexanoate; 5-(methoxymethyl)-6˜{H}-1,3,4-thiadiazin-2-amine; 5-(4-phenoxybutyl)-6˜{H}-1,3,4-thiadiazin-2-amine; 5-[2-(4-phenylphenoxy)ethyl]-6˜{H}-1,3,4-thiadiazin-2-amine; 5-[2-(4-methylphenoxy)ethyl]-6˜{H}-1,3,4-thiadiazin-2-amine; 5-(phenoxymethyl)-6˜{H}-1,3,4-thiadiazin-2-amine; 5-(3-phenoxypropyl)-6˜{H}-1,3,4-thiadiazin-2-amine; 5-(6-phenoxyhexyl)-6˜{H}-1,3,4-thiadiazin-2-amine; 5-(6-phenylhexyl)-6˜{H}-1,3,4-thiadiazin-2-amine; 5-(2-amino-6˜{H}-1,3,4-thiadiazin-5-yl)-˜{N}-phenyl-pentanamide; 5-[2-[(4-methoxyphenyl)methoxy]ethyl]-6˜{H}-1,3,4-thiadiazin-2-amine; 5-[2-(4-propoxyphenyl)ethyl]-6˜{H}-1,3,4-thiadiazin-2-amine; 4-[2-(2-amino-6˜{H}-1,3,4-thiadiazin-5-yl)ethoxymethyl]benzonitrile; 5-[2-(1-methylbutoxy)ethyl]-6˜{H}-1,3,4-thiadiazin-2-amine; 5-[2-(p-tolylmethoxy)ethyl]-6˜{H}-1,3,4-thiadiazin-2-amine; 5-[2-[(4-phenylphenyl)methoxy]ethyl]-6˜{H}-1,3,4-thiadiazin-2-amine; 5-(2-hexoxyethyl)-6˜{H}-1,3,4-thiadiazin-2-amine; 5-[2-(2-methoxyethoxy)ethyl]-6˜{H}-1,3,4-thiadiazin-2-amine; 5-(2-phenoxyethyl)-6˜{H}-1,3,4-thiadiazin-2-amine; 5-[2-(4-bromophenoxy)ethyl]-6˜{H}-1,3,4-thiadiazin-2-amine; 5-(2-naphthyl)-6˜{H}-1,3,4-thiadiazin-2-amine; 5-(1-naphthyl)-4˜{H}-1,3,4-thiadiazin-2-amine; 5-(4-butoxyphenyl)-6˜{H}-1,3,4-thiadiazin-2-amine; 5-(3-butoxyphenyl)-6˜{H}-1,3,4-thiadiazin-2-amine; 5-(3,4-dichlorophenyl)-6˜{H}-1,3,4-thiadiazin-2-amine; ethyl 3-(2-amino-6˜{H}-1,3,4-thiadiazin-5-yl)benzoate; 5-(5-methoxypentyl)-6˜{H}-1,3,4-thiadiazin-2-amine; 5-(3-methoxypropyl)-6˜{H}-1,3,4-thiadiazin-2-amine; 5-(3,5-dimethylphenyl)-6˜{H}-1,3,4-thiadiazin-2-amine; ethyl 4-(2-amino-4˜{H}-1,3,4-thiadiazin-5-yl)benzoate; 5-heptyl-4˜{H}-1,3,4-thiadiazin-2-amine; isopropyl 2-(2-amino-6˜{H}-1,3,4-thiadiazin-5-yl)acetate; 5,6-dihydro-4˜{a}˜{H}-benzo[h][4,1,2]benzothiadiazin-3-amine; 5,6,7,8-tetrahydro-1˜{H}-4,1,2-benzothiadiazin-3-amine; 5-(5-phenoxypentyl)-6˜{H}-1,3,4-thiadiazin-2-amine; 5-(4-phenylbutyl)-6˜{H}-1,3,4-thiadiazin-2-amine; 6-butoxy-5,6,7,8-tetrahydro-4˜{a}˜{H}-4,1,2-benzothiadiazin-3-amine; 5-(4-cyclohexylphenyl)-6˜{H}-1,3,4-thiadiazin-2-amine; 5-(4-propylphenyl)-6˜{H}-1,3,4-thiadiazin-2-amine; 5-(4-isopropylphenyl)-6˜{H}-1,3,4-thiadiazin-2-amine; butyl 2-(2-amino-6˜{H}-1,3,4-thiadiazin-5-yl)acetate; 5-(2-benzyloxyethyl)-6˜{H}-1,3,4-thiadiazin-2-amine; 5-[2-(cyclohexoxy)ethyl]-6˜{H}-1,3,4-thiadiazin-2-amine; 5-(2-butoxyethyl)-6˜{H}-1,3,4-thiadiazin-2-amine; 5-(2-isopropoxyethyl)-6˜{H}-1,3,4-thiadiazin-2-amine; 5-(2-pyridyl)-6˜{H}-1,3,4-thiadiazin-2-amine; 4-(2-amino-6˜{H}-1,3,4-thiadiazin-5-yl)benzonitrile; 5-(4-methoxyphenyl)-6˜{H}-1,3,4-thiadiazin-2-amine; (2-amino-6˜{H}-1,3,4-thiadiazin-5-yl)methyl propanoate; methyl 2-(2-amino-6˜{H}-1,3,4-thiadiazin-5-yl)acetate; benzyl 2-(2-amino-6˜{H}-1,3,4-thiadiazin-5-yl)acetate; 5-(3-methoxyphenyl)-6˜{H}-1,3,4-thiadiazin-2-amine; 5-(4-chlorophenyl)-6˜{H}-1,3,4-thiadiazin-2-amine; 5-(3-chlorophenyl)-6˜{H}-1,3,4-thiadiazin-2-amine; 5-(p-tolyl)-6˜{H}-1,3,4-thiadiazin-2-amine; 5-(m-tolyl)-6˜{H}-1,3,4-thiadiazin-2-amine; 5-(2-methoxyethyl)-6˜{H}-1,3,4-thiadiazin-2-amine; 5-phenyl-6˜{H}-1,3,4-thiadiazin-2-amine; ethyl 2-(2-amino-6˜{H}-1,3,4-thiadiazin-5-yl)acetate; 6-(2-butoxyethyl)-4˜{H}-1,3,4-thiadiazin-2-amine; 5-[4-[(2-Bromophenyl)methoxy]butyl]-6H-1,3,4-thiadiazin-2-amine; 5-[4-[(3-Vinylphenyl)methoxy]butyl]-6H-1,3,4-thiadiazin-2-amine; 5-[4-[[3-[(E)-Prop-1-enyl]phenyl]methoxy]butyl]-6H-1,3,4-thiadiazin-2-amine; 5-[4-[(3-allylphenyl)methoxy]butyl]-6H-1,3,4-thiadiazin-2-amine; 5-[4-[(3-Prop-1-ynylphenyl)methoxy]butyl]-6H-1,3,4-thiadiazin-2-amine; 4-[4-(2-Amino-6H-1,3,4-thiadiazin-5-yl)butoxymethyl]benzonitrile; 3-[4-(2-Amino-6H-1,3,4-thiadiazin-5-yl)butoxymethyl]benzonitrile; 5-[6-(4-Bromo-2,6-difluoro-phenyl)hexyl]-6H-1,3,4-thiadiazin-2-amine; 5-[4-[(2,3,4-Trifluorophenyl)methoxy]butyl]-6H-1,3,4-thiadiazin-2-amine; 5-[4-[(2,3-Difluorophenyl)methoxy]butyl]-6H-1,3,4-thiadiazin-2-amine; 5-[4-[(3-Bromo-5-fluoro-phenyl)methoxy]butyl]-6H-1,3,4-thiadiazin-2-amine; 5-[4-[(2,4,6-Trifluorophenyl)methoxy]butyl]-6H-1,3,4-thiadiazin-2-amine; 5-[4-[(2,6-Difluorophenyl)methoxy]butyl]-6H-1,3,4-thiadiazin-2-amine; 5-[4-[(2,6-Difluoro-4-methoxy-phenyl)methoxy]butyl]-6H-1,3,4-thiadiazin-2-amine; 5-[4-[(4-Bromo-3-fluoro-phenyl)methoxy]butyl]-6H-1,3,4-thiadiazin-2-amine; 5-[4-(1,3-Benzothiazol-2-ylmethoxy)butyl]-6H-1,3,4-thiadiazin-2-amine; 5-[4-[(2-Nitrophenyl)methoxy]butyl]-6H-1,3,4-thiadiazin-2-amine; 5-[4-(Tetrahydrofuran-2-ylmethoxy)butyl]-6H-1,3,4-thiadiazin-2-amine; 5-[4-[(2-Methylcyclopropyl)methoxy]butyl]-6H-1,3,4-thiadiazin-2-amine; 5-[4-[(2,4-Dimethylphenyl)methoxy]butyl]-6H-1,3,4-thiadiazin-2-amine; 5-[4-[(2-Chlorophenyl)methoxy]butyl]-6H-1,3,4-thiadiazin-2-amine; 5-[4-[(5-Methyl-2-thienyl)methoxy]butyl]-6H-1,3,4-thiadiazin-2-amine; Ethyl 3-[3-[4-(2-amino-6H-1,3,4-thiadiazin-5-yl)butoxymethyl]phenyl]propanoate; 2-[4-(2-Amino-6H-1,3,4-thiadiazin-5-yl)butoxymethyl]benzonitrile;

and tautomers thereof and salts thereof, particularly pharmaceutically acceptable salts thereof.

In one embodiment, the compound for use as an anti-fungal agent is selected from: 5-(5-pentoxypentyl)-6˜{H}-1,3,4-thiadiazin-2-amine; 5-[3-(2-phenylethoxy)propyl]-6˜{H}-1,3,4-thiadiazin-2-amine; 5-[4-(4-ethylphenoxy)butyl]-6˜{H}-1,3,4-thiadiazin-2-amine; 5-[5-(2-pyridyloxy)pentyl]-6˜{H}-1,3,4-thiadiazin-2-amine; 5-[2-(2-phenoxyethoxy)ethyl]-6˜{H}-1,3,4-thiadiazin-2-amine; 5-[4-[[2-(2-phenylethyl)phenyl]methoxy]butyl]-6˜{H}-1,3,4-thiadiazin-2-amine; 5-[4-[(3,5-dichlorophenyl)methoxy]butyl]-6˜{H}-1,3,4-thiadiazin-2-amine; 5-[4-[(4-chloro-2-fluoro-phenyl)methoxy]butyl]-6˜{H}-1,3,4-thiadiazin-2-amine; 5-[4-[(2-cyclopropylphenyl)methoxy]butyl]-6˜{H}-1,3,4-thiadiazin-2-amine; 5-[4-[[2-(trifluoromethyl)phenyl]methoxy]butyl]-6˜{H}-1,3,4-thiadiazin-2-amine; 5-[4-[(2,5-difluorophenyl)methoxy]butyl]-6˜{H}-1,3,4-thiadiazin-2-amine; 5-[4-[(2-chloro-6-fluoro-phenyl)methoxy]butyl]-6˜{H}-1,3,4-thiadiazin-2-amine; 5-[4-[(3-butylphenyl)methoxy]butyl]-6˜{H}-1,3,4-thiadiazin-2-amine; 5-[4-[(4-bromophenyl)methoxy]butyl]-6˜{H}-1,3,4-thiadiazin-2-amine; 5-[4-[(4-chlorophenyl)methylsulfanyl]butyl]-6˜{H}-1,3,4-thiadiazin-2-amine; 5-[4-(2-phenylethoxy)butyl]-6˜{H}-1,3,4-thiadiazin-2-amine; 4-(2-amino-6˜{H}-1,3,4-thiadiazin-5-yl)butyl acetate; 5-[4-[(3-ethylphenyl)methoxy]butyl]-6˜{H}-1,3,4-thiadiazin-2-amine; 5-[4-[[3-(2-phenylethyl)phenyl]methoxy]butyl]-6˜{H}-1,3,4-thiadiazin-2-amine; 5-[4-[3-(2,4-difluorophenyl)propoxy]butyl]-6˜{H}-1,3,4-thiadiazin-2-amine; 5-[4-[(3-phenylphenyl)methoxy]butyl]-6˜{H}-1,3,4-thiadiazin-2-amine; 5-[4-[[3-(trifluoromethyl)phenyl]methoxy]butyl]-6˜{H}-1,3,4-thiadiazin-2-amine; 5-[4-[(4-chloro-3-fluoro-phenyl)methoxy]butyl]-6˜{H}-1,3,4-thiadiazin-2-amine; 5-[4-[(3,5-difluorophenyl)methoxy]butyl]-6˜{H}-1,3,4-thiadiazin-2-amine; 5-[4-[(3-phenoxyphenyl)methoxy]butyl]-6˜{H}-1,3,4-thiadiazin-2-amine; 5-[4-[(2,4-dichlorophenyl)methoxy]butyl]-6˜{H}-1,3,4-thiadiazin-2-amine; 5-[4-[(3-bromophenyl)methoxy]butyl]-6˜{H}-1,3,4-thiadiazin-2-amine; 5-[4-[(3-cyclopropylphenyl)methoxy]butyl]-6˜{H}-1,3,4-thiadiazin-2-amine; 5-[4-[(4-fluoro-3-methyl-phenyl)methoxy]butyl]-6˜{H}-1,3,4-thiadiazin-2-amine; 5-[4-[(4-fluorophenyl)methoxy]butyl]-6˜{H}-1,3,4-thiadiazin-2-amine; 5-[4-[(4-methoxyphenyl)methoxy]butyl]-6˜{H}-1,3,4-thiadiazin-2-amine; 5-[4-[(2,4-difluorophenyl)methoxy]butyl]-6˜{H}-1,3,4-thiadiazin-2-amine; 5-[4-[(3-chlorophenyl)methoxy]butyl]-6˜{H}-1,3,4-thiadiazin-2-amine; 5-[4-(m-tolylmethoxy)butyl]-6˜{H}-1,3,4-thiadiazin-2-amine; 5-[4-[(4-butylphenyl)methoxy]butyl]-6˜{H}-1,3,4-thiadiazin-2-amine; 5-[4-[[4-(trifluoromethyl)phenyl]methoxy]butyl]-6˜{H}-1,3,4-thiadiazin-2-amine; 5-[4-[(4-chlorophenyl)methoxy]butyl]-6˜{H}-1,3,4-thiadiazin-2-amine; 5-[5-(4-butylphenoxy)pentyl]-6˜{H}-1,3,4-thiadiazin-2-amine; 5-[2-[(4-butylphenyl)methoxy]ethyl]-6˜{H}-1,3,4-thiadiazin-2-amine; 5-[2-[3-(4-methoxyphenyl)propoxy]ethyl]-6˜{H}-1,3,4-thiadiazin-2-amine; 5-[5-(4-ethylphenoxy)pentyl]-6˜{H}-1,3,4-thiadiazin-2-amine; 5-[2-(4-methoxybutoxy)ethyl]-6˜{H}-1,3,4-thiadiazin-2-amine; 5-[2-(3-phenylpropoxy)ethyl]-6˜{H}-1,3,4-thiadiazin-2-amine; butyl 4-(2-amino-6˜{H}-1,3,4-thiadiazin-5-yl)benzoate; 5-[5-(3-ethylphenoxy)pentyl]-6˜{H}-1,3,4-thiadiazin-2-amine; 5-(4-butoxyphenyl)-4-methyl-1,3,4-thiadiazin-2-amine; 5-(4-benzyloxybutyl)-6˜{H}-1,3,4-thiadiazin-2-amine; 5-(4-methoxyphenyl)-4-methyl-1,3,4-thiadiazin-2-amine; 5-(benzofuran-2-yl)-4-methyl-1,3,4-thiadiazin-2-amine; 4-(2-amino-4-methyl-1,3,4-thiadiazin-5-yl)benzonitrile; ethyl 4-(2-amino-4-methyl-1,3,4-thiadiazin-5-yl)benzoate; 5-(3,4-dichlorophenyl)-4-methyl-1,3,4-thiadiazin-2-amine; 4-methyl-5-(p-tolyl)-1,3,4-thiadiazin-2-amine; 5-[2-(4-pentoxyphenyl)ethyl]-6˜{H}-1,3,4-thiadiazin-2-amine; 5-(5-benzyloxypentyl)-6˜{H}-1,3,4-thiadiazin-2-amine; 5-[4-(benzofuran-2-yl)butyl]-6˜{H}-1,3,4-thiadiazin-2-amine; 5-(5-methoxypentyl)-4-methyl-3˜{H}-1,3,4-thiadiazin-2-imine; 5-(4-chlorophenyl)-4-methyl-1,3,4-thiadiazin-2-amine; 2-[5-(2-amino-6˜{H}-1,3,4-thiadiazin-5-yl)pentyl]isoindoline-1,3-dione; 5-[5-(4-fluorophenoxy)pentyl]-6˜{H}-1,3,4-thiadiazin-2-amine; 5-[(˜{E})-hept-1-enyl]-6˜{H}-1,3,4-thiadiazin-2-amine; 4-(5-methoxypentyl)-5-methyl-1,3,4-thiadiazin-2-amine; 5-[5-(2-pyridyl)pentyl]-6˜{H}-1,3,4-thiadiazin-2-amine; 5-[5-(˜{N}-methylanilino)pentyl]-6˜{H}-1,3,4-thiadiazin-2-amine; 5-(6-methoxyhexyl)-6˜{H}-1,3,4-thiadiazin-2-amine; 5-(7-phenoxyheptyl)-6˜{H}-1,3,4-thiadiazin-2-amine; 5-(7-methoxyheptyl)-6˜{H}-1,3,4-thiadiazin-2-amine; 5-(4-methoxybutyl)-6˜{H}-1,3,4-thiadiazin-2-amine; 6-(2-amino-6˜{H}-1,3,4-thiadiazin-5-yl)hexanoic acid; ethyl 6-(2-amino-6˜{H}-1,3,4-thiadiazin-5-yl)hexanoate; 5-(methoxymethyl)-6˜{H}-1,3,4-thiadiazin-2-amine; 5-(4-phenoxybutyl)-6˜{H}-1,3,4-thiadiazin-2-amine; 5-[2-(4-phenylphenoxy)ethyl]-6˜{H}-1,3,4-thiadiazin-2-amine; 5-[2-(4-methylphenoxy)ethyl]-6˜{H}-1,3,4-thiadiazin-2-amine; 5-(phenoxymethyl)-6˜{H}-1,3,4-thiadiazin-2-amine; 5-(3-phenoxypropyl)-6˜{H}-1,3,4-thiadiazin-2-amine; 5-(6-phenoxyhexyl)-6˜{H}-1,3,4-thiadiazin-2-amine; 5-(6-phenylhexyl)-6˜{H}-1,3,4-thiadiazin-2-amine; 5-(2-amino-6˜{H}-1,3,4-thiadiazin-5-yl)-˜{N}-phenyl-pentanamide; 5-[2-[(4-methoxyphenyl)methoxy]ethyl]-6˜{H}-1,3,4-thiadiazin-2-amine; 5-[2-(4-propoxyphenyl)ethyl]-6˜{H}-1,3,4-thiadiazin-2-amine; 4-[2-(2-amino-6˜{H}-1,3,4-thiadiazin-5-yl)ethoxymethyl]benzonitrile; 5-[2-(1-methylbutoxy)ethyl]-6˜{H}-1,3,4-thiadiazin-2-amine; 5-[2-(p-tolylmethoxy)ethyl]-6˜{H}-1,3,4-thiadiazin-2-amine; 5-[2-[(4-phenylphenyl)methoxy]ethyl]-6˜{H}-1,3,4-thiadiazin-2-amine; 5-(2-hexoxyethyl)-6˜{H}-1,3,4-thiadiazin-2-amine; 5-[2-(2-methoxyethoxy)ethyl]-6˜{H}-1,3,4-thiadiazin-2-amine; 5-(2-phenoxyethyl)-6˜{H}-1,3,4-thiadiazin-2-amine; 5-[2-(4-bromophenoxy)ethyl]-6˜{H}-1,3,4-thiadiazin-2-amine; 5-(2-naphthyl)-6˜{H}-1,3,4-thiadiazin-2-amine; 5-(1-naphthyl)-4˜{H}-1,3,4-thiadiazin-2-amine; 5-(4-butoxyphenyl)-6˜{H}-1,3,4-thiadiazin-2-amine; 5-(3-butoxyphenyl)-6˜{H}-1,3,4-thiadiazin-2-amine; 5-(3,4-dichlorophenyl)-6˜{H}-1,3,4-thiadiazin-2-amine; ethyl 3-(2-amino-6˜{H}-1,3,4-thiadiazin-5-yl)benzoate; 5-(5-methoxypentyl)-6˜{H}-1,3,4-thiadiazin-2-amine; 5-(3-methoxypropyl)-6˜{H}-1,3,4-thiadiazin-2-amine; 5-(3,5-dimethylphenyl)-6˜{H}-1,3,4-thiadiazin-2-amine; ethyl 4-(2-amino-4˜{H}-1,3,4-thiadiazin-5-yl)benzoate; 5-heptyl-4˜{H}-1,3,4-thiadiazin-2-amine; isopropyl 2-(2-amino-6˜{H}-1,3,4-thiadiazin-5-yl)acetate; 5,6-dihydro-4˜{a}˜{H}-benzo[h][4,1,2]benzothiadiazin-3-amine; 5,6,7,8-tetrahydro-1˜{H}-4,1,2-benzothiadiazin-3-amine; 5-(5-phenoxypentyl)-6˜{H}-1,3,4-thiadiazin-2-amine; 5-(4-phenylbutyl)-6˜{H}-1,3,4-thiadiazin-2-amine; 6-butoxy-5,6,7,8-tetrahydro-4˜{a}˜{H}-4,1,2-benzothiadiazin-3-amine; 5-(4-cyclohexylphenyl)-6˜{H}-1,3,4-thiadiazin-2-amine; 5-(4-propylphenyl)-6˜{H}-1,3,4-thiadiazin-2-amine; 5-(4-isopropylphenyl)-6˜{H}-1,3,4-thiadiazin-2-amine; butyl 2-(2-amino-6˜{H}-1,3,4-thiadiazin-5-yl)acetate; 5-(2-benzyloxyethyl)-6˜{H}-1,3,4-thiadiazin-2-amine; 5-[2-(cyclohexoxy)ethyl]-6˜{H}-1,3,4-thiadiazin-2-amine; 5-(2-butoxyethyl)-6˜{H}-1,3,4-thiadiazin-2-amine; 5-(2-isopropoxyethyl)-6˜{H}-1,3,4-thiadiazin-2-amine; 5-(2-pyridyl)-6˜{H}-1,3,4-thiadiazin-2-amine; 4-(2-amino-6˜{H}-1,3,4-thiadiazin-5-yl)benzonitrile; 5-(4-methoxyphenyl)-6˜{H}-1,3,4-thiadiazin-2-amine; (2-amino-6˜{H}-1,3,4-thiadiazin-5-yl)methyl propanoate; methyl 2-(2-amino-6˜{H}-1,3,4-thiadiazin-5-yl)acetate; benzyl 2-(2-amino-6˜{H}-1,3,4-thiadiazin-5-yl)acetate; 5-(3-methoxyphenyl)-6˜{H}-1,3,4-thiadiazin-2-amine; 5-(4-chlorophenyl)-6˜{H}-1,3,4-thiadiazin-2-amine; 5-(3-chlorophenyl)-6˜{H}-1,3,4-thiadiazin-2-amine; 5-(p-tolyl)-6˜{H}-1,3,4-thiadiazin-2-amine; 5-(m-tolyl)-6˜{H}-1,3,4-thiadiazin-2-amine; 5-(2-methoxyethyl)-6˜{H}-1,3,4-thiadiazin-2-amine; 5-phenyl-6˜{H}-1,3,4-thiadiazin-2-amine; ethyl 2-(2-amino-6˜{H}-1,3,4-thiadiazin-5-yl)acetate; 6-(2-butoxyethyl)-4˜{H}-1,3,4-thiadiazin-2-amine;

and tautomers thereof and salts thereof, particularly pharmaceutically acceptable salts thereof.

The compound for use as an anti-fungal agent may preferably be selected from: 5-[4-(4-ethylphenoxy)butyl]-6˜{H}-1,3,4-thiadiazin-2-amine; 5-[5-(2-pyridyloxy)pentyl]-6˜{H}-1,3,4-thiadiazin-2-amine; 5-[2-(2-phenoxyethoxy)ethyl]-6˜{H}-1,3,4-thiadiazin-2-amine; 5-[4-[(3-bromophenyl)methoxy]butyl]-6˜{H}-1,3,4-thiadiazin-2-amine; 5-[4-[(2,4-difluorophenyl)methoxy]butyl]-6˜{H}-1,3,4-thiadiazin-2-amine; 5-(4-benzyloxybutyl)-6˜{H}-1,3,4-thiadiazin-2-amine; 5-(5-benzyloxypentyl)-6˜{H}-1,3,4-thiadiazin-2-amine; 5-(5-methoxypentyl)-4-methyl-3˜{H}-1,3,4-thiadiazin-2-imine; 5-[5-(4-fluorophenoxy)pentyl]-6˜{H}-1,3,4-thiadiazin-2-amine; 5-(2-hexoxyethyl)-6˜{H}-1,3,4-thiadiazin-2-amine; 5-(5-methoxypentyl)-6˜{H}-1,3,4-thiadiazin-2-amine; ethyl 4-(2-amino-4˜{H}-1,3,4-thiadiazin-5-yl)benzoate; 5-(5-phenoxypentyl)-6˜{H}-1,3,4-thiadiazin-2-amine and 5-(2-butoxyethyl)-6˜{H}-1,3,4-thiadiazin-2-amine, and tautomers thereof and salts thereof, particularly pharmaceutically acceptable salts thereof

The compound of Formula (I) may be as depicted or may be in the form of a tautomer. A tautomeric form of a compound of Formula (I) is as shown in Formula (Ix)

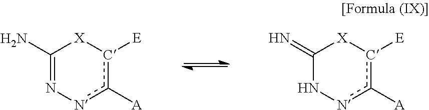

In another aspect, the invention also provides a compound of Formula (I) as described herein. In a preferred aspect, the invention provides a compound of Formula (I) wherein X, N′, C′, and E are as described herein, and further wherein A is -L-T, wherein L and T are as described herein. In another preferred aspect, the invention provides a compound of Formula (I) wherein X, N′, C′, and E are as described herein, and further wherein A is -T, wherein T is as described herein. In a more preferred aspect the invention provides a compound of Formula (I) wherein X, N′, C′, and E are as described herein, and further wherein A is -L-T, wherein L is as described herein and wherein T is aryl, heteroaryl, cycloalkyl or heterocyclyl, preferably aryl, wherein T is unsubstituted or is substituted by 1, 2 or 3 groups V, as described herein.

In one embodiment, when A is T, T is aryl, heteroaryl, cycloalkyl or heterocyclyl on the proviso that T is not benzofuran, napthofuran or 2H-chromen-2-one.

In one aspect, the invention provides a compound of Formula (I) wherein X, N′, C′ and E are as described herein, wherein A is -L-T wherein T is as defined herein and further wherein L is a linear alkylene or alkenylene moiety comprising from 5 to 12 carbon atoms, preferably from 5 to 8 carbon atoms; wherein L is unsubstituted or substituted as described herein and wherein L optionally terminates in and/or is interrupted by, a heteromoiety as described herein.

In one aspect, the invention provides a compound of Formula (I) wherein X, N′, C′, and E are as described herein, wherein A is -L-T wherein T is as defined herein and further wherein L is a C₅-C₁₂ alkylene or C₅-C₁₂ alkenylene moiety, preferably a C₅-C₈ alkylene or C₅-C₈ alkenylene moiety; wherein L is unsubstituted or substituted as described herein and wherein L terminates in and/or is interrupted by a heteromoiety as described herein.

In one aspect, the invention provides a compound of Formula (I) wherein X, N′, C′, and E are as described herein, wherein A is -L-T wherein T is as defined herein and further wherein L is a C₅-C₁₂ alkylene moiety, wherein L is unsubstituted and wherein L terminates in a heteromoiety O and/or is interrupted by a heteromoiety —O—.

In one aspect, the invention provides a compound of Formula (I) wherein X, N′, C′, and E are as described herein, wherein A is -L-T wherein L is a linear C₅-C₁₂ alkylene or C₅-C₁₂ alkenylene moiety, preferably a C₅-C₈ alkylene or C₅-C₈ alkenylene moiety; wherein L is unsubstituted or substituted as described herein and wherein L terminates in and/or is interrupted by a heteromoiety as described herein; and wherein T is unsubstituted aryl or aryl substituted with 1, 2 or 3 groups V as described herein.

In one aspect, the invention provides a compound of Formula (I) wherein X, N′, C′, and E are as described herein, wherein A is -L-T wherein L is a C₅-C₁₂ alkylene moiety, wherein L is unsubstituted and wherein L terminates in a heteromoiety —O— and/or is interrupted by a heteromoiety —O—; and wherein T is unsubstituted aryl or aryl substituted with 1, 2 or 3 groups V as described herein.

In one aspect, the invention provides a compound of Formula (I) wherein X, N′, C′, and E are as described herein, wherein A is -T wherein T is aryl substituted with 1, 2 or 3 groups V as described herein, and further wherein at least one group V is selected from —C(O)O(C₁-C₄ alkyl).

Preferably the compound is not 2-(2-(2-amino-2H-1,3,4-thiadiazin-5-yl)ethyl)isoindoline-1,3-dione; 2-(1-(2-amino-2H-1,3,4-thiadiazin-5-yl)ethyl)isoindoline-1,3-dione; or 5-benzyl-6H-1,3,4-thiadiazin-2-amine.

In one embodiment, the compound is not: 2-(2-(2-amino-2H-1,3,4-thiadiazin-5-yl)ethyl)isoindoline-1,3-dione; 2-(1-(2-amino-2H-1,3,4-thiadiazin-5-yl)ethyl)isoindoline-1,3-dione; 5-benzyl-6H-1,3,4-thiadiazin-2-amine; 2-amino-5-benzyl-1,3,4-thiadiazine; 5-(3-nitrobenzyl)-6H-1,3,4-thiadiazin-2-amine; 5-(3-methylpentan-3-yl)-6H-1,3,4-thiadiazin-2-amine; or 5-[2-(5-nitro-2-furanyl)ethenyl]-6H-1,3,4-thiadiazin-2-amine.

The compounds as described herein can be prepared by any suitable method. Many synthetic schemes suitable for producing the compounds as described herein would be known to the skilled person. For example, the general synthesis of the diazin-2-ylamine heterocyclic core can be (mite readily achieved

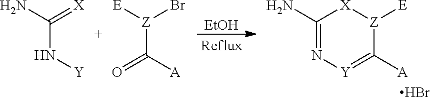

For example, commercially available thiosemicarbazide may be reacted with the appropriate α-bromoketone to give the desired amino thiadiazine as a solid in good yields.

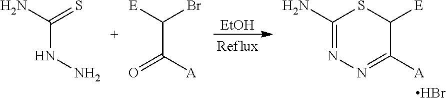

In the generalised reaction schemes above, the starting materials may be synthesized by known techniques, or commercially obtained. Where required, a pharmaceutically acceptable salt can easily be formed from a compound as described herein via standard reactions.

The invention also provides a pharmaceutical composition comprising a compound as described herein together with a pharmaceutically acceptable carrier, diluent and/or excipient. Typically, the composition contains up to 85 wt % of a compound as described herein. More typically, it contains up to 50 wt % of a compound as described herein. Preferred pharmaceutical compositions are sterile and pyrogen free. Further, the pharmaceutical compositions provided by the invention typically contain a compound which is a substantially pure optical isomer.

The present invention also provides prodrugs of the compounds as described herein. A prodrug is an analogue of a compound as described herein which will be converted in vivo to the desired active compound. Examples of suitable prodrugs include compounds of formula (I) which have been modified at a carboxylic acid group to form an ester, or at hydroxyl group to form an ester or carbamate. Other suitable methods will be known to those skilled in the art. Further suitable prodrugs include those in which a nitrogen atom of a compound of formula (I) is quaternised by addition of an ester or alkyl ester group. For example, the nitrogen atom of an amine group, for example an amine group bonded to the diazine ring of Formula (I), may be quaternised by addition of a —CH₂—O—COR group, wherein R is typically methyl or tert-butyl.

The composition may be prepared by bringing into association a compound as described herein with the carrier, diluent and/or excipient. In general, such formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein in conjunction or association with a pharmaceutically acceptable carrier or vehicle.

The excipient, diluent and/or the carrier, or, if more than one be present, each of the excipients, diluents and/or carriers, must be acceptable in the sense of being compatible with the other ingredients of the formulation and not deleterious to the recipient.

The invention further provides a product comprising a compound as described herein or a composition as described herein and further comprising a second antifungal agent. The combination of a compound as described herein or a composition as described herein with a second antifungal agent may, for example, be more effective than either agent alone.

The second antifungal agent may typically be selected from the group consisting of the polyenes, the azoles, the echinocandins and flucytosine. For example, the second antifungal agent may be selected from Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, Amorolfin, Butenafine, Naftifine, Terbinafine, Anidulafungin, Caspofungin, and Micafungin.

As described herein, the present invention provides a product comprising (i) a as described herein or a composition as described herein and (ii) a second antifungal agent. The compound or composition and the second antifungal agent may be provided in a single formulation, or they may be separately formulated. Where formulated together, the two active agents may be provided as a composition comprising (i) a compound as described herein and (ii) a second antifungal compound; and (iii) a pharmaceutically acceptable carrier or diluent. Where separately formulated, the two agents may be administered simultaneously or separately.

The compound, composition or product as described herein may be provided in the form of a kit, optionally comprising instructions to enable the kit to be used in the methods described herein or details regarding which subjects the method may be used for. The kit may comprise (i) a compound and/or composition as described herein and (ii) a second antifungal agent as defined herein. In this case, the compound/composition and the antifungal agent may be for administration simultaneously or separately, either immediately after one another or at different times. The kit may comprise instructions for such administration.

The compound, composition or product as described herein is typically formulated as a composition for administration with a pharmaceutically acceptable carrier or diluent.

The formulations include those suitable for oral, inhaled, rectal, nasal, topical (including eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral, parenteral, inhaled or topical administration, especially oral or parenteral administration and more especially parenteral administration, especially intravenous administration.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For formulations for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable.

Tablets may also be coated by methods well known in the art. A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

The compound, composition or product as described herein may be formulated for inhaled (aerosolised) administration as a solution or suspension. The compound, composition or product as described herein may be administered by a metered dose inhaler (MDI) or a nebulizer such as an electronic or jet nebulizer. Alternatively, the compound, composition or product as described herein may be formulated for inhaled administration as a powdered drug, such formulations may be administered from a dry powder inhaler (DPI). When formulated for inhaled administration, the compound, composition or product as described herein may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the compound, composition or product as described herein is delivered as a nebulized aerosol, the reference to particle diameters defines the MMAD of the droplets of the aerosol. The MMAD can be measured by any suitable technique such as laser diffraction.

For topical application to the skin, the compounds may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used. Parenteral formulations will generally be sterile.

For the avoidance of doubt, the compounds and compounds for use as described herein may be used or administered in the form of a solvate.

The compounds, compositions and products as described herein are therapeutically useful. The present invention therefore provides a compound, composition or product as described herein, for use in medicine. The present invention also provides a compound, composition or product as described herein, for use in treating the human or animal body. In particular, the compounds, compositions and products as described herein are useful in the treatment or prevention of fungal infection.

The present invention therefore provides a compound, composition or product as described herein for use in the prevention or treatment of fungal infection.

The compound may be administered in combination with a second antifungal agent as described herein. Therefore, the invention also provides a compound or composition as described herein for use in the treatment or prevention of fungal infection in combination with a second antifungal agent.

The present invention also provides use of a compound, composition or product as described herein in the manufacture of a medicament for treating the human or animal body. The invention further provides use of a compound, composition or product as described herein in the manufacture of a medicament for use in the prevention or treatment of fungal infection. The invention further provides use of a compound or composition as described herein in the manufacture of a medicament for use in a method for the prevention or treatment of fungal infection, wherein the method further comprises administration of a second antifungal agent.

The present invention also provides a method of treatment of the human or animal body comprising administration of a compound, composition or product as described herein to a subject in need thereof. There is also provided a method for the prevention or treatment of fungal infection, the method comprising administering to a subject in need of such treatment an effective amount of a compound, composition or product as described herein. Also provided is a method for the prevention or treatment of a fungal infection, the method comprising administering to a subject in need of such treatment an effective amount of a compound, composition or product as described herein wherein the method further comprises administration of a second antifungal agent.

The fungal infection is typically one caused by a pathogenic fungus, such as a fungus selected from Candida, Aspergillus (e.g. Aspergillus fumigatus and Aspergillus flavus), Cryptococcus (e.g. Cryptococcus neoformans, Cryptococcus laurentii, Cryptococcus albidus Cryptococcus gattii), Histoplasma (e.g. Histoplasma capsulatum), Pneumocystis (e.g. Pneumocystis jirovecii and Pneumocystis carinii), Stachybotrys (e.g. Stachybotrys chartarum), Trichophyton (e.g. T rubrum, T mentagrophytes, Trichophyton concentricum, T interdigitale), Absidia (e.g. Absidia coerulea, Absidia corymbifera, Absidia cylindrospora, Absidia ginsan, Absidia glauca, Absidia spinosa), Rhizopus (e.g. Rhizopus oryzae), Fusarium (e.g. Fusarium graminearum, Fusarium oxysporum, Fusarium verticillioides, Fusarium proliferatum, Fusani solani), and Scedosporium (e.g. Scedosporium prolificans).

For example, the fungal infection may be one caused by a pathogenic fungus, such as a fungus selected from Candida, Aspergillus (e.g. Aspergillus fumigatus and Aspergillus flavus), Cryptococcus (e.g. Cryptococcus neoformans, Cryptococcus laurentii, Cryptococcus albidus Cryptococcus gattii), Histoplasma (e.g. Histoplasma capsulatum), Pneumocystis (e.g. Pneumocystis jirovecii and Pneumocystis carinii), Stachybotrys (e.g. Stachybotrys chartarum), Trichophyton (e.g. T rubrum, T mentagrophytes, Trichophyton concentricum, T interdigitale), Absidia (e.g. Absidia coerulea, Absidia corymbifera, Absidia cylindrospora, Absidia ginsan, Absidia glauca, Absidia spinosa), Rhizopus (e.g. Rhizopus oryzae), Fusarium (e.g. Fusarium oxysporum, Fusarium proliferatum, Fusani solani), and Scedosporium (e.g. Scedosporium prolificans). Preferably, the fungal infection is one caused by Aspergillus or Candida, in particular caused by C. albicans, C. parapsilosis, C. tropicalis, C. glabrata, C. krusei, A. niger, A. fumigatus, A. terreus, A. flavus, A. terreus 49, or A. fumigatus 210, e.g. Candida for example C. albicans, C. parapsilosis, C. tropicalis, C. glabrata or C. krusei.

Preferably, the compound described herein is active against two or more, e.g. two, three or four, different fungi, for example two or more (e.g. 2, 3 or 4) of the fungi described above. Thus, the treatment of the invention may be the prevention or treatment of fungal infection caused by two or more (e.g. 2, 3 or 4) different fungi.

A therapeutically or prophylactically effective amount of the compound, composition or product as described herein is administered to a subject. In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a fungus.

The dose of the compound, composition or product as described herein (“the agent”) to be administered to the subject may be determined according to various parameters, especially according to the compound used; the age, weight and condition of the subject to be treated; the route of administration; and the required regimen. A physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g. The precise amount of the agent which is therapeutically effective, and the most efficacious route for therapeutic administration, is readily determined by one of ordinary skill in the art by comparing the blood level of the agent to the concentration required to have a therapeutic effect.

In a further aspect of the invention there is provided a process for the preparation of a pharmaceutical or veterinary composition, the process comprising admixing a compound as described herein, a pharmaceutically acceptable excipient or carrier and optionally a second antibiotic agent.

The compounds described herein may also be used in a method of controlling a fungal disease of a plant, which comprises applying to the locus of the plant a compound of formula (I) as described herein or an agriculturally acceptable salt thereof, and optionally a second antifungal agent.

The compounds, compositions and products of the invention may, for example, be applied to the seeds of the plants, to the medium (e.g. soil or water) in which the plants are grown, or to the foliage of the plants.

The compounds, compositions and products of the invention are preferably used in the treatment or prevention of fungal diseases. Examples of fungal diseases of plants which can be controlled using the compounds of the invention include fungal diseases caused by the following plant pathogens: Blumeria graminis; Colletotrichium trifolii; Fusarium graminearium; Fusarium solani; Fusarium sporotrichoides; Leptosphaeria nodorum; Magnaporthe grisea; Mycosphaerella graminicola; Neurospora crassa; Phytophthora capsici; Phytophthora infestans; Plasmopara viticola; Puccinia coronata; Puccinia graminis; Pyricularia oryzae; Pythium ultimum; Rhizoctonia solani; Trichophyton rubrum; and Ustilago maydis.

The present invention includes a composition comprising a compound as described herein, or an agriculturally acceptable salt thereof, and an agriculturally acceptable carrier or diluent. In one embodiment of the invention, the composition further comprises a second antifungal agent. Said agricultural composition typically contains up to 85 wt % of a compound of the invention. More typically, it contains up to 50 wt % of a compound of the invention. When used in an agricultural composition, the skilled person will readily be able to determine suitable levels of administration. As examples, the antifungal agent(s) can be used at a level of from 5 g to 10 kg per hectare, for example from 10 g to 5 kg per hectare, for example from 100 g to 2 kg per hectare.

Suitable agriculturally acceptable salts include salts with agriculturally acceptable acids, both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Salts may also be formed with agriculturally acceptable bases such as alkali metal (e.g. sodium or potassium) and alkaline earth metal (e.g. calcium or magnesium) hydroxides and organic bases such as alkyl amines, aralkyl amines or heterocyclic amines. A preferred agriculturally acceptable salt is the hydrochloride salt.

The compounds described herein, and optional second antifungal agents, may be applied in combination with inert carriers or diluents, as in aqueous sprays, granules and dust formulations in accordance with established practice in the art. An aqueous spray is usually prepared by mixing a wettable powder or emulsifiable concentrate formulation of a compound of the invention with a relatively large amount of water to form a dispersion.

Wettable powders may comprise an intimate, finely divided mixture of a compound of the invention, an inert solid carrier and a surface-active agent. The inert solid carrier is usually chosen from among the attapulgite clays, the kaolin clays, the montmorillonite clays, the diatomaceous earths, finely divided silica and purified silicates. Effective surfactants, which have wetting, penetrating and dispersing ability are usually present in a wettable powder formulation in proportions of from 0.5 to 10 percent by weight. Among the surface active agents commonly used for this purpose are the sulfonated lignins, naphthalenesulfonates and condensed naphthalenesulfonates, alkylbenzenesulfonates, alkyl sulfates and non-ionic surfactants such as products of condensation of ethylene oxide with alkylphenols. Emulsifiable concentrates may comprise a solution of a compound of the invention in a liquid carrier which is a mixture of a water-immiscible solvent and a surfactant, including an emulsifier. Useful solvents include aromatic hydrocarbon solvents such as the xylenes, alkylnaphthalenes, petroleum distillates, terpene solvents, ether-alcohols and organic ester solvents. Suitable emulsifiers, dispersing and wetting agents may be selected from the same classes of products which are employed in formulating wettable powders.

The fungicide formulations desirably contain from 0.1 percent to 95 percent by weight of the compound of the invention, or in the case of a combination of antifungal agents the total weight of antifungal agent, and from 0.1 to 75 percent of an inert carrier or surfactant. The direct application to plant seeds prior to planting may be accomplished in some instances by mixing either a powdered solid compound of the invention or a dust formulation with seed to obtain a substantially uniform coating which is very thin and represents only one or two percent by weight or less, based on the weight of the seed. In some instances, however, a non-phytotoxic solvent such as methanol is conveniently employed as a carrier to facilitate the uniform distribution of the compound of the invention on the surface of the seed.

When a compound as described ehrein, or in the case of a combination of antifungal agents one of the antifungal agents used, is to be applied to the soil, as for pre-emergence protection, granular formulations or dusts are sometimes more convenient than sprays. A typical granular formulation comprises a compound of the invention dispersed on an inert carrier such as coarsely ground clay, or clay which has been converted to granules by treatment of a rolling bed of the powdered material with a small amount of liquid in a granulating drum. In the usual process for preparing granular formulations, a solution of the active compound is sprayed on the granules while they are being agitated in a suitable mixing apparatus, after which the granules are dried with a current of air during continued agitation. Dust formulations customarily employ essentially the same inert diluents as wettable powders and granules, but are well-mixed in powder form and do not usually contain emulsifiers. Dusts may contain some surface active agents to facilitate uniform distribution of the active ingredient in the formulation and to improve the uniformity and adhesion of the dust coating on seeds and plants. The colloidal dispersion of dust formulations in the air is usually prevented by incorporation of a minor amount of an oily or waxy material in the formulation to cause agglomeration of colloidal size particles. In this way the dust may be applied to seeds or plants without generation of an air-polluting aerosol.

The following Examples illustrate the invention. They do not however, limit the invention in any way. In this regard, it is important to understand that the particular assay used in the Examples section is designed only to provide an indication of biological activity. There are many assays available to determine biological activity, and a negative result in one assay is therefore not determinative.

### EXAMPLES

General Methods

Preparative column chromatography was performed on Merck silica gel 60 (230-400 mesh) or Carlo Erba silica gel 60A (40-63 μm). Preparative HPLC was performed on a Gilson system equipped with a UV detector in accordance to the experimental details specified below. The mobile phases used were either acidic (0.1% TFA/methanol) or basic (50 mM ammonium bicarbonate/ammonia (aq)/methanol). The column used was an XBridge C18, 5 μm, OBD™ 19×50 mm. The purest fractions were collected, concentrated and dried under vacuum. Analytical HPLC/MS was performed using an Agilent 1100 Series Liquid Chromatograph/Mass Selective Detector (MSD) (Single Quadrupole) equipped with an electrospray interface and a UV diode array detector. NMR spectra were recorded on a Varian Mercury plus at 25° C. at 400 MHz for ¹H and 101 MHz for ¹³C. Data is presented as follows: chemical shift (ppm), multiplicity (s=singlet, d=doublet, t=triplet, m=multiplet, br=broad, app=apparent), coupling constant (Hz), integration. The solvent residual peak was used as an internal standard (CD₃OD ¹H: 3.31 ppm, ¹³C: 49 ppm; DMSO-d₆ ¹H: 2.50 ppm and CDCl₃ ¹H: 7.26 ppm). The compounds prepared were given IUPAC names obtained from the software Accelrys draw 4.2 and Dotmatics. In addition, the commercial or trivial names were used for many of the commercial available starting materials and reagents. Yields are calculated on the assumption that the amino thiadiazine compounds are isolated as the free base except when explicitly indicated in the title. However, the isolated compounds could also fully or partially be HBr (or HCl) salts.

### Example 1

### 5-(2-Methoxy-ethyl)-6H-[1,3,4]thiadiazin-2-ylamine

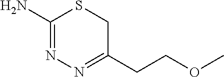

### A) Preparation of 1-Chloro-4-methoxy-butan-2-one

To a solution of anhydrous AlCl₃ (4.87 g, 36.6 mmol) in 50 mL dry CH₂Cl₂ at 0° C. was added dropwise a CH₂Cl₂ solution of 3-chloro-2-ethoxy methoxy-propene (5.0 g, 36.6 mmol) and the reaction mixture was allowed to stir for 2 h at RT. The reaction was monitored by TLC and after completion of the reaction mixture was quenched with 200 mL ice-cold water and then subjected to a standard ether work up to yield 4 g (80%) of the desired product as a brown liquid which was used crude in the next step.

### B) Preparation of 5-(2-Methoxy-ethyl)-6H-[1,3,4]thiadiazin-2-ylamine

To a solution of 1-chloro-4-methoxy butan-2-one (4.0 g, 29.3 mmol) in 40 mL acetonitrile was added thiosemicarbazide (2.67 g, 29.3 mmol) at RT. Upon complete addition the reaction mixture was raised to reflux and maintained at reflux temperature for 8 h. The reaction was monitored by TLC and after completion of the reaction solvent was evaporated under vacuum and the crude reaction mixture was dissolved in THF and washed with saturated aqueous NaHCO₃ solution. Then solvent was evaporated and purified by column chromatography using methanol-CH₂Cl₂ (9:1) to yield 1.1 g (22%) of 5-(2-methoxy-ethyl)-6H-[1,3,4]thiadiazin-2-ylamine as a buff solid.

### Example 2

### 5-m-tolyl-6H-[1,3,4]thiadiazin-2-yl amine hydrobromide

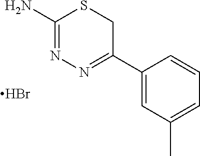

To a solution of 3-methylphenacylbromide (1.0 g, 4.3 mmol) in ethanol (20 mL) at 0° C. was added thiosemicarbazide (400 mg, 4.3 mmol) and upon complete addition the mixture was allowed to warm to RT and stirred overnight. The resulting slurry was cooled to −20° C. and the precipitate was collected by filtration, washed with cold ethanol and dried in vacuo. The pale yellow solid was suspended in 20 mL of ethanol containing 1 mL of 48% aqueous hydrobromic acid. The mixture was heated to reflux for 30 min and was then cooled to RT overnight. The precipitate was filtered and purified by column chromatography on silica gel using Hexane:EtOAc (49:1) to give 950 mg (70%) of 5-m-tolyl-6H-[1,3,4]thiadiazin-2-yl amine hydrobromide.

### Example 3

### 5-(3-chloro-phenyl)-6H-[1,3,4]thiadiazin-2-yl amine hydrobromide

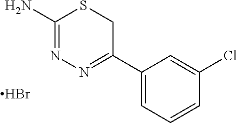 [SMILES: Br ; NC1=NN=C(c2cccc(Cl)c2)CS1]

### A) Preparation of 2-bromo-1-(3-chloro-phenyl)-ethanone

To the stirred solution of 1-(3-chloro-phenyl)-ethanone (5.0 g, 32.3 mmol) in chloroform (75 mL) was added bromine (1.66 mL, 32.2 mmol) at 5-10° C. After addition the temperature of the reaction mixture was slowly raised to room temperature and stirred at room temperature for 1 h. The reaction was monitored by TLC and upon completion the reaction mixture was diluted with 10% Na₂S₂O₃ solution (100 mL) and extracted with CH₂Cl₂ (50 mL). The organic layer was washed with water (400 mL), dried and concentrated to give 2-bromo-1-(3-chloro-phenyl)-ethanone as a crude product 7.2 g (96%) which was used for the next step without purification.

### B) Preparation of 5-(3-Chloro-phenyl)-6H-[1,3,4]thiadiazin-2-yl amine hydrobromide

To the stirred solution of 2-bromo-1-(3-chloro-phenyl)-ethanone (4.0 g (crude), 17.0 mmol) in ethanol (40 mL) at 0° C. was added thiosemicarbazide (1.0 g, 12.0 mmol) portionwise. After addition temperature of the mixture was slowly raised to room temperature and stirred at room temperature for 8 h. The reaction was monitored by TLC and after completion of the reaction mixture was cooled to −20° C. and the precipitate was collected by filtration, washed with cold ethanol and dried in vacuo. The pale yellow solid was again suspended in 20 mL of ethanol and 1 mL of 48% aqueous HBr solution. The mixture was heated to reflux for 30 min and then cooled to room temperature for overnight. The precipitate was filtered and purified by column chromatography on silica gel using Hexane:EtOAc (49:1) to give 5-(3-Chloro-phenyl)-6H-[1,3,4]thiadiazin-2-yl amine hydrobromide 750 mg (14%) as a yellow solid.

### Example 4

### 5-(3-methoxy-phenyl)-6H-[1,3,4]thiadiazin-2-ylamine hydrobromide

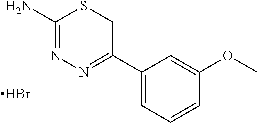

To a solution of 3-methoxyphenacylbromide (1.0 g, 4.4 mmol) in ethanol (20 mL) at 0° C. was added thiosemicarbazide (397 mg, 4.4 mmol) and upon complete addition the mixture was allowed to warm to RT and stirred overnight. The resulting slurry was cooled to −20° C. and the precipitate was collected by filtration, washed with cold ethanol and dried in vacuo. The pale yellow solid was suspended in 20 mL of ethanol containing 1 mL of 48% aqueous hydrobromic acid. The mixture was heated to reflux for 30 min and was then cooled to RT overnight. The precipitate was filtered and purified by column chromatography on silica gel using Hexane:EtOAc (49:1) to give 395 mg (30%) of 5-(3-methoxy-phenyl)-6H-[1,3,4]thiadiazin-2-ylamine hydrobromide as an off white solid.

### Example 5

### (2-Amino-6H-[1,3,4]thiadiazin-5-yl)-acetic acid benzyl ester

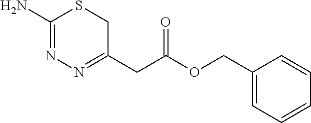 [SMILES: NC1=NN=C(CC(=O)OCc2ccccc2)CS1]

Sodium hydride (60%; 168 mg, 4.2 mmol) was added portion wise to a stirred solution of benzyl alcohol (0.3 mL, 2.8 mmol) in dry THF(10 mL) at 0° C. and stirred 30min at room temperature. A solution of (2-amino-6H-[1,3,4]thiadiazin-5-yl-acetic acid ester (600 mg, 2.8 mmol) in THF(8 mL) was added slowly to the reaction mixture at 0° C. and stirred for 1 h at room temperature. The reaction mixture was quenched with saturated ammonium chloride (10 ml) and extracted with ethyl acetate (10×3 ml), the combined organic layers was washed with brine (15×1 mL), dried over anhydrous sodium sulfate and concentrated under vacuo. The crude compound was purified by preparative TLC using ethyl acetate (20%) in pet ether as eluent to afford 44 mg (6%) of (2-Amino-6H-[1,3,4]thiadiazin-5-yl)-acetic acid benzyl ester as a yellow solid.

The synthesis was repeated again with another 600 mg of (2-amino-6H-[1,3,4]thiadiazin-5-yl-acetic acid ester (600 mg, 2.8 mmol) to isolate 61 mg (8.%) of (2-Amino-6H-[1,3,4]thiadiazin-5-yl)-acetic acid benzyl ester. The 2 batches were combined, dissolved into dichloromethane and evaporated to obtain 103 mg of 2-Amino-6H-[1, 3] thidiazin-5-yl-aceticacid benzyl ester as yellow powder [TLC system: Hexane:Ethyl acetate (5:5); R_{f} value: 0.56].

### Example 6

### (2-amino-6H-[1,3,4]thiadiazin-5-yl)-acetic acid ethyl ester. hydrochloride

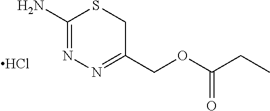

### A) Preparation of propionic acid 3-chloro-2-oxo-propyl ester

To a solution of dichloro acetone (5.0 g, 39.3 mmol) and propionic acid (3.0 g, 39.3 mmol) in DMF (25 mL) at RT was added sodium bicarbonate (3.3 g, 39.3 mmol). The reaction was stirred for 24 h and after that reaction mixture was quenched with water and then subjected to a standard ethyl acetate work-up to yield 6.0 g of the crude product and was used for next step without further purification.

### B) Preparation of (2-amino-6H-[1,3,4]thiadiazin-5-yl)-acetic acid ethyl ester hydrochloride

To a solution of Propionic acid 3-chloro-2-oxo-propyl ester (4.0 g, 24.0 mmol) in CH₃CN (20 mL) at RT was added thiosemicarbazide (2.2 g, 24.0 mmol) in CH₃CN and after the addition the mixture was stirred for overnight. The reaction was monitored by TLC and after completion of the reaction solvent was evaporated and purified by column chromatography on silica gel using MeOH:CH₂Cl₂ (7:93) to give 900 mg (15%) of (2-amino-6H-[1,3,4]thiadiazin-5-yl)-acetic acid ethyl ester hydrochloride as a buff solid.

### Example 7

### 5-(2-isopropoxy-ethyl)-6H-[1,3,4]thiadiazin-2 ylamine hydrobromide

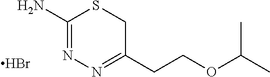

### A) Preparation of 4-isopropoxy-butan-2-one

To the stirred solution of methyl vinyl ketone (10.0 g, 0.14 mol) and isopropyl alcohol (8.41 g, 0.14 mol) was added two drops of conc. H₂SO₄ at 0° C. and after addition temperature of the reaction mixture was raised to RT and was stirred for 1 h. The reaction was monitored by TLC and after completion of the reaction ethyl acetate was added and was then subjected to a standard ethyl acetate work-up to yield crude product as a colourless oil. The crude material was pure enough for next step of reaction.

### B) Preparation of 1-bromo-4-isopropoxy-butan-2-one

To a solution of 4-isopropoxy-butan-2-one (3.64 g, 28.0 mmol) in dry methanol (50 mL) was added bromine (1.5 mL, 28.0 mmol) at 4° C. and the reaction mixture was kept at that temperature for 1.5 h, in that time color of the bromine was disappeared. Then 100 mL 1M K₂CO₃ was added, the mixture was concentrated in vacuo and extrated twice with 200 mL of diethyl ether/toluene (1:1). The extracts were washed twice with 40 mL of 1M K₂CO₃, dried, and the solvent was evaporated. The residue was dissolved in 200 mL of THF and 100 mL of 1M H₂SO₄ and the mixture was heated to reflux for 1.5 h. Then the mixture was concentrated in vacuo and extracted with cyclohexane/diethyl ether (1:1). The extracts were washed with 2M KHCO₃, dried, and the solvent was evaporated to give the crude bromo ketone as a colourless liquid. The crude material was pure enough for next step of reaction.

### C) Preparation of 5-(2-Isopropoxy-ethyl)-6H-[1,3,4]thiadiazin-2 ylamine hydrobromide

To a solution of 1-bromo-4-isopropoxy-butan-2-one (700 mg, 3.3 mmol) in EtOH (10 mL) at RT was added thiosemicarbazide (300 mg, 3.3 mmol) and was stirred overnight. The solvent was evaporated in vacuo and followed by purification by column chromatography on silica gel using MeOH:CH₂Cl₂ (1:4) to give 270 mg (29%) of 5-(2-Isopropoxy-ethyl)-6H-[1,3,4]thiadiazin-2 ylamine hydrobromide as a yellow solid

### Example 8

### 5-(2-butoxy-ethyl)-6H-[1,3,4]thiadiazin-2-ylamine hydrobromide

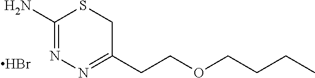

### A) Preparation of 4-butoxy-butan-2-one

To the stirred solution of methyl vinyl ketone (10.0 g, 11.7 ml, 0.14 mol) and n-butanol (10.5 g, 13.0 ml, 0.14 mol) was added conc. H₂SO₄ (2.5 ml) and water (2.5 ml) at 0° C. and upon complete addition the reaction temperature was allowed to raise to RT and was then stirred at RT for 1 hr. The reaction was monitored by TLC and after completion of the reaction ethyl acetate was added and was then subjected to a standard ethyl acetate work-up to yield crude product. The crude material was pure enough for next step of reaction.

### B) Preparation of 1-bromo-4-butoxy-butan-2-one

To a solution of 4-butoxy-butan-2-one (14.2 g, 98.0 mmol) in dry methanol (56.0 mL) was added bromine (5.1 ml, 98.0 mmol) at 4° C. and the reaction mixture was kept at that temperature for 1.5 h, in that time color of the bromine was disappeared. Then aqueous K₂CO₃ solution (1M, 170 ml) was added, the mixture was concentrated in vacuum and extracted twice with 200 ml of ethyl acetate. The extracts were washed twice with 70 ml of 1M K₂CO₃ solution, dried, and the solvent was evaporated. The residue was dissolved in 680 ml of THF and 170 ml of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give 2.8 g of crude bromo ketone. The crude material was pure enough for next step of reaction.

### C) Preparation of 5-(2-butoxy-ethyl)-6H-[1,3,4]thiadiazin-2-ylamine hydrobromide

To a solution of 1-bromo-4-butoxy-butan-2-one (6.70 g, 30.0 mmol) in EtOH (40 ml) at RT was added thiosemicarbazide (2.70 g, 30.0 mmol) and was stirred for overnight. The reaction was monitored by TLC and after completion of the reaction, mixture was filtered and the solid mass was purified by column chromatography on silica gel using MeOH:CH₂Cl₂ (1:4) to give 1.0 g (16%) of the desired product as a yellow solid.

### Example 9

### 5-(2-cyclohexyloxy-ethyl)-6H-[1,3,4]thiadiazin-2-ylamine. Hydrobromide

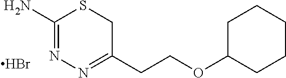 [SMILES: Br ; NC1=NN=C(CCOC2CCCCC2)CS1]

### A) Preparation of 4-Cyclohexyloxy-butan-2-one

To the stirred solution of methyl vinyl ketone (3.5 g, 0.05 mol) and cyclohexanol (5.0 g, 0.05 mol) was added concentrated H₂SO₄ (0.1 mL) at 0° C. and after addition temperature of the reaction mixture was raised to RT and was stirred for 3 h. The reaction was monitored by TLC and after completion of the reaction ethyl acetate was added and then subjected to a standard ethyl acetate work-up to yield crude product (6.65 g). The crude material was pure enough for next step of reaction.

### B) Preparation of 1-bromo-4-cyclohexyloxy-butan-2-one

To a solution of 4-cyclohexyl-butan-2-one (5.0 g, 29.4 mmol) in dry methanol (20 mL) was added bromine (1.5 mL, 29.4 mmol) at 4° C. and the reaction mixture was kept at that temperature for 1.5 h, in that time color of the bromine was disappeared. Then 60 mL 1M K₂CO₃ was added, the mixture was concentrated in vacuum and extracted twice with 200 mL of diethyl ether/toluene (1:1). The extracts were washed twice with 100 mL of 1M K₂CO₃, dried, and the solvent was evaporated. The residue was dissolved in 120 mL of THF and 60 mL of 1M H₂SO₄ and the mixture was heated to reflux for 3 h. Then the mixture was concentrated in vacuum and extracted with ethyl acetate. The extracts were washed with 2M KHCO₃, dried, and the solvent was evaporated to give 3.5 g of crude bromo ketone. The crude material was pure enough for next step of reaction.

### C) Preparation of 5-(2-cyclohexyloxy-ethyl)-6H-[1,3,4]thiadiazin-2-ylamine hydrobromide

To a solution of 1-bromo-4-cyclohexyloxy-butan-2-one (735 mg, 2.95 mmol) in EtOH (7.5 mL) at RT was added thiosemicarbazide (270 mg, 2.96 mmol) and the reaction was stirred overnight. The reaction was monitored by TLC and after completion of the reaction solvent was evaporated and was purified by column chromatography on silica gel using MeOH:CH₂Cl₂ (5:95) to give 120 mg 5-(2-cyclohexyloxy-ethyl)-6H-[1,3,4]thiadiazin-2-ylamine hydrobromide as off-white solid.

### Example 10

### 5-(2-benzyloxy-ethyl)-6H-[1,3,4]thiadiazin-2-ylamine. hydrobromide

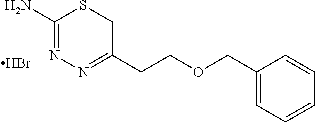 [SMILES: Br ; NC1=NN=C(CCOCc2ccccc2)CS1]

### A) Preparation of 4-benzyloxy-butan-2-one

To the stirred solution of methyl vinyl ketone (10.0 g, 0.14 mmol) and benzyl alcohol (15.4 g, 0.14 mmol) was added two drops of conc. H₂SO₄ at 0° C. Upon complete addition the temperature of the reaction mixture was raised to RT and was stirred for 1 h. The reaction was monitored by TLC and after completion of the reaction ethyl acetate was added and was then subjected to a standard ethyl acetate work-up to yield crude product. The crude material was pure enough for next step of reaction.

### B) Preparation of 4-benzyloxy-1-bromo-butan-2-one

To a solution of 4-benzyloxy-butan-2-one (5.0 g, 28.0 mmol) in dry methanol (50 mL) was added bromine (1.5 mL, 28.0 mmol) at 4° C. and the reaction mixture was kept at that temperature for 1.5 h, during which time color of the bromine dissipated. Then 100 mL 1M K₂CO₃ was added, the mixture was concentrated in vacuo and extracted twice with 200 mL of diethyl ether/toluene (1:1). The extracts were washed twice with 40 mL of 1M K₂CO₃, dried, and the solvent was evaporated. The residue was dissolved in 200 mL of THF and 100 mL of 1M H₂SO₄ and the mixture was heated to reflux for 1.5 h. Then the mixture was concentrated in vacuo and extracted with cyclohexane/diethyl ether (1:1). The extracts were washed with 2M KHCO₃, dried, and the solvent was evaporated to give 2.8 g of crude bromo ketone. The crude material was pure enough for next step of reaction.

### C) Preparation of 5-(2-benzyloxy-ethyl)-6H-[1,3,4]thiadiazin-2-ylamine. hydrobromide

To the stirred solution of 4-benzyloxy-1-bromo-butan-2-one (1.6 g, 6.22 mmol) in EtOH (15 mL) at RT was added thiosemicarbazide (560 mg, 6.21 mmol) and was stirred overnight. The reaction was monitored by TLC and after completion of the reaction solvent was evaporated and was purified by column chromatography on silica gel using ethyl acetate:methanol (9:1) to give 5-(2-benzyloxy-ethyl)-6H-[1,3,4]thiadiazin-2-ylamine hydrobromide (900 mg) as off-white solid

### Example 11

### (2-amino-6H-[1,3,4]thiadiazin-5-yl) acetic acid butyl ester

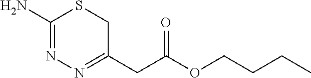

Sodium hydride (60%; 290 mg, 7.45 mmol) was added portion wise to a stirred solution of n-butanol (0.43 mL, 4.97 mmol) in 10 mL of dry THF at 0° C. and stirred for 30 min at room temperature. A solution of (2-Amino-6H-[1,3,4]thiadiazin-5-yl-acetic acid ester (1.0g, 4.97 mmol) in THF (8 mL) was added drop wise to the reaction mixture at 0° C. and stirred for 1 h at room temperature. The reaction mixture was quenched with saturated ammonium chloride (30 ml) and extracted with ethyl acetate (20×3 ml), the combined organic layers were washed with brine (20×1 ml), dried over anhydrous sodium sulfate and concentrated in vacuo to give 800 mg of crude compound. 400 mg of this material was purified twice by preparative TLC using ethyl acetate (20%) in pet ether as eluent to afford 51 mg (9%) of 2-Amino-6H-[1,3] thidiazin-5-yl]-acetic acid butyl ester as a pale yellow solid. [TLC system: Hexane:Ethyl acetate (1:1); Rf value: 0.56].

### Example 12

### 5-(4-propyl-phenyl)-6H-[1,3,4]thiadiazin-2-ylamine. hydrobromide

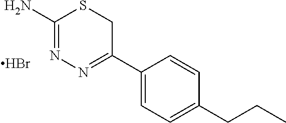 [SMILES: Br ; CCCc1ccc(C2=NN=C(N)SC2)cc1]

### A) Preparation of 2-bromo-1-(4-propyl-phenyl)-ethanone

To the stirred solution of 4-propyl acetophenone (5.0 g, 30.0 mmol) in CHCl₃ (50 mL) was added bromine (4.9 g, 30.0 mmol) in CHCl₃ (10 mL) at 0° C. and the mixture was stirred for another 30 min at the same temperature. The reaction was monitored by TLC and after completion of the reaction, sodium thiosulphate solution was added and was then subjected to a standard CH₂Cl₂ work-up to yield crude residue which was purified by column chromatography on silica gel using hexane:ethyl acetate (9:1) to give 4.7 g of the desired product as a liquid.

### B) Preparation of 5-(4-propyl-phenyl)-6H-[1,3,4]thiadiazin-2-ylamine hydrobromide

To the stirred solution of 2-bromo-1-(4-propyl-phenyl)-ethanone (4.0 g, 16.0 mmol) in EtOH (30 mL) was added thiosemicarbazide (1.5 g, 16.0 mmol) at RT and was stirred for overnight. The reaction was monitored by TLC and after completion of the reaction solvent was evaporated and the solid mass was purified by column chromatography on silica gel using MeOH:CH₂Cl₂ (1:9) to give the desired product (1.40 g) as a pink color solid.

### Example 13

### 5-(4-cyclohexyl-phenyl)-6H-[1,3,4]thiadiazine-2-ylamine.hydrobromide

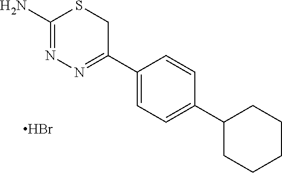 [SMILES: NC1=NN=C(c2ccc(C3CCCCC3)cc2)CS1 ; Br]

### A) Preparation of 2-Bromo-1-(4-cyclohexyl-phenyl)-ethanone

To the stirred solution of 4-cyclohexyl acetophenone (2.0 g, 9.8 mmol) in THF (30 mL) was added a catalytic amount of AlCl3 and bromine (2.0 g, 12.8 mmol) at 0° C. and the mixture was stirred for another 30 min at the same temperature. The reaction was monitored by TLC and after completion of the reaction sodium thiosulphate solution was added and was then subjected to a standard CH₂Cl₂ work-up to yield crude residue which was purified by column chromatography on silica gel using hexane:ethyl acetate (95:5) to give 2.1 g of the desired product as a liquid.

### B) Preparation of 5-(4-Cyclohexyl-phenyl)-6H-[1,3,4]thiadiazin-2 ylamine hydrobromide

To the solution of 2-bromo-1-(4-cyclohexyl-phenyl)-ethanone (2.0 g, 7.1 mmol) in EtOH (30 mL) was added thiosemicarbazide (650 mg, 13.4 mmol) at RT and was stirred for overnight. The reaction was monitored by TLC and after completion of the reaction solvent was evaporated and the solid mass was purified by column chromatography on silica gel using MeOH:CH₂Cl₂ (5:95) to give the pure desired product (900 mg) as a orange solid.

### Example 14

### 6-Butoxy-5,6,7,8-tetrahydro-4aH-benzo[1,3,4]thiadiazin-3-ylamine

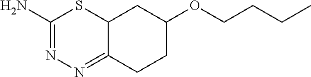 [SMILES: CCCCOC1CCC2=NN=C(N)SC2C1]

### A) Preparation of 1,4-dioxa-spiro[4.5]decan-8-ol

Sodium borohydride (370 mg, 9.6 mmol) was added portion wise to a stirred solution of 1,4-dioxa-spiro[4.5]decan-8-one (1.0 g, 6.4 mmol) in methanol (10 mL) over a period of 15 min at 0° C. Reaction mass was warmed to room temperature and stirred for 2 h. Methanol was evaporated and the residue diluted with water. Extracted with ethyl acetate, the organic layer dried over anhydrous sodium sulfate and evaporated to afford 0.9 g (90%) of 1, 4-dioxa-spiro[4.5]decan-8-ol as a brown oil. [TLC system: 3:7 Ethyl acetate/Pet ether; R_{f}value: 0.15]

### B) Preparation of 8-butoxy-1,4-dioxa-spiro[4.5]decane

Sodium hydride (60% in mineral oil; 1.0 g, 25.0 mmol) was added portion wise to a solution of 1, 4-dioxa-spiro[4.5]decan-8-ol (1.0 g, 6.3 mmol) in dimethylformamide (15 mL) over a period of 15 min at 0° C. and then stirred at room temperature for 30 min. Cooled to 0° C. and a solution of butyl iodide (2.3 g, 12.6 mmol) in dimethylformamide (10 mL) was added dropwise over a period of 15 min. Heated at reflux over night. Cooled to room temperature and the residue was partitioned between water ethyl acetate. The organic layer was washed with water, brine, dried over anhydrous sodium sulfate and concentrated in vacuum. The crude compound was purified by column chromatography over silica gel (60-120 mesh) using 5% ethyl acetate in pet ether as eluent to afford 360 mg (26.7%) of 8-butoxy-1,4-dioxa-spiro[4.5] decane as a brown oil. [TLC system: 1:1 Ethyl acetate/Pet ether; R_{f}value: 0.6]

### C) Preparation of 4-butoxy-cyclohexanone

2N Hydrochloric acid (10 mL) was added to a stirred solution of 8-butoxy-1,4-dioxa-spiro[4.5] decane (1.3 g, 6.1 mmol) in acetone (10 mL) and stirred for 1 h. The reaction mixture was partitioned between water and ethyl acetate. The organic layer was separated, washed with saturated sodium bicarbonate solution, brine, dried over sodium sulfate, concentrated in vacuo to afford 1.0 g (97%) of 4-butoxy-cyclohexanone as a brown oil. [TLC system: 1.5:8.5 Ethyl acetate/Pet ether; R_{f}value: 0.5]

### D) Preparation of 2-bromo-4-butoxy-cyclohexanone

Bromine (940 mg, 5.9 mmol) in chloroform (5 mL) was added dropwise to a stirred solution of 4-butoxy-cyclohexanone (1.0 g, 5.9 mmol) in chloroform (70 mL) over a period of 15 min at 0° C. Warmed to room temperature and stirred for 3 h. Diluted with water, the organic layer was separated and dried over anhydrous sodium sulfate. The solvent was evaporated and dried in vacuo to afford 800 mg (54.5%) of 2-bromo-4-butoxy-cyclohexanone as dark brown oil. [TLC system: 1.5:8.5 Ethyl acetate/Pet ether; R_{f}value: 0.65]

### E) Preparation of 6-butoxy-5,6,7,8-tetrahydro-4aH-benzo[1,3,4]thiadiazin-3-ylamine

Thiosemicarbazide (300 mg, 3.2 mmol) was added to a solution of 2-bromo-4-butoxy-cyclohexanone (80 mg, 3.2 mmol) in acetonitrile (10 mL) and stirring continued for 3 h at room temperature. The precipitated solid was filtered and washed with diethyl ether. The compound was purified by recrystallization from 1:3 methanol/diethyl ether to afford 150 mg (19.3%) of 6-butoxy-5,6,7,8-tetrahydro-4aH-benzo[1,3,4]thiadiazin-3-ylamine as a white solid. [TLC system: 1:9 Methanol/Chloroform; R_{f}value: 0.4]

### Example 15. 5-(4-phenyl-butyl)-6H-[1,3,4]thiadiazin-3-ylamine hydrobromide

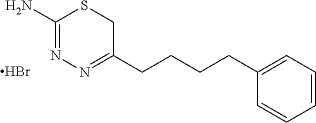 [SMILES: Br ; NC1=NN=C(CCCCc2ccccc2)CS1]

### A) Preparation of 2-acetyl-5-phenyl-pentanoic acid ethyl ester

Sodium (1.6 g, 69.5 mmol) was dissolved in ethanol (36 ml) and to this solution ethyl aceto acetate (2.50 ml, 19.6 mmol) was added and the reaction mixture was heated to reflux. At the reflux condition (3-bromo-propyl)-benzene (5.0 g, 21.8 mmol) was added portion wise in 3 h and reflux was continued for further 9 h. The reaction was monitored by TLC and after completion of the reaction, mixture was filtered and the filtrate was evaporated to give the crude material, which was used for the next step without purification as NMR and mass show desired compound has formed.

### B) Preparation of 6-phenyl-hexan-2-one

A solution of 2-acetyl-5-phenyl-pentanoic acid ethyl ester (10.0 g, 40.0 mmol) in 5% NaOH (56.5 ml) solution was allowed to stir for 5 h at RT. The reaction was monitored by TLC and after completion of the reaction, mixture was acidified with 50% H₂SO₄ (pH=2). Then the mixture was concentrated until mass becomes half, cooled the reaction mixture to 0° C. To this mixture saturated K₂CO₃ solution was added and the mixture was subjected to a standard ether work up to give the desired product as a liquid which was used for the next step without purification.

### C) Preparation of 1-bromo-6-phenyl-hexan-2-one

To a solution of 6-phenyl-hexan-2-one (3 g, 17.0 mmol) in dry methanol (12 mL) was added bromine (0.87 mL, 17.0 mmol) at 4° C. and the reaction mixture was kept at that temperature for 1.5 h, in that time color of the bromine was disappeared. Then 50 mL 1M K₂CO₃ was added, the mixture was concentrated in vacuo and extrated twice with 50 mL of ethyl acetate. The extracts were washed twice with 20 mL of 1M K₂CO₃, dried, and the solvent was evaporated. The residue was dissolved in 100 mL of THF and 50 mL of 1M H₂SO₄ and the mixture was heated to reflux for 1.5 h. Then the mixture was concentrated in vacuo and extracted with ethyl acetate. The extracts were washed with 2M KHCO₃, dried, and the solvent was evaporated to give 2.8 g of crude bromo ketone. The crude material was pure enough for next step of reaction.

### D) Preparation of 5-(4-phenyl-butyl)-6H-[1,3,4]thiadiazin-2-ylamine. hydrobromide

To the stirred solution of 1-bromo-6-phenyl-hexan-2-one (900 mg, 3.55 mmol) in ethanol (15 ml) was added thiosemicarbazide (320 mg, 3.51 mmol) at RT and was stirred for another 12 h at the same temperature. The reaction was monitored by TLC and after completion of the reaction, solvent was distilled off and the crude residue was purified by column chromatography using MeOH:CHCl₃ (1:9) to give 400 mg of the desired product with small impurities and was further purified by preparative HPLC to give pure desired product (340 mg) as a white solid.

### Example 16

### 5-(5-phenoxy-pentyl-6H-[1,3,4]thiadiazine-2-ylamine. hydrobromide

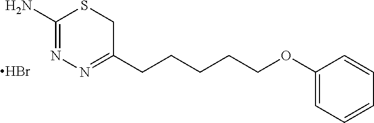 [SMILES: Br ; NC1=NN=C(CCCCCOc2ccccc2)CS1]

### A) Preparation of (5-bromo-pentyloxy)-benzene

To the stirred solution of phenol (20.0 g, 0.21 mol) in water (71 ml) was added 1,5-dibromo pentane (61.0 g, 0.26 mol) and the reaction mixture was heated to reflux and at the reflux condition aqueous NaOH (9.3 g in 21 ml water) solution was added. After addition reflux was continued for another 4.5 h. The progress of reaction was monitored by TLC and after completion of the reaction, mixture was cooled to RT and the upper layer was separated and discarded and the lower layer was washed with benzene. The combined benzene layers were washed with dilute NaOH solution and water. The organic layers were dried and then evaporated under reduced pressure to give crude desired product (61.0 g) and was purified by column chromatography using hexane to give 28.0 g of the pure desired product as a liquid.

### B) Preparation of 6-phenoxy-hexanenitrile

To the stirred solution of (5-bromo-pentyloxy)-benzene (28.0 g, 115.0 mmol) in ethanol (114 ml) was added aqueous KCN solution (9.0 g, 138.0 mmol) and the mixture was heated to reflux for 28 h. The progress of the reaction was monitored by TLC and after completion of the reaction, mixture was cooled to RT and then evaporated the ethanol and the aqueous layer was extracted with ethyl acetate and the ethyl acetate layer was washed with dilute NaOH solution and the organic layer was dried and evaporated to give desired product as a crystalline solid (20.0 g).

### C) Preparation of 6-phenoxy hexanoic acid

To the stirred solution of 6-phenoxy-hexanenitrile (8.6 g, 45.0 mmol) in ethanol (40 ml) was added aqueous NaOH solution (3.6 g in 20 ml water) and after addition the reaction mixture was heated to reflux for overnight. The progress of the reaction was monitored by TLC and after completion of the reaction, mixture was cooled to 0° C. and pH of the reaction mixture was adjusted to 4 by adding 6N HCl and was then subjected to a standard CH₂Cl₂ work up to give 9.4 g of the desired product as a white crystalline solid.

### D) Preparation of 6-phenoxy-hexanoyl chloride

To the stirred solution of 6-phenoxy hexanoic acid (5.0 g, 24.0 mmol) in dry toluene (50 ml) was added thionyl chloride (2.1 ml, 28.8 mmol, in 0.5 ml DMF) at 0° C. and after addition temperature of the reaction mixture was slowly raised to RT and then refluxed for 3 h. The progress of the reaction was monitored by TLC and after completion of the reaction solvent was evaporated to give 5.0 g of the desired product as a black liquid and was proceed further with the crude material.

### E) Preparation of 1-bromo-7-phenoxy-heptan-2-one

To the stirred solution of 6-phenoxy-hexanoyl chloride (5 g, 22.0 mmol) in CH₂Cl₂ was added an ethereal solution of CH₂N₂ (88.0 mmol, prepared from nitrosomethylurea) at −10° C. After 1 h at this temperature, the solvent was removed at 0° C. by means of a stream of N₂. The crude diazoketone was dissolved in CH₂Cl₂ (ml) and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (2:98) as the eluting solvent to give desired product (2.8 g) as crystalline solid.

### F) Preparation of 5-(5-phenoxy-pentyl)-6H-[1,3,4]thiadiazin-2-ylamine hydrobromide

To the stirred solution of 1-bromo-7-phenoxy-heptan-2-one (894 mg, 9.8 mmol) in ethanol (30 ml) was added thiosemicarbazide (2.80 g, 9.8 mmol) at RT and was stirred for another 12 h at the same temperature. The reaction was monitored by TLC and after completion of the reaction, solvent was distilled off and the crude residue was purified by column chromatography using MeOH:CH₂Cl₂ (1:9) to give 400 mg of the desired product with small impurities and was further purified by preparative HPLC to give pure desired (1.2 g) product as a grey solid.

### Example 17

### 10,10a-dihydro-9H-1-thia-3,4-diaza-phenanthren-2-ylamine hydrobromide

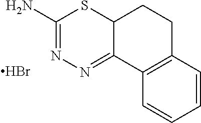 [SMILES: Br ; NC1=NN=C2c3ccccc3CCC2S1]

### A) Preparation of 2-Bromo-3,4-dihydro-211-naphthalen-1-one

To the stirred solution of 3,4-dihydro-2H-naphthalen-1-one (5.0 g, 34.0 mmol) in dry ether (20 ml) was added NBS (6.4 g, 0.36 mol) and NH₄OAc (260 mg, 3.40 mmol) in portion wise at 0° C. and was then stirred for 1 h at the same temperature. After that temperature of the reaction mixture was slowly raised to RT and was then stirred at the same temperature for another 1 h. The reaction was monitored by TLC and after completion of the reaction mixture was filtered and the filtrate was washed with water and brine. The organic layer was dried over Na₂SO₄ and the filtrate was concentrated to give 5.4 g of the desired crude product, which was used for the next step without further purification.

### B) Preparation of 10,10a-dihydro-9H-1-thia-3,4-diaza-phenanthren-2-ylamine hydrobromide

To the stirred solution of 2-bromo-3, 4-dihydro-2H-naphthalen-1-one (5.4 g, 23.0 mmol) in ethanol (50 ml) was added thiosemicarbazide (1.60 g, 17.9 mmol) at RT and was stirred for another 12 h at the same temperature. The reaction was monitored by TLC and after completion of the reaction, solvent was distilled off and the crude residue was purified by column chromatography using MeOH:CHCl₃ (1:9) to give pure desired product (1.2 g) as a brown solid.

### Example 18

### (2-Amino-6H-[1,3,4]thiadiazin-5-yl)-acetic acid isopropyl ester

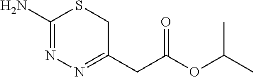

### A) 5-(2-Chloro-1-hydroxy-ethylidene)-2,2-dimethyl-[1,3]dioxane-4,6-dione

Pyridine (8.22 g, 69.44 mmol) was added to a solution of 2,2-Dimethyl-[1,3]dioxane-4,6-dione (5.0 g, 34.72 mmol) in dichloromethane (50 mL) followed by drop wise addition of chloroacetyl chloride (7.84 g, 69.44 mmol). The reaction mass was stirred for 2½ h at room temperature. The volatiles were evaporated in vacuo to get 5.2 g (60%) of 5-(2-chloro-1-hydroxy-ethylidene)-2, 2-dimethyl-[1, 3]-dioxane-4, 6-dione 20a, which was used for the subsequent step without any further purification. [TLC system: 1:9 Methanol/Chloroform; R_{f}value: 0.25]

### B) 4-Chloro-3-oxo-butyric acid isopropyl ester

A solution of 20a (5.1 g, 23.18 mmol) in isopropanol (50 mL) was refluxed for 3 h. Excess isopropanol was evaporated to get crude which was purified by column chromatography over silica gel (60-120 mesh) using 8% ethyl acetate in pet ether as eluent to afford 1.1 g (27%) of 4-Chloro-3-oxo-butyric acid isopropyl ester 20b. [TLC system: 1.5:8.5 Ethyl acetate/Pet ether; R_{f}value: 0.75]

### C) (2-Amino-6H-[1,3,4]thiadiazin-5-yl)-acetic acid isopropyl ester

20b (0.5 g, 2.80 mmol) was dissolved in acetonitrile (10 mL) and thiosemicarbazide (0.25 g, 2.80 mmol) and stirred for 2 h at room temperature. A white precipitate formed, the solid was filtered and washed with acetonitrile and then with ether. The solid was dissolved in water, basified with sodium bicarbonate solution, extracted with ethyl acetate and the extracts dried over anhydrous sodium sulfate. Concentration in vacuo afforded 0.25 g (42%) of (2-amino-6H-[1, 3, 4]-thiadiazin-5-yl)-acetic acid isopropyl ester as a solid. [TLC system: 1:9 Methanol/Chloroform; R_{f}value: 0.4]

### Example 19

### 5-heptyl-4H-[1,3,4]thiadiazin-2-ylamine

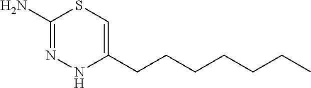

### A) Preparation of 2-acetyl-octanoic acid ethyl ester

Sodium (1.2 g, 54.5 mmol) was dissolved in ethanol (27 ml) and to this solution ethyl aceto acetate (6.90 ml, 54.5 mmol) was added and the reaction mixture was heated to reflux. At the reflux condition 1-bromo hexane (10.0 g, 60.0 mmol) was added dropwise in 3 h and reflux was continued for further 9 h. The reaction was monitored by TLC and after completion of the reaction, mixture was filtered and the filtrate was evaporated to give the crude material (4.0 g), which was used for the next step without purification as NMR and mass show desired compound has formed.

### B) Preparation of nonan-2-one

A solution of 2-acetyl-octanoic acid ethyl ester (4.0 g, 18.6 mmol) in 5% aqueous NaOH solution (22.6 ml) was allowed to stir for 5 h at RT. The reaction was monitored by TLC and after completion of the reaction, mixture was acidified with 50% H₂SO₄ (pH=2). Then the mixture was concentrated until mass becomes half, cooled the reaction mixture to 0° C. To this mixture saturated K₂CO₃ solution was added and the mixture was subjected to a standard ether work up to give 1.0 g of the desired product as a liquid, which was used for the next step without purification.

### C) Preparation of 1-bromo-nonan-2-one

To a solution of 6 nonan-2-one (800 mg, 5.60 mmol) in dry methanol (4 ml) was added bromine (0.90 g, 0.30 ml, 5.6 mmol) at 4° C. and the reaction mixture was kept at that temperature for 1.5 h, in that time color of the bromine was disappeared. Then 50 ml 1M K₂CO₃ was added, the mixture was concentrated in vacuo and extracted twice with 50 ml of ethyl acetate. The extracts were washed twice with 20 ml of 1M K₂CO₃, dried, and the solvent was evaporated. The residue was dissolved in 100 ml of THF and 50 ml of 1M H₂SO₄ and the mixture was heated to reflux for 1.5 h. Then the mixture was concentrated in vacuo and extracted with ethyl acetate. The extracts were washed with 2M KHCO₃, dried, and the solvent was evaporated to give 600 mg of crude bromo ketone. The crude material was pure enough for next step of reaction.

### D) Preparation of 5-heptyl-4H-[1,3,4]thiadiazin-2-ylamine

To the stirred solution of 1-bromo-nonan-2-one (1.0 g, 11.4 mmol) in ethanol (25 ml) was added thiosemicarbazide (2.5 g, 11.4 mmol) at RT and was stirred for another 12 h at the same temperature. The reaction was monitored by TLC and after completion of the reaction, solvent was distilled off and the crude residue was purified by column chromatography using MeOH:CHCl₃ (5:95) to give 230 mg of the desired product with small impurities and was further purified by preparative HPLC to give 100 mg of the pure desired product as a dark brown semi solid.

### Example 20

### 4-(2-amino-4H-[1,3,4]thiadiazin-5-yl)-benzoic acid ethyl ester

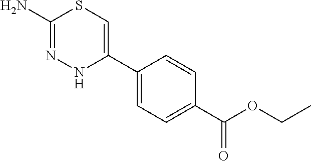 [SMILES: CCOC(=O)c1ccc(C2=CSC(N)=NN2)cc1]

### A) Preparation of 4-(2-bromo-acetyl)-benzoic acid ethyl ester

To the stirred solution of 4-acetyl-benzoic acid ethyl ester (3.0 g, 15.6 mmol) in dry CCl₄ (150 ml) was added bromine (0.75 ml, 14.8 mmol, in 15 ml CCl₄) very slowly at −10° C. to 0° C. After addition temperature of the reaction mixture was slowly raised to RT and stirred at RT for 1 h. The reaction was monitored by TLC and after completion of the reaction aqueous sodium thiosulphate solution was added to the reaction mixture and was subjected to a standard CH₂Cl₂ work up to give 4.40 g of the desired product as a liquid.

### B) Preparation of 4-(2-amino-4H-[1,3,4]thiadiazin-5-yl)-benzoic acid ethyl ester

To the stirred solution of 4-(2-bromo-acetyl)-benzoic acid ethyl ester (4.4 g, 16.2 mmol) in ethanol (200 ml) was added thiosemicarbazide (1.47 g, 16.2 mmol) at RT and was stirred for another 12 h at the same temperature. The reaction was monitored by TLC and after completion of the reaction, solvent was distilled off and the crude residue (4.0 g) was purified by column chromatography using MeOH:CHCl₃ (1:9) to give pure desired product (200 mg) as a light brown solid.

### Example 21

### 5-(4-phenyl-butyl)-6H-[1,3,4]thiadiazin-2-ylamine. hydrobromide

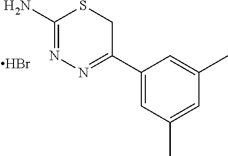

### A) Preparation of 1-(3,5-dimethyl-phenyl)-ethanone

To the stirred solution of 1-iodo-3, 5-dimethyl benzene (2.0 g, 8.61 mmol) in 40 ml anhydrous DMF (degassed by argon) was added LiCl (1,80 g, 43.00 mmol), Pd(dba)₃ (98.6 mg, 0.11 mmol), EtNⁱPr₂ (2.95 ml, 17.23 mmol) and acetic anhydride (2.5 ml). The flask was degassed by argon and the mixture was stirred for 7 h at 100° C. The reaction was monitored by TLC and after completion of the reaction, mixture was cooled and then subjected to a standard ether work up to give 1.4 g of the desired product as a liquid and was used for the next step without purification as NMR and mass show desired compound has formed.

### B) Preparation of 2-bromo-1-(3,5-dimethyl-phenyl)-ethanone

To the stirred solution of 1-(3,5-dimethyl-phenyl)-ethanone (1.40 g, 9.4 mmol) in dry CCl₄ (100 ml) was added bromine (0.50 ml, 9.4 mmol, in 10 ml CCl₄) very slowly at −10° C. to 0° C. After addition temperature of the reaction mixture was slowly raised to RT and stirred at RT for 1 h. The reaction was monitored by TLC and after completion of the reaction sodium thiosulphate solution was added to the reaction mixture and was subjected to a standard CH₂Cl₂ work up to give 1.0 g of the desired product as a liquid and was used for the next step without further purification as the NMR the crude shows desired compound has formed.

### C) Preparation of 5-(3,5-dimethyl-phenyl)-6H-[1,3,4]-thiadiazin-2-ylamine

To the stirred solution of 2-bromo-1-(3,5-dimethyl-phenyl)-ethanone (1.0 g, 3.60 mmol) in ethanol (25 ml) was added thiosemicarbazide (328 mg, 3.60 mmol) at RT and was stirred for another 12 h at the same temperature. The reaction was monitored by TLC and after completion of the reaction, solvent was distilled off and the crude residue was purified by column chromatography using MeOH:CHCl₃ (1:9) to give 200 mg of the desired product as a white solid.

### Example 22

### 5-(3-methoxy-propyl)-6H-[1,3,4]thiadiazin-2-ylamine. hydrobromide

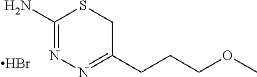

### A) Preparation of 4-methoxy-butan-1-ol

To the stirred solution of NaH (13.5 g, 50%, 0.14 mol) in THF (250 mL) was added 1, 4-butane diol (25.0, 0.277 mol) at 0° C. under nitrogen atmosphere for 20 min and then MeI was added drop wise at the same temperature. After addition temperature of the reaction mixture was slowly raised to RT and stirred at RT for 16 h. The reaction was monitored by TLC and after completion of the reaction, mixture was cooled to 0° C. and then added acetic acid to adjust pH to neutral. The whole reaction mixture was concentrated, filtered and the filtrate was distilled to give the desired product (7.50 g) as a pale yellow liquid.

### B) Preparation of 4-methoxy-butyric acid

To the stirred solution of 4-methoxy-butan-1-ol (14.2 g, 98.0 mmol) in acetone (200 mL) was added Jones reagent (70 mL) and the mixture was stirred at RT for 30 min. The reaction was monitored by TLC and after completion of the reaction; mixture was quenched by isopropyl alcohol until color become green. Then mixture was filtered and acetone was distilled off from the filtrate. After that mixture was saturated with aqueous solution of NaCl and was subjected to a standard ether work up to give the crude residue (18.1 g) as a yellow liquid.

### C) Preparation of 4-methoxy-butyryl chloride

To the stirred solution of 4-methoxy-butyric acid (18.0 g, 0.15 mol) in toluene (100 mL) was added thionyl chloride (11.3 mL, 0.15 mol) at 0° C. After addition temperature of the reaction mixture was slowly raised to RT and then refluxed at 120° C. for 3 h. The reaction was monitored by TLC and after completion of the reaction; solvent was distilled off to give 19.0 g crude; which was directly used for the next step without purification.

### D) Preparation of 1-bromo-5-methoxy-pentan-2-one

To the stirred solution of 4-methoxy-butyryl chloride (9.0 g, 65.8 mmol) in ether (30 mL) was added an ethereal solution of CH₂N₂ (0.26 mmol, prepared from nitrosomethylurea) at 0° C. After 1 h at this temperature, the solvent was removed at 0° C. by means of a stream of N₂. The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (20:80) as the eluting solvent to give desired product (4.0 g) as a brown liquid.

### E) Preparation of 5-(3-methoxy-propyl)-6H-[1,3,4]thiadiazin-2-ylamine. hydrobromide

To a solution of 1-bromo-5-methoxy-pentan-2-one (4.0 g, 20.5 mmol) in EtOH (40 ml) at RT was added thiosemicarbazide (1.70 g, 18.4 mmol) and was stirred for overnight. The reaction was monitored by TLC and after completion of the reaction; mixture was filtered and the solid mass (2.3 g) was purified by column chromatography on silica gel using MeOH:CHCl₃ (5:95) to give 250 mg of the desired product as a yellow solid.

### Example 23

### 5-(5-methoxy-pentyl)-6H-[1,3,4]thiadiazin-2-ylamine. hydrobromide

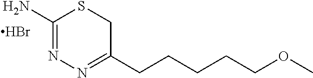

### A) Preparation of 6-methoxy-hexan-1-ol

To the stirred solution of hexane-1, 6-diol (50.0 g, 0.42 mol) in THF (750 mL) was added sodium hydride (considered 50% in oil, 20.3 g, 0.84 mol) portion wise over a period of 30 min at 0° C. and stirred for 30 min at the same temperature. After that methyl iodide was added dropwise over a period of 20 min and then stirred at RT for 16 h. The reaction was monitored by TLC and after completion of the reaction; mixture was cooled to 0° C. followed by addition of ice-cold water. The reaction mixture was concentrated, dissolved the crude in 500 mL of water and then extracted with ethyl acetate. The combined organic layers were dried over Na₂SO₄ and evaporated to give the crude material (52.4 g), which was used for the next step without purification as GCMS shows desired compound has formed.

### B) Preparation of 6-methoxy-hexanoic acid

To the stirred solution of 6-methoxy-hexan-1-ol (52.4 g, 0.39 mol) in acetone (500 mL) was added dropwise a Jones reagent (approx. 150 mL; which was prepared by diluting a solution of 53.4 g of CrO₃ in 46 mL of conc. H₂SO₄ with water to a volume of 200 mL at 0° C.) until the color of the reaction turns reddish brown (color of Jones reagent). Then isopropanol was added to destroy the excess reagent until the color of the reaction mixture is green; solvent was evaporated to give the crude residue; which was dissolved in water and extracted with CH₂Cl_{2.} The combined organic layers were then dried over Na₂SO₄ to give the crude residue (47.4 g). The crude residue was then purified by column chromatography using ethyl acetate as eluent to give the pure desired product (35.0 g) as a liquid.

### C) Preparation of 1-bromo-7-methoxy-heptan-2-one

To the stirred solution of 6-methoxy-hexanoic acid (10.0 g, 68.4 mmol) in dry toluene (100 mL) in presence of catalytic amount of DMF was added thionyl chloride (6.1 mL, 82.1 mmol) at 0° C. After addition temperature of the reaction mixture was slowly raised to RT and then refluxed at 110° C. for 3 h. The reaction was monitored by TLC and after completion of the reaction; solvent was distilled off and the crude was dissolved in dry ether (50 mL). To this solution was added an ethereal solution of CH₂N₂ (prepared from nitrosomethylurea) at 0° C. After 1 h at this temperature, the solvent was removed at 0° C. by means of a stream of N_{2.} The crude diazoketone was dissolved in dry CH₂Cl₂ and the solution was cooled to −78° C. A saturated solution of HBr in CH₂Cl₂ was then added dropwise, care being taken that excess HBr solution was not added. When the diazoketone has been consumed (TLC analysis), the temperature of the reaction mixture was lowered to −78° C. and a stream of N₂ was passed through the solution until the solvent volume had been reduced to about one-third of the original. The remainder of the solvent was then removed in vacuo at RT to give the desired crude product (4.1 g) as a liquid and used for the next step without purification as GCMS shows desired compound has formed.

### D) Preparation of 5-(5-methoxy-pentyl)-6H-[1,3,4]thiadiazin-2-ylamine. hydrobromide

To the stirred solution of crude 1-bromo-7-methoxy-heptan-2-one (75% purity, 14.3 g, 64.0 mmol) in EtOH (35 mL) at RT was added thiosemicarbazide (4.50 g, 48.0 mmol) and stirred for overnight. The reaction was monitored by TLC and after completion of the reaction; mixture was filtered and solid mass was obtained. We have repeated the last two batches with same scale and purified together by column chromatography on silica gel using MeOH-CH₂Cl₂ (MeOH ranges from 5-7%) to give the desired product (7.0 g) as an off-white solid.

### Example 24

### 3-(2-amino-6H-[1,3,4]thiadiazin-5-yl)-benzoic acid ethyl ester hydrobromide

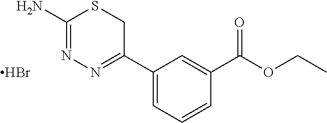 [SMILES: Br ; CCOC(=O)c1cccc(C2=NN=C(N)SC2)c1]

### A) Preparation of 3-acetyl-benzoic acid ethyl ester

To the stirred solution of 3-acetyl-benzoic acid (1.0 g, 6.09 mmol) in toluene (50 mL) was added thionyl chloride (0.55 mL, 7.5 mmol) at 0° C. After addition temperature of the reaction mixture was slowly raised to RT and then refluxed at 120° C. for 3 h. The reaction was monitored by TLC and after completion of the reaction; ethanol was added dropwise at 0° C. After addition temperature of the reaction mixture was slowly raised to RT and stirred at RT for 48 h. After that ethanol was removed from the reaction mixture and was subjected to standard ethyl acetate work up to give the desired product (900 mg) as a liquid.

### B) Preparation of 3-(2-bromo-acetyl)-benzoic acid ethyl ester

To the stirred solution of 3-acetyl-benzoic acid ethyl ester (900 mg, 4.68 mmol) in dry CCl₄ (150 ml) was added bromine (0.20 mL, 4.20 mmol, in 10 ml CCl₄) very slowly at −10° C. to 0° C. After addition temperature of the reaction mixture was slowly raised to RT and then stirred at RT for 1 h. The reaction was monitored by TLC and after completion of the reaction aqueous sodium thiosulphate solution was added to the reaction mixture and was subjected to a standard CH₂Cl₂ work up to give 1.0 g of the desired product as a liquid and was used for the next step without purification.

### C) Preparation of 3-(2-amino-6H-[1,3,4]thiadiazin-5-yl)-benzoic acid ethyl ester

To the stirred solution of 3-(2-bromo-acetyl)-benzoic acid ethyl ester (1.0 g, 3.68 mmol) in ethanol (100 ml) was added thiosemicarbazide (336 mg, 3.68 mmol) at RT and was stirred for another 12 h at the same temperature. The reaction was monitored by TLC and after completion of the reaction, solvent was distilled off and the crude residue was purified by column chromatography using MeOH:CHCl₃ (5:95) to give the desired product (250 mg) as an orange solid.

### Example 25

### 5-[3-(butoxy-phenyl)-6H-[1,3,4]thiadiazin-2-ylamine

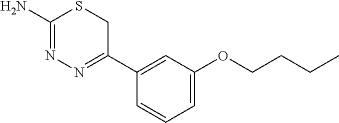 [SMILES: CCCCOc1cccc(C2=NN=C(N)SC2)c1]

### A) Preparation of 1-(3-butoxy-phenyl)-ethanone

To the stirred solution of 1-(3-hydroxy-phenyl)-ethanone (5.0 ml, 36.7 mmol) in DMF (50 ml) was added K₂CO₃ (6.1 g, 44.0 mmol), [18] crown ether (catalytic) and butyl bromide (4.7 mL, 44.0 mmol) slowly at 0° C. After addition temperature of the reaction mixture was slowly raised to RT and stirred at the RT for 2 h. The progress of reaction was monitored by TLC and after completion of the reaction, water was added and was subjected to a standard ethyl acetate work up to give the crude residue; which was purified by column chromatography using hexane:ethyl acetate (9:1) to give the desired product (7.0 g) as a solid.

### B) Preparation of 2-bromo-1-(3-butoxy-phenyl)-ethanone

To the stirred solution of 1-(3-butoxy-phenyl)-ethanone (5.0 g, 26.0 mmol) in dry CHCl₃ (50 ml) was added bromine (1.30 mL, 26.0 mmol, in 15 ml CHCl₃) very slowly at −10° C. to 0° C. After addition temperature of the reaction mixture was slowly raised to RT and stirred at RT for 1 h. The reaction was monitored by TLC and after completion of the reaction sodium thiosulphate solution was added to the reaction mixture and was subjected to a standard ethyl acetate work up to give 1.0 g of the desired product as a liquid and was used for the next step without purification as the NMR of the crude shows desired compound has formed.

### C) Preparation of 5-[3-(butoxy-phenyl)-6H-[1,3,4]thiadiazin-2-ylamine

To the stirred solution of 2-bromo-1-(3-butoxy-phenyl)-ethanone (2.4 g, 8.98 mmol) in ethanol (25 ml) was added thiosemicarbazide (820 mg, 8.98 mmol) at RT and was stirred for another 12 h at the same temperature. The reaction was monitored by TLC and after completion of the reaction, solvent was distilled off and the crude residue was purified by column chromatography using MeOH:CH₂Cl₂ (1:9) to give pure desired product (200 mg) as yellow solid.

### Example 26

### 5-(4a, 8a-dihydro-naphthalen-1-yl)-6H-[1,3,4]thiadiazin-2-ylamine

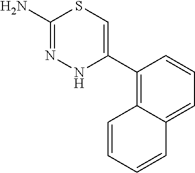 [SMILES: NC1=NNC(c2cccc3ccccc23)=CS1]

### A) Preparation of 2-bromo-1-naphthalen-1-yl-ethanone

To the stirred solution of 1-naphthalen-1-yl-ethanone (3.0 g, 17.62 mmol) in dry CCl₄ (200 ml) was added bromine (0.9 mL, 17.62 mmol, in 15 ml CCl₄) very slowly at −10° C. to 0° C. After addition temperature of the reaction mixture was slowly raised to RT and stirred at RT for 1 h. The reaction was monitored by TLC and after completion of the reaction sodium thiosulphate solution was added to the reaction mixture and was subjected to a standard CH₂Cl₂ work up to give crude desired product (4.5 g) and was used for the next step without purification as the NMR of the crude shows desired compound was formed.

### B) Preparation of 5-(4a, 8a-dihydro-naphthalen-1-yl)-6H-[1,3,4]thiadiazin-2-ylamine

To a solution of 2-bromo-1-naphthalen-1-yl-ethanone (4.5 g, 18.06 mmol) in EtOH (40 ml) at RT was added thiosemicarbazide (1.6 g, 18.06 mmol) and was stirred for overnight. The reaction was monitored by TLC and after completion of the reaction; mixture was filtered and the solid mass was purified by column chromatography on silica gel using MeOH:CH₂Cl₂ (1:9) to give 3.0 g of the desired product as a yellow solid.

### Example 27

### 5-naphthalen-2-yl-6H-[1,3,4]thiadiazin-2-ylamine. hydrobromide

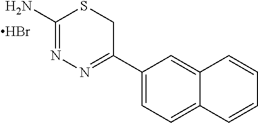 [SMILES: Br ; NC1=NN=C(c2ccc3ccccc3c2)CS1]

### A) Preparation of 2-bromo-1-naphthalen-2-yl-ethanone

To the stirred solution of 1-naphthalen-2-yl-ethanone (5.0 g, 29.0 mmol) in dry CHCl₃ (50 ml) was added bromine (1.5 mL, 29.0 mmol, in 15 ml CHCl₃) very slowly at −10° C. to 0° C. After addition temperature of the reaction mixture was slowly raised to RT and stirred at RT for 1 h. The reaction was monitored by TLC and after completion of the reaction sodium thiosulphate solution was added to the reaction mixture and was subjected to a standard ethyl acetate work up to give 1.0 g of the desired product as a liquid and was purified by column chromatography using 15% ethyl acetate-hexane to give the pure desired product (4.6 g) as a liquid.

### B) Preparation of 5-naphthalen-2-yl-6H-[1,3,4]thiadiazin-2-ylamine. hydrobromide

To the stirred solution of 2-bromo-1-naphthalen-2-yl-ethanone (4.60 g, 18.0 mmol) in EtOH (40 ml) at RT was added thiosemicarbazide (1.68 g, 18.0 mmol) and was stirred for overnight. The reaction was monitored by TLC and after completion of the reaction; mixture was filtered and the solid mass was purified by column chromatography on silica gel using MeOH:CH₂Cl₂ (1:9) to give 1.6 g of the desired product as a yellow solid.

### Example 28

### 5-[2-(4-bromo-phenoxy)-ethyl]-6H-[1,3,4]thiadiazin-2-ylamine

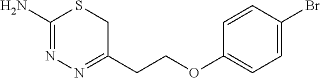 [SMILES: NC1=NN=C(CCOc2ccc(Br)cc2)CS1]

### A) Preparation of 4-phenoxy-butan-2-one

To the stirred solution of phenol (112.8 g, 1.2 mol) in CH₃CN at −78° C. was added methyl vinyl ketone (10 mL, 0.12 mol) and PMe₃ (1M in THF, 6 mL, 6.0 mmol). After addition temperature of the reaction mixture was slowly raised to 45° C. and stirred at the same temperature for 20 h. The reaction was monitored by TLC and after completion of the reaction, water was added to the mixture and was subjected to a standard ethyl acetate work up to give the crude product; which was purified by column chromatography using hexane:ethyl acetate (9:1) to give the desired product (1.4 g) as a liquid.

### B) Preparation of 1-bromo-4-(4-bromo-phenoxy)-butan-2-one

To the stirred solution of 4-phenoxy-butan-2-one (1.0 g, 6.0 mmol) in dry methanol (4 mL) was added bromine (0.30 mL, 6.0 mmol) at 0° C. and after that temperature of the reaction mixture was slowly raised to RT and kept at that temperature for 1.5 h, in that time color of the bromine was disappeared. Then MeOH was distilled off and dissolved the residue in 1M KHCO₃ (12 mL) solution and then extracted with ethylacetate and concentrated. The residue was dissolved in 200 mL of THF (24 mL) and then 1M H₂SO₄ (12 mL) was added and the mixture was heated to reflux for 1 h. After that mixture was concentrated in vacuo and then extracted with ethyl acetate. The extract was washed with 2M KHCO₃ solutions, dried, and the solvent was evaporated to give 1.12 g of crude bromo ketone. The crude material was pure enough for next step of reaction.

### C) Preparation of 5-[2-(4-bromo-phenoxy)-ethyl]-6H-[1,3,4]thiadiazin-2-ylamine

To the stirred solution of 1-bromo-4-(4-bromo-phenoxy)-butan-2-one (470 mg, 1.90 mmol) in EtOH (4.7 ml) at RT was added thiosemicarbazide (177 mg, 1.9 mmol) and was stirred for overnight. The reaction was monitored by TLC and after completion of the reaction; mixture was filtered and solid mass (640 mg) was purified by column chromatography on silica gel using MeOH:CH₂Cl₂ (1:9) to give desired product (35 mg) as green solid.

### Example 29. 5-(2-phenoxy-ethyl)-6H-[1,3,4]thiadiazin-2-ylamine. hydrobromide

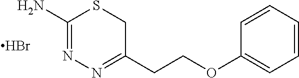 [SMILES: Br ; NC1=NN=C(CCOc2ccccc2)CS1]

A) Preparation of 1-bromo-4-phenoxy-butan-2-one

To the stirred solution of 3-phenoxy-propionic acid (6.0 g, 36.0 mmol) in toluene (30 mL) was added thionyl chloride (3.2 mL, 43.2 mmol) at 0° C. After addition temperature of the reaction mixture was slowly raised to RT and then refluxed at 120° C. for 3 h. The reaction was monitored by TLC and after completion of the reaction; solvent was distilled off. The crude was dissolved in ether and to this solution was added an ethereal solution of CH₂N₂ (prepared from nitrosomethylurea) at 0° C. After 1 h at this temperature, the solvent was removed at 0° C. by means of a stream of N_{2.} The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (10:90) as the eluting solvent to give desired product (2.30 g) as a brown liquid and was used for the next step without purification.

### B) Preparation of 5-(2-phenoxy-ethyl)-6H-[1,3,4]thiadiazin-2-ylamine. hydrobromide

To a solution of 1-bromo-4-phenoxy-butan-2-one (400 mg, 1.65 mmol) in EtOH (8 ml) at RT was added thiosemicarbazide (150 mg, 1.65 mmol) and was stirred for overnight. The reaction was monitored by TLC and after completion of the reaction; mixture was filtered and the solid mass was purified by column chromatography on silica gel using MeOH:CH₂Cl₂ (10:90) to give 230 mg of the desired product as a yellow solid.

### Example 30

### 5-[2-(2-methoxy-ethoxy)-ethyl]-6H-[1,3,4]thiadiazin-2-ylamine.hydrobromide

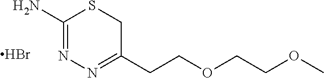

### A) Preparation of 3-(2-methoxy-ethoxy)-propionic acid methyl ester

To the stirred solution of 2-methoxy ethanol (10.0 g, 0.13 mol) in methyl acrylate (11.0 g, 0.13 mol) was added sodium (300 mg) at 0° C. and was stirred at RT for 16 h under nitrogen atmosphere. The reaction was monitored by TLC and after completion of the reaction; mixture was diluted with ether and was washed with water. The ether layer was dried (Na₂SO₄) and concentrated to give 13.0 g of the desired product as a liquid and was used for next reaction without purification as the NMR shows desired compound has formed.

### B) Preparation of 3-(2-methoxy-ethoxy)-propionic acid

To the stirred solution of 3-(2-methoxy-ethoxy)-propionic acid methyl ester (10.0 g, 0.061 mol) in ethanol (100 mL) was added aqueous solution of NaOH (3.20 g, 0.08 mol, in 100 mL water) at RT and was stirred for 16 h at RT. The reaction was monitored by TLC and after completion of the reaction; ethanol was evaporated and the pH of the reaction mixture was adjusted to 4.5 using 6N HCl. After that reaction was subjected to a standard chloroform work up to give the desired product (8.3 g) as a liquid and was used for next reaction without purification, as the NMR shows desired compound has formed.

### C) Preparation of 3-(2-methoxy-ethoxy)-propionyl chloride

To the stirred solution of 3-(2-methoxy-ethoxy)-propionic acid (6.0 g, 36.0 mmol) in toluene (30 mL) was added thionyl chloride (3.2 mL, 43.2 mmol) at 0° C. After addition temperature of the reaction mixture was slowly raised to RT and then refluxed at 120° C. for 3 h. The reaction was monitored by TLC and after completion of the reaction; solvent was distilled off and was directly used for the next step.

### D) Preparation of 5-[2-(2-methoxy-ethoxy)-ethyl]-6H-[1,3,4]thiadiazin-2-ylamine.hydrobromide

To the stirred solution of 3-(2-methoxy-ethoxy)-propionyl chloride (5.0 g, 0.034 mol) in ether (30 mL) was added an ethereal solution of CH₂N₂ (prepared from nitrosomethylurea) at 0° C. After 1 h at this temperature, the solvent was removed at 0° C. by means of a stream of N₂. The crude diazoketone was dissolved in ether and the solution was cooled to −70° C. A saturated solution of HBr gas in CH₂Cl₂ was then added dropwise, care being taken that excess HBr solution was not added. When diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The crude reaction mixture was dissolved in EtOH (30 mL) and thiosemicarbazide was added to it. After addition mixture was stirred for overnight at RT. The reaction was monitored by TLC and after completion of the reaction; mixture was filtered and the solid mass was purified by column chromatography on silica gel using MeOH:CHCl₃ (1:9) to give desired product (400 mg) as brown sticky solid.

### Example 31

### 5-(2-hexyloxy-ethyl)-6H-[1,3,4]thiadiazin-2-ylamine. hydrobromide

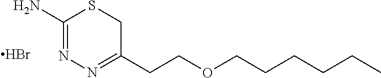 [SMILES: Br ; CCCCCCOCCC1=NN=C(N)SC1]

### A) Preparation of 4-hexyloxy-butan-2-one

To the stirred solution of methyl vinyl ketone (3.80 g, 4.50 mL, 53.8 mmol) and n-hexanol (5.0 g, 48.9 mmol) was added 50% H₂SO₄ (1.0 mL) at 0° C. and after addition temperature of the reaction mixture was slowly raised to RT and was stirred at RT for 1 h. The reaction was monitored by TLC and after completion of the reaction ether was added and was then subjected to a standard ether work-up to yield desired product (9.0 g) as a liquid.

### B) Preparation of 1-bromo-4-hexyloxy-butan-2-one

To a solution of 4-hexyloxy-butan-2-one (9.0 g, 52.0 mmol) in dry methanol (30 mL) was added bromine (2.4 mL, 47.0 mmol) at 0° C. and the reaction mixture was kept at that temperature for 1.5 h, in that time color of the bromine was disappeared. Then aqueous K₂CO₃ solution (1M, 170 mL) was added, the mixture was concentrated in vacuum and extracted twice with 200 mL of ethyl acetate. The extracts were washed twice with 70 mL of 1M K₂CO₃ solutions, dried, and the solvent was evaporated. The residue was dissolved in 680 mL of THF and 170 mL of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give 4.40 g of crude bromo ketone. The crude material was pure enough for next step of reaction.

### C) Preparation of 5-(2-hexyloxy-ethyl)-6H-[1,3,4]thiadiazin-2-ylamine. hydrobromide

To the stirred solution of 1-bromo-4-hexyloxy-butan-2-one (4.4 g, 17.5 mmol) in EtOH (40 ml) at RT was added thiosemicarbazide (1.60 g, 17.5 mmol) and was stirred for overnight. The reaction was monitored by TLC and after completion of the reaction; mixture was filtered and the solid mass was purified by column chromatography on silica gel using MeOH:CH₂Cl₂ (1:9) to give 1.2 g of the desired product as an off-white solid.

### Example 32

### 5-[2-(biphenyl-4-ylmethoxy)-ethyl]-6H-[1,3,4]-thiadiazin-2-ylamine.hydrobromide

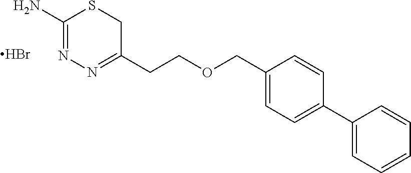 [SMILES: Br ; NC1=NN=C(CCOCc2ccc(-c3ccccc3)cc2)CS1]

### A) Preparation of 4-(biphenyl-4-ylmethoxy)-butan-2-one

To the stirred solution of biphenyl-4-methanol (3.0 g, 16.2 mmol) and methyl vinyl ketone (1.14 g, 16.2 mmol) was added 50% H₂SO₄ (1.0 mL) at 0° C. and after addition temperature of the reaction mixture was slowly raised to RT and was stirred at RT for 1 h. The reaction was monitored by TLC and after completion of the reaction ether was added and was then subjected to a standard ether work-up to yield desired product (580 mg) as a liquid.

### B) Preparation of 4-(biphenyl-4-ylmethoxy)-1-bromo-butan-2-one

To a solution of 4-(biphenyl-4-ylmethoxy)-butan-2-one (1.58 g, 6.2 mmol) in dry methanol (6 mL) was added bromine (0.31 mL, 6.2 mmol) at 0° C. and the reaction mixture was kept at that temperature for 1.5 h, in that time color of the bromine was disappeared. Then aqueous K₂CO₃ solution (1M, 18 mL) was added, the mixture was concentrated in vacuum and extracted twice with 100 mL of ethyl acetate. The extracts were washed twice with 30 mL of 1M K₂CO₃ solutions, dried, and the solvent was evaporated. The residue was dissolved in 36 mL of THF and 18 mL of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give 1.93 g of crude bromo ketone. The crude material was pure enough for next step of reaction.

### C) Preparation of 5-[2-(biphenyl-4-ylmethoxy)-ethyl]-6H-[1,3,4]-thiadiazin-2-ylamine.hydrobromide

To the stirred solution of 4-(biphenyl-4-ylmethoxy)-1-bromo-butan-2-one (2.59 g, 7.8 mmol) in EtOH (25 ml) at RT was added thiosemicarbazide (710 mg, 7.8 mmol) and was stirred for overnight. The reaction was monitored by TLC and after completion of the reaction; mixture was filtered and the solid mass was purified by column chromatography on silica gel using MeOH:CH₂Cl₂ (1:9) to give the desired product (50 mg) as white solid.

### Example 33

### 5-[2-(4-methyl-benzyloxy)-ethyl]-6H-[1,3,4]thiadiazin-2-ylamine.hydrobromide

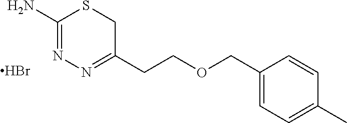 [SMILES: Br ; Cc1ccc(COCCC2=NN=C(N)SC2)cc1]

### A) Preparation of 4-(4-methyl-benzyloxy)-butan-2-one

To the stirred solution of methyl vinyl ketone (5.0 g, 71.0 mmol) and p-tolyl methanol (8.7 g, 71.0 mmol) was added 50% H₂SO₄ (2.0 mL) at 0° C. and after addition temperature of the reaction mixture was slowly raised to RT and was stirred at RT for 1 h. The reaction was monitored by TLC and after completion of the reaction ether was added and was then subjected to a standard ether work-up to yield desired product (4.0 g) as a liquid.

### B) Preparation of 1-bromo-4-(4-methyl-benzyloxy)-butan-2-one

To a solution of 4-(4-methyl-benzyloxy)-butan-2-one (3.5 g, 18.0 mmol) in dry methanol (15 mL) was added bromine (2.9 g, 18.0 mmol) at 0° C. and the reaction mixture was kept at that temperature for 1.5 h, in that time color of the bromine was disappeared. Then aqueous K₂CO₃ solution (1M, 170 mL) was added, the mixture was concentrated in vacuum and extracted twice with 200 mL of ethyl acetate. The extracts were washed twice with 70 mL of 1M K₂CO₃ solutions, dried, and the solvent was evaporated. The residue was dissolved in 680 mL of THF and 170 mL of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give crude material. The crude material was then purified by column chromatography using 6% ethyl acetate-hexane to give pure desired product (2.4 g) as a liquid.

### C) Preparation of 5-[2-(4-methyl-benzyloxy)-ethyl]-6H-[1,3,4]thiadiazin-2-ylamine.hydrobromide

To the stirred solution of 1-bromo-4-(4-methyl-benzyloxy)-butan-2-one (2.4 g, 8.8 mmol) in EtOH (30 ml) at RT was added thiosemicarbazide (800 mg, 8.8 mmol) and was stirred for overnight. The reaction was monitored by TLC and after completion of the reaction; mixture was filtered and the solid mass was purified by column chromatography on silica gel using MeOH:CH₂Cl₂ (1:9) to give 700 mg of the desired product as a white solid.

### Example 34

### 5-[2-(1-methyl-butoxy)-ethyl]-6H-[1,3,4]thiadiazin-2-ylamine. hydrobromide

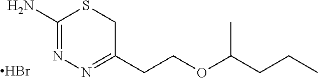

### A) Preparation of 4-(1-methyl-butoxy)-butan-2-one

To the stirred solution of methyl vinyl ketone (7.1 g, 8.33 mL, 0.1 mol) and pentan-2-ol (8.12 g, 0.09 mol) was added 50% H₂SO₄ (1.0 mL) at 0° C. and after addition temperature of the reaction mixture was slowly raised to RT and was stirred at RT for 1 h. The reaction was monitored by TLC and after completion of the reaction ether was added and was then subjected to a standard ether work-up to yield desired product (11.0 g) as a liquid.

### B) Preparation of 1-bromo-4-(1-methyl-butoxy)-butan-2-one

To a solution of 4-(1-methoxy-butoxy)-butan-2-one (11.0 g, 69.5 mmol) in dry methanol (44 mL) was added bromine (3.53 mL) at 0° C. and the reaction mixture was kept at that temperature for 1.5 h, in that time color of the bromine was disappeared. Then aqueous K₂CO₃ solution (1M, 132 mL) was added, the mixture was concentrated in vacuum and extracted twice with 150 mL of ethyl acetate. The extracts were washed twice with 70 mL of 1M K₂CO₃ solutions, dried, and the solvent was evaporated. The residue was dissolved in 680 mL of THF and 132 mL of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give 10.7 g of crude bromo ketone. The crude material was pure enough for next step of reaction.

### C) Preparation of 5-[2-(1-methyl-butoxy)-ethyl]-6H-[1,3,4]thiadiazin-2-ylamine. hydrobromide

To the stirred solution of 1-bromo-4-(1-methyl-butoxy)-butan-2-one (10.72 g, 45.0 mmol) in EtOH (100 ml) at RT was added thiosemicarbazide (4.12 g, 45.0 mmol) and was stirred for overnight. The reaction was monitored by TLC and after completion of the reaction; mixture was filtered and the solid mass was purified by column chromatography on silica gel using MeOH:CH₂Cl₂ (1:9) to give 100 mg of the desired product as a light yellow semi solid.

### Example 35

### 4-[2-(2-amino-6H-[1,3,4]thiadiazin-5-yl)-ethoxymethyl]-benzonitrile.hydrobromide

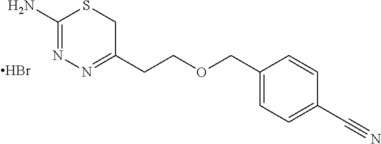 [SMILES: Br ; N#Cc1ccc(COCCC2=NN=C(N)SC2)cc1]

### A) Preparation of 4-(3-oxo-butoxymethyl)-benzonitrile

To the stirred solution of methyl vinyl ketone (1.60 g, 1.87 mL, 22.71 mmol) and 4-hydroxymethyl-benzonitrile (2.5 g, 20.65 mmol) was added 50% H₂SO₄ (1.0 mL) at 0° C. and after addition temperature of the reaction mixture was slowly raised to RT and was stirred at RT for 1 h. The reaction was monitored by TLC and after completion of the reaction ether was added and was then subjected to a standard ether work-up to yield desired product (2.3 g) as a liquid.

### B) Preparation of 4-(4-bromo-3-oxo-butoxymethyl)-benzonitrile

To a solution of 4-(3-oxo-butoxymethyl)-benzonitrile (2.0 g, 9.84 mmol) in dry methanol (10 mL) was added bromine (0.5 mL, 9.8 mmol) at 0° C. and the reaction mixture was kept at that temperature for 1.5 h, in that time color of the bromine was disappeared. Then aqueous K₂CO₃ solution (1M, 50 mL) was added, the mixture was concentrated in vacuum and extracted twice with 50 mL of ethyl acetate. The extracts were washed twice with 20 mL of 1M K₂CO₃ solutions, dried, and the solvent was evaporated. The residue was dissolved in 200 mL of THF and 50 mL of 1M H₂SO₄ and the mixture was heated to reflux for 1.5 h. Then the mixture was concentrated in vacuum and extracted with ethyl acetate. The extracts were washed with 2M KHCO₃, dried, and the solvent was evaporated to give 2.0 g of crude bromo ketone. The crude material was pure enough for next step of reaction.

### C) Preparation of 4-[2-(2-amino-6H-[1,3,4]thiadiazin-5-yl)-ethoxymethyl]-benzonitrile.hydrobromide

To the stirred solution of 4-(4-bromo-3-oxo-butoxymethyl)-benzonitrile (2.0 g, 7.09 mmol) in EtOH (20 ml) at RT was added thiosemicarbazide (640 mg, 7.09 mmol) and was stirred for overnight. The reaction was monitored by TLC and after completion of the reaction; mixture was filtered and the solid mass was purified by column chromatography on silica gel using MeOH:CH₂Cl₂ (1:9) to give 300 mg of the desired product as a yellow solid.

### Example 36

### 5-[2-(4-propoxy-phenyl)-ethyl]-6H-[1,3,4]thiadiazin-2-ylamine. Hydrobromide

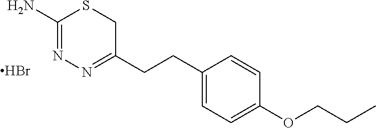 [SMILES: Br ; CCCOc1ccc(CCC2=NN=C(N)SC2)cc1]

### A) Preparation of 4-(4-propoxy-phenyl)-butan-2-one

To the stirred solution of 4-(4-hydroxy-phenyl)-butan-2-one (10 g, 62.0 mmol) in DMF was added Cs₂CO₃ (20.20 g, 62.0 mmol) and propyl bromide (5.60 mL, 62.1 mmol) at RT and was stirred for 18 h. The reaction was monitored by TLC and after completion of the reaction, mixture was poured in 2N HCl and was subjected to a standard ethyl acetate work up to give the desired product (14.0 g) as a liquid.

### B) Preparation of 1-bromo-4-(4-propoxy-phenyl)-butan-2-one

A solution of 4-(4-propoxy-phenyl)-butan-2-one (2.0 g, 9.71 mmol) in dry CH₂Cl₂ (100 mL) and methanol (50 mL) was treated with tetrabutylammonium tribromide (5.15 g, 10.68 mmol) and stirred at RT for 18 h. The reaction was monitored by TLC and after completion of the reaction, mixture was evaporated and the compound was purified by column chromatography using ethylacetate:hexane (1:9) to give the desired product (1.0 g) as a liquid.

### C) Preparation of 5-[2-(4-propoxy-phenyl)-ethyl]-6H-[1,3,4]thiadiazin-2-ylamine. Hydrobromide

To a solution of 1-bromo-4-(4-propoxy-phenyl)-butan-2-one (600 mg, 2.11 mmol) in EtOH (10 ml) at RT was added thiosemicarbazide (191.8 mg, 2.11 mmol) and was stirred for overnight. The reaction was monitored by TLC and after completion of the reaction; mixture was filtered and the solid mass was purified by column chromatography on silica gel using MeOH:CH₂Cl₂ (5:95) to give desired product (180 mg) as a yellow solid.

### Example 37

### 5-[2-(4-methoxy-benzyloxy)-ethyl]-6H-[1,3,4]thiadiazin-2-ylamine. hydrobromide

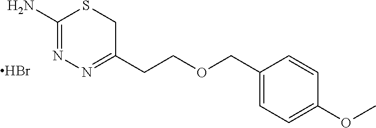 [SMILES: Br ; COc1ccc(COCCC2=NN=C(N)SC2)cc1]

### A) Preparation of 4-(4-methoxy-benzyloxy)-butan-2-one

To the stirred solution of (4-methoxy-phenyl)-methanol (19.70 g, 0.14 mol) in methyl acrylate (10.0 g, 0.14 mol) was added 2 mL aqueous H₂SO₄ (50%) at 0° C. under nitrogen atmosphere and stirred at the same temperature for 3 h. The reaction was monitored by TLC and after completion of the reaction; mixture was diluted with ethyl acetate and washed with brine. The ethyl acetate layer was dried (Na₂SO₄) and concentrated to give the crude material. The crude material was then purified by column chromatography using hexane-ethyl acetate (1:9) to give the desired product (17.5 g) as a liquid.

### B) Preparation of 1-bromo-4-(4-methoxy-benzyloxy)-butan-2-one

To the stirred solution of 4-(4-methoxy-benzyloxy)-butan-2-one (3.0 g, 14.4 mmol) in CH₂Cl₂ (100 mL) and MeOH (50 mL) was added tetrabutyl ammonium tribromide (7.65 g, 15.8 mmol) at 0° C. and allowed to stir for 16 h at the same temperature. The reaction was monitored by TLC and after completion of the reaction; mixture was concentrated and purified by column chromatography using hexane-ethyl acetate (92:8) to give the desired product (450 mg) as a liquid.

### C) Preparation of 5-[2-(4-methoxy-benzyloxy)-ethyl]-6H-[1,3,4]thiadiazin-2-ylamine. hydrobromide

To the stirred solution of 1-bromo-4-(4-methoxy-benzyloxy)-butan-2-one (450 mg, 1.56 mmol) in EtOH (10 ml) at RT was added thiosemicarbazide (140 mg, 1.56 mmol) and stirred for overnight. The reaction was monitored by TLC and after completion of the reaction; mixture was filtered and the solid mass was purified by column chromatography on silica gel using MeOH:CH₂Cl₂ (4:96) to give 110 mg of the desired product as a grey solid.

### Example 38

### 5-(2-amino-6H-[1,3,4]thiadiazin-5-yl)-pentanoic acid phenylamide.hydrobromide

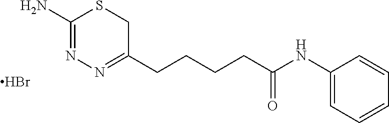 [SMILES: Br ; NC1=NN=C(CCCCC(=O)Nc2ccccc2)CS1]

### A) Preparation of 5-phenylcarbamoyl-pentanoic acid methyl ester

To the stirred solution of aniline (6.25 g, 6.1 mL, 0.067 mol) in CH₂Cl₂ (50 mL) was added methyl adipoyl chloride (10.0 g, 8.70 mL, 0.056 mol) at 0° C. After addition reaction mixture was stirred for 10 min at 0° C. and then stirred for 30 min at RT. The reaction was monitored by TLC and after completion of the reaction; mixture was washed with aqueous sodium bicarbonate solution. The organic layer was dried and purified by column chromatography using hexane:ethyl acetate (9:1) to give 13.0 g of the desired product as a liquid.

### B) Preparation of 5-phenylcarbamoyl-pentanoic acid

To the stirred solution of 5-phenylcarbamoyl-pentanoic acid methyl ester (12.0 g, 0.051 mol) in ethanol (480 mL) and water (12 mL) was added lithium hydroxide (3.8 g, 0.15 mol) slowly at 0° C. After addition temperature of the reaction mixture was slowly raised to RT and stirred at RT for 2 h. The reaction was monitored by TLC and after completion of the reaction ethanol was evaporated. Then water was added to the residue and pH of the reaction mixture was adjusted to acidic by using dilute HCl. The reaction mixture was extracted with ethyl acetate and concentrated under vacuum to give 875 mg of the desired product.

### C) Preparation of 5-phenylcarbamoyl-pentanoyl chloride

To the stirred solution of 5-phenylcarbamoyl-pentanoic acid (8.7 g, 39.5 mmol) in toluene (45 mL) was added thionyl chloride (5.6 g, 3.5 mL, 47.4 mmol) at 0° C. After addition temperature of the reaction mixture was slowly raised to RT and then refluxed at 120° C. for 3 h. The reaction was monitored by TLC and after completion of the reaction; solvent was distilled off and directly used for the next step.

### D) Preparation of 7-bromo-6-oxo-heptanoic acid phenyl amide

To the stirred solution of 5-phenylcarbamoyl-pentanoyl chloride (crude from the above reaction mixture) in dry ether (50 mL) was added an ethereal solution of CH₂N₂ (prepared from nitrosomethylurea) at 0° C. After 1 h at this temperature, the solvent was removed at 0° C. by means of a stream of N₂. The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuo at RT. The residue was subjected to a column chromatography on silica gel using EtOAc-hexane (1:1) as the eluting solvent to give desired product (1.30 g) as a light brown solid and used for the next step without purification.

### E) Preparation of 5-(2-amino-6H-[1,3,4]thiadiazin-5-yl)-pentanoic acid phenylamide.hydrobromide

To the stirred solution of 7-bromo-6-oxo-heptanoic acid phenyl amide (1.35 g, 4.5 mmol) in EtOH (15 ml) at RT was added thiosemicarbazide (413 mg, 4.5 mmol) and stirred for overnight. The reaction was monitored by TLC and after completion of the reaction; mixture was filtered and the solid mass was purified by column chromatography on silica gel using MeOH:CH₂Cl₂ (1:9) to give 200 mg of the desired product as off-white solid.

### Example 39

### 5-(6-phenyl-hexyl)-6H-[1,3,4]-thiadiazin-2-ylamine. hydrobromide

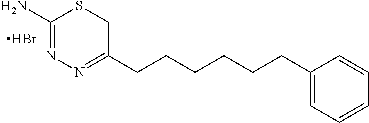 [SMILES: Br ; NC1=NN=C(CCCCCCc2ccccc2)CS1]

### A) Preparation of 1-bromo-8-phenyl-octan-2-one

To the stirred solution of 7-phenyl-heptanoic acid (1.0 g, 4.84 mmol) in toluene (15 mL) was added thionyl chloride (0.43 mL, 5.81 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuum at RT. The residue was subjected to column chromatography on silica gel using EtOAc-hexane (1:9) as the eluting solvent to give desired product (800 mg) as a liquid.

### B) Preparation of 5-(6-phenyl-hexyl)-6H-[1,3,4]-thiadiazin-2-ylamine. hydrobromide

To the stirred solution of 1-bromo-8-phenyl-octan-2-one (800 mg, 2.82 mmol) in EtOH (10 ml) at RT was added thiosemicarbazide (260 mg, 2.82 mmol) and stirred for overnight. The reaction was monitored by TLC and after completion of the reaction; mixture was filtered and the solid mass was purified by column chromatography on silica gel using MeOH:CH₂Cl₂ (1:9) to give 230 mg of the desired product as white solid.

### Example 40

### 5-(6-phenoxy-hexyl)-6H-[1,3,4]thiadiazin-2-ylamine. hydrobromide

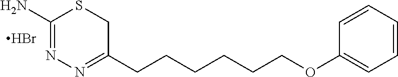 [SMILES: Br ; NC1=NN=C(CCCCCCOc2ccccc2)CS1]

### A) Preparation of (6-bromo-hexyloxy)-benzene

To the stirred solution of phenol (4.8 g, 0.051 mol) in water (100 ml) was added 1,5-dibromo-hexane (10 g, 0.041 mol) and the reaction mixture was heated to reflux and at the reflux condition aqueous NaOH (1.8 g in 10 ml water) solution was added. After addition reflux was continued for another 4.5 h. The progress of reaction was monitored by TLC and after completion of the reaction, mixture was cooled to RT and the upper layer was separated and discarded and the lower layer was washed with benzene. The combined benzene layers were washed with dilute NaOH solution and water. The organic layers were dried and then evaporated under reduced pressure to give crude desired product and was purified by column chromatography using hexane to give 4.6 g of the pure desired product as a liquid.

### B) Preparation of 7-phenoxy-heptanenitrile

To the stirred solution of (6-bromo-hexyloxy)-benzene (4.6 g, 17.9 mmol) in ethanol (60 mL) was added aqueous KCN solution (1.4 g, 21.46 mmol) and the mixture was heated to reflux for 14 h. The progress of the reaction was monitored by TLC and after completion of the reaction, mixture was cooled to RT and then evaporated the ethanol and the aqueous layer was extracted with ethyl acetate and the ethyl acetate layer was washed with dilute NaOH solution and the solvent was dried and evaporated to give 3.7 g desired product.

### C) Preparation of 7-phenoxy-heptanoic acid

To the stirred solution of 7-phenoxy-heptanenitrile (3.7 g, 18.23 mmol) in ethanol (20 ml) was added aqueous NaOH solution (1.1 g in 10 ml water) and after addition the reaction mixture was heated to reflux for overnight. The progress of the reaction was monitored by TLC and after completion of the reaction, mixture was cooled to 0° C. and pH of the reaction mixture was adjusted to 4 by adding 6N HCl and then subjected to a standard ethyl acetate work up to give the crude product and was purified by column chromatography using ethyl acetate-hexane (7:3) to give 3.0 g pure desired product.

### D) Preparation of 1-bromo-8-phenoxy-octan-2-one

To the stirred solution of 7-phenoxy-heptanoic acid (3.0 g, 13.5 mmol) in toluene (30 mL) was added thionyl chloride (1.5 mL, 20.27 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea (15.8 g, 54.05 mmol)) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (20:80) as the eluting solvent to give desired product (2.8 g) as a liquid.

### E) Preparation of 5-(6-phenoxy-hexyl)-6H-[1,3,4]thiadiazin-2-ylamine. hydrobromide

To a solution of 1-bromo-8-phenoxy-octan-2-one (2.8 g, 9.36 mmol) in EtOH (50 ml) at RT was added thiosemicarbazide (682 mg, 7.49 mmol) and was stirred for overnight. The reaction was monitored by TLC and after completion of the reaction; mixture was filtered and the solid mass was purified by column chromatography on silica gel using MeOH:CH₂Cl₂ (1:9) to give desired product (500 mg) as white solid.

### Example 41

### 5-(3-phenoxy-propyl)-6H-[1,3,4]thiadiazin-2-ylamine. hydrobromide

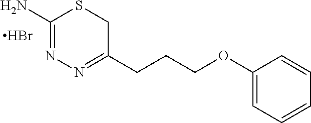 [SMILES: Br ; NC1=NN=C(CCCOc2ccccc2)CS1]

### A) Preparation of 1-bromo-5-phenoxy-pentan-2-one

To the stirred solution of 4-phenoxy-butyric acid (5.0 g, 27.7 mmol) in toluene (50 mL) was added thionyl chloride (2.5 mL, 33.0 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (3:7) to give desired product (2.2 g).

### B) Preparation of 5-(3-phenoxy-propyl)-6H-[1,3,4]thiadiazin-2-ylamine. hydrobromide

To the stirred solution of 1-bromo-5-phenoxy-pentan-2-one (2.20 g, 8.55 mmol) in EtOH (20 ml) at RT was added thiosemicarbazide (780 mg, 8.55 mmol) and stirred for overnight. The reaction was monitored by TLC and after completion of the reaction; mixture was filtered and the solid mass was purified by column chromatography on silica gel using MeOH:CH₂Cl₂ (1:9) to give desired product (300 mg) as off-white solid.

### Example 42

### 5-phenoxymethyl-6H-[1,3,4]thiadiazin-2-ylamine. hydrobromide

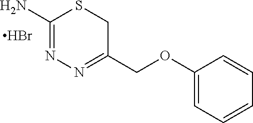 [SMILES: Br ; NC1=NN=C(COc2ccccc2)CS1]

### A) Preparation of 1-bromo-3-phenoxy-propan-2-one

To the stirred solution of phenoxy-acetic acid (5.0 g, 32.8 mmol) in toluene (60 mL) was added thionyl chloride (3.0 mL, 39.4 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (20:80) as the eluting solvent to give desired product (5.5 g).

### B) Preparation of 5-phenoxymethyl-6H-[1,3,4]thiadiazin-2-ylamine. hydrobromide

To the stirred solution of 1-bromo-3-phenoxy-propan-2-one (5.50 g, 24.0 mmol) in EtOH (60 ml) at RT was added thiosemicarbazide (1.75 g, 19.2 mmol) and stirred for overnight. The reaction was monitored by TLC and after completion of the reaction; mixture was filtered and the solid mass was purified by column chromatography on silica gel using MeOH:CHCl₃ (1:9) to give 120 mg pure desired product as white solid.

### Example 43

### 5-(2-p-tolyloxy-ethyl)-6H-[1,3,4]thiadiazin-2-ylamine. hydrobromide

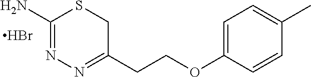 [SMILES: Br ; Cc1ccc(OCCC2=NN=C(N)SC2)cc1]

A) Preparation of 3-p-tolyloxy-propionitrile

4-Methyl phenol (2.0 g, 1.9 mL, 0.018 mol), acrylonitrile (14.4 mL, 0.216 mol) and benzyltrimethyl ammonium hydroxide (0.5 mL) were taken in a RBF and allowed to reflux for 22 h. The reaction was monitored by TLC and after completion of the reaction; mixture was diluted with two volumes of chloroform, turbidity was appeared and filtered. The filtrate was washed with 5% aqueous sodium hydroxide solution; dilute hydrochloric acid, water and brine. The organic layer was dried over Na₂SO₄ and the dry solution was evaporated under reduced pressure to give the desired product (1.90 g) as a liquid.

### B) Preparation of 3-p-tolyloxy-propionic acid

To the stirred solution of 3-p-tolyloxy-propionitrile (10.0 g, 62.0 mmol) was added conc. HCl (400 mL) and water (200 mL) and the mixture was refluxed for 2 h. The reaction was monitored by TLC and after completion of the reaction; mixture was cooled to RT and subjected to a standard ethyl acetate work-up to give 3.5 g desired product.

### C) Preparation of 3-p-tolyloxy-propionyl chloride

To the stirred solution of 3-p-tolyloxy-propionic acid (7.0 g, 38.8 mmol) in dry toluene (70 mL) was added thionyl chloride (3.5 mL, 46.6 mmol) at 0° C. After addition temperature of the reaction mixture was slowly raised to RT and then refluxed at 120° C. for 3 h. The reaction was monitored by TLC and after completion of the reaction; solvent was distilled off and directly used for the next step.

### D) Preparation of 1-bromo-4-p-tolyloxy-butan-2-one

To the stirred solution of 3-p-tolyloxy-propionyl chloride (crude from the above reaction mixture) in dry ether (50 mL) was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuo at RT. The residue was subjected to a column chromatography on silica gel using EtOAc-hexane (1:1) as the eluting solvent to give desired product (2.40 g) as a solid and used for the next step without purification.

### E) Preparation of 5-(2-p-tolyloxy-ethyl)-6H-[1,3,4]thiadiazin-2-ylamine. hydrobromide

To the stirred solution of 1-bromo-4-p-tolyloxy-butan-2-one (2.40 g, 9.4 mmol) in EtOH (24 ml) at RT was added thiosemicarbazide (860 mg, 9.4 mmol) and stirred for overnight. The reaction was monitored by TLC and after completion of the reaction; mixture was filtered and the solid mass was purified by column chromatography on silica gel using MeOH:CH₂Cl₂ (1:9) to give 350 mg of the desired product as an off-white solid.

### Example 44

### 5-[2-(biphenyl-4-yloxy)-ethyl]-6H-[1,3,4]thiadiazin-2-ylamine. hydrobromide

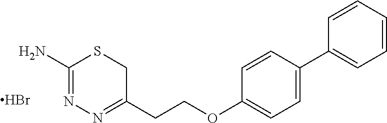 [SMILES: Br ; NC1=NN=C(CCOc2ccc(-c3ccccc3)cc2)CS1]

### A) Preparation of 3-(biphenyl-4-yloxy)-propionitrile

Biphenyl-4-ol (2.0 g, 11.0 mmol), acrylonitrile (1.3 mL, 0.14 mol) and benzyltrimethyl ammonium hydroxide (0.2 mL) were taken in a RBF and allowed to reflux for 22 h. The reaction was monitored by TLC and after completion of the reaction; mixture was diluted with two volumes of chloroform, turbidity was appeared and filtered. The filtrate was washed with 5% aqueous sodium hydroxide solution; dilute hydrochloric acid, water and brine. The organic layer was dried over Na₂SO₄ and the dry solution was evaporated under reduced pressure to give the desired product (1.30 g) as a liquid.

### B) Preparation of 3-(biphenyl-4-yloxy)-propionic acid

To the stirred solution of 3-(biphenyl-4-yloxy)-propionitrile (7.0 g) was added conc. HCl (280 mL) and water (140 mL) and the mixture was refluxed for 2 h. The reaction was monitored by TLC and after completion of the reaction; mixture was cooled to RT and subjected to a standard ethyl acetate work-up to give crude product. The crude product was then purified by column chromatography using ethyl acetate-hexane (3:7) to give pure desired product (1.1 g).

### C) Preparation of 4-(biphenyl-4-yloxy)-1-bromo-butan-2-one

To the stirred solution of 3-(biphenyl-4-yloxy)-propionic acid (1.1 g, 4.55 mmol) in toluene (30 mL) was added thionyl chloride (0.5 mL, 6.82 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (20:80) as the eluting solvent to give desired product (2.8 g) as a liquid.

### D) Preparation of 5-[2-(biphenyl-4-yloxy)-ethyl]-6H-[1,3,4]thiadiazin-2-ylamine. hydrobromide

To the stirred solution of 4-(biphenyl-4-yloxy)-1-bromo-butan-2-one (1.30 g, 4.08 mmol) in EtOH (20 ml) at RT was added thiosemicarbazide (299 mg, 3.26 mmol) and stirred for overnight. The reaction was monitored by TLC and after completion of the reaction; mixture was filtered and the solid mass was purified by column chromatography on silica gel using MeOH:CH₂Cl₂ (1:9) to give (500 mg) desired product as light yellow solid.

### Example 45

### 5-(4-phenoxy-butyl)-6H-[1,3,4]thiadiazin-2-ylamine

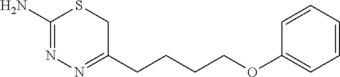 [SMILES: NC1=NN=C(CCCCOc2ccccc2)CS1]

### A) Preparation of (4-bromo-butoxy)-benzene

To the stirred solution of phenol (10.7 g, 0.117 mol) was added 1,4-dibromo-butane (17.6 mL, 0.146 mol) and the mixture was heated to reflux and at the reflux condition aqueous NaOH (4.2 g in 40 ml water) solution was added. After addition reflux was continued for another 4.5 h. The progress of the reaction was monitored by TLC and after completion of the reaction, mixture was cooled to RT and upper layer was separated and discarded and the lower layer was washed with ethyl acetate. The combined ethyl acetate layers were washed with dilute NaOH solution and water. The organic layers were dried and then evaporated under reduced pressure to give crude desired product (26.1 g) and used for the next step without purification.

### B) Preparation of 5-phenoxy-pentanenitrile

To the stirred solution of (4-bromo-butoxy)-benzene (10.0 g, 43.6 mmol) in ethanol (100 mL) was added aqueous KCN solution (3.4 g, 52.3 mmol in 40 mL water) and the mixture was heated to reflux for 28 h. The progress of the reaction was monitored by TLC and after completion of the reaction, mixture was cooled to RT and evaporated the ethanol and the aqueous layer was then subjected to a standard ethyl acetate work up to give the crude material. The crude material was purified by column chromatography using ethyl acetate-hexane (1:9) to give (3.1 g) the desired product as a liquid.

### C) Preparation of 5-phenoxy-pentanoic acid

To the stirred solution of 5-phenoxy-pentanenitrile (3.1 g, 0.017 mol) in water (30 ml) was added conc. HCl (120 mL) and after addition the reaction mixture was heated to reflux for 2 h. The progress of the reaction was monitored by TLC and after completion of the reaction, mixture was cooled to 0° C. and subjected to a standard ethyl acetate work up to give the desired product (3.4 g) as a solid.

### D) Preparation of 1-bromo-6-phenoxy-hexan-2-one

To the stirred solution of 5-phenoxy-pentanoic acid (3.4 g, 17.5 mmol) in toluene (35 mL) was added thionyl chloride (1.56 mL, 21.0 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (3:7) as the eluting solvent to give desired product (2.5 g) as a liquid.

### E) Preparation of 5-(4-phenoxy-butyl)-6H-[1,3,4]thiadiazin-2-ylamine

To the stirred solution of 1-bromo-6-phenoxy-hexan-2-one (2.50 g, 9.25 mmol) in EtOH (20 ml) at RT was added thiosemicarbazide (843 mg, 9.25 mmol) and stirred for overnight. The reaction was monitored by TLC and after completion of the reaction; mixture was filtered and the solid mass was purified by column chromatography on silica gel using MeOH:CH₂Cl₂ (1:9) to give 170 mg of the desired product as off-white solid.

### Example 46

### 5-phenoxymethyl-6H-[1,3,4]thiadiazin-2-ylamine

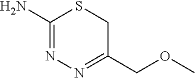

### A) Preparation of 1-bromo-3-methoxy-propan-2-one

To the stirred solution of methoxy acetic acid (5.0 g, 55.0 mmol) in dry benzene (80 mL) was added thionyl chloride (4.5 mL, 61.0 mmol) and pyridine (1 drop) at 0° C. After addition temperature of the reaction mixture was slowly raised to RT and then refluxed at 80° C. for 30 minute. The reaction was monitored by TLC and after completion of the reaction; solvent was distilled off to get a crude reaction mixture. The crude reaction mixture was then dissolved in dry ether (50 mL) was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuum at RT. The residue was subjected to a column chromatography on silica gel using EtOAc-hexane (1:1) to give desired product (4.0 g) as a yellow liquid and used for the next step without purification.

### B) Preparation of 5-phenoxymethyl-6H-[1,3,4]thiadiazin-2-ylamine

To the stirred solution of 1-bromo-3-methoxy-propan-2-one (3.50 g, 21.0 mmol) in EtOH (40 ml) at RT was added thiosemicarbazide (1.72 g, 18.8 mmol) and stirred for overnight. The reaction was monitored by TLC and after completion of the reaction; mixture was filtered and the solid mass was purified by column chromatography on silica gel using MeOH:CH₂Cl₂ (5:95) to give 120 mg of the desired product as green solid.

### Example 47

### 6-(2-amino-6H-[1,3,4]thiadiazin-5-yl)-hexanoic acid ethyl ester.hydrobromide

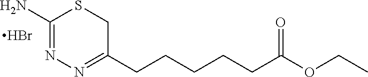 [SMILES: Br ; CCOC(=O)CCCCCC1=NN=C(N)SC1]

### A) Preparation of heptanedioic acid monoethyl ester

A solution of potassium hydroxide (2.6 g, 46.2 mmol) in absolute alcohol (40 mL) was slowly added to diethyl pimelate (10 g, 46.2 mmol) at 50° C. and after addition mixture was stirred for overnight at the same temperature. The reaction was monitored by TLC and after completion of the reaction; mixture was concentrated to yield a white solid. This solid was then washed with hexane to give the desired product (9.0 g) as a white solid and used for the next step.

### B) Preparation of 8-bromo-7-oxo-octanoic acid ethyl ester

To the stirred solution of heptanedioic acid monoethyl ester (2.0 g, 8.83 mmol) in dry benzene (20 mL) at 0° C. was added a solution of oxalyl chloride (0.83 mL, 9.7 mmol) in dry benzene (5 mL) and the mixture was stirred at 0° C. for1 h. After that mixture was concentrated under vacuum and dissolved the residue in ether. To this was added an ethereal solution of diazomethane dropwise at 0° C. and the mixture was stirred for 1 h at 0° C. After that solvent was evaporated under nitrogen and the residue was dissolved in dry CH₂Cl₂ and cooled to −78° C. Then saturated solution of HBr in CH₂Cl₂ (20 mL) was added slowly and allowed to stir for 1 h at −78° C. The reaction was monitored by TLC and after completion of the reaction; mixture was concentrated to yield desired compound (2.1 g) as a liquid and used for the next step without purification as NMR and LCMS show desired compound has formed.

### C) Preparation of 6-(2-amino-6H-[1,3,4]thiadiazin-5-yl)-hexanoic acid ethyl ester.hydrobromide

To the stirred solution of 8-bromo-7-oxo-octanoic acid ethyl ester (12.0 g, 45.2 mmol) in EtOH (60 ml) at RT was added thiosemicarbazide (4.1 g, 45.2 mmol) and stirred for overnight. The reaction was monitored by TLC and after completion of the reaction; mixture was filtered and the solid mass was purified by column chromatography on silica gel using MeOH:CH₂Cl₂ (1:9) to give pure desired product (2.7 g) as white solid.

### Example 48

### 6-(2-amino-6H-[1,3,4]thiadiazin-5-yl)-hexanoic acid. trifluoroacetic acid

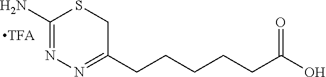 [SMILES: NC1=NN=C(CCCCCC(=O)O)CS1 ; "·TFA"]

### A) Preparation of potassium salt of heptanedioic acid monoethyl ester

A solution of potassium hydroxide (2.6 g, 46.2 mmol) in absolute alcohol (40 mL) was slowly added to diethyl pimelate (10.0 g, 46.2 mmol) at 50° C. and after addition mixture was stirred for overnight at the same temperature. The reaction was monitored by TLC and after completion of the reaction; mixture was concentrated to yield a white solid. This solid was then washed with hexane to give the desired product (9.0 g) as a white solid and used for the next step.

### B) Preparation of 8-bromo-7-oxo-octanoic acid ethyl ester

To the stirred solution of heptanedioic acid monoethyl ester (2.0 g, 8.83 mmol) in dry benzene (20 mL) at 0° C. was added a solution of oxalyl chloride (0.83 mL, 9.7 mmol) in dry benzene (5 mL) and the mixture was stirred at 0° C. for1 h. After that mixture was concentrated under vacuum and dissolved the residue in ether. To this was added an ethereal solution of diazomethane dropwise at 0° C. and the mixture was stirred for 1 h at 0° C. After that solvent was evaporated under nitrogen and the residue was dissolved in dry CH₂Cl₂ and then cooled to −78° C. Then saturated solution of HBr in CH₂Cl₂ (20 mL) was added slowly and allowed to stir for 1 h at −78° C. The reaction was monitored by TLC and after completion of the reaction; mixture was concentrated to yield desired compound (2.1 g) as a liquid and used for the next step without purification as NMR and LCMS show desired compound has formed.

### C) Preparation of 6-(2-amino-6H-[1,3,4]thiadiazin-5-yl)-hexanoic acid ethyl ester.hydrobromide

To the stirred solution of 6-(2-amino-6H-[1,3,4]thiadiazin-5-yl)-hexanoic acid ethyl ester (1.0 g, 3.88 mmol) in THF (10 mL) and water (10 mL) at 0° C. was added NaHCO₃ (720 mg, 8.55 mmol) portion wise and after that Boc2O (1.6 mL, 6.99 mmol) was added slowly at the same temperature. Then the mixture was allowed to stir for overnight at RT. The reaction was monitored by TLC and after completion of the reaction; mixture was subjected to a standard ethyl acetate work-up to give the desired product (2.1 g) as a solid.

### D) Preparation of 6-(2-tert-butoxycarbonylamino-6H-[1,3,4]thiadiazin-5-yl)-hexanoic acid ethyl ester

To the stirred solution of 6-(2-amino-6H-[1,3,4]thiadiazin-5-yl)-hexanoic acid ethyl ester (1.0 g, 3.88 mmol) in THF (10 mL) and water (10 mL) at 0° C. was added NaHCO₃ (720 mg, 8.55 mmol) portion wise and after that Boc₂O (1.6 mL, 6.99 mmol) was added slowly at the same temperature. Then the mixture was allowed to stir for overnight at RT. The reaction was monitored by TLC and after completion of the reaction; mixture was subjected to a standard ethyl acetate work-up to give the desired product (2.1 g) as a solid.

### E) Preparation of 6-(2-tert-butoxycarbonylamino-6H-[1,3,4]thiadiazin-5-yl)-hexanoic acid

To 6-(2-tert-butoxycarbonylamino-6H-[1,3,4]thiadiazin-5-yl)-hexanoic acid ethyl ester (2.1 g, 5.88 mmol) was added NaOH solution (0.35 g in 5 mL water) and the mixture was allowed to stir for 4 h at RT. The reaction was monitored by TLC and after completion of the reaction; mixture was cooled to 0° C. and adjust the pH to 2 with 1N HCl and the mixture was subjected to a standard ethyl acetate work up to give the desired product (600 mg) as a brown solid.

### F) Preparation of 6-(2-amino-6H-[1,3,4]thiadiazin-5-yl)-hexanoic acid. trifluoroacetic acid

To the stirred solution of 6-(2-tert-butoxycarbonylamino-6H-[1,3,4]thiadiazin-5-yl)-hexanoic acid (200 mg, 0.6 mmol) at 0° C. was added trifluoro acetic acid (1 mL). After addition temperature of the reaction mixture was slowly raised to RT and stirred at RT for 3h. The reaction was monitored by TLC and after completion of the reaction; mixture was concentrated under vacuum to give desired product (110 mg) a white solid.

### Example 49

### 5-(4-methoxy-butyl)-6H-[1,3,4]thiadiazin-2-ylamine.hydrobromide

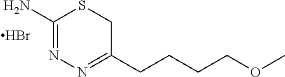

### A) Preparation of 5-methoxy-pentan-1-ol

To the ice cooled solution of NaH (5.7 g, 0.24 mol) in dry THF (250 mL) was added 1,5-pentanediol (25 g, 0.24 mmol) in THF (550 mL) over a period of 30 minutes. After addition mixture was then stirred for another 30 min at the same temperature. After that MeI (40.8 g, 0.28 mmol) in THF was added slowly to the reaction mixture at the same temperature and the mixture was quenched with saturated NH₄Cl solution at cooling temperature and the mixture was subjected to a standard ethyl acetate work up to give the desired product as liquid (1.0 g).

### B) Preparation of 5-methoxy-pentanoic acid

To the stirred solution of 5-methoxy-pentan-1-ol (18.0 g) in acetone (100 mL) was added Jones reagent (which was prepared by addition of CrO₃ (75 g) to the cooled solution of H₂SO₄ (65 mL) followed by water addition slowly at 0° C.) slowly at 0° C. until the reaction mixture becomes green in color. Then solvent was evaporated under vacuum and subjected the reaction mixture to a standard CH₂Cl₂ work up to give desired product (6.4 g) as a liquid.

### C) Preparation of 1-bromo-6-methoxy-hexan-2-one

To the stirred solution of 5-methoxy-pentanoic acid (6.4 g, 48.0 mmol) in dry toluene (45 mL) was added thionyl chloride (4.3 mL, 57.0 mmol) at 0° C. After addition temperature of the reaction mixture was slowly raised to RT and then refluxed at 120° C. for 3 h. The reaction was monitored by TLC and after completion of the reaction; solvent was distilled off to give the crude material. The crude material was then dissolved in dry ether (50 mL) and to the solution was added an ethereal solution of CH₂N₂ (prepared from nitrosomethylurea) at 0° C. After 1 h at this temperature, the solvent was removed at 0° C. by means of a stream of N₂. The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuum at RT. The residue was subjected to a column chromatography on silica gel using EtOAc-hexane (1:1) as the eluting solvent to give the desired product (6.90 g) as a light brown solid and used for the next step without purification.

### D) Preparation of 5-(4-methoxy-butyl)-6H-[1,3,4]thiadiazin-2-ylamine.hydrobromide

To the stirred solution of 1-bromo-6-methoxy-hexan-2-one (6.90 g, 33.0 mmol) in EtOH (25 ml) at RT was added thiosemicarbazide (3.02 g, 33.0 mmol) and stirred for overnight. The reaction was monitored by TLC and after completion of the reaction; mixture was filtered and the solid mass was purified by column chromatography on silica gel using MeOH:CH₂Cl₂ (1:9) to give 275 mg of the desired product as off-white solid.

### Example 50

### 5-(7-methoxy-heptyl)-6H-[1,3,4]thiadiazin-2-ylamine. hydrobromide

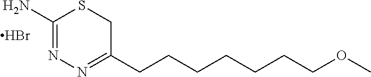 [SMILES: Br ; COCCCCCCCC1=NN=C(N)SC1]

### A) Preparation of 8-methoxy-octanoic acid

A mixture of 8-bromo octanoic acid (5.0 g, 22.4mmole) and Na metal (1.5 g, 67.23 mmole) in dry methanol (50 ml) was stirred at reflux for 6 h under nitrogen atmosphere. The reaction was monitored by TLC and after completion of the reaction; mixture was poured into water (100 ml), acidified with 6N HCl and subjected for a standard ether work up to give the desired product (4.49 g) as a liquid.

### B) Preparation of 1-bromo-9-methoxy-nonan-2-one

To the stirred solution of 8-methoxy-octanoic acid (4.4 g, 25.0 mmol) in toluene (100 mL) was added thionyl chloride (2.75 mL, 37.8 mmol) at 0° C. After addition temperature of the reaction mixture was slowly raised to RT and then refluxed at 120° C. for 3 h. The reaction was monitored by TLC and after completion of the reaction; solvent was distilled off. The crude mixture was then dissolved in ether and to this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in ether and the solution was cooled to −70° C. A saturated solution of HBr gas in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo at RT. The residue was subjected to column chromatography on silica gel using EtOAc-hexane (20:80) as the eluting solvent to give desired product (4.6 g) as a liquid.

### C) Preparation of 5-(7-methoxy-heptyl)-6H-[1,3,4]thiadiazin-2-ylamine. hydrobromide

To the stirred solution of 1-bromo-9-methoxy-nonan-2-one (4.4 g, 17.5 mmol) in EtOH (150 ml) at RT was added thiosemicarbazide (1.5 g, 17.5 mmol) and stirred for overnight. The reaction was monitored by TLC and after completion of the reaction; mixture was filtered and the solid mass was purified by column chromatography on silica gel using MeOH:CH₂Cl₂ (1:9) to give 450 mg of the desired product as a yellow solid.

### Example 51

### 5-(7-Phenoxy-heptyl)-6H-[1,3,4]thiadiazin-2-ylamine.hydrobromide

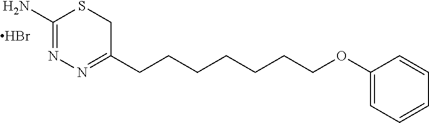 [SMILES: Br ; NC1=NN=C(CCCCCCCOc2ccccc2)CS1]

### A) Preparation of (7-bromo-heptyloxy)-benzene

To the stirred solution of phenol (3.30 mL, 37.8 mmol) was added 1,7-dibromo-heptane (10.8 g, 34.1 mmol) and the mixture was heated to reflux and at the reflux condition aqueous NaOH (1.5 g in 30 ml water) solution was added. After addition reflux was continued for another 12 h. The progress of the reaction was monitored by TLC and after completion of the reaction mixture was cooled to RT. The upper layer was separated and discarded; the lower layer was extracted with hexane. The combined hexane layers were washed with dilute NaOH solution and water. The organic layer was then dried and evaporated to give the crude residue, which was purified by column chromatography using hexane to give the pure desired product (7.0 g) as a colorless liquid.

### B) Preparation of 8-phenoxy-octanenitrile

To the stirred solution of (7-bromo-heptyloxy)-benzene (1.0 g, 3.70 mmol) in ethanol (10 mL) was added aqueous KCN solution (0.3 g, 4.5 mmol in 7 mL water) and the mixture was heated to reflux for 28 h. The progress of the reaction was monitored by TLC and after completion of the reaction mixture was cooled to RT. Then solvent was evaporated and the aqueous layer was subjected to a standard ethyl acetate work up to give the desired product (900 mg) as a liquid.

### C) Preparation of 8-phenoxy-octanoic acid

To the stirred solution of 8-phenoxy-octanenitrile (5.1 g, 23.4 mmol) in water (40 ml) was added conc. HCl (200 mL) and after addition the reaction mixture was heated to reflux for 2 h. The progress of the reaction was monitored by TLC and after completion of the reaction, mixture was cooled to 0° C. and subjected to a standard ethyl acetate work up to give the desired product (6.0 g) as a solid.

### D) Preparation of 1-bromo-9-phenoxy-nonan-2-one

To the stirred solution of 8-phenoxy-octanoic acid (3.75 g, 15.8 mmol) in dry toluene (40 mL) was added thionyl chloride (1.4 mL, 19.0 mmol) at 0° C. After addition temperature of the reaction mixture was slowly raised to RT and then refluxed at 120° C. for 3 h. The reaction was monitored by TLC and after completion of the reaction; solvent was distilled off and the crude was dissolved in dry ether (50 mL). To this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuo at RT to give the desired crude product (4.1 g) as a light brown gummy solid.

### E) Preparation of 5-(7-phenoxy-heptyl)-6H-[1,3,4]-thiadiazin-2-ylamine. Hydrobromide

To the stirred solution of 1-bromo-9-phenoxy-nonan-2-one (3.5 g, 11.2 mmol) in EtOH (35 mL) at RT was added thiosemicarbazide (1.0 g, 11.2 mmol) and stirred for overnight. The reaction was monitored by TLC and after completion of the reaction; mixture was filtered and the solid mass was purified by column chromatography on silica gel using MeOH:CH₂Cl₂ (1:9) to give 350 mg of the desired product as an off-white solid.

### Example 52

### 5-(6-Methoxy-hexyl)-6H-[1,3,4]thiadiazin-2-ylamine.hydrobromide

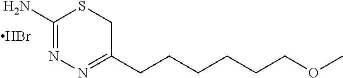

### A) Preparation of 1-bromo-6-methoxy-hexane

To the stirred solution of sodium (0.5 g, 21.0 mmol) in methanol (200 mL) was added 1, 6-dibromo hexane (10.0 g, 40.9 mmol) in MeOH (50 mL) over a period of 20 min. After addition mixture was heated to reflux for 5 h. The reaction was monitored by TLC and after completion of the reaction; methanol was distilled off and the residue was dissolved in water and subjected to a standard CH₂Cl₂ work up to yield desired product (8.3 g) as a pale yellow liquid.

### B) Preparation of 7-methoxy-heptanenitrile

To the stirred solution of 1-bromo-6-methoxy-hexane (8.0 g, 41.0 mmol) in ethanol (100 mL) was added aqueous KCN solution (2.6 g, 40.0 mmol in 10 mL water) and the mixture was heated to reflux for 24 h. The progress of the reaction was monitored by TLC and after completion of the reaction mixture was cooled to RT. Then solvent was evaporated and the aqueous layer was subjected to a standard ethyl acetate work up to give the desired product (5.9 g) as yellow liquid.

### C) Preparation of 7-methoxy-heptanoic acid

To the stirred solution of 7-methoxy-heptanenitrile (2.0 g, 14.1 mmol) in ethanol (20 ml) was added NaOH (0.85 g, 21.2 mmol) and after addition mixture was heated to reflux for 6 h. The progress of the reaction was monitored by TLC and after completion of the reaction ethanol was evaporated. The residue was dissolved in water (10 mL) followed by addition of 6 N HCl until pH 4-5. The whole reaction mixture was then subjected to a standard chloroform work up to give the desired product (1.2 g) as a liquid.

### D) Preparation of 1-bromo-8-methoxy-octan-2-one

To the stirred solution of 7-methoxy-heptanoic acid 2.5 g, 15.6 mmol) in dry toluene (25 mL) was added thionyl chloride (1.4 mL, 18.4 mmol) at 0° C. After addition temperature of the reaction mixture was slowly raised to RT and then refluxed at 120° C. for 3 h. The reaction was monitored by TLC and after completion of the reaction; solvent was distilled off and the crude was dissolved in dry ether (50 mL). To this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuo at RT to give 3.2 g desired crude product.

### E) Preparation of 5-(6-methoxy-hexyl)-6H-[1,3,4]thiadiazin-2-ylamine.hydrobromide

To the stirred solution of 1-bromo-8-methoxy-octan-2-one (3.2 g, 13.5 mmol) in EtOH (35 mL) at RT was added thiosemicarbazide (1.23 g, 13.5 mmol) and stirred for overnight. The reaction was monitored by TLC and after completion of the reaction; mixture was filtered and the solid mass was purified by column chromatography on silica gel using MeOH:CHCl₃ (1:9) to give 130 mg of the desired product as white solid.

### Example 53

### 5-[5-(Methyl-phenyl-amino)-pentyl]-6H-[1,3,4]thiadiazin-2-ylamine.hydrobromide

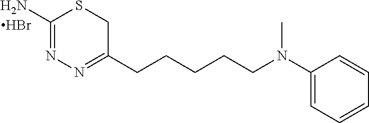 [SMILES: Br ; CN(CCCCCC1=NN=C(N)SC1)c1ccccc1]

### A) Preparation of 6-(methyl-phenyl-amino)-hexanoic acid ethyl ester

A mixture of phenyl-methyl amine (5.06 mL, 46.0 mmol) and 6-bromo-hexanoic acid ethyl ester (9.1 mL, 51.0 mmol) and 2, 6-lutidine (6.2 mL, 53.6 mol) in acetonitrile (60 mL) was refluxed for 18 h. The solvent was distilled off and the residue was dissolved in ethyl acetate, washed with water and dried over Na₂SO_{4.}The solvent was evaporated and the residue was purified by column chromatography using hexane-ethyl acetate (9:1) to give the pure desired product as a liquid (10.6 g).

### B) Preparation of 6-(methyl-phenyl-amino)-hexanoic acid

6-(methyl-phenyl-amino)-hexanoic acid ethyl ester (8.0 g, 32.0 mmol) was dissolved in methanol (25 mL) and aqueous NaOH (1.4 g, 35.0 mmol in 25 mL water). The reaction mixture was then refluxed for 3 h. The progress of the reaction was monitored by TLC and after completion of the reaction; solvent was evaporated and then acidified by dilute HCl until pH=3. The mixture was then subjected to a standard ethyl acetate work up to give the crude residue, which was purified by column chromatography using ethyl acetate-hexane (1:9) to give the desired product (8.0 g) as a liquid.

### C) Preparation of 1-bromo-7-(methyl-phenyl-amino)-heptan-2-one

To the stirred solution of 6-(methyl-phenyl-amino)-hexanoic acid (2.5 g, 11.3 mmol) in dry CH₂Cl₂ (40 mL) was added oxalyl chloride (3.9 mL, 45.0 mmol) at 0° C. After addition temperature of the reaction mixture was slowly raised to RT and then stirred at RT for 8 h. The reaction was monitored by TLC and after completion of the reaction; solvent was distilled off and the crude was dissolved in dry ether (50 mL). To this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuum at RT to give the desired crude product (2.5 g) as a liquid.

### D) Preparation of 5-[5-(methyl-phenyl-amino)-pentyl]-6H-[1,3,4]thiadiazin-2-ylamine

To the stirred solution of 1-bromo-7-(methyl-phenyl-amino)-heptan-2-one (2.5 g, 8.38 mmol) in EtOH (60 mL) at RT was added thiosemicarbazide (764 mg, 8.38 mmol) and stirred for overnight. The reaction was monitored by TLC and after completion of the reaction; mixture was filtered and the solid mass was purified by column chromatography on silica gel using MeOH:CH₂Cl₂ (1:9) to give 400 mg of the desired product as blue sticky solid.

### Example 54

### 5-(5-Pyridin-2-yl-pentyl)-6H-[1,3,4]thiadiazin-2-ylamine

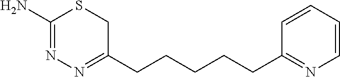 [SMILES: NC1=NN=C(CCCCCc2ccccn2)CS1]

### A) Preparation of 2-(5-bromo-pentyl)-pyridine

To the stirred solution of diisopropyl amine (3.0 mL, 24.6 mmol) in THF (10 mL) at −78° C. was added n-BuLi (1.2 M, 20.5 mL) dropwise and is allowed to stand for 0° C. slowly. To this pale yellow solution at 0° C. was added HMPA (4.3 mL, 24.6 mmol) an the total reaction mixture was allowed to stir for 15 min and to this 2-picoline (2.45 mL, 24.6 mmol) in THF was added at 0° C. and stirred for 30 min at 0° C. To a stirred solution of compound 1,4-dibromo butane (2.95 mL, 24.6 mmol) in THF (20 mL) was added the above reaction mixture at 0° C. and is allowed to stirred at RT for overnight. Then pH of the reaction mixture was adjusted to neutral with 10% aqueous HCl and the two layers were separated. The aqueous layer was basified with solid KOH and extracted with ether. The combined ether layer was dried to give the crude material, which was purified by column chromatography using 30% hexane-ethyl acetate to give the pure desired product (2.9 g) as liquid.

### B) Preparation of 6-pyridin-2-yl-hexanenitrile

To the stirred solution of 2-(5-bromo-pentyl)-pyridine (8.8 g, 38.5 mmol) in ethanol (90 mL) was added aqueous KCN solution (3.0 g, 46.2 mmol in 35 mL water) and the mixture was heated to reflux for 28 h. The progress of the reaction was monitored by TLC and after completion of the reaction mixture was cooled to RT. Then solvent was evaporated and the aqueous layer was subjected to a standard ethyl acetate work up to give the desired product (3.3 g) as a liquid.

### C) Preparation of 6-pyridin-2-yl-hexanoic acid

To the stirred solution of 6-pyridin-2-yl-hexanenitrile (3.3 g, 18.9 mmol) in ethanol (30 ml) was added aqueous NaOH (1.5 g, 37.8 mmol in 15 mL water) and after addition mixture was heated to reflux for 6 h. The progress of the reaction was monitored by TLC and after completion of the reaction ethanol was evaporated. The residue was dissolved in water (10 mL) followed by addition of 6 N HCl until pH 4-5. The whole reaction mixture was then subjected to a standard chloroform work up to give desired product (2.6 g) as a brown color solid.

### D) Preparation of 1-bromo-7-pyridin-2-yl-heptan-2-one

To the stirred solution of 6-pyridin-2-yl-hexanoic acid (2.6 g, 13.5 mmol) in dry CH₂Cl₂ (30 mL) was added oxalyl chloride (4.64 mL, 54.0 mmol) at 0° C. After addition mixture was stirred for 1 h at 0° C. The reaction was monitored by TLC and after completion of the reaction; solvent was distilled off and the crude was dissolved in dry ether (50 mL). To this solution was added an ethereal solution of CH₂N₂ (prepared from nitrosomethylurea) at 0° C. After 1 h at this temperature, the solvent was removed at 0° C. by means of a stream of N₂. The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuum at RT to give crude desired product (500 mg) as a liquid.

### E) Preparation of 5-(5-pyridin-2-yl-pentyl)-6H-[1,3,4]thiadiazin-2-ylamine

To the stirred solution of 1-bromo-7-pyridin-2-yl-heptan-2-one (470 mg, 1.73 mmol) in EtOH (10 mL) at RT was added thiosemicarbazide (160 mg, 1.73 mmol) and stirred for overnight. The reaction was monitored by TLC and after completion of the reaction; mixture was filtered and the solid mass was purified by column chromatography on silica gel using MeOH:CH₂Cl₂ (1:9) to give pure desired product (60 mg) as off-white solid.

### Example 55

### 4-(5-methoxy-pentyl)-5-methyl-4H-[1,3,4]-thiadiazin-2-ylamine

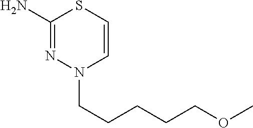

### A) Preparation of 5-methoxy-pentan-1-ol

To the stirred solution of NaH (5.7 g, 0.24 mol) in dry THF (200 mL) at 0° C. was added pentane-1, 5-diol (25.0 g, 0.24 mol, in 250 mL dry THF) and the mixture was stirred for 10 min. Then methyl iodide in dry THF (100 mL) was added to the reaction mixture at 0° C. After addition temperature of the reaction mixture was slowly raised to RT and then stirred at RT for 8 h. The progress of the reaction was monitored by TLC and after completion of the reaction; mixture was cooled to 0° C. and saturated aqueous ammonium chloride solution was added to the reaction mixture. The reaction mixture was then subjected to a standard ethyl acetate work up to give crude residue (23.0 g) as a liquid; which was used for the next step without further purification, as GCMS was consistent with the desired product.

### B) Preparation of 5-methoxy-pentanal

A solution of DMSO (30 mL, 0.42 mol) in CH₂Cl₂ (230 mL) was cooled to 78° C. and then oxalyl chloride (18.4 mL, 0.21 mol in CH₂Cl₂ (80 mL)) was added slowly. After 10 minute of stirring a solution of 5-methoxy-pentan-1-ol (23.0 g, 0.19 mol) in CH₂Cl₂ (80 mL) was added slowly over a period of 30 min at −78° C. Then triethyl amine (136 mL, 0.97 mol) was added in 5 minute and the reaction mixture was allowed to come to RT. The progress of reaction was monitored by TLC and after completion of the reaction water was added and the mixture was subjected to a standard CH₂Cl₂ work-up to give the crude desired product (2.5 g) as a liquid. The crude was used for the next step without purification.

### C) Preparation of (E)-2-(5-methoxypentylidene)hydrazinecarbothioamide

To the stirred solution of 5-methoxy-pentanal (500 mg, 4.30 mmol) and thiosemicarbazide (390 mg, 4.30 mmol) in methanol (10 mL) was added a catalytic amount of Pd/C and the mixture was stirred at RT for 8 h under hydrogen atmosphere. The reaction was monitored by TLC and after completion of the reaction mixture was filtered and concentrated under vacuum to give the crude residue; which was purified by column chromatography using 2% MeOH in CH₂Cl₂ to give the pure desired product (500 mg) as a solid.

### D) Preparation of 2-(5-methoxypentyl)-hydrazinecarbothioamide

To the stirred solution of (E)-2-(5-methoxypentylidene)hydrazinecarbothioamide (500 mg, 2.60 mmol) in MeOH (10 mL) was added NaBH₄ (220 mg, 3.2 mmol) at 0° C. and the resulting mixture was stirred at RT for 14 h. The reaction was monitored by TLC and TLC shows starting was present then we heated the reaction mixture and found no change in TLC. After that mixture was concentrated under vacuum and the crude was purified by column chromatography using 2% MeOH in CH₂Cl₂ to give 100 mg desired product and rest of the compound remains as starting material (400 mg).

### E) Preparation of 4-(5-methoxy-pentyl)-5-methyl-4H-[1,3,4]-thiadiazin-2-ylamine

To the stirred solution of 2-(5-methoxypentyl)-hydrazinecarbothioamide (1.0 g, 5.0 mmol) in ethanol (10 mL) was added 1-bromo-propan-2-one (850 mg, 6.0 mmol) and the mixture was stirred at RT for 8 h. The reaction was monitored by TLC and after completion of the reaction; mixture was concentrated and the residue was purified by column chromatography using 5% MeOH in CHCl₃ to give pure desired product (50 mg) as a liquid.

### Example 56. ((E)-5-Hept-1-enyl)-6H-[1,3,4]thiadiazin-2-ylamine.hydrobromide

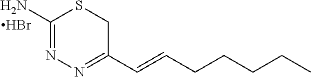

### A) Preparation of (E)-1-bromo-non-3-en-2-one

To the stirred solution of diisopropyl amine (10.0 mL, 69.0 mmol) in 80 mL dry THF was cooled to −78° C. under nitrogen atmosphere, and n-BuLi (32 mL, 64.6 mL, 2.2 M in hexane) was added dropwise. A solution of dibromomethane (11.2 g, 64.4 mmol) in THF (50 mL) was cooled to −90° C. with diethyl ether/dry ice in another flask. The lithium diisopropylamide solution was added dropwise to the stirred dibromomethane solution. After 5 min, a solution of (E)-oct-2-enoic acid ethyl ester (5.0 g, 29.4 mmol) in THF (50 mL) was added dropwise to the reaction mixture and after an additional 10 minute, n-BuLi (22 mL, 44.04 mmol) was added. Then after 5 minute the reaction mixture was added via cannula, to a rapidly stirring solution of 40 mL of acetyl chloride in 300 mL of absolute ethanol cooled to −78° C. The mixture was diluted with 1.5 1 of ether washed with aqueous solutions of cold 10% H₂SO₄ (300 mL×2), 5% NaHCO₃ (150 mL) and brine (100 mL), dried over Na₂SO₄ and concentrated under vacuum to give crude residue (7.5 g); which was used for the next step without purification as NMR was consistent with the desired product.

### B) Preparation of (E)-5-hept-1-enyl)-6H-[1,3,4]thiadiazin-2-ylamine.hydrobromide

To the stirred solution of (E)-1-bromo-non-3-en-2-one (6.3 g, 28.7 mmol) in ethanol (50 mL) was added thiosemicarbazide (2.6 g, 28.7 mmol) at RT and the mixture was stirred for 12 h. The reaction was monitored by TLC and after completion of the reaction; solvent was evaporated and purified by column chromatography using CH₂Cl_{2:}MeOH (9:1) to give the desired product with non-separable impurities (500 mg) and was purified again by preparative HPLC to give the pure desired product (50 mg) as a brown semi-solid.

### Example 57

### 5-[5-(4-Fluoro-phenoxy)-pentyl]-6H-[1,3,4]thiadiazin-2-ylamine hydrobromide.

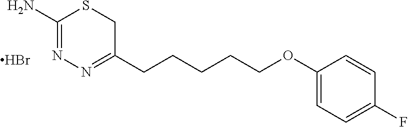 [SMILES: Br ; NC1=NN=C(CCCCCOc2ccc(F)cc2)CS1]

### A) Preparation of 1-(5-bromo-pentyloxy)-4-fluoro-benzene

To the stirred solution of 4-fluorophenol (5.0 g, 45.0 mmol) in water (20 ml) was added 1, 5-dibromo pentane (7.6 mL, 55.0 mmol) and the reaction mixture was heated to reflux and at the reflux condition aqueous NaOH (1.6 g in 20 ml water) solution was added slowly. After addition reflux was continued for another 8 h. The progress of the reaction was monitored by TLC and after completion of the reaction, mixture was cooled to RT. The upper layer of the mixture was separated and discarded and the lower layer was washed with ethyl acetate. The combined ethyl acetate layers were washed with dilute NaOH solution and water. The organic layers were dried and then evaporated under reduced pressure to give crude product; which was purified by column chromatography using hexane-ethyl acetate (98:2) to give the pure desired product (7.0 g) as a liquid.

### B) Preparation of 6-(4-fluoro-phenoxy)-hexanenitrile

To the stirred solution of 1-(5-bromo-pentyloxy)-4-fluoro-benzene (8.5 g, 32.0 mmol) in ethanol (100 ml) was added aqueous KCN solution (1.7 g, 26.0 mmol; in 35 mL of water) and the mixture was heated to reflux for 28 h. The progress of the reaction was monitored by TLC and after completion of the reaction, mixture was cooled to RT and then evaporated the ethanol layer and the aqueous layer was extracted with ethyl acetate. The combine organic layers were washed with aqueous NaOH and the organic layer was then dried and evaporated to give desired product as a white semi-solid (6.8 g).

### C) Preparation of 6-(4-fluoro-phenoxy)-hexanoic acid

To the stirred solution of 6-(4-fluoro-phenoxy)-hexanenitrile (3.5 g, 18.3 mmol) in ethanol (50 ml) was added aqueous NaOH solution (1.1 g in 15 mL of water) and after addition the reaction mixture was heated to reflux for 8 h. The progress of the reaction was monitored by TLC and after completion of the reaction, mixture was cooled to 0° C. and pH of the reaction mixture was adjusted to 3 by adding dilute HCl and was then subjected to a standard CH₂Cl₂ work up to give 3.5 g of the desired product as a semi-solid.

### D) Preparation of 1-bromo-7-(4-fluoro-phenoxy)-heptan-2-one

To the stirred solution of 6-(4-fluoro-phenoxy)-hexanoic acid (3.2 g, 14.1 mmol) in dry toluene (50 ml) was added thionyl chloride (1.5 ml, 21.1 mmol, in 0.1 ml DIVIF) at 0° C. and after addition temperature of the reaction mixture was slowly raised to RT and then refluxed for 3 h. The progress of the reaction was monitored by TLC and after completion of the reaction solvent was evaporated and was dissolved in CH₂Cl_{2.} To this solution was added an ethereal solution of CH₂N₂ (88.0 mmol, prepared from nitrosomethylurea) at −10° C. After 1 h at this temperature, the solvent was removed at 0° C. by means of a stream of N₂.The crude diazoketone was dissolved in CH₂Cl₂ (ml) and the solution was cooled to −70° C. A saturated solution of HBr in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed in vacuo to give crude desired product (3.2 g) as a brownish liquid and was used for the next step without purification.

### E) Preparation of 5-[5-(4-fluoro-phenoxy)-pentyl]-6H-[1,3,4]thiadiazin-2-ylamine

To the stirred solution of 1-bromo-7-(4-fluoro-phenoxy)-heptan-2-one (3.5 g, 11.0 mmol) in ethanol (40 mL) was added thiosemicarbazide (1.0 g, 11.0 mmol) at RT and the mixture was stirred for 12 h. The reaction was monitored by TLC and after completion of the reaction; solvent was evaporated and purified by column chromatography using CH₂Cl₂:MeOH (9:1) to give the desired product (120 mg) as a white solid.

### Example 58. 2-[5-(2-Amino-6H-[1,3,4]thiadiazin-5-yl)-pentyl]-isoindole-1,3-dione.hydrobromide

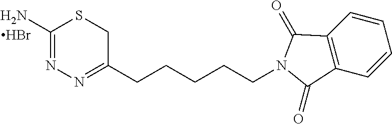 [SMILES: Br ; NC1=NN=C(CCCCCN2C(=O)c3ccccc3C2=O)CS1]

### A) Preparation of 6-(1,3-dioxo-1,3-dihydro-isoindo1-2-yl)-hexanoic acid

6-Amino-hexanoic acid (880 mg, 6.75 mmol) and isobenzofuran-1, 3-dione (1.0 g, 6.75 mmol) in 1,4-dioxane were heated at 160° C. for 12 h in a sealed tube. The reaction was monitored by TLC and after completion of the reaction; solvent was evaporated to give a desired product (1.2 g) as a white color solid and used for the next step without purification.

### B) Preparation of 2-(7-bromo-6-oxo-heptyl)-isoindole-1,3-dione

To the stirred solution of 6-(1,3-dioxo-1,3-dihydro-isoindo1-2-yl)-hexanoic acid (4.0 g, 15.3 mmol) in dry CH₂Cl₂ (80 mL) was added oxalyl chloride (5.2 mL, 61.2 mmol) at 0° C. After addition temperature of the reaction mixture was slowly raised to RT and then stirred at RT for 8 h. The reaction was monitored by TLC and after completion of the reaction; solvent was distilled off and the crude was dissolved in dry ether (100 mL). To this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −70° C. A saturated solution of HBr in CH₂Cl₂ was then added dropwise, care being taken that excess HBr solution was not added. When the diazoketone has been consumed (TLC analysis), the temperature of the reaction mixture was lowered to −75° C. and a stream of N₂ was passed through the solution until the solvent volume had been reduced to about one-third of the original. The remainder of the solvent was then removed in vacuum at RT to give the desired crude product (5.2 g) as a brown color gummy liquid.

### C) Preparation of 2-[5-(2-amino-6H-[1,3,4]thiadiazin-5-yl)-pentyl]-isoindole-1,3-dione.hydrobromide

To the stirred solution of 2-(7-bromo-6-oxo-heptyl)-isoindole-1, 3-dione (5.20 g, 15.5 mmol) in ethanol (150 mL) was added thiosemicarbazide (1.40 g, 15.5 mmol) at RT and the mixture was stirred for 12 h. The reaction was monitored by TLC and after completion of the reaction; solvent was evaporated and purified by column chromatography using CH₂Cl_{2:}MeOH (9:1) to give the desired product (900 mg) as an off-white solid.

### Example 59

### 5-(4-chloro-phenyl)-4-methyl-4H-[1,3,4]thiadiazin-2-ylamine

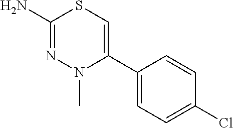

### A) Preparation of 5-(4-chloro-phenyl)-4-methyl-4H-[1,3,4]-thiadiazin-2-ylamine

To the stirred solution of 2-bromo-1-(4-chloro-phenyl)-ethanone (220 mg, 0.95 mmol) in ethanol (5 mL) was added 2-methylhydrazinecarbothioamide (100 mg, 0.95 mmol) at RT and the mixture was stirred for 12 h. The reaction was monitored by TLC and after completion of the reaction; solvent was evaporated and purified by column chromatography using CH₂Cl_{2:}MeOH (9:1) to give the desired product (40 mg) as an off-white solid.

### Example 60

### 5-(5-Methoxy-pentyl)-4-methyl-4H-[1,3,4]thiadiazin-2-ylamine

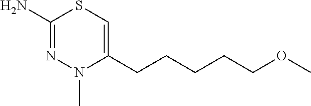

### A) Preparation of 5-(5-methoxy-pentyl)-4-methyl-4H-[1,3,4]thiadiazin-2-ylamine

To the mixture of 1-bromo-propan-2-one (1.0 g, 4.4 mmol) in ethanol (10 mL) was added 2-methylhydrazinecarbothioamide (470 mg, 4.4 mmol) at RT and the mixture was stirred for 10 h at room temperature. The reaction was monitored by TLC and after completion of the reaction mixture was concentrated and purified by column chromatography using ethyl MeOH-CH₂Cl₂ (1:9) to give the desired product (90 mg) as a brown sticky solid.

### Example 61

### 5-(4-Benzofuran-2-yl-butyl)-6H-[1,3,4]thiadiazin-2-ylamine.hydrobromide

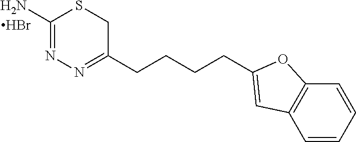 [SMILES: Br ; NC1=NN=C(CCCCc2cc3ccccc3o2)CS1]

### A) Preparation of 6-benzofuran-2-yl-1-bromo-hexan-2-one

To the stirred solution of 5-benzofuran-2-yl-pentanoic acid (2.5 g, 11.46 mmol) in toluene (200 ml) at 0° C. was added thionyl chloride (2.0 g, 17.2 mmol) and a catalytic amount of DMF. After addition temperature of the reaction mixture was slowly raised to room temperature and then refluxed for 3 h. Reaction was monitored by TLC and after completion of the reaction solvent was evaporated. To the remaining residue dry ether was added and cooled the reaction mass to 0 OC. To this solution was added an ethereal solution of CH₂N₂ (prepared from nitrosomethylurea) at 0° C. After 1 h at this temperature, the solvent was removed at 0° C. by means of a stream of N₂.The crude diazoketone was dissolved in dry CH₂Cl₂ and the solution was cooled to −78° C. A saturated solution of HBr in CH₂Cl₂ was then added dropwise, care being taken that excess HBr solution was not added. When the diazoketone has been consumed (TLC analysis), the temperature of the reaction mixture was lowered to −78° C. and a stream of N₂ was passed through the solution until the solvent volume had been reduced to about one-third of the original. The remainder of the solvent was then removed in vacuum at RT to give the crude desired product (2.5 g) as a liquid and used for the next step without purification as GCMS shows desired compound has formed.

### B) Preparation of 5-(4-benzofuran-2-yl-butyl)-6H-[1,3,4]thiadiazin-2-ylamine. Hydrobromide

To the stirred solution of crude 6-benzofuran-2-yl-1-bromo-hexan-2-one (2.5 g, 8.46 mmol) in EtOH (150 mL) at RT was added thiosemicarbazide (620 mg, 6.77 mmol) and stirred for overnight. The reaction was monitored by TLC and after completion of the reaction; mixture was filtered and solid mass was obtained; which was purified by column chromatography on silica gel using MeOH-CH₂Cl₂ (MeOH ratio ranges from 5-7%) to give the desired product (550 mg) as a grey solid.

### Example 62

### 5-(5-Benzyloxy-pentyl)-6H-[1,3,4]thiadiazin-2-ylamine.hydrobromide

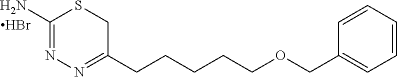 [SMILES: Br ; NC1=NN=C(CCCCCOCc2ccccc2)CS1]

### A) Preparation of 7-Benzyloxy-1-bromo-heptan-2-one

To the stirred solution 6-benzyloxy hexanoic acid (10.0 g, 44.8 mmol) in dry toluene (100 ml) was added thionyl chloride (6.4 g, 53.8 mmol) at 0° C. and after addition temperature of the reaction mixture was slowly raised to RT and then refluxed for 3 h. The progress of the reaction was monitored by TLC and after completion of the reaction solvent was evaporated and was dissolved in CH₂Cl_{2.} To this solution was added an ethereal solution of CH₂N₂ (prepared from N-nitrosomethylurea) at −10° C. After 1 h at this temperature, the solvent was removed at 0° C. by means of a stream of N₂.The crude diazoketone was dissolved in CH₂Cl₂ (ml) and the solution was cooled to −70° C. A saturated solution of HBr in CH₂Cl₂ was then added dropwise, care being taken that excess HBr solution was not added. When the diazoketone has been consumed (TLC analysis), the temperature was lowered to −75° C. and a stream of N₂ was passed through the solution until the solvent volume had been reduced to about one-third of the original. The remainder of the solvent was then removed under vacuum to give crude desired product (4.2 g) and used for the next step without purification.

### B) Preparation of 5-(5-Benzyloxy-pentyl)-6H-[1,3,4]-thiadiazin-2-ylamine. Hydrobromide

To the stirred solution of 7-benzyloxy-1-bromo-heptan-2-one (2.6 g, 8.68 mmol) in ethanol (26 mL) was added thiosemicarbazide (790 mg, 8.65 mmol) at room temperature and the mixture was stirred for 12 h. The reaction was monitored by TLC and after completion of the reaction; solvent was evaporated and purified by column chromatography using CH₂Cl₂:MeOH (9:1) to give the desired product (900 mg) as a pink solid.

### Example 63

### 5-(4-(benzyloxy)butyl)-6H-1,3,4-thiadiazin-2-amine hydrobromide

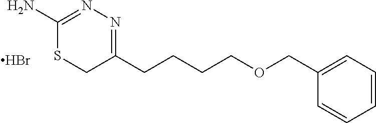 [SMILES: Br ; NC1=NN=C(CCCCOCc2ccccc2)CS1]

### A) Preparation of 6-(benzyloxy)-1-bromohexan-2-one

Tetra-n-butylammonium tribromide (7.2 g, 16 mmol) was added to 6-(benzyloxy)hexan-2-one (3.0 g, 14.56 mmoles) in dichloromethane/Methanol (2:1, 66 mL) at 0° C. The resulting reaction mixture was stirred for 16 h at room temperature. The reaction progress was monitored by TLC. After completion of the reaction, the reaction mass was quenched with 1 drop of water and then concentrated under vacuum to afford crude 6-(benzyloxy)-1-bromohexan-2-one. Crude compound was purified by flash chromatography (siligel 100-200 mesh; Ethyl acetate/pet ether 10:90) to afford pure 6-(benzyloxy)-1-bromohexan-2-one (1.8 g; 43.5%) as brown color semi solid. [TLC system: Ethyl acetate:pet ether (2:8); Rf value: 0.4].

### B) Preparation of 5-(4-(benzyloxy)butyl)-6H-1,3,4-thiadiazin-2-amine hydrobromide

Thiosemicarbazide (577 mg, 6.338 mmol) was added to 6-(benzyloxy)-1-bromohexan-2-one (1.8 g, 6.338 mmoles) in ethanol (25 mL) at room temperature. The resulting reaction mixture was stirred for 16 h at room temperature. Then the reaction mass was heated to 40° C. for 24 hr. The reaction progress was monitored by TLC. On completion of the reaction, the reaction mass was concentrated under vacuum to afford crude compound. Crude compound was purified by flash chromatography (siligel 100-200 mesh; Ethyl acetate/pet ether 50:50) then Ethyl acetate/methanol 80:20) to afford pure 5-(4-(benzyloxy)butyl)-6H-1,3,4-thiadiazin-2-amine hydrobromide (550 mg; 31.3%) as off white solid. [TLC system: Methanol:DCM (2:8); Rf value: 0.2].

### Example 64

### 5-(2-(2-Phenoxyethoxy) ethyl)-6H-1,3,4-thiadiazin-2-amine hydrochloride

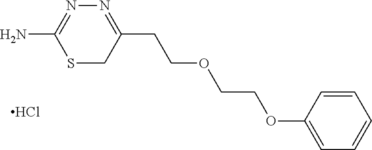 [SMILES: Cl ; NC1=NN=C(CCOCCOc2ccccc2)CS1]

### A) Preparation of 1-Chloro-4-(2-phenoxyethoxy)butan-2-one

A solution of 1.7 M tert-butylmagnesium chloride in tetrahydrofuran (2.6 mL, 4.44 mmol) was added to a stirred solution of methyl 3-(2-phenoxyethoxy) propanoate (250 mg, 1.11 mmol), sodium chloroacetate (194 mg, 1.67 mmol) and triethylamine (0.23 mL, 1.67 mmol) in tetrahydrofuran (10 mL) at 0° C. The reaction mixture was stirred at same temperature for 15 minutes. The reaction mixture was allowed to come to room temperature and stirred for 16 h. The reaction mixture was cooled to 0° C., acidified with 5N hydrochloric acid and added water (25 mL) followed by dichloromethane (30 mL) and stirred for 10min. The organic product was extracted with dichloromethane (50 mL×2). The combined organic layers were washed with water (40 mL), brine solution (40 mL), dried over anhydrous sodium sulfate and solvent was concentrated in vacuo to afford 200 mg (74.62%) of 1-chloro-4-(2-phenoxyethoxy)butan-2-one 06-075d as brownish liquid. [TLC system: Ethyl acetate:Pet-ether (3:7); Rf value: 0.5].

### B) Preparation of 5-(2-(2-Phenoxyethoxy)ethyl)-6H-1,3,4-thiadiazin-2-amine

Thiosemicarbazide (67 mg, 0.74 mmol) was added to a solution of 1-chloro-4-(2-phenoxyethoxy)butan-2-one (200 mg, 0.82 mmol) in acetonitrile (10 mL) and heated at 75-80° C. for 2 h, the mixture was cooled to room temperature, solid was precipitated out, which was collected by filtration and washed with acetonitrile followed by diethyl ether, ethyl acetate and dried in vacuo to afford 80 mg (30.76%) of 5-(2-(2-phenoxyethoxy)ethyl)-6H-1,3,4-thiadiazin-2-amine hydrochloride as off white solid. [TLC system: Dichloromethane:Methanol (0.5:9.5); Rf value: 0.1].

### Example 65

### 5-(5-(Pyridin-2-yloxy) pentyl)-6H-1, 3, 4-thiadiazin-2-amine hydro chloride

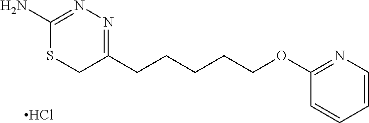 [SMILES: NC1=NN=C(CCCCCOc2ccccn2)CS1 ; Cl]

### A) Preparation of Ethyl 6-(pyridin-2-yloxy) hexanoate

Ethyl 6-bromohexanoate (2.34 g, 10.51 mmol) was added to a solution 2-hydroxypyridine (1.0 g, 10.51 mmol) and potassium hydroxide (2.35 g, 42.06 mmol) in dimethylsulfoxide (10 mL) at room temperature. The reaction mixture was stirred at room temperature 16 h. Reaction was quenched with ice cooled water (50 mL), the organic product was extracted with ethyl acetate (60 mL×2). The combined organic layers were washed with water (40 mL), brine solution (40 mL), dried over anhydrous sodium sulfate and solvent was concentrated in vacuo to give crude compound. The crude compound was purified by flash chromatography to afford 300 mg (12.05%) of ethyl 6-(pyridin-2-yloxy) hexanoate as colorless liquid. [TLC system: Ethyl acetate:Pet-ether (1:4); Rf value: 0.54].

### B) Preparation of 1-Chloro-7-(pyridin-2-yloxy) heptan-2-one

A solution of 1.7 M tert-butylmagnesium chloride in tetrahydrofuran (1.9 mL, 3.21 mmol) was added to a solution of ethyl 6-(pyridin-2-yloxy) hexanoate (190 mg, 0.80 mmol), sodium chloroacetate (140 mg, 1.20 mmol) and triethylamine (0.17 mL, 1.20 mmol) in tetrahydrofuran (10 mL) at 0° C. The reaction mixture was stirred at same temperature for 15min and warmed to room temperature stirred for 16 h. The reaction mixture was cooled to 0° C., acidified with 8N hydrochloric acid and added water (25 mL) followed by dichloromethane (30 mL) and stirred for 10minutes. The reaction mixture was basified with saturated sodium bicarbonate solution, the organic product was extracted with dichloromethane (50 mL×2). The combined organic layers were washed with water (40 mL), brine solution (40 mL), dried over anhydrous sodium sulfate and solvent was concentrated in vacuo to afford 190 mg (98.44%) of 1-chloro-7-(pyridin-2-yloxy) heptan-2-one as brownish liquid. [TLC system: Ethyl acetate:Pet-ether (1:9); Rf value: 0.28].

### C) Preparation of 5-(5-(Pyridin-2-yloxy) pentyl)-6H-1, 3, 4-thiadiazin-2-amine hydro chloride

Thiosemicarbazide (63 mg, 0.7 mmol) was added to a solution of 1-chloro-7-(pyridin-2-yloxy) heptan-2-one (185 mg, 0.76 mmol) in acetonitrile (10 mL) and heated at 75-80° C. for 2 h. The reaction mixture was cooled to room temperature, filtered the precipitated solid, and washed with acetonitrile followed by diethyl ether. Finally the solid was washed with pentane and dried in vacuo to afford 140 mg (65.72%) of 5-(5-(pyridin-2-yloxy) pentyl)-6H-1, 3, 4-thiadiazin-2-amine hydro chloride as Cream solid. [TLC system: Ethyl acetate:Pet-ether (1:4); Rf value: 0.1].

### Example 66

### 5-(4-(4-Ethylphenoxy)butyl)-6H-1,3,4-thiadiazin-2-amine hydrochloride

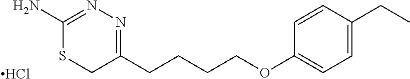 [SMILES: CCc1ccc(OCCCCC2=NN=C(N)SC2)cc1 ; Cl]

### A) Preparation of Ethyl 5-(4-ethylphenoxy)pentanoate

Ethyl 5-bromopentanoate (2.05 g, 9.83 mmol) was added was added to a stirred suspension of 4-ethylphenol (1.0 g, 8.196 mmol) and potassium carbonate (1.24 g, 9.01 mmol) in dimethylformamide (10 mL) at room temperature and the mixture was heated at 60° C. for 6 h. The reaction mixture was quenched with ice cooled water (50 mL), the organic product was extracted with ethyl acetate (60 mL×2). The combined organic layers were washed with ice cold water (40 mL×2), brine solution (40 mL), dried over anhydrous sodium sulfate and solvent was concentrated in vacuo to give crude compound. The crude compound was purified by flash chromatography over silica gel (100-200 mesh) using 10-12% ethylacetate in pet ether as eluent to afford 900 mg (45.0%) of ethyl 5-(4-ethylphenoxy)pentanoate as yellow color liquid. [TLC system: Ethylacetate:Pet-ether (1:9); Rf value: 0.6].

### B) Preparation of 1-Chloro-6-(4-ethylphenoxy)hexan-2-one

A solution of 1.7 M tert-butylmagnesium chloride in tetrahydrofuran (5.53 mL, 16.0 mmol) was added to a stirred solution of ethyl 5-(4-ethylphenoxy) pentanoate (1.0 g, 4.0 mmol), sodium chloroacetate (698 mg, 6.0 mmol) and triethylamine (0.842 mL, 6.0 mmol) in tetrahydrofuran (50 mL) at 0° C. The reaction mixture was stirred at same temperature for 15 minutes. The mixture allowed to come to room temperature and stirred for 16 h. The reaction mixture was cooled to 0° C., acidified with 5N hydrochloric acid and water (30 mL) was added, followed by dichloromethane (50 mL) and stirred for 10 minutes. The aqueous layer was extracted with dichloromethane (80 mL×2). The combined organic layers were washed with water (40 mL), brine solution (40 mL), dried over anhydrous sodium sulfate and solvent was concentrated in vacuo to afford 600 mg (60.0%) of 1-chloro-6-(4-ethylphenoxy)hexan-2-one as yellow solid. [TLC system: Ethyl acetate:Pet-ether (1:9); Rf value: 0.5].

### C) Preparation of 5-(4-(4-ethylphenoxy)butyl)-6H-1,3,4-thiadiazin-2-amine hydrochloride

Thiosemicarbazide (193 mg, 2.12 mmol) was added to a solution of 1-chloro-6-(4-ethylphenoxy)hexan-2-one (600 mg, 2.36 mmol) in acetonitrile (20 mL) and heated at 75-80° C. for 2 h. The reaction mixture was cooled to room temperature, filtered the precipitated solid and washed with acetonitrile followed by diethyl ether. Finally the solid was washed with ethylacetate and dried in vacuo to afford 200 mg (25.94%) of 5-(4-(4-ethylphenoxy)butyl)-6H-1,3,4-thiadiazin-2-amine hydrochloride as off white solid. [TLC system: Dichloromethane:Methanol (0.5:9.5); Rf value: 0.1].

### Example 67

### 6-(2-Butoxyethyl)-4H-[1,3,4]thiadiazin-2-ylamine.hydrobromide

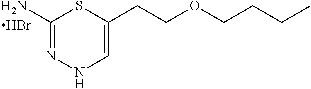

### A) Preparation of 4-butoxy-butan-1-ol

To the stirred solution of butane-1, 4-diol (20.0 g, 0.22 mol) in dry DMF (200 mL) was added NaH (60%, 10.6 g, 0.44 mol) portion wise over a period of 30 min at 0° C. After addition mixture was stirred for 30 min then butyl bromide (30.4 g, 0.22 mol) was added dropwise over 20 min at 0° C. and stirred at RT for 16 h. The progress of the reaction was monitored by TLC and after completion of the reaction; mixture was cooled to 0° C. and saturated aqueous ammonium chloride solution was added to the reaction mixture. The reaction mixture was then subjected to a standard ethyl acetate work up to give crude residue (33.0 g); which was used for the next step without further purification, as GCMS was consistent with the desired product.

### B) Preparation of 4-butoxy-butyraldehyde

A solution of DMSO (14.5 mL, 0.2 mol) in CH₂Cl₂ (100 mL) was cooled to −78° C. and oxalyl chloride (13.2 mL, 0.15 mol in CH₂Cl₂ (50 mL)) was added slowly. After 10 min of stirring a solution of 4-butoxy-butan-1-ol (15.0 g, 0.1 mol) in CH₂Cl₂ (100 mL) was added slowly over a period of 30 min at −78° C. Then triethyl amine (51.6 g, 0.51 mol) was added followed by 300 mL of water and the mixture was subjected to a standard CH₂Cl₂ work up to give the crude residue (11.2 g); which was used for the next step without purification, as GCMS was consistent with the desired product.

### C) Preparation of 2-bromo-4-butoxy-butyraldehyde

To the stirred cold (0° C.) solution of 4-butoxy-butyraldehyde (10.0 g, 0.7 mol) in dry ether (100 mL) was added portion wise a bromine-dioxane complex (17.2 g, 0.7 mol) and stirred at RT for 16 h. The reaction was monitored by TLC and after completion of the reaction mixture was diluted with ethyl acetate (100 mL) and then washed with 5% aqueous Na2CO3 solution (100 mL), water and dried to give the crude material (13.2 g) which was used for the next step without purification.

### D) Preparation of 6-(2-butoxy-ethyl)-4H-[1,3,4]thiadiazin-2-ylamine.hydrobromide

To the stirred solution of 2-bromo-4-butoxy-butyraldehyde (13.2 g, 59.1 mmol) in EtOH (100 mL) at RT was added thiosemicarbazide (4.32 g, 47.3 mmol) and stirred for overnight. The reaction was monitored by TLC and after completion of the reaction; mixture was filtered and the solid mass was purified by column chromatography on silica gel using MeOH:CH₂Cl₂ (1:9) to give 170 mg of the desired product as brown solid.

### Example 68

### 5-(4-Benzyloxybutyl)-6H-1,3,4-thiadiazin-2-amine

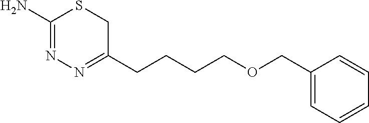 [SMILES: NC1=NN=C(CCCCOCc2ccccc2)CS1]

### a) 3-(Benzyloxy)propan-1-ol

The title compound was prepared according to a modified literature procedure.[Biochemistry, 2001, 40(41), 12254-12265] Potassium hydroxide (6.60 g, 0.100 mol, 85%) was mixed with 1,3-propanediol (18.1 mL, 0.25 mol) and the reaction was stirred for 1.5 h until all potassium hydroxide was dissolved (the temperature was increased from 20° C. to 40° C.). The temperature was then increased to 90° C. and benzyl chloride (11.5 mL, 0.100 mol) was added slowly. The temperature was kept at 90° C. for additional 15 min. The temperature was then increased to 130° C. and the reaction stirred for 2 h. The reaction was cooled to room temperature and water (100 mL) was added. The aqueous phase was extracted with ethyl acetate (3×50 mL). The combined organic layers were washed with water (2×50 mL), dried over sodium sulfate, filtered and concentrated to yield 15.1 g of the crude product. MS (ESI⁺): m/z 167 [M+H]⁺. The crude product was used in the next step without further purification. (The product is commercially available, CAS 4799-68-2).

### b) 3-(Benzyloxypropyl) methanesulfonate

The title compound was prepared according to a modified literature procedure.[Biochemistry, 2001, 40(41), 12254-12265] 3-(Benzyloxy)propan-1-ol (15.1 g, 90.8 mmol) was mixed with pyridine (36 mL). Methanesulfonyl chloride (8.06 mL, 105 mmol) was then slowly added at 0° C. and the reaction stirred for 3 h at 0° C. Water (100 mL) was added and the aqueous layer was exacted with ethyl acetate (4×50 mL). The combined organic layers were washed with 4 M HCl (50 mL), water (50 mL), saturated aqueous NaHCO₃ (50 mL), and water (50 mL), dried over sodium sulfate, filtered and concentrated to yield 24.4 g of crude product. Some pyridine remained but the crude product was taken to the next step without further purification.

### c) 3-Iodopropoxymethylbenzene

The title compound was prepared according to a modified literature procedure. [Biochemistry, 2001, 40(41), 12254-12265] Sodium iodide (17.9 g, 120 mmol) was mixed with acetone (100 mL) at room temperature. Then 3-(benzyloxypropyl) methanesulfonate (9.78 g, 40.0 mmol) dissolved in acetone (20 mL) was added and the reaction was stirred at room temperature overnight. Water (100 mL) was added to dissolve the formed precipitate. The mixture was concentrated in order to remove some of the acetone (about 80 mL). The mixture was extracted with ethyl acetate (3×100 mL). The combined organic layers were washed with saturated aqueous Na₂S₂O₃ (50 mL), water (100 mL), dried over sodium sulfate, and concentrated to yield 9.23 g of the crude product.

### d) 6-Benzyloxyhexan-2-one

The title compound was prepared according to a modified literature procedure. [Biochemistry, 2001, 40(41), 12254-12265] Methyl acetyl acetate (3.5 mL, 32 mmol) was dissolved in dimethoxyethane (22 mL). Sodium methoxide (1.75 g, 32.0 mmol) was then added and the reaction stirred at room temperature for 30 min. Then 3-iodopropoxymethylbenzene (5.52 g, 20.0 mmol) dissolved in dimethoxyethane (5 mL) was added and the reaction mixture was heated at 85° C. overnight. After cooling to room temperature, 2 M aqueous sodium hydroxide (60 mL) was added and the reaction was heated at reflux for another 2 h. The reaction mixture was allowed to cool to room temperature and 50% sulfuric acid (12.5 mL) was added (pH=2-3). The reaction was then heated at reflux for 2 h. Water (100 mL) was added and the mixture was extracted with ethyl acetate (3×50 mL). The combined organic layers were washed with 10% aqueous NaHCO₃ (50 mL), and water (2×50 mL), dried over sodium sulfate, filtered and concentrated. The residue was purified with column chromatography (heptane/ethyl acetate 80:20) to give 3.64 g (88%) of the title compound. MS (ESI⁺): m/z 207 [M+H]⁺.

### e) 6-Benzyloxy-1-bromo-hexan-2-one

6-Benzyloxyhexan-2-one (412 mg, 2.00 mmol) was dissolved in dichloromethane (12 mL) and methanol (6 mL). Tetra-n-butylammonium tribromide (1.06 g, 2.20 mmol) was then added at 0° C. The reaction was stirred at room temperature for 3 days. The reaction was quenched with a drop of water and the solvent was evaporated. The product was filtered through a silica column (heptane/ethyl acetate 9:1). The fractions containing the product were combined to yield 502 mg of the product as a mixture of isomers. MS (ESI⁺): m/z 285 [M+H]⁺.

### f) 5-(4-Benzyloxybutyl)-6H-1,3,4-thiadiazin-2-amine

Thiosemicarbazide (46.0 mg, 0.50 mmol) was mixed with ethanol (2 mL) and 6-benzyloxy-1-bromo-hexan-2-one (142 mg, 0.50 mmol) was added. The reaction was stirred at room temperature overnight. The temperature was increased to 40° C. and stirred for another 24 h. The crude material was purified with column chromatography (heptane/ethyl acetate 50:50 to 100% ethyl acetate and then ethyl acetate/methanol 80:20) to yield 29 mg (21%) of the product.

### Example 69

### 5-[4-[(2,4-Difluorophenyl)methoxy]butyl-]-6H-1,3,4-thiadiazin-2-amine

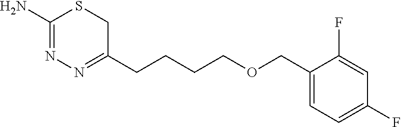 [SMILES: NC1=NN=C(CCCCOCc2ccc(F)cc2F)CS1]

### a) 6-Iodohexan-2-one

Sodium iodide (5.10 g, 34.0 mmol, 2.1 equiv.) was added to a solution of 6-chloro-2-hexanone (2.15 g, 16.0 mmol, 1 equiv.) in acetone (75 mL). The mixture was heated to 60° C. and was stirred at that temperature overnight. After cooling the reaction mixture to room temperature, the precipitate was filtered off and the solvent was removed at reduced pressure. The residue was dissolved in ethyl acetate (200 mL) and water (240 mL) was added. The phases were separated, the organic phase was dried over sodium sulfate and the solvent was removed at reduced pressure to yield 3.14 g (87%) of the title compound. The crude product was used in subsequent reactions without further purification. MS (ESI⁺): m/z 227 [M+H]⁺.

### b) 6-[(2,4-Difluorophenyl)methoxy]hexan-2-one

2,4-Difluorobenzyl alcohol (98%, 390 mg, 2.65 mmol, 1.5 equiv.) was mixed with potassium hydroxide (85%, 175 mg, 1.5 equiv.). The neat reaction was stirred at room temperature for 1 h followed by the addition of 6-iodo-2-hexanone (400 mg, 1.77 mmol, 1 equiv.). After stirring the reaction at room temperature overnight it was then quenched with water and extracted with ethyl acetate (3×). The organic phases were washed with brine, dried over sodium sulfate, and the solvent was removed at reduced pressure. The residue was purified by column chromatography (heptane/ethyl acetate 9:1) and the product was isolated in 181 mg (42%) yield; MS (ESI⁻): m/z 243 [M+H]⁺.

### c) 1-Bromo-6-[(2,4-difluorophenyl)methoxy]hexan-2-one

6-[(2,4-Difluorophenyl)methoxy]hexan-2-one (180 mg, 0.74 mmol, 1 equiv.) was dissolved in dichloromethane (6 mL) and methanol (3 mL). Tetra-n-butylammonium tribromide (394 mg, 1.1 equiv.) was thereafter added and the reaction was stirred at room temperature overnight. The reaction was quenched with 1 drop of water and the solvent was removed at reduced pressure. The material was dried under a flow of air for 15 min and the crude mixture was thereafter used directly in the subsequent step.

### d) 5-[4-[(2,4-Difluorophenyl)methoxy]butyl]-6H-1,3,4-thiadiazin-2-amine

The crude mixture was dissolved in ethanol (3 mL), thiosemicarbazide (75 mg, 0.82 mmol, 1.1 equiv.) was added and the solution was stirred at room temperature overnight. The solvent was removed at reduced pressure and the residue was purified by column chromatography (ethyl acetate then ethyl acetate:methanol 9:1). The product was thereafter washed with ethyl acetate and isolated in 30 mg yield (13% over 2 steps, c-d).

### Example 70

### 5-[4-[[4-(Trifluoromethyl)phenyl]methoxy]butyl]-6H-1,3,4-thiadiazin-2-amine

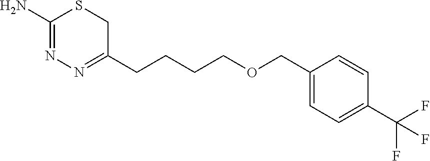 [SMILES: NC1=NN=C(CCCCOCc2ccc(C(F)(F)F)cc2)CS1]

### a) 6-[[4-(Trifluoromethyl)phenyl]methoxy]hexan-2-one

Prepared as described for 6-[(2,4-difluorophenyl)methoxy]hexan-2-one (Example 69b). The reaction was performed in 0.5 mL toluene. Starting from 4-(Trifluoromethyl)benzyl alcohol (292 mg, 1.66 mmol, 1.5 equiv.) and 6-iodohexan-2-one (250 mg, 1.11 mmol, 1 equiv.) yielded 93 mg (31%) of the product. MS (ESI⁻): m/z 275 [M+H]⁺.

### b) 1-Bromo-6-[[4-(trifluoromethyl)phenyl]methoxy]hexan-2-one

Prepared as described for 1-bromo-6-[(2,4-difluorophenyl)methoxy]hexan-2-one, (Example 69c). Starting from 6[[4-(trifluoromethyl)phenyl]methoxy]hexan-2-one (93 mg, 0.34 mmol, 1 equiv.) and tetra-n-butylammonium tribromide (180 mg, 0.373 mmol, 1.1 equiv.) yielded a crude mixture that was used without further purification in step c.

### c) 5-[4-[[4-(Trifluoromethyl)phenyl]methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[[4-(trifluoromethyl)phenyl]methoxy]hexan-2-one gave 18 mg (16% over 2 steps, b-c) of the title compound.

### Example 71

### 5-[4-[(3-Chlorophenyl)methoxy]butyl]-6H-1,3,4-thiadiazin-2-amine

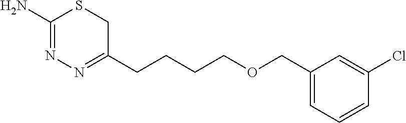 [SMILES: NC1=NN=C(CCCCOCc2cccc(Cl)c2)CS1]

### a) 6-[(3-Chlorophenyl)methoxy]hexan-2-one

Prepared as described for 6-[(2,4-difluorophenyl)methoxy]hexan-2-one (Example 69b). Starting from 3-chlorobenzyl alcohol (386 mg, 2.65 mmol, 1.5 equiv.) and 6-iodohexan-2-one (400 mg, 1.77 mmol, 1 equiv.) yielded 163 mg (38%) of the product. MS (ESI⁺): m/z 241 [M+H]⁺.

### b) 1-Bromo-6-[(3-chlorophenyl)methoxy]hexan-2-one

Prepared as described for 1-bromo-6-[(2,4-difluorophenyl)methoxy]hexan-2-one, (Example 69c). Starting from 6-[(3-chlorophenyl)methoxy]hexan-2-one (163 mg, 0.68 mmol, 1 equiv.) and tetra-n-butylammonium tribromide (359 mg, 0.74 mmol, 1.1 equiv.) yielded a crude mixture that was used without further purification in step c.

### c) 5-[4-[(3-Chlorophenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[(3-chlorophenyl)methoxy]hexan-2-one gave 35 mg yield (17% over 2 steps, b-c) of the title compound in.

### Example 72

### 5-[4-[(4-Fluoro-3-methyl-phenyl)methoxy]butyl]-6H-1,3,4-thiadiazin-2-amine

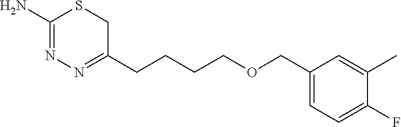 [SMILES: Cc1cc(COCCCCC2=NN=C(N)SC2)ccc1F]

### a) 6-[(4-Fluoro-3-methyl-phenyl)methoxy]hexan-2-one

Prepared as described for 6-[(2,4-difluorophenyl)methoxy]hexan-2-one (Example 69b). The reaction was performed in 0.5 mL toluene. Starting from 4-fluoro-3-methylbenzyl alcohol (250 mg, 1.78 mmol, 1.5 equiv.) and 6-iodohexan-2-one (269 mg, 1.19 mmol, 1 equiv.) yielded 220 mg (39%) of the product. MS (ESI⁺): m/z 239 [M+H]⁺.

### b) 1-Bromo-6-[(4-fluoro-3-methyl-phenyl)methoxy]hexan-2-one

Prepared as described for 1-bromo-6-[(2,4-difluorophenyl)methoxy]hexan-2-one, (Example 69c). Starting from 6-[(4-fluoro-3-methyl-phenyl)methoxy]hexan-2-one (220 mg, 0.92 mmol, 1 equiv.) and tetra-n-butylammonium tribromide (490 mg, 1.02 mmol, 1.1 equiv.) yielded a crude mixture that was used without further purification in step c.

### c) 5-[4-[(4-Fluoro-3-methyl-phenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[(4-fluoro-3-methyl-phenyl)methoxy]hexan-2-one gave 26 mg (9% over 2 steps, b-c) of the title compound in.

### Example 73

### 5-[4-[(3-Bromophenyl)methoxy]butyl]-6H-1,3,4-thiadiazin-2-amine

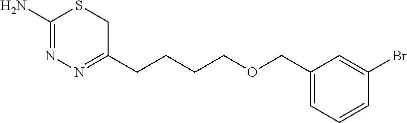 [SMILES: NC1=NN=C(CCCCOCc2cccc(Br)c2)CS1]

### a) 6-Hydroxyhexan-2-one

The title compound was prepared according to a literature procedure. [Org. Lett. 2011, 13(16), 4328-4331] Tetrahydropyran-2-one (4.17 mL, 44.9 mmol, 1 equiv.) was dissolved in diethylether (45 mL) under an inert atmosphere. The solution was thereafter cooled to −78° C. and methyl lithium (1.6 M in diethyl ether, 31.5 mL, 50.4 mmol, 1.1 equiv.) was slowly added dropwise and the solution was stirred at −78° C. for an additional 45 min. The reaction was thereafter quenched with saturated aqueous ammonium chloride (22.5 mL, 164.7 mmol) and the reaction mixture was allowed to reach room temperature. The product was extracted with ethyl acetate, the organic phases were washed with brine and dried over sodium sulfate. The solvent was removed under reduced pressure. The reaction was repeated 2 times. The three different batches were pooled, dissolved in ethyl acetate and filtered thought a pad of silica. The solvent was removed at reduced pressure to afford 12.1 g (73%) of the title compound. MS (ESI⁺): m/z 117 [M+H]⁺.

### b) 6-[(3-Bromophenyl)methoxy]hexan-2-one

The title compound was prepared according to a modified literature procedure. [Tetrahedron 2012, 68(1), 370-375] 6-Hydroxyhexan-2-one (4.07 g, 35.0 mmol, 1.25 equiv.), 1-bromo-3-(bromomethyl)benzene (7.0 g, 28 mol, 1 equiv.) and N,N-diisopropylethylamine (9.76 mL, 56.0 mmol, 2 equiv.) were mixed under an inert atmosphere. The reaction was thereafter heated to 150° C. and stirred at that temperature for 4.5 h. Ethyl acetate and 10% NaHSO₄ in water were added and the product was extracted with ethyl acetate (x3). The combined organic phases were dried over sodium sulfate and the solvent was removed under reduced pressure. The amount of 3-bromobenzyl alcohol formed (by-product) was estimated to be 15% from LC/MS (if wt. %, approx. 1.27 g). The benzylic alcohol formed as a by-product was silylated to simplify the purification by column chromatography. Thus, the crude was dissolved in dichloromethane (50 mL). Then, tert-Butyldimethylsilyl chloride (1.13 g, 7.47 mmol, 1.1 equiv. with respect to the estimated amount of 3-bromobenzyl alcohol) was added followed by imidazole (508 mg, 7.47 mmol, 1.1 equiv. with respect to the estimated amount of 3-bromobenzyl alcohol). The reaction was followed by LC/MS. After 5 min 90% of the benzylic alcohol was silylated and another portion of tert-butyldimethylsilyl chloride (147 mg) and imidazole (66 mg) was added. After 5 min only trace of 3-bromobenzyl alcohol was still observed. Finally, tert-butyldimethylsilyl chloride (15 mg), imidazole (7 mg) were added and the reaction was stirred for an additional 10 min before work-up. The solid was filtered off, water was added and the product was extracted with dichloromethane. The dichloromethane was finally filtered through a pad of silica. The product was eluted using ethyl acetate and the solvent was removed under reduced pressure. The title compound was purified by column chromatography (dichloromethane/heptane/ethyl acetate 65:30:5) to afford 5.4 g (68%) of the product. MS (ESI⁺): m/z 285, 287 [M+H]⁺.

### c) 1-Bromo-6-[(3-bromophenyl)methoxy]hexan-2-one

Prepared as described for 1-bromo-6-[(2,4-difluorophenyl)methoxy]hexan-2-one, (Example 69c). Starting from 6-[(3-bromophenyl)methoxy]hexan-2-one (200 mg, 0.70 mmol, 1 equiv.) and tetra-n-butylammonium tribromide (372 mg, 0.77 mmol, 1.1 equiv.) yielded a crude mixture that was used in step d without further purification.

### d) 5-[4-[(3-Bromophenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). The reaction was performed using the crude mixture from step c containing 1-bromo-6-[(3-bromophenyl)methoxy]hexan-2-one. After stirring the reaction mixture overnight, the product precipitated out of solution and the solid was washed twice with ethyl acetate. Purification by column chromatography was not necessary and the product was isolated in 75 mg yield (30% over 2 steps, c-d).

### Example 74

### 5-[4-[(4-Bromophenyl)methoxy]butyl]-6H-1,3,4-thiadiazin-2-amine

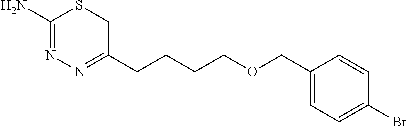 [SMILES: NC1=NN=C(CCCCOCc2ccc(Br)cc2)CS1]

### a) 6-[(4-Bromophenyl)methoxy]hexan-2-one

Prepared as described for 6-[(2,4-difluorophenyl)methoxy]hexan-2-one (Example 69b). The reaction was performed in 3 batches. Starting two batches from 4-bromobenzyl alcohol (2.03 g, 10.6 mmol, 1.5 equiv.) and 6-iodohexan-2-one (1.60 g, 7.08 mmol, 1 equiv.) in 2 mL toluene and the third batch from 4-bromobenzyl alcohol (507 mg, 2.65 mmol, 1.5 equiv.) and 6-iodohexan-2-one (400 mg, 1.77 mmol, 1 equiv.) in 0.5 mL yielded 1.19 g (26%) of the product after a combined final purification. MS (ESI⁺): m/z 285, 287 [M+H]⁺.

### b) 1-Bromo-6-[(4-bromophenyl)methoxy]hexan-2-one

Prepared as described for 1-bromo-6-[(2,4-difluorophenyl)methoxy]hexan-2-one, (Example 69c). Starting from 6-[(4-bromophenyl)methoxy]hexan-2-one (200 mg, 0.700 mmol, 1 equiv.) and tetra-n-butylammonium tribromide (372 mg, 0.770 mmol, 1.1 equiv.) yielded a crude mixture that was used without further purification in step c.

### c) 5-[4-[(4-Bromophenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[(4-bromophenyl)methoxy]hexan-2-one to afford 36 mg (14% over 2 steps, b-c) of the title compound.

### Example 75

### 5-[4-[(2-Bromophenyl)methoxy]butyl]-6H-1,3,4-thiadiazin-2-amine

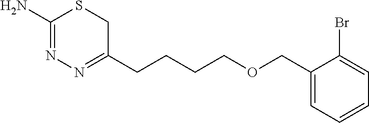 [SMILES: NC1=NN=C(CCCCOCc2ccccc2Br)CS1]

### a) 6-[(2-Bromophenyl)methoxy]hexan-2-one

The title compound was prepared according to a modified literature procedure. [Tetrahedron 2012, 68(1), 370-375] 6-Hydroxyhexan-2-one (4.07 g, 35.0 mmol), 1-bromo-2-(bromomethyl)benzene (7.00 g, 28.0 mmol) and N,N-diisopropylethylamine (9.76 mL, 56.0 mmol) were mixed under an inert atmosphere. The reaction was thereafter heated to 150° C. and was stirred at that temperature for 4.5 h. Ethyl acetate and 10% NaHSO₄ in water were added and the product was extracted with ethyl acetate (×3). The organic phases were dried over magnesium sulfate and the solvent was removed under reduced pressure to give 3.24 g of the crude product. The amount of 2-bromobenzyl alcohol formed (by-product) was estimated to be 13% from LC/MS (if wt. %, approx. 375 mg). The benzylic alcohol formed as a by-product was silylated to simplify the purification by column chromatography. The crude was dissolved in dichloromethane (50 mL) and tert-butyldimethylsilyl chloride (1.12 g, 7.45 mmol, 1.1 equiv. with respect to 2-bromobenzyl alcohol) was added followed by imidazole (507 mg, 7.45 mmol, 1.1 equiv. with respect to 2-bromobenzyl alcohol). The reaction was followed by LC/MS. After stirring at room temperature for 30 min, another portion of tert-butyldimethylsilyl chloride (1.12 g, 7.45 mmol) and imidazole (507 mg, 7.45 mmol) were added. After stirring for an additional 30 min, LC/MS showed full conversion of 2-bromobenzyl alcohol. The solid was filtered off, water was added and the product was extracted with dichloromethane. The dichloromethane solution was finally filtered through a pad of silica. The product was eluted with ethyl acetate and the solvent was removed under reduced pressure. The title compound was purified by column chromatography (dichloromethane/heptane/ethyl acetate 65:30:5) to give 4.84 g (59%) of the product. MS (ESI⁺): m/z 285 [M+H]⁺.

### b) 1-Bromo-6-[(2-bromophenyl)methoxy]hexan-2-one

6-[(2-Bromophenyl)methoxy]hexan-2-one (450 mg, 1.58 mmol) was dissolved in dichloromethane (10 mL) and methanol (5 mL). Tetra-n-butylammonium tribromide (761 mg, 1.58 mmol) was added and the reaction was left to stir at room temperature overnight. LC/MS analysis was thereafter performed, 1 drop of water was added and the reaction mixture was left to stir for a few min. The solvent was removed under reduced pressure and the product was filtered through a silica column (ethyl acetate:heptane 1:9). The fractions containing the product were combined to yield 540 mg of the product as a mixture of isomers. MS (ESI⁺): 365 m/z [M+H]⁺.

### c) 5-[4-[(2-Bromophenyl)methoxy]butyl]-6H-1,3,4-thiadiazin-2-amine

1-Bromo-6-[(2-bromophenyl)methoxy]hexan-2-one (346 mg, 0.950 mmol) was dissolved in ethanol (2 mL) containing HBr (48% in water, 0.06 mL, 1.05 mmol) and thiosemicarbazide (95.3 mg, 1.05 mmol) was added. The reaction was stirred at room temperature for 30 min. A white solid precipitated out of solution. The ethanol was removed under vacuum and the resulting solid was purified on silica (ethyl acetate to ethyl acetate: methanol 9:1). The collected fractions were concentrated and the solid formed was washed with ethyl acetate (3×1 mL) and dried to give 116 mg (34%) of the title compound.

### Example 76

### 5-[4-[(4-Chloro-2-fluoro-phenyl)methoxy]butyl]-6H-1,3,4-thiadiazin-2-amine

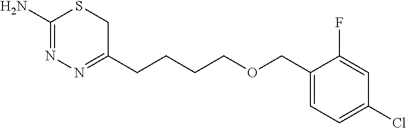 [SMILES: NC1=NN=C(CCCCOCc2ccc(Cl)cc2F)CS1]

### a) 6-[(4-Chloro-2-fluoro-phenyl)methoxy]hexan-2-one

Prepared as described for 6-[(2,4-difluorophenyl)methoxy]hexan-2-one (Example 69b). Starting from (4-chloro-2-fluoro-phenyl)methanol (533 mg, 3.32 mmol, 1.5 equiv.) and 6-iodohexan-2-one (500 mg, 2.21 mmol, 1 equiv.) yielded 210 mg (37%) of the product. MS (ESI⁺): m/z 259 [M+H]⁺.

### b) 1-Bromo-6-[(4-chloro-2-fluoro-phenyl)methoxy]hexan-2-one

Prepared as described for 1-bromo-6-[(2,4-difluorophenyl)methoxy]hexan-2-one, (Example 69c). Starting from 6-[(4-chloro-2-fluoro-phenyl)methoxy]hexan-2-one (210 mg, 0.812 mmol, 1 equiv.) and tetra-n-butylammonium tribromide (430 mg, 0.893 mmol, 1.1 equiv.) yielded a crude mixture that was used without further purification in step c.

### c) 5-[4-[(4-Chloro-2-fluoro-phenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[(4-chloro-2-fluoro-phenyl)methoxy]hexan-2-one afforded the title compound in 28 mg yield (10% over 2 steps, b-c).

### Example 77

### 5-[4-[(3,5-Dichlorophenyl)methoxy]butyl]-6H-1,3,4-thiadiazin-2-amine

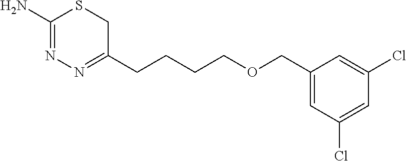 [SMILES: NC1=NN=C(CCCCOCc2cc(Cl)cc(Cl)c2)CS1]

### a) 6-[(3,5-Dichlorophenyl)methoxy]hexan-2-one

Prepared as described for 6-[(2,4-difluorophenyl)methoxy]hexan-2-one (Example 69b). The reaction was performed in toluene (0.5 mL). Starting from (3,5-dichlorophenyl)methanol (587 mg, 3.32 mmol, 1.5 equiv.) and 6-iodohexan-2-one (500 mg, 2.21 mmol, 1 equiv.) yielded 143 mg (23%) of the product. MS (ESI⁺): m/z 275 [M+H]⁺.

### b) 1-Bromo-6-[(3,5-dichlorophenyl)methoxy]hexan-2-one

Prepared as described for 1-bromo-6-[(2,4-difluorophenyl)methoxy]hexan-2-one, (Example 69c). Starting from 6-[(3,5-dichlorophenyl)methoxy]hexan-2-one (143 mg, 0.520 mmol, 1 equiv.) and tetra-n-butylammonium tribromide (318 mg, 0.660 mmol, 1.3 equiv.) yielded a crude mixture that was used without further purification in step c.

### c) 5-[4-[(3,5-Dichlorophenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step b containing 1-bromo-6-[(3,5-dichlorophenyl)methoxy]hexan-2-one afforded the title compound in 11 mg yield (5% over 2 steps, b-c).

### Example 78

### 5-[4-[(3-Ethylphenyl)methoxy]butyl]-6H-1,3,4-thiadiazin-2-amine

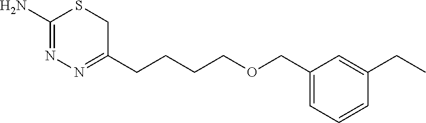 [SMILES: CCc1cccc(COCCCCC2=NN=C(N)SC2)c1]

### a) Methyl 3-ethylbenzoate

Methyl 3-bromobenzoate (1.00 g, 4.65 mmol, 1 equiv.) and [1,1′-bis(diphenylphosphino)ferrocene]dichloropalladium(II), complex with dichloromethane (PdCl₂(dppf).CH₂Cl_{2, 95} mg, 0.12 mmol, 2.5 mol %) were dissolved in tetrahydrofuran (10 mL). K₃PO₄ (4.65 mL, 2.0 M in water, 9.30 mmol, 2 equiv.) was thereafter added followed by triethylborane (4.65 mL, 1.0 M in hexanes, 4.65 mmol, 1 equiv.) and the mixture was heated to reflux. After stirring at reflux overnight the reaction mixture was allowed to reach room temperature and the solid was filtered off. To the solution was added ethyl acetate and water and the product was extracted with ethyl acetate (×2). The combined organic phases were dried over sodium sulfate and the solvent was removed under reduced pressure. The product was purified by column chromatography (heptane/ethyl acetate 19:1) to give 733 mg (96%) of the product. MS (ESI⁺): m/z 165 [M+H]⁺.

### b) (3-Ethylphenyl)methanol

Methyl 3-ethylbenzoate (733 mg, 4.46 mmol, 1 equiv.) was dissolved in tetrahydrofuran (3 mL) and the solution was cooled to 0° C. Lithium aluminum hydride (4.55 mL, 1 M solution in tetrahydrofuran, 4.55 mmol, 1.02 equiv.) was thereafter added and the solution was stirred at 0° C. for 2 h. The reaction mixture was diluted with more tetrahydrofuran, saturated aqueous potassium sodium tartrate (73 mL) was added and the mixture was left to stir at room temperature overnight. The solid was thereafter filtered off and the product was extracted with diethyl ether (×3). The combined organic phases were dried over sodium sulfate and concentrated at reduced pressure to yield 642 mg (quantitative) of the title compound. The crude product was used in the next step without further purification. MS (ESI⁺): m/z 119 [M−OH]⁺.

### c) 6-[(3-Ethylphenyl)methoxy]hexan-2-one

Prepared as described for 6-[(2,4-difluorophenyl)methoxy]hexan-2-one (Example 69b). Starting from (3-ethylphenyl)methanol (500 mg, 3.67 mmol, 1.5 equiv.) and 6-iodohexan-2-one (554 mg, 2.45 mmol, 1 equiv.) yielded 194 mg (34%) of the product. MS (ESI⁺): m/z 235 [M+H]⁺.

### d) 1-Bromo-6-[(3-ethylphenyl)methoxy]hexan-2-one

Prepared as described for 1-bromo-6-[(2,4-difluorophenyl)methoxy]hexan-2-one, (Example 69c). Starting from 6-[(3-ethylphenyl)methoxy]hexan-2-one (194 mg, 0.828 mmol, 1 equiv.) and tetra-n-butylammonium tribromide (439 mg, 0.911 mmol, 1.1 equiv.) yielded a crude mixture that was used without further purification in step e.

### e) 5-[4-[(3-Ethylphenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step d containing 1-bromo-6-[(3-ethylphenyl)methoxy]hexan-2-one afforded the title compound in 26 mg yield (10% over 2 steps, d-e).

### Example 79

### 5-[4-[(3-Vinylphenyl)methoxy]butyl]-6H-1,3,4-thiadiazin-2-amine

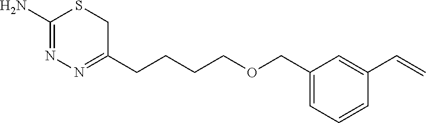 [SMILES: C=Cc1cccc(COCCCCC2=NN=C(N)SC2)c1]

### a) 6-[(3-Bromophenyl)methoxy]hexan-2-one

The title compound was prepared as described in Example 73b.

### b) 6-[(3-Vinylphenyl)methoxy]hexan-2-one

The title compound was prepared according to a modified literature procedure. [J. Org. Chem. 2006, 71(26), 9681-9686] To potassium vinyltrifluoroborate (244 mg, 1.82 mmol, 1.3 equiv.), palladium(II) acetate (16 mg, 0.070 mmol, 5 mol %), cesium carbonate (1.37 g, 4.21 mmol) and triphenylphospine (37 mg, 0.14 mmol, 10 mol %) was added a solution of 6-[(3-bromophenyl)methoxy]hexan-2-one (400 mg, 1.4 mmol, 1 equiv.) dissolved in tetrahydrofuran (3 mL) followed by water (0.30 mL) under an inert atmosphere. The reaction mixture was heated to 80° C. and was left to stir at that temperature overnight. Ethyl acetate and water were added and the mixture was filtered through a pad of Celite in order to remove the solid. The two phases were separated and the product was extracted two additional times with ethyl acetate. The combined organic phases were washed with brine, dried over sodium sulfate and concentrated at reduced pressure. The title compound was purified by column chromatography (heptane/ethyl acetate 9:1) to afford 148 mg (45%) of the title compound. MS (ESI⁺): m/z 233 [M+H]⁺.

### c) Trimethyl-[1-methylene-5-[(3-vinylphenyl)methoxy]pentoxy]silane

6-[(3-Vinylphenyl)methoxy]hexan-2-one (145 mg, 0.620 mmol, 1 equiv.) was dissolved in tetrahydrofuran (2.5 mL) under an inert atmosphere and the solution was cooled to −78° C. Lithium diisopropylamide (1.0 M in tetrahydrofuran/hexanes, 1.0 mL, 1.0 mmol, 1.6 equiv.) was added dropwise and the reaction mixture was left to stir at −78° C. for 25 min. A solution of chloro(trimethyl)silane (81 mg, 0.750 mmol) in tetrahydrofuran (0.5 mL) was added dropwise and the reaction mixture was stirred at −78° C. for another 25 min. The reaction was then quenched with saturated aqueous NaHCO₃ and the mixture was allowed to reach room temperature. Ethyl acetate, water and brine were added, the two phases were separated and the product was extracted with ethyl acetate (×2). The combined organic phases were dried over sodium sulfate and the solvent was removed under reduced pressure to yield 188 mg (99%) of the crude product. MS (ESI⁺): m/z 305 [M+H]⁺. The crude product was used in the next step without further purification.

### d) 1-Bromo-6-[(3-vinylphenyl)methoxy]hexan-2-one

The crude mixture from step c (188 mg) was dissolved in tetrahydrofuran (6 mL), the reaction vessel was put under an inert atmosphere and the solution was cooled to 0° C. N-Bromosuccinimide (in total 98 mg, 0.55 mmol, 0.9 equiv.) dissolved in tetrahydrofuran was added in portions until full conversion of the starting material was achieved according to LC/MS. The reaction was thereafter quenched with saturated aqueous NaHCO₃ solution and the mixture was allowed to reach room temperature. Ethyl acetate, water and brine was added, the two phases were separated and the product was extracted with ethyl acetate two additional times. The combined organic phases were dried over sodium sulfate and the solvent was removed at reduced pressure. The title compound was purified by column chromatography (heptane/ethyl acetate 4:1) and was isolated in 98 mg (50% over 2 steps, c-d) yield. MS (ESI⁺): m/z 311 [M+H]⁺.

### e) 5-[4-[(3-Vinylphenyl)methoxy]butyl]-6H-1,3,4-thiadiazin-2-amine

1-bromo-6-[(3-vinylphenyl)methoxy]hexan-2-one (98 mg, 0.31 mmol, 1 equiv.) was dissolved in ethanol (3 mL) containing HBr (48 wt. % in water, 36 μL, 0.31 mmol, 1 equiv.) and thiosemicarbazide (32 mg, 0.35 mmol, 1.1 equiv.) was thereafter added. The reaction was stirred at room temperature and was followed by LC/MS. After 1 h, the starting material was consumed, the solvent was removed at reduced pressure and the product was purified by column chromatography (ethyl acetate to ethyl acetate/methanol 9:1). The product was thereafter dissolved in ethyl acetate and a smaller amount of solid was filtered off. The solution was concentrated at reduced pressure and the title compound was isolated in 32 mg (33%) yield.

### Example 80

### 5-[4-[[3-[(E)-Prop-1-enyl]phenyl]methoxy]butyl]-6H-1,3,4-thiadiazin-2-amine/5-[4-[(3-allylphenyl)methoxy]butyl]-6H-1,3,4-thiadiazin-2-amine

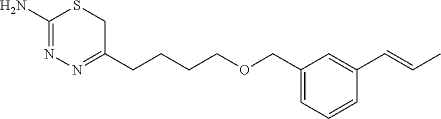 [SMILES: C/C=C/c1cccc(COCCCCC2=NN=C(N)SC2)c1]

### a) 6-[(3-Bromophenyl)methoxy]hexan-2-one

The title compound was prepared as described in Example 73b.

### b) 6-[[3-[(E)-Prop-1-enyl]phenyl]methoxy]hexan-2-one/6-[(3-allylphenyl)methoxy]hexan-2-one

Prepared as described for 6-[(3-prop-1-ynylphenyl)methoxy]hexan-2-one, (Example 82b, vide infra). Two separate reactions were run and combined for work-up and purification. The combined reaction mixtures were filtered through a pad of Celite before extraction. The first reaction was started from potassium allyltrifluoroborate (31 mg, 0.21 mmol, 1.2 equiv.), 6-[(3-bromophenyl)methoxy]hexan-2-one (50 mg, 0.18 mmol, 1 equiv.) and PdCl₂(dppf).CH₂Cl₂ (7 mg, 0.009 mmol, 5 mol %) and the second reaction was started from potassium allyltrifluoroborate (270 mg, 1.82 mmol, 1.3 equiv.), 6-[(3-bromophenyl)methoxy]hexan-2-one (400 mg, 1.40 mmol, 1 equiv.) and PdCl₂(dppf).CH₂Cl₂ (57 mg, 0.070 mmol, 5 mol %). The reactions gave 68 mg (17%) of the title compounds. ¹H NMR analysis revealed that the isolated material consisted of a 3:2 mixture of 6-[[3-[(E)-prop-1-enyl]phenyl]methoxy]hexan-2-one (A) and 6-[(3-allylphenyl)methoxy]hexan-2-one (B). ¹H NMR (400 MHz, CDCl₃ ) δ=7.36-7.08 (m, A:4H, B:4H), 6.40 (dm, J=15.7 Hz, A:1H), 6.25 (dq, J=15.7, 6.5 Hz, A:1H), 5.97 (ddt, J=16.9, 10.1, 6.7 Hz, B:1H), 5.09 (dm, J=16.9 Hz, B:1H), 5.07 (dm, J=10.1 Hz, B:1H), 4.47 (s, A:2H, B:2H), 3.50-3.44 (m, A:2H, B:2H), 3.39 (d, J=6.7 Hz, B:2H), 2.45 (t, J=7.1 Hz, A:2H, B:2H), 2.12 (s, A:3H, B:3H), 1.88 (dd, J=6.5, 1.6 Hz, A:3H), 1.72-1.57 (m, A:4H, B:4H). MS (ESI⁺): m/z 247 [M+H]⁺.

### c) Trimethyl-[1-methylene-5-[[3-[(E)-prop-1-enyl]phenyl]methoxy]pentoxy]silane/[5-[(3-allylphenyl)methoxy]-1-methylene-pentoxy]-trimethyl-silane

Prepared as described for trimethyl-[1-methylene-5-[(3-vinylphenyl)methoxy]pentoxy]silane, (Example 79c). Starting from a 3:2 mixture of 6-[[3-[(E)-prop-1-enyl]phenyl]methoxy]hexan-2-one and 6-[(3-allylphenyl)methoxy]hexan-2-one (63 mg, 0.26 mmol, 1 equiv.), lithium diisopropylamide (1.0 M in tetrahydrofuran/hexanes, 0.38 mL, 0.38 mmol, 1.5 equiv.) and chloro(trimethyl)silane (33 mg, 0.0.31 mmol, 1.2 equiv.) yielded 94 mg of the crude product which was used without further purification in the next step. MS (ESI⁺): m/z 319 [M+H]⁺.

### d) 1-Bromo-6-[[3-[(E)-prop-1-enyl]phenyl]methoxy]hexan-2-one/6-[(3-allylphenyl)methoxy]l-1-bromo-hexan-2-one

Prepared as described for 1-bromo-6-[(3-vinylphenyl)methoxy]hexan-2-one, (Example 79d). Starting from the crude mixture of trimethyl-[1-methylene-5-[[3-[(E)-prop-1-enyl]phenyl]methoxy]pentoxy]silane and [5-[(3-allylphenyl)methoxy]-1-methylene-pentoxy]-trimethyl-silane from step c (94 mg) and N-bromosuccinimide (in total 26 mg, 0.15 mmol, 0.6 equiv.) yielded 45 mg (54% over 2 steps, c-d) of a mixture of the two title compounds. MS (ESI⁺): m/z 325 [M+H]⁺.

### e) 5-[4-[[3-[(E)-Prop-1-enyl]phenyl]methoxy]butyl]-6H-1,3,4-thiadiazin-2-amine/5-[4-[(3-allylphenyl)methoxy]butyl]-6H-1,3,4-thiadiazin-2-amine

Prepared as described for 5-[4-[(3-vinylphenyl)methoxy]butyl]-6H-1,3,4-thiadiazin-2-amine, (Example 79e). Starting from the mixture of 1-bromo-6-[[3-[(E)-prop-1-enyl]phenyl]methoxy]hexan-2-one and 6-[(3-allylphenyl)methoxy]-1-bromo-hexan-2-one (41 mg, 0.13 mmol, 1 equiv.) from step d, thiosemicarbazide (16 mg, 0.18 mmol, 1.4 equiv.) and HBr (48 wt. % in water, 7.2 μL, 0.060 mmol, 0.5 equiv.) yielded 10 mg (25%) of a 2:1 mixture of 5-[4-[[3-[(E)-prop-1-enyl]phenyl]methoxy]butyl]-6H-1,3,4-thiadiazin-2-amine (A) and 5-[4-[(3-allylphenyl)methoxy]butyl]-6H-1,3,4-thiadiazin-2-amine (B).

### Example 81

### 5-[4-[(3-Cyclopropylphenyl)methoxy]butyl]-6H-1,3,4-thiadiazin-2-amine

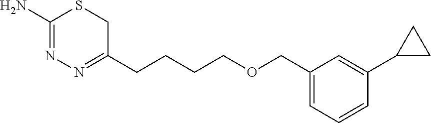 [SMILES: NC1=NN=C(CCCCOCc2cccc(C3CC3)c2)CS1]

### a) 6-[(3-Bromophenyl)methoxy]hexan-2-one

The title compound was prepared as described in Example 73b.

### b) 6-[(3-Cyclopropylphenyl)methoxy]hexan-2-one

The title compound was prepared according to a modified literature procedure. [J. Org. Chem. 2009, 74(10), 3626-3631] To palladium(II) acetate (16 mg, 0.07 mmol, 10 mol %), 2-dicyclohexylphosphino-2′,6′-diisopropoxybiphenyl (RuPhos, 65 mg, 0.14 mmol, 20 mol %), potassium cyclopropyltrifluoroborate (259 mg, 1.05 mmol, 1.5 equiv.) and K₂CO₃ (982 mg, 7.10 mmol, 10 equiv.) was added 6-[(3-bromophenyl)methoxy]hexan-2-one (200 mg, 0.701 mmol, 1 equiv.) in toluene (3.5 mL) followed by water (0.35 mL) under an inert atmosphere. The reaction mixture was heated to 80° C. and was stirred at that temperature overnight. More water and ethyl acetate were added and the two phases were filtered through a pad of Celite. The phases were separated and the product was extracted two additional times with ethyl acetate. The combined organic phases were washed with brine, dried over sodium sulfate and the solvent was removed under reduced pressure. The title compound was purified by column chromatography (heptane/ethyl acetate 9:1) to give 156 mg (90%) of the title compound. MS (ESI⁺): m/z 247 [M+H]⁺.

### c) 1-Bromo-6-[(3-cyclopropylphenyl)methoxy]hexan-2-one

Prepared as described for 1-bromo-6-[(2,4-difluorophenyl)methoxy]hexan-2-one, (Example 69c). Starting from 6-[(3-cyclopropylphenyl)methoxy]hexan-2-one (156 mg, 0.633 mmol, 1 equiv.) and tetra-n-butylammonium tribromide (336 mg, 0.697 mmol, 1.1 equiv.) yielded a crude mixture that was used without further purification in step d.

### d) 5-[4-[(3-Cyclopropylphenyl)methoxy]butyl]-6H-1,3,4-thiadiazin-2-amine

Prepared as described for 5-[4-[(2,4-difluorophenyl)methoxy]butyl]-6H-1,3,4-thiadiazin-2-amine, (Example 69d). Starting from the crude mixture from step c containing 1-bromo-6-[(3-cyclopropylphenyl)methoxy]hexan-2-one afforded 46 mg (23% over 2 steps, c-d) of the title compound.

### Example 82

### 5-[4-[(3-Prop-1-ynylphenyl)methoxy]butyl]-6H-1,3,4-thiadiazin-2-amine

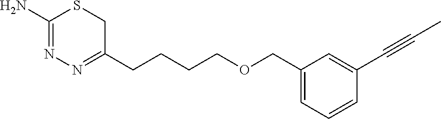 [SMILES: CC#Cc1cccc(COCCCCC2=NN=C(N)SC2)c1]

### a) 6-[(3-Bromophenyl)methoxy]hexan-2-one

The title compound was prepared as described in Example 73b.

### b) 6-[(3-Prop-1-ynylphenyl)methoxy]hexan-2-one

The title compound was prepared according to a modified literature procedure. [J. Org. Chem., 2003, 68 (14), 5534-5539] To potassium propynyltrifluoroborate (384 mg, 2.63 mmol, 1.5 equiv.), [1,1′-bis(diphenylphosphino)ferrocene]dichloropalladium(II), complex with dichloromethane (PdCl₂(dppf).CH₂Cl_{2, 143} mg, 0.180 mmol, 5 mol %) and cesium carbonate (1.714 g, 5.26 mmol, 3 equiv.) was added a solution of 6-[(3-bromophenyl)methoxy]hexan-2-one (500 mg, 1.75 mmol, 1 equiv.) in tetrahydrofuran (15 mL) followed by water (1.5 mL) under an inert atmosphere. The reaction mixture was thereafter heated to 80° C. and was left to stir at that temperature overnight. Ethyl acetate and water were added and the two phases were separated and the product was extracted two additional times with ethyl acetate. The combined organic phases were washed with brine, dried over sodium sulfate and concentrated under reduced pressure. The title compound was purified by column chromatography (heptane/ethyl acetate 9:1) to afford 393 mg (92%) of the title compound. MS (ESI⁺): m/z 245 [M+H]⁺.

### c) Trimethyl-[1-methylene-5-[(3-prop-1-ynylphenyl)methoxy]pentoxy]silane

n-Butyllithium (1.6 M in hexanes, 0.50 mL, 0.80 mmol, 1 equiv.) was added to diisopropylamine (0.12 mL, 0.88 mmol, 1.1 equiv.) dissolved in tetrahydrofuran (1.5 mL) at room temperature. The solution was stirred for 30 minutes and was then added dropwise to a solution of 6-[(3-prop-1-ynylphenyl)methoxy]hexan-2-one (196 mg, 0.800 mmol, 1 equiv.) in tetrahydrofuran (3 mL) cooled to −78° C. The reaction mixture was stirred at this temperature for 40 minutes, whereafter chloro(trimethyl)silane (0.11 mL, 0.88 mmol, 1.1 equiv.) in tetrahydrofuran (0.5 mL) was added. The reaction was allowed to reach room temperature and after 1 h saturated aqueous NaHCO₃ (1 mL) was added followed by diethyl ether. The organic phase was washed twice with water, dried over sodium sulfate and concentrated at reduced pressure. The crude mixture containing the title compound (approximately 50% conversion, estimated by comparison of the integrals below the peaks for 6-[(3-prop-1-ynylphenyl)methoxy]hexan-2-one and trimethyl-[1-methylene-5-[(3-prop-1-ynylphenyl)methoxy]pentoxy]silane in LC/MS analysis at 305-180 nm) was stored in the freezer for 3 days. Analysis thereafter revealed that the silyl enol ether decomposed to a large extent during storage. The above procedure was therefore repeated. Lithium diisopropylamide was prepared as described above from diisopropylamine (97 mg, 0.96 mmol, 1.2 equiv.) and n-butyllithium (1.6 M in hexanes, 0.55 mL, 0.88 mmol, 1.1 equiv.) and was added dropwise to the crude mixture dissolved in tetrahydrofuran (3 mL) at −78° C. Chloro(trimethyl)silane (174 mg, 1.60 mmol) was added after 20 min as a solution in tetrahydrofuran (0.5 mL). Saturated aqueous NaHCO₃ (2 mL) was then added and the reaction mixture was allowed to reach room temperature, whereafter ethyl acetate was added followed by water. The organic phase was washed with water and brine, dried over sodium sulfate and was concentrated at reduced pressure (approximately 50% conversion¹). The above procedure was repeated a second time. Lithium diisopropylamide was prepared as described above from diisopropylamine (179 mg, 1.76 mmol, 2.2 equiv.) and n-butyllithium (1.6 M in hexanes, 1.0 mL, 1.6 mmol, 2 equiv.) and was added dropwise to the crude mixture dissolved in tetrahydrofuran (5 mL) at −78° C. Chloro(trimethyl)silane (261 mg, 2.41 mmol, 3 equiv.) was added after 15 min as a solution in tetrahydrofuran (0.5 mL). Saturated aqueous NaHCO₃ (2.5 mL) was added and the reaction mixture was allowed to reach room temperature. Ethyl acetate was then added followed by water. The organic phase was washed with water and brine, dried over sodium sulfate and was concentrated at reduced pressure. The crude product was used without further purification in the next step. MS (ESI⁺): m/z 317 [M+H]⁺.

### d) 1-Bromo-6-[(3-prop-1-ynylphenyl)methoxy]hexan-2-one

The crude mixture from step c was dissolved in tetrahydrofuran (15 mL) and the solution was cooled to 0° C. N-Bromosuccinimide dissolved in tetrahydrofuran was added in two portions (portion 1:14 mg, 0.080 mmol, 0.1 equiv., in 0.5 mL tetrahydrofuran; portion 2:60 mg, 0.34 mmol, 0.4 equiv., in 1 mL tetrahydrofuran). 15 min after the second addition of N-bromosuccinimide, saturated aqueous NaHCO₃ (5 mL) was added and the reaction mixture was allowed to reach room temperature. Ethyl acetate and water was then added. The organic phase was washed with water and brine and dried over sodium sulfate. The title compound was purified by column chromatography (heptane/ethyl acetate 9:1) to give 20 mg (8%) of the product. MS (ESI⁺): m/z 340, 342 [M+18]⁺.

### d) 5-[4-[(3-Prop-1-ynylphenyl)methoxy]butyl]-6H-1,3,4-thiadiazin-2-amine

1-bromo-6-[(3-prop-1-ynylphenyl)methoxy]hexan-2-one (20 mg, 0.050 mmol) was dissolved in ethanol (0.75 mL) and thiosemicarbazide (4 mg, 0.05 mmol) was thereafter added. The reaction was stirred at room temperature and was followed by LC/MS. After 2 hours HCl (1.25 M in EtOH, 4 μL, 0.005 mmol, 0.1 equiv.) was added and after an additional 30 min another portion of HCl (1.25 M in EtOH, 34 μL, 0.09 mmol, 0.1 equiv.) was added and the reaction was left to stir overnight at room temperature. The solvent was thereafter removed at reduced pressure and the title compound was purified by column chromatography (ethyl acetate/methanol 9:1) to yield 8 mg (51%) of the product.

### Example 83

### 5-[4-[(4-Fluorophenyl)methoxy]butyl]-6H-1,3,4-thiadiazin-2-amine hydrobromide

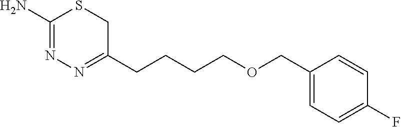 [SMILES: NC1=NN=C(CCCCOCc2ccc(F)cc2)CS1]

### a) 6-[(4-Fluorophenyl)methoxy]hexan-2-one

To (4-fluorophenyl)methanol (541 uL, 4.95 mmol) was added potassium hydroxide (327 mg, 4.95 mmol) and the reaction was stirred for 50 min before 6-iodohexan-2-one (700 mg, 3.10 mmol) was added. The reaction was then stirred at room temperature overnight. Water and dichloromethane were added and the layers separated. The aqueous phase was extracted with dichloromethane (3×). The combined organic phases were washed with brine, dried over magnesium sulfate, filtered and evaporated. The crude material was purified by column chromatography (heptane/ethyl acetate 90:10 to 80:20) to give 285 mg (41%) of the title compound. MS (ESI⁺): m/z 225 [M+H]⁺.

### b) 1-Bromo-6-[(4-fluorophenyl)methoxy]hexan-2-one

To a solution of 6-[(4-fluorophenyl)methoxy]hexan-2-one (285 mg, 1.27 mmol) in dichloromethane (8 mL) and methanol (4 mL) was added tetra-n-butylammonium tribromide (674 mg, 1.40 mmol) and the reaction stirred at room temperature for 5 h. The solvents were evaporated and the crude was dissolved in ethyl acetate and washed with water (3×) and brine, dried over magnesium sulfate, filtered and evaporated to give 373 mg of the crude product after drying. MS (ESI⁺): m/z 303, 305 [M+H]⁺.

### c) 5-[4-[(4-Fluorophenyl)methoxy]butyl]-6H-1,3,4-thiadiazin-2-amine hydrobromide,

To a solution of 1-bromo-6-[(4-fluorophenyl)methoxy]hexan-2-one (373 mg, 1.23 mmol) in ethanol (6 mL) was added HBr (48% in water) (207 mg, 1.23 mmol) followed by thiosemicarbazide (112 mg, 1.23 mmol) and the reaction stirred for 1.5 h at room temperature. The precipitated product was filtered off and washed with ethanol. The product was recrystallized from ethanol to give 25.1 mg (5%) of the title compound.

### Example 84

### 4-[4-(2-Amino-6H-1,3,4-thiadiazin-5-yl)butoxymethyl]benzonitrile hydrobromide

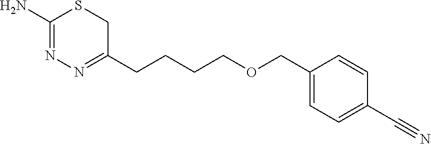 [SMILES: N#Cc1ccc(COCCCCC2=NN=C(N)SC2)cc1]

### a) 4-(5-Oxohexoxymethyl)benzonitrile

Potassium hydroxide (1.01 g, 18.0 mmol) was added to a solution of 6-iodohexan-2-one (5.36 g, 22.5 mmol) and the mixture was stirred for 30 min at room temperature before 4-(hydroxymethyl)benzonitrile (2.00 g, 15.0 mmol) was added to the reaction mixture. The reaction was stirred overnight at room temperature. Water was added and the mixture was extracted with ethyl acetate (3×30 mL), and the organic layers were combined, dried over magnesium sulfate and concentrated under reduced pressure. The crude product was purified by column chromatography (dichloromethane to 10% acetone in dichloromethane) to give 1.60 g (46%) of the title compound. MS (ESI⁺): m/z 232 [M+H]⁺.

### b) 4-[(6-Bromo-5-oxo-hexoxy)methyl]benzonitrile

Tetra-n-butylammonium tribromide (386 mg, 0.80 mmol) was added in one portion to a solution of 4-(5-oxohexoxymethyl)benzonitrile (500 mg, 2.16 mmol) in dichloromethane (16 mL) and methanol (8 mL). The reaction mixture was stirred for 4 h at room temperature. Additional tetra-n-butylammonium tribromide (761 mg, 158 mmol) was added and the reaction was stirred overnight at room temperature. The mixture was extracted with ethyl acetate (3×30 mL) and wash with water (5×20 mL), and the organic layers were combined, washed with brine and dried over magnesium sulfate and concentrated under reduced pressure. The product was filtered through a silica column (heptane/ethyl acetate 2:3). The fractions containing the product were combined to yield 546 mg of the product as a mixture of isomers. MS (ESI⁺): m/z 327, 329 [M+H]⁺.

### c) 4-[4-(2-Amino-6H-1,3,4-thiadiazin-5-yl)butoxymethyl]benzonitrile hydrobromide

Thiosemicarbazide (95.3 mg, 1.05 mmol) was added to a solution of 4-[(6-bromo-5-oxo-hexoxy)methyl]benzonitrile (295 mg, 0.95 mmol) and HBr (48 wt. % in water, 0.12 mL, 1.05 mmol) in ethanol (4 mL). The reaction mixture was stirred for 30 min at room temperature. Then the solvent was removed under reduced pressure. The desired product was recrystallized from methanol to give 150 mg (41%) of the title compound.

### Example 85

### 3-[4-(2-Amino-6H-1,3,4-thiadiazin-5-yl)butoxymethyl]benzonitrile hydrobromide

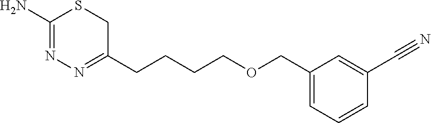 [SMILES: N#Cc1cccc(COCCCCC2=NN=C(N)SC2)c1]

### a) 3-(5-Oxohexoxymethyl)benzonitrile

Prepared as described for 4-(5-oxohexoxymethyl)benzonitrile, (Example 84b). Starting from 3-(Hydroxymethyl)benzonitrile (1.60 g, 12.0 mmol) and 6-iodohexan-2-one (4.29 mg, 19.0 mmol) yielded 1.55 mg (57%) of the product. MS (ESI⁺): m/z 249 [M+18]⁻, 232 [M+H]⁻.

### b) 3-[(6-Bromo-5-oxo-hexoxy)methyl]benzonitrile

Prepared as described for 4-[(6-bromo-5-oxo-hexoxy)methyl]benzonitrile, Example 84b. Starting from 3-(5-oxohexoxymethyl)benzonitrile (500 mg, 2.16 mmol) gave 580 mg of the product as a mixture of isomers. MS (ESI⁺): m/z 327, 329 [M+18]⁺.

### c) 3-[4-(2-Amino-6H-1,3,4-thiadiazin-5-yl)butoxymethyl]benzonitrile hydrobromide

Prepared as described for 4-[4-(2-amino-6H-1,3,4-thiadiazin-5-yl)butoxymethyl]benzonitrile, (Example 84c). Starting from 3-[(6-bromo-5-oxo-hexoxy)methyl]benzonitrile (295 mg, 0.95 mmol) afforded 158 mg (44%) of the product.

### Example 86

### 5-[6-(4-Bromo-2,6-difluoro-phenyl)hexyl]-6H-1,3,4-thiadiazin-2-amine hydrobromide

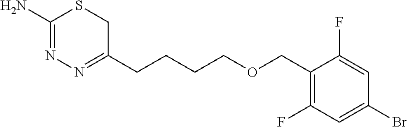 [SMILES: NC1=NN=C(CCCCOCc2c(F)cc(Br)cc2F)CS1]

### a) 6-[(4-Bromo-2,6-difluoro-phenyl)methoxy]hexan-2-one

Potassium hydroxide (0.377 g, 6.73 mmol) was added to a solution of 6-iodohexan-2-one (80% purity) (2.374 g, 8.40 mmol) in toluene (0.5 mL), followed by the addition of 4-bromo-2,6-difluorobenzylalcohol (1.25 g, 5.60 mmol). The solution mixture was stirred overnight at room temperature. Water (20 mL) was added to the solution mixture. The mixture was extracted with ethyl acetate (3×30 mL), and the organic layers were combined, washed with brine, dried over magnesium sulfate, filtered and concentrated under reduced pressure. The crude was purified by column chromatography (dichloromethane/heptane 5:1 to dichloromethane/acetone 4:1) to give 774 mg (43%) of the product. MS (ESI⁺): m/z 321, 323 [M+H]⁺.

### b) 1-Bromo-6-[(4-bromo-2,6-difluoro-phenyl)methoxy]hexan-2-one

Tetra-n-butylammonium tribromide was added to a solution of 6-[(4-bromo-2,6-difluoro-phenyl)methoxy]hexan-2-one (500 mg, 1.56 mmol) in dichloromethane (6 mL) and methanol (3 mL). The solution mixture was stirred for 5 h at room temperature. The mixture was extracted with ethyl acetate (3×30 mL), and the organic layers were combined and washed with brine, dried over magnesium sulfate, and concentrated under reduced pressure. The product was filtered through a silica column (ethyl acetate/heptane 1:9). The fractions containing the product were combined to yield 602 mg of the product as a mixture of isomers. MS (ESI⁺): m/z 418 [M+18]⁺, 401 [M+H]⁺

### c) 5-[6-(4-Bromo-2,6-difluoro-phenyl)hexyl]-6H-1,3,4-thiadiazin-2-amine hydrobromide

Thiosemicarbazide (90.7 mg, 0.990 mmol) was added to a solution (pH=2) of 1-bromo-6-[(4-bromo-2,6-difluoro-phenyl)methoxy]hexan-2-one (360 mg, 0.900 mmol) and HBr (48 wt. % in water, 0.11 mL, 0.99 mmol) in ethanol (4 mL). The reaction mixture was stirred for 45 min at room temperature. The solvent was removed under reduced pressure and the residue was recrystallized from methanol and acetone to give 29 mg (7%) of the product after drying. ¹H NMR (400 MHz, DMSO-d₆) 6 7.54-7.48 (m, 2H), 4.46 (s, 2H), 3.70 (s, 2H), 3.43 (t, J=6.0 Hz, 2H), 2.50 (t, 2H, partially obscured by the solvent peak), 1.65-1.47 (m, 4H). MS (ESI⁺): m/z 392, 394 [M+H]⁻.

### Example 87

### 5-[4-[(2-Chloro-6-fluoro-phenyl)methoxy]butyl]-6H-1,3,4-thiadiazin-2-amine

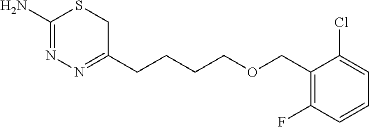 [SMILES: NC1=NN=C(CCCCOCc2c(F)cccc2Cl)CS1]

### a) 6-[(2-Chloro-6-fluoro-phenyl)methoxy]hexan-2-one

6-Fluoro-2-chlorobenzyl alcohol (310 mg, 1.93 mmol) and potassium hydroxide (153 mg, 2.32 mmol) were mixed in 0.35 ml of toluene. The reaction was stirred at room temperature for 30 min followed by the addition of 6-iodo-2-hexanone (436 mg, 1.93 mmol). The reaction was stirred at room temperature and was thereafter quenched with water and extracted with ethyl acetate (3×10 mL). The organic layer were washed with brine, dried over sodium sulfate, and the solvent removed under reduced pressure. The residue was purified with column chromatography (heptane/ethyl acetate 9:1) to give 410 mg (84%) of the product. MS (ESI⁺): m/z 259 [M+H]⁺.

### b) 1-Bromo-6-[(2-chloro-6-fluoro-phenyl)methoxy]hexan-2-one

6-[(2-Chloro-6-fluoro-phenyl)methoxy]hexan-2-one (418 mg, 1.62 mmol) was dissolved in methanol (10 mL) and then bromine (0.09 mL, 1.70 mmol) was added dropwise at 0° C. during 1 h. The reaction mixture was then allowed to reach room temperature and stirred overnight. The reaction mixture was concentrated to dryness and the residue was taken up in ethyl acetate (25 mL). The organic phase was washed with water (2×10 mL) and brine (1×10 mL). The organic layer was separated and dried over magnesium sulfate and concentrated to dryness to give 389 mg of the crude product, which was taken to the next step without further purification.

### c) 5-[4-[(2-Chloro-6-fluoro-phenyl)methoxy]butyl]-6H-1,3,4-thiadiazin-2-amine

Thiosemicarbazide (117 mg, 1.29 mmol) and 1-bromo-6-[(2-chloro-6-fluoro-phenyl)methoxy]hexan-2-one (455 mg, 1.17 mmol) were mixed in ethanol (3 mL) and stirred at room temperature overnight. The reaction was concentrated to dryness and the residue was taken up in ethyl acetate (80 mL) and the organic layer was washed with water (2×20 mL) and saturated brine solution (1×20 mL). The organic layer was dried over sodium sulfate before it was concentrated to dryness. The crude was then purified by column chromatography (100% ethyl acetate then ethyl acetate/methanol 9:1) to give 21.4 mg (6%) of the product.

### Example 88

### 5-[4-[(2,5-Difluorophenyl)methoxy]butyl]-6H-1,3,4-thiadiazin-2-amine

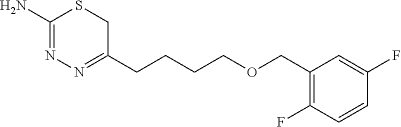 [SMILES: NC1=NN=C(CCCCOCc2cc(F)ccc2F)CS1]

### a) 6-[(2,5-Difluorophenyl)methoxy]hexan-2-one

Prepared as described for 6-[(2-chloro-6-fluoro-phenyl)methoxy]hexan-2-one, (Example 87a). Starting from (2,5-difluorophenyl)methanol (432 mg, 3.00 mmol) and 6-iodohexan-2-one (678 mg, 3.00 mmol) yielded 310 mg (43%) of the product. MS (ESI⁺): m/z 243 [M+H]⁺.

### b) 1-Bromo-6-[(2,5-difluorophenyl)methoxy]hexan-2-one

Prepared as described for 1-bromo-6-[(2-chloro-6-fluoro-phenyl)methoxy]hexan-2-one, (Example 87b). Starting from 6-[(2,5-difluorophenyl)methoxy]hexan-2-one (310. mg, 1.28 mmol) and bromine (0.07 mL, 1.34 mmol) gave 344 mg of the crude product.

### c) 5-[4-[(2,5-Difluorophenyl)methoxy]butyl]-6H-1,3,4-thiadiazin-2-amine

Prepared as described for 5-[4-[(2-chloro-6-fluoro-phenyl)methoxy]butyl]-6H-1,3,4-thiadiazin-2-amine, (Example 87c). Starting from 1-bromo-6-[(2,5-difluorophenyl)methoxy]hexan-2-one (344 mg, 1.07 mmol) gave 20.2 mg (6%) of the product.

### Example 89

### 5-[4-[(4-Chloro-3-fluoro-phenyl)methoxy]butyl]-6H-1,3,4-thiadiazin-2-amine

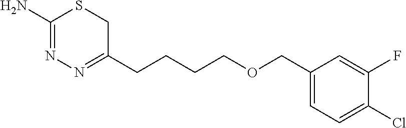 [SMILES: NC1=NN=C(CCCCOCc2ccc(Cl)c(F)c2)CS1]

### a) 6-[(4-Chloro-3-fluoro-phenyl)methoxy]hexan-2-one

Prepared as described for 6-[(2-chloro-6-fluoro-phenyl)methoxy]hexan-2-one, (Example 87a). Starting from (4-chloro-3-fluoro-phenyl)methanol (433.5 mg, 2.70 mmol) and 6-iodohexan-2-one (610.4 mg, 2.70 mmol) yielded 256 mg (37%) of the product. MS (ESI⁺): m/z 259 [M+H]⁺.

### b) 1-Bromo-6-[(4-chloro-3-fluoro-phenyl)methoxy]hexan-2-one

Prepared as described for 1-bromo-6-[(2-chloro-6-fluoro-phenyl)methoxy]hexan-2-one, (Example 87b). Starting from 6-[(4-chloro-3-fluoro-phenyl)methoxy]hexan-2-one (256 mg, 0.99 mmol) and bromine (0.05 mL, 1.04 mmol) gave 310 mg of the crude product.

### c) 5-[4-[(4-Chloro-3-fluoro-phenyl)methoxy]butyl]-6H-1,3,4-thiadiazin-2-amine

Prepared as described for 5-[4-[(2-chloro-6-fluoro-phenyl)methoxy]butyl]-6H-1,3,4-thiadiazin-2-amine, (Example 87c). Starting from 1-bromo-6-[(4-chloro-3-fluoro-phenyl)methoxy]hexan-2-one (310 mg, 0.92 mmol) gave 25.1 mg (8%) of the product. ¹H NMR (400 MHz, CD₃OD)

### Example 90

### 5-[4-[(3,5-Difluorophenyl)methoxy]butyl]-6H-1,3,4-thiadiazin-2-amine

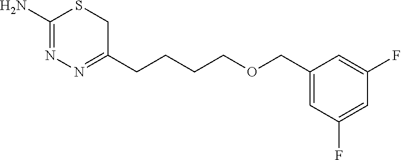 [SMILES: NC1=NN=C(CCCCOCc2cc(F)cc(F)c2)CS1]

### a) 6-[(3,5-Difluorophenyl)methoxy]hexan-2-one

Prepared as described for 6-[(2-chloro-6-fluoro-phenyl)methoxy]hexan-2-one, (Example 87a). Starting from (3,5-difluorophenyl)methanol (288.2 mg, 2.0 mmol) and 6-iodohexan-2-one (452.1 mg, 2.0 mmol) yielded 137 mg (28%) of the product. MS (ESI⁺): m/z 243 [M+H]⁺.

### b) 1-Bromo-6-[(3,5-difluorophenyl)methoxy]hexan-2-one

Prepared as described for 1-bromo-6-[(2-chloro-6-fluoro-phenyl)methoxy]hexan-2-one, (Example 87b). Starting from 6-[(3,5-difluorophenyl)methoxy]hexan-2-one (137 mg, 0.57 mmol) and bromine (0.03 mL, 0.59 mmol) gave 154 mg of the crude product.

### c) 5-[4-[(3,5-Difluorophenyl)methoxy]butyl]-6H-1,3,4-thiadiazin-2-amine

Prepared as described for 5-[4-[(2-chloro-6-fluoro-phenyl)methoxy]butyl]-6H-1,3,4-thiadiazin-2-amine, (Example 87c). Starting from 1-bromo-6-[(3,5-difluorophenyl)methoxy]hexan-2-one (154.2 mg, 0.48 mmol) gave 8.0 mg (5%) of the product.

### Example 91

### 5-[4-[[3-(Trifluoromethyl)phenyl]methoxy]butyl]-6H-1,3,4-thiadiazin-2-amine

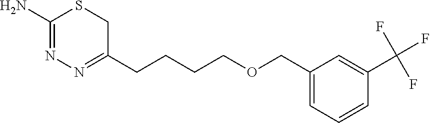 [SMILES: NC1=NN=C(CCCCOCc2cccc(C(F)(F)F)c2)CS1]

### a) 6-[[3-(Trifluoromethyl)phenyl]methoxy]hexan-2-one

Prepared as described for 6-[(2-chloro-6-fluoro-phenyl)methoxy]hexan-2-one, (Example 87a). Starting from [3-(trifluoromethyl)phenyl]methanol (475.6 mg, 2.70 mmol) and 6-iodohexan-2-one (610 mg, 2.70 mmol) yielded 144 mg (19%) of the product. MS (ESI⁺): m/z 275 [M+H]⁺.

### b) 1-Bromo-6-[[3-(trifluoromethyl)phenyl]methoxy]hexan-2-one

Prepared as described for 1-bromo-6-[(2-chloro-6-fluoro-phenyl)methoxy]hexan-2-one, (Example 87b). Starting from 6[[3-(trifluoromethyl)phenyl]methoxy]hexan-2-one (299 mg, 1.09 mmol) and bromine (0.06 mL, 1.14 mmol) gave 343 mg of the crude product.

### c) 5-[4-[[3-(Trifluoromethyl)phenyl]methoxy]butyl]-6H-1,3,4-thiadiazin-2-amine

1-Bromo-6[[3-(trifluoromethyl)phenyl]methoxy]hexan-2-one (343 mg, 0.97 mmol) and thiosemicarbazide (97.3 mg, 1.07 mmol) were mixed with ethanol (3 mL) Then HBr-HOAc 1:1 (0.01 mL) was added and the reaction was stirred at room temperature overnight. The reaction was concentrated to dryness and the residue was taken up in ethyl acetate (50 mL) and the organic phase was washed with 2×10 mL water then 1×10 mL saturated brine solution. The organic layer was then separated and dried over sodium sulfate before it was concentrated to dryness. The crude was purified by column chromatography (eluting first with 100% ethyl acetate and then heptane/methanol 9:1) to give 52.4 mg of the product.

### Example 92

### 5-[4-[(4-Chlorophenyl)methoxy]butyl]-6H-1,3,4-thiadiazin-2-amine

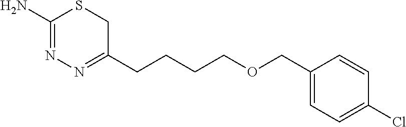 [SMILES: NC1=NN=C(CCCCOCc2ccc(Cl)cc2)CS1]

### a) 6-[(4-Chlorophenyl)methoxy]hexan-2-one

Prepared as described for 6-[(2-chloro-6-fluoro-phenyl)methoxy]hexan-2-one, (Example 87a). Starting from (4-chlorophenyl)methanol (214 mg, 1.5 mmol) and 6-iodohexan-2-one (226 mg, 1.0 mmol) yielded 214 mg (89%) of the product.

### b) 1-Bromo-6-[(4-chlorophenyl)methoxy]hexan-2-one

6-[(4-Chlorophenyl)methoxy]hexan-2-one (214 mg, 0.90 mmol) was dissolved in dichloromethane (60 mL) and methanol (3 mL). Tetra-n-butylammonium tribromide (482 mg, 1.00 mmol) was added and the reaction mixture stirred at room temperature for three days. The reaction was quenched with a drop of water and the solvent was evaporated to give 287 mg of the crude product, which was used in the next step without further purification.

### c) 5-[4-[(4-Chlorophenyl)methoxy]butyl]-6H-1,3,4-thiadiazin-2-amine

Prepared as described for 5-[4-[(2-chloro-6-fluoro-phenyl)methoxy]butyl]-6H-1,3,4-thiadiazin-2-amine, (Example 87c). Starting from 1-bromo-6-[(4-chlorophenyl)methoxy]hexan-2-one (287 mg, 0.90 mmol) gave 4.5 mg (1.4%) of the product.

### Example 93

### 5-[4-(m-Tolylmethoxy)butyl]-6H-1,3,4-thiadiazin-2-amine

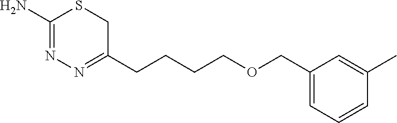 [SMILES: Cc1cccc(COCCCCC2=NN=C(N)SC2)c1]

### a) 6-(m-Tolylmethoxy)hexan-2-one

3-Methyl-benzyl alcohol (183 mg, 1.50 mmol) was mixed with potassium hydroxide (99.0 mg 1.50 mmol) and stirred neat at room temperature for 30 min. Then 6-iodo-2-hexanone (226 mg, 1.00 mmol) was added and the reaction was stirred at room temperature overnight. The reaction was quenched with water and extracted with 3×10 mL of ethyl acetate. The organic layer was washed with 2×10 ml of brine, dried over sodium sulfate and concentrated. The residue was purified by column chromatography (heptane/ethyl acetate 85:15) to give 199 mg (90%) of product. MS (ESI⁺): m/z 221 [M+H]⁺.

### b) 1-Bromo-6-(m-tolylmethoxy)hexan-2-one

Prepared as described for 1-bromo-6-[(4-chlorophenyl)methoxy]hexan-2-one, (Example 92b). Starting from 6-(m-tolylmethoxy)hexan-2-one (199 mg, 0.90 mmol) and tetra-n-butylammonium tribromide (482 mg, 1.00 mmol) gave 277 mg of the crude product.

### c) 5-[4-(m-Tolylmethoxy)butyl]-6H-1,3,4-thiadiazin-2-amine

Prepared as described for 5-[4-[(2-chloro-6-fluoro-phenyl)methoxy]butyl]-6H-1,3,4-thiadiazin-2-amine, (Example 87c). Starting from 1-bromo-6-(m-tolylmethoxy)hexan-2-one (277 mg, 0.90 mmol) gave 21 mg (8%) of the product.

### Example 94

### 5-[4-[(2,3,4-Trifluorophenyl)methoxy]butyl]-6H-1,3,4-thiadiazin-2-amine

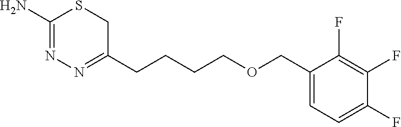 [SMILES: NC1=NN=C(CCCCOCc2ccc(F)c(F)c2F)CS1]

### a) 6-[(2,3,4-Trifluorophenyl)methoxy]hexan-2-one

To a solution of 6-iodohexan-2-one (1.439 g, 6.37 mmol) in toluene (0.50 mL) was added potassium hydroxide (357 mg, 6.37 mmol, crushed pellets) and the resulting suspension was stirred for 30 min. Then, (2,3,4-trifluorophenyl)methanol (860 mg, 5.31 mmol) was added The mixture was stirred vigorously at room temperature overnight. Water (20 mL) was then added and the product was extracted with ethyl acetate (3×25 mL). The combined organic phases were washed with brine, dry over sodium sulfate and concentrated at reduced pressure. In order to simplify the purification the (2,3,4-trifluorophenyl)methanol (starting material) still remaining in the reaction mixture was silylated. The crude was dissolved in dichloromethane (10 mL), tert-butyldimethylsilyl chloride (600 mg, 3.98 mmol) was added followed by addition of imidazole (271 mg, 3.98 mmol). The reaction was monitored by LC/MS and after 30 min LC/MS showed full conversion of (2,3,4-trifluorophenyl)methanol. The solid was filtered off. Water was added and the product was extracted with dichloromethane. The combined organic phases were dried over sodium sulfate and the solution was finally filtered through pad of silica. The product was eluted with ethyl acetate and the solution was concentrated at reduced pressure. The product was purified by column chromatography (heptane/ethyl acetate 9:1) to afford 538 mg (39%) of the product. MS (ESI⁺): m/z 261 [M+H]⁺.

### b) 1-Bromo-6-[(2,3,4-trifluorophenyl)methoxy]hexan-2-one

6-[(2,3,4-trifluorophenyl)methoxy]hexan-2-one (211 mg, 0.81 mmol) was dissolved in dichloromethane (6 mL) and methanol (3 mL) . Tetra-n-butylammonium tribromide (430.5 mg, 0.89 mmol) was added and the reaction was left to stir at room temperature overnight. LC/MS analysis was thereafter performed, 1 drop of water was added and the reaction mixture was left to stir for a few min. The solvent was thereafter removed under reduced pressure and the crude was dried further under a stream of nitrogen. The product was filtered through a silica column (ethyl acetate:heptane 1:9). The fractions containing the product were combined to yield 257 mg of the product as a mixture of isomers. MS (ESI⁺): m/z 339, 341 [M+H]⁺.

### c) 5-[4-[(2,3,4-Trifluorophenyl)methoxy]butyl]-6H-1,3,4-thiadiazin-2-amine

1-Bromo-6-[(2,3,4-trifluorophenyl)methoxy]hexan-2-one (257 mg, 0.76 mmol) was dissolved in ethanol (3 mL) containing HBr (48 wt. % in water, 0.05 mL, 0.83 mmol) and thiosemicarbazide (76.0 mg, 0.83 mmol) was thereafter added. The reaction was stirred at room temperature for 30 min. A white solid precipitated out of solution. The ethanol was removed and the solid material was purified on by column chromatography (ethyl acetate to ethyl acetate/methanol 9:1). The product was thereafter washed with ethyl acetate (3×1 mL) and was isolated in 70 mg (28%) yield.

### Examples 95-104 were prepared as described for 5-[4-[(2,3,4-Trifluorophenyl)methoxy]butyl]-6H-1,3,4-thiadiazin-2-amine, example 94

### Example 95

### 5-[4-[(2,3-Difluorophenyl)methoxy]butyl]-6H-1,3,4-thiadiazin-2-amine

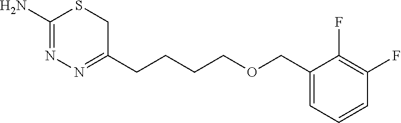 [SMILES: NC1=NN=C(CCCCOCc2cccc(F)c2F)CS1]

### a) 6-[(2,3-Difluorophenyl)methoxy]hexan-2-one

Starting from (2,3-difluorophenyl)methanol (800 mg, 5.47 mmol) 6-iodohexan-2-one (1.48 g, 6.57 mmol) gave 504 mg (38%) of the product. MS (ESI⁺): m/z 243 [M+H]⁺.

### b) 1-Bromo-6-[(2,3-difluorophenyl)methoxy]hexan-2-one

Starting from 6-[(2,3-difluorophenyl)methoxy]hexan-2-one (200 mg, 0.83 mmol) gave 244 mg of the product as a mixture of isomers. MS (ESI⁺): m/z 321, 323 [M+H]⁺.

### c) 5-[4-[(2,3-Difluorophenyl)methoxy]butyl]-6H-1,3,4-thiadiazin-2-amine

Starting from 1-bromo-6-[(2,3-difluorophenyl)methoxy]hexan-2-one (244 mg, 0.76 mmol) gave 20 mg (8%) of the product.

### Example 96

### 5-[4-[(3-Bromo-5-fluoro-phenyl)methoxy]butyl]-6H-1,3,4-thiadiazin-2-amine

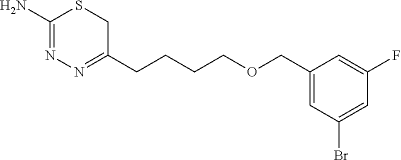 [SMILES: NC1=NN=C(CCCCOCc2cc(F)cc(Br)c2)CS1]

### a) 6-[(3-Bromo-5-fluoro-phenyl)methoxy]hexan-2-one

Starting from (3-bromo-5-fluoro-phenyl)methanol (400 mg, 1.95 mmol) and 6-iodohexan-2-one (662 mg, 2.93 mmol) gave 490 mg (83%) of the product. MS (ESI⁺): m/z 303, 305 [M+H]⁺.

### b) 1-Bromo-6-[(3-bromo-5-fluoro-phenyl)methoxy]hexan-2-one

Starting from 6-[(3-bromo-5-fluoro-phenyl)methoxy]hexan-2-one (490 mg, 1.62 mmol) gave 517 mg of crude that was used in the next step.

### c) 5-[4-[(3-Bromo-5-fluoro-phenyl)methoxy]butyl]-6H-1,3,4-thiadiazin-2-amine

Starting from 1-bromo-6-[(3-bromo-5-fluoro-phenyl)methoxy]hexan-2-one (518 mg, 1.36 mmol) gave 51 mg (10%) of the product.

### Example 97

### 5-[4-[(2,4,6-Trifluorophenyl)methoxy]butyl]-6H-1,3,4-thiadiazin-2-amine

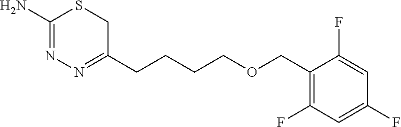 [SMILES: NC1=NN=C(CCCCOCc2c(F)cc(F)cc2F)CS1]

### a) 6-[(2,4,6-Trifluorophenyl)methoxy]hexan-2-one

Starting from (2,4,6-trifluorophenyl)methanol (800 mg, 4.93 mmol) and 6-iodohexan-2-one (1.67 g, 7.4 mmol) gave 444 mg (35%) of the product. MS (ESI⁺): m/z 261 [M+H]⁺.

### b) 1-Bromo-6-[(2,4,6-trifluorophenyl)methoxy]hexan-2-one

Starting from 6-[(2,4,6-trifluorophenyl)methoxy]hexan-2-one (444 mg, 1.71 mmol) gave 430 mg of the product as a mixture of isomers. MS (ESI⁺): m/z 339 [M+H]⁺.

### c) 5-[4-[(2,4,6-Trifluorophenyl)methoxy]butyl]-6H-1,3,4-thiadiazin-2-amine

Starting from 1-bromo-6-[(2,4,6-trifluorophenyl)methoxy]hexan-2-one (430 mg, 1.27 mmol) gave 46 mg (11%) of the product.

### Example 98

### 5-[4-[(2,6-Difluorophenyl)methoxy]butyl]-6H-1,3,4-thiadiazin-2-amine

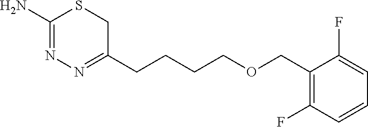 [SMILES: NC1=NN=C(CCCCOCc2c(F)cccc2F)CS1]

### a) 6-[(2,6-Difluorophenyl)methoxy]hexan-2-one

Starting from (2,6-difluorophenyl)methanol (0.62 mL, 5.55 mmol) and 6-iodohexan-2-one (1.88 g, 8.33 mmol) gave 655 mg (49%) of the product. MS (ESI⁺): m/z 243 [M+H]⁺.

### b) 1-Bromo-6-[(2,6-difluorophenyl)methoxy]hexan-2-one

Starting from 6-[(2,6-difluorophenyl)methoxy]hexan-2-one (655 mg, 2.71 mmol) gave 698 mg of the product as a mixture of isomers. MS (ESI⁺): m/z 321, 323 [M+H]⁺.

### c) 5-[4-[(2,6-Difluorophenyl)methoxy]butyl]-6H-1,3,4-thiadiazin-2-amine

Starting from 1-bromo-6-[(2,6-difluorophenyl)methoxy]hexan-2-one (698 mg, 2.17 mmol) gave 65 mg (10%) of the product.

### Example 99

### 5-[4-[(2,6-Difluoro-4-methoxy-phenyl)methoxy]butyl]-6H-1,3,4-thiadiazin-2-amine

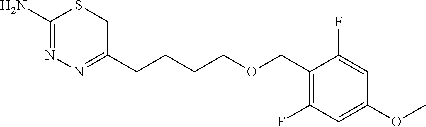 [SMILES: COc1cc(F)c(COCCCCC2=NN=C(N)SC2)c(F)c1]

### a) 6-[(2,6-Difluoro-4-methoxy-phenyl)methoxy]hexan-2-one

Starting from (2,6-difluoro-4-methoxy-phenyl)methanol (0.77 mL, 6.89 mmol) and 6-iodohexan-2-one (2.921 g, 10.34 mmol) afforded 900 mg (27%) of the product. MS (ESI⁺): m/z 157 [M+H]⁺.

### b) 1-Bromo-6-[(2,6-difluoro-4-methoxy-phenyl)methoxy]hexan-2-one

Starting from 6-[(2,6-difluoro-4-methoxy-phenyl)methoxy]hexan-2-one (900 mg, 3.31 mmol) gave 940 mg of the product as a mixture of isomers. MS (ESI⁺): m/z 157 [M+H]⁺.

### c) 5-[4-[(2,6-Difluoro-4-methoxy-phenyl)methoxy]butyl]-6H-1,3,4-thiadiazin-2-amine

Starting from 1-bromo-6-[(2,6-difluoro-4-methoxy-phenyl)methoxy]hexan-2-one (940 mg, 2.68 mmol afforded 120 mg (13%) of the product.

### Example 100

### 5-[4-[(4-Bromo-3-fluoro-phenyl)methoxy]butyl]-6H-1,3,4-thiadiazin-2-amine

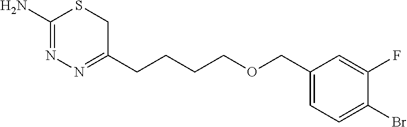 [SMILES: NC1=NN=C(CCCCOCc2ccc(Br)c(F)c2)CS1]

### a) 6-[(4-Bromo-3-fluoro-phenyl)methoxy]hexan-2-one

Starting from (4-bromo-3-fluoro-phenyl)methanol (0.77 mL, 3.59 mmol) and 6-iodohexan-2-one (1.28 g, 5.38 mmol) gave 496 mg (46%) of the product. MS (ESI⁺): m/z 303 [M+H]⁺.

### b) 1-Bromo-6-[(4-bromo-3-fluoro-phenyl)methoxy]hexan-2-one

Starting from 6-[(4-bromo-3-fluoro-phenyl)methoxy]hexan-2-one (496 mg, 1.64 mmol) gave 540 mg of the product as a mixture of isomers. MS (ESI⁺): m/z 383 [M+H]⁺.

### c) 5-[4-[(4-Bromo-3-fluoro-phenyl)methoxy]butyl]-6H-1,3,4-thiadiazin-2-amine

Starting from 1-bromo-6-[(4-bromo-3-fluoro-phenyl)methoxy]hexan-2-one (540 mg, 1.41 mmol) gave 116 mg (22%) of the product.

### Example 101

### 5-[4-(1,3-Benzothiazol-2-ylmethoxy)butyl]-6H-1,3,4-thiadiazin-2-amine

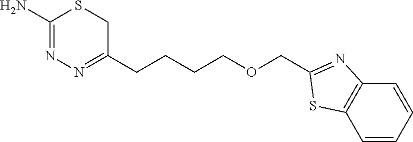 [SMILES: NC1=NN=C(CCCCOCc2nc3ccccc3s2)CS1]

### a) 6-(1,3-Benzothiazol-2-ylmethoxy)hexan-2-one

Starting from 2-hydroxymethylbenzothiazole (1.30 g, 7.87 mmol) and 6-iodohexan-2-one (2.72 g, 10.23 mmol) gave 248 mg (12%) of the product. MS (ESI⁺): m/z 264 [M+H]⁺.

### b) 6-(1,3-Benzothiazol-2-ylmethoxy)-1-bromo-hexan-2-one

Starting from 6-(1,3-benzothiazol-2-ylmethoxy)hexan-2-one (248 mg, 0.94 mmol) gave 138 mg (43%) of the product as a mixture of isomers. MS (ESI⁺): m/z 344 [M+H]⁺.

### c) 5-[4-(1,3-Benzothiazol-2-ylmethoxy)butyl]-6H-1,3,4-thiadiazin-2-amine

Starting from 6-(1,3-benzothiazol-2-ylmethoxy)-1-bromo-hexan-2-one (138 mg, 0.34 mmol) gave 34 mg (30%) of the product.

### Example 102

### 5-[4-[(2-Nitrophenyl)methoxy]butyl]-6H-1,3,4-thiadiazin-2-amine

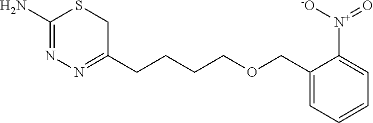

### a) 6-[(2-Nitrophenyl)methoxy]hexan-2-one

Starting from 2-nitrobenzyl alcohol (2.0g, 13.0 mmol) and 6-iodohexan-2-one (4.52 g, 17.0 mmol) gave 247 mg (8%) of the product. MS (ESI⁺): m/z 252 [M+H]⁺.

### b) 1-Bromo-6-[(2-nitrophenyl)methoxy]hexan-2-one

Starting from 6-[(2-nitrophenyl)methoxy]hexan-2-one (247 mg, 0.98 mmol) gave 206 mg (63%) of the product as a mixture of isomers. MS (ESI⁺): m/z 347 [M+H]⁺.

### c) 5-[4-[(2-Nitrophenyl)methoxy]butyl]-6H-1,3,4-thiadiazin-2-amine

Starting from 1-bromo-6-[(2-nitrophenyl)methoxy]hexan-2-one (206 mg, 0.620 mmol) gave 44 mg (22%) of the product.

### Example 103

### 5-[4-(Tetrahydrofuran-2-ylmethoxy)butyl]-6H-1,3,4-thiadiazin-2-amine

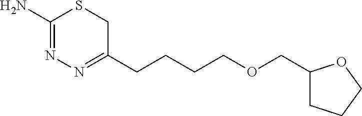 [SMILES: NC1=NN=C(CCCCOCC2CCCO2)CS1]

### a) 6-(Tetrahydrofuran-2-ylmethoxy)hexan-2-one

Starting from tetrahydrofurfuryl alcohol (0.60 g, 5.9 mmol) and 6-iodohexan-2-one (1.59 g, 7.0 mmol) gave 355 mg (30%) of the product. MS (ESI⁺): m/z 201 [M+H]⁺.

### b) 1-Bromo-6-(tetrahydrofuran-2-ylmethoxy)hexan-2-one

Starting from 6-(tetrahydrofuran-2-ylmethoxy)hexan-2-one (355 mg, 1.77 mmol) gave 77 mg (15%) of the product as a mixture of isomers. MS (ESI⁺): m/z 279, 281 [M+H]⁺.

### c) 5-[4-(Tetrahydrofuran-2-ylmethoxy)butyl]-6H-1,3,4-thiadiazin-2-amine

Starting from 1-bromo-6-(tetrahydrofuran-2-ylmethoxy)hexan-2-one (77 mg, 0.28 mmol) gave 15 mg (20%) of the product.

### Example 104

### 5-[4-[(2-Methylcyclopropyl)methoxy]butyl]-6H-1,3,4-thiadiazin-2-amine

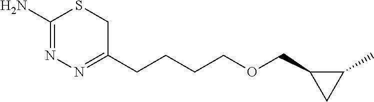 [SMILES: C[C@@H]1C[C@H]1COCCCCC1=NN=C(N)SC1]

### a) 6-[(2-Methylcyclopropyl)methoxy]hexan-2-one

Starting from 2-methylcyclopropanemethanol (0.60 g, 6.9 mmol) and 6-iodohexan-2-one (1.89 g, 8.40 mmol) gave 440 mg (34%) of the product. MS (ESI⁺): m/z 201 [M+H]⁺.

### b) 1-Bromo-6-[(2-methylcyclopropyl)methoxy]hexan-2-one

Starting from 6-[(2-methylcyclopropyl)methoxy]hexan-2-one (440 mg, 2.39 mmol) gave 110 mg (18%) of the product as a mixture of isomers. MS (ESI⁺): m/z 185 [M+H]⁺.

### c) 5-[4-[(2-Methylcyclopropyl)methoxy]butyl]-6H-1,3,4-thiadiazin-2-amine

Starting from 1-bromo-6-[(2-methylcyclopropyl)methoxy]hexan-2-one (110 mg, 0.42 mmol) gave 76 mg (71%) of the product as an 4:1 trans/cis mixture.

### Example 105

### 5-[4-[(2,4-Dimethylphenyl)methoxy]butyl]-6H-1,3,4-thiadiazin-2-amine

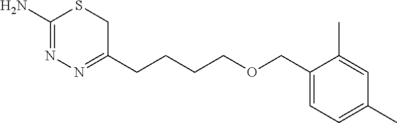 [SMILES: Cc1ccc(COCCCCC2=NN=C(N)SC2)c(C)c1]

### a) 2,4-dimethylbenzyl methanesulfonate

(2,4-dimethylphenyl)methanol (1.72 g, 12.7 mmol) and triethyl amine (5.1 mL, 38.1 mmol) were dissolved in dichloromethane (50 mL) and the solution was cooled to 0° C. A solution of methanesulfonyl chloride (1.18 mL, 15.2 mmol) in dichloromethane (20 mL) was added dropwise for a period of 20 minutes. The reaction mixture was thereafter allowed to reach room temperature, and was stirred at that temperature for 2 hours. The excess of methanesulfonyl chloride was quenched by addition of methanol (5.0 mL) and stirring for an additional 10 minutes. The reaction mixture was extracted with dilute HCl (5%, 100 mL), and thereafter washed with sodium bicarbonate (5% solution, 2×50 mL) and with brine (2×100 mL). The dichloromethane phased was dried over anhydrous sodium sulfate and evaporated to dryness, yielding 2.57 g of the title product, which was used without further purification in the subsequent step.

### b) 1-((hex-5-en-1-yloxy)methyl)-2,4-dimethylbenzene

To a solution of the 5-hexene-1-ol (1.84 g, 18.0 mmol) in dimethylformamide (40 mL), was added at room temperature NaH (60% dispersion in oil, 0.72 g, 18 mmol) in two portions. After stirring the reaction mixture at room temperature for 30 minutes, a solution of 2,4-dimethylbenzyl methanesulfonate (2.57 g) in dimethylformamide (20 mL) was added dropwise. The reaction mixture was thereafter stirred at room temperature for an additional 18 hours. Water was then added (100 mL) and the reaction mixture was stirred for 5 minutes before extraction with ethyl acetate (100 mL) was performed. The organic phases were washed with brine (2×100 mL), dried over anhydrous sodium sulfate and concentrated at reduced pressure. The title compound was purified by column chromatography (hexane to hexane/ethyl acetate 95:5) and was isolated in 1.68 g (61%) yield.

### c) 1-bromo-6-((2,4-dimethylbenzyl)oxy)hexan-2-ol

To a cold solution of 1-((hex-5-en-1-yloxy)methyl)-2,4-dimethylbenzene (1.09 g, 5.0 mmol) in dimethyl sulfoxide (7.0 mL) and water (2.0 mL), was added N-bromosuccinimide (1.8 g, 10 mmol) in one portion. The reaction mixture was allowed to reach room temperature and was stirred at that temperature for 4 hours. The reaction mixture was thereafter extracted with ethyl acetate (100 mL).

The organic phases were washed with brine (2×100 mL), was dried over anhydrous sodium sulfate and concentrated at reduced pressure. The title compound was purified by column chromatography (hexane to hexane/ethyl acetate 3:1) and was isolated in 0.91 g (58%) yield.

### d) 1-bromo-6-((2,4-dimethylbenzyl)oxy)hexan-2-one

Pyridinium chlorochromate (2.45 g, 11.3 mmol) was dissolved in dichloromethane (30 mL) after stirring the mixture for 10 minutes. This solution was added in one portion to a solution of 1-bromo-6-((2,4-dimethylbenzyl)oxy)hexan-2-ol (0.85 g, 2.7 mmol). The reaction mixture was stirred at room temperature for 5 hours, whereafter silica gel (30 g) was added and the solvent was removed at reduced pressure. The dried silica gel was loaded on to a silica gel column and the title product was purified by column chromatography (hexane to hexane/ethyl acetate 3:1) and was isolated in 0.71 g (85%) yield.

### e) 5-[4-[(2,4-Dimethylphenyl)methoxy]butyl]-6H-1,3,4-thiadiazin-2-amine

Prepared as described for 5-[4-[(2,3,4-Trifluorophenyl)methoxy]butyl]-6H-1,3,4-thiadiazin-2-amine, example 94c. Starting from 1-bromo-6-[(2,4-dimethylphenyl)methoxy]hexan-2-one (189 mg, 0.60 mmol) gave 37 mg (20%) of the product.

### Example 106

### 5-[4-[(2-Chlorophenyl)methoxy]butyl]-6H-1,3,4-thiadiazin-2-amine

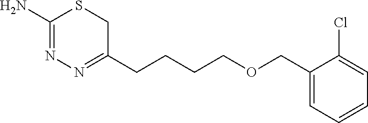 [SMILES: NC1=NN=C(CCCCOCc2ccccc2Cl)CS1]

### a) 2-chlorobenzyl methanesulfonate

Prepared as described for 2,4-dimethylbenzyl methanesulfonate, example 38a. Starting from 1.81 g (12.7 mmol) of (2-chlorophenyl)methanol gave 2.66 g of the product.

### b) 1-chloro-2-((hex-5-en-1-yloxy)methyl)benzene

Prepared as described for 1-((hex-5-en-1-yloxy)methyl)-2,4-dimethylbenzene, example 105b. Starting from 2.66 g of 2-chlorobenzyl methanesulfonate gave 1.56 g (55%) of the product.

### c) 1-bromo-6-((2-chlorobenzyl)oxy)hexan-2-ol

Prepared as described for 1-bromo-6-((2,4-dimethylbenzyl)oxy)hexan-2-ol, example 105c. Starting from 1.12 g (5.0 mmol) of 1-chloro-2-((hex-5-en-1-yloxy)methyl)benzene gave 0.94 g (59%) of the product.

### d) 1-bromo-6-((2-chlorobenzyl)oxy)hexan-2-one

Prepared as described for 1-bromo-6-((2,4-dimethylbenzyl)oxy)hexan-2-one, example 105d. Starting from 0.90 g (2.8 mmol) of 1-bromo-6-((2-chlorobenzyl)oxy)hexan-2-ol gave 0.78 g (87%) of the product.

### e) 5-[4-[(2-Chlorophenyl)methoxy]butyl]-6H-1,3,4-thiadiazin-2-amine

Prepared as described for 5-[4-[(2,3,4-Trifluorophenyl)methoxy]butyl]-6H-1,3,4-thiadiazin-2-amine, example 94c. Starting from 1-bromo-6-[(2-chlorophenyl)methoxy]hexan-2-one (94 mg, 0.29 mmol) gave 22 mg (24%) of the product.

### Example 107

### 5-[4-[(5-Methyl-2-thienyl)methoxy]butyl]-6H-1,3,4-thiadiazin-2-amine

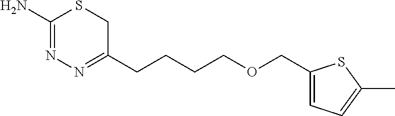 [SMILES: Cc1ccc(COCCCCC2=NN=C(N)SC2)s1]

### a) 6-[(5-Methyl-2-thienyl)methoxy]hexan-2-one

(5-Methyl-2-thienyl)methanol (425 mg, 3.32 mmol) was dissolved in toluene (0.50 mL). Potassium hydroxide (186 mg, 3.32 mmol) was added and the mixture was stirred at room temperature for 30 min. 6-Iodohexan-2-one (500 mg, 2.21 mmol) was added and the solution stirred at room temperature overnight. Water was added and the reaction extracted with dichloromethane. The crude was purified by column chromatography on silica (heptane/ethyl acetate 75:25) to give 187 mg, (37%) of the title compound. MS (ESI⁺): m/z 244 [M+18]⁺

### b) Trimethyl-[1-methylene-5-[(5-methyl-2-thienyl)methoxy]pentoxy]silane

In a small vial with septum cooled to 0° C., was added n-butyllithium (1.6 M, 0.51 mL, 0.82 mmol) to a solution of diisopropylamine (0.13 mL, 0.90 mmol) dissolved in tetrahydrofuran (1.5 mL). After 30 minutes of stirring at this temperature, the solution was added dropwise to a solution of 6-[(5-methyl-2-thienyl)methoxy]hexan-2-one (185 mg, 0.82 mmol) in tetrahydrofuran (3 mL) (microwave vial) at −78° C. After 40 minutes of stirring at this temperature, trimethylsilyl chloride (0.21 mL, 1.63 mmol) in tetrahydrofuran (0.5 mL) at −78° C. was added. The reaction was allowed to reach room temperature and after 1 h saturated aqueous NaHCO₃ (0.5 mL) was added followed by diethyl ether (60 mL). The organic phase was washed with water twice and dried over sodium carbonate, filtered and the solvent was evaporated. Taken to the next step without further purification.

### c) 1-Bromo-6-[(5-methyl-2-thienyl)methoxy]hexan-2-one

To an ice cold solution of trimethyl-[1-methylene-5-[(5-methyl-2-thienyl)methoxy]pentoxy]silane (214 mg, 0.72 mmol) in tetrahydrofuran (10 mL) was added N-Bromosuccinimide (134 mg, 0.75 mmol). After stirring at 0° C. for 10 min, LC/MS reveal full conversion and the mixture was poured out into a 50% saturated aqueous solution of NaHCO₃ (10 mL) and extracted with ethyl acetate (3×). The combined organic layers were dried over magnesium sulfate and concentrated at reduced pressure. The crude was purified by column chromatography (10 to 20% ethyl acetate in n-heptane) to afford 111 mg (51%) of the title compound. MS (ESI⁻): m/z 322, 324 [M+18]⁺

### d) 5-[4-[(5-Methyl-2-thienyl)methoxy]butyl]-6H-1,3,4-thiadiazin-2-amine

Ethanol (4 mL) and dichloromethane (4 mL) was added to 1-bromo-6-[(5-methyl-2-thienyl)methoxy]hexan-2-one (111 mg, 0.36 mmol) followed by HBr (48 wt. % in water, 0.04 mL, 0.360 mmol) and thiosemicarbazide (33.1 mg, 0.360 mmol). The reaction was diluted with dichloromethane and washed with brine, dried over magnesium sulfate, filtered and the solvent was evaporated. The crude was purified by column chromatography (4 to 7% methanol in dichloromethane) to give 19 mg (18%) of the title compound.

### Example 108

### 5-[4-[(4-Chlorophenyl)methylsulfanyl]butyl]-6H-1,3,4-thiadiazin-2-amine

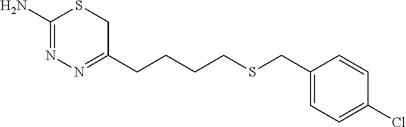 [SMILES: NC1=NN=C(CCCCSCc2ccc(Cl)cc2)CS1]

### a) 6-[(4-Chlorophenyl)methylsulfanyl]hexan-2-one

(4-Chlorophenyl)methanethiol (720 mg, 4.54 mmol) was mixed with toluene (0.5 mL) and potassium hydroxide (358 mg, 5.45 mmol) was added. The reaction was stirred at room temperature for 30 min before 6-iodohexan-2-one (0.66 mL, 4.54 mmol) was added. The reaction was then stirred at room temperature overnight. The reaction mixture was concentrated to dryness and the residue was taken up in ethyl acetate (120 mL) and the organic phase was washed with water (2×30 mL) then saturated brine solution (1×30 mL). The organic layer was separated and dried over sodium sulfate and concentrated to dryness. The crude was then purified by flash column chromatography (heptane/ethyl acetate 9:1) to give 1.09 g (87%) of the product. MS (ESI⁺): m/z 257 [M+H]⁺.

### b) 1-Bromo-6-[(4-chlorophenyl)methylsulfanyl]hexan-2-one

6-[(4-Chlorophenyl)methylsulfanyl]hexan-2-one (500 mg, 1.95 mmol) was dissolved in methanol (10 mL) and bromine (0.11 mL, 2.04 mmol) was added dropwise at 0° C. during 1 h. The reaction was allowed to reach room temperature and was stirred overnight. The reaction was concentrated to dryness and the residue was taken up in ethyl acetate (25 mL) and the organic phase was washed with water (2×10 mL) then saturated brine solution (1×10 mL). The organic layer was separated and dried over magnesium sulfate. The solution was concentrated to dryness to give 495 mg of crude product, which was used in the next step without further purification.

### c) 5-[4-[(4-Chlorophenyl)methylsulfanyl]butyl]-6H-1,3,4-thiadiazin-2-amine

Thiosemicarbazide (147 mg, 1.62 mmol) and 1-bromo-6-[(4-chlorophenyl)methylsulfanyl]hexan-2-one (493 mg, 1.47 mmol) were mixed in ethanol (3 mL) and the reaction stirred at room temperature overnight. The reaction mixture was concentrated to dryness and the residue was taken up in ethyl acetate (60 mL). The organic layer was washed with water (2×10 mL) then saturated brine solution (1×10 mL). The organic layer was then separated and dried over sodium sulfate before it was concentrated to dryness. The crude was then purified by flash column chromatography (ethyl acetate 100% to ethyl acetate/methanol 9:1) to give 69 mg (14%) of the product.

### Example 109

### 5-[5-(3-Ethylphenoxy)pentyl]-6H-1,3,4-thiadiazin-2-amine

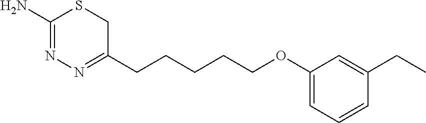 [SMILES: CCc1cccc(OCCCCCC2=NN=C(N)SC2)c1]

### a) 1-(5-Bromopentoxy)-3-ethyl-benzene

To a stirred solution of 3-ethylphenol (2.75 g, 22.5 mmol) in water (20 mL) was added 1,5-dibromopentane (3.83 mL, 28.1 mmol) and the reaction mixture was heated to reflux. Aqueous sodium hydroxide (1.6 g in 20 mL water solution) was added slowly at reflux. After the addition, reflux was continued for another 8 h. The progress of the reaction was monitored by TLC and after completion of the reaction, mixture was cooled to room temperature. The upper layer of the mixture separated and discarded and the lower layer was washed with ethyl acetate. The combined ethyl acetate layers were washed with diluted sodium hydroxide solution and water. The organic layers were dried and then concentrated. The crude product was purified by column chromatography (heptane/ethyl acetate 98:2) to give 6.00 g (98%) of the title compound. MS (ESI⁺): m/z 271 [M+H]⁺.

### b) 6-(3-Ethylphenoxy)hexanenitrile

To the stirred solution of 1-(5-bromopentoxy)-3-ethyl-benzene (6.00 g, 22.1 mmol) in ethanol (70 mL) was added aqueous potassium cyanide (1.50 g in 20 mL of water). After addition the reaction mixture was heated at reflux for 48 h. The progress of the reaction was monitored by TLC and after completion of the reaction, mixture was cooled to room temperature and concentrated. The water phase was extracted with ethyl acetate. The combined ethyl acetate layers were washed with diluted sodium hydroxide solution and water. The organic layers were dried and concentrated. The residue was purified by column chromatography (heptane/ethyl acetate 98:2) to give 5.00 g (quantitative) of the title compound. MS (ESI⁺): m/z 218 [M+H]⁺.

### c) 6-(3-Ethylphenoxy)hexanoic acid

To the stirred solution of 6-(3-ethylphenoxy)hexanenitrile (4.10 g, 18.9 mmol) in ethanol (50 mL) was added aqueous a sodium hydroxide solution (1.13 g in 15 mL of water) and after addition the reaction mixture was heated to reflux for 8 h. The progress of the reaction was monitored by TLC and after completion of the reaction, the mixture was cooled to 0° C. and the pH of the reaction mixture was adjusted to 3 by adding dilute HCl. The upper layer of the mixture was separated and discarded and the lower layer was washed with ethyl acetate. The combined ethyl acetate layers were washed with dilute sodium hydroxide solution and water. The organic layers were dried and concentrated under reduced pressure. Purification by column chromatography (heptane/ethyl acetate 98:2) afforded 0.477 g (12%) of the desired product. MS (ESI⁺): m/z 237 [M+H]⁺.

### d) 1-Bromo-7-(3-ethylphenoxy)heptan-2-one

To the stirred solution of 6-(3-ethylphenoxy)hexanoic acid (0.323 g, 1.37 mmol) in dry toluene (4.8 mL) was added oxalyl chloride (0.18 mL, 2.05 mmol) at 0° C. and the reaction mixture was stirred at 0° C. for 1 h. After completion of the reaction the solvent was evaporated and the crude was dissolved in tetrahydrofuran (4.8 mL). To this solution was added a solution of trimethylsilyldiazomethane (1.7 mL, 3.42 mmol, 2 M in hexane) at 0° C. and the temperature was increased to room temperature and the reaction mixture was stirred at room temperature for 1 h. The solvent was removed under nitrogen. The residue was dissolved in dry dichloromethane and the resultant mixture was cooled to 0° C. To the reaction mixture was then added a solution of 1:1 (v/v) 45% HBr (2 mL) and glacial acetic acid (2 mL). The mixture was diluted with water and extracted with ethyl acetate (3×). The extracts were combined, washed with water and brine, dried over magnesium sulfate and filtered. The filtrate was concentrated at reduced pressure and co-evaporated with toluene (2×) to give 0.32 g (75%) of the product.

### e) 5-[5-(3-Ethylphenoxy)pentyl]-6H-1,3,4-thiadiazin-2-amine

To a stirred solution of 1-bromo-7-(3-ethylphenoxy)heptan-2-one (0.32 g, 1.02 mmol) in ethanol (4 mL) was added thiosemicarbazide (0.093 g, 1.02 mmol) at room temperature and the mixture was stirred for 12 h. The reaction was monitored by HPLC and after completion of the reaction, the solvent was evaporated and the crude product purified by column chromatography (dichloromethane/methanol 9:1) to give 0.083 g (27%) of the title compound.

### Example 110

### 5-[5-(4-Ethylphenoxy)pentyl]-6H-1,3,4-thiadiazin-2-amine

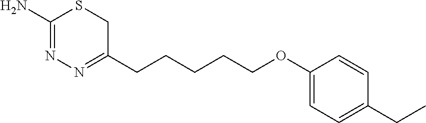 [SMILES: CCc1ccc(OCCCCCC2=NN=C(N)SC2)cc1]

### a) 1-(5-Bromopentoxy)-4-ethyl-benzene

Prepared as described for 1-(5-bromopentoxy)-3-ethyl-benzene, (Example 109a). Starting from 4-ethylphenol (1.22 g, 10.0 mmol) and 1,5-dibromopentane (1.7 mL, 12.5 mmol) gave 2.71 g (quantitative) of the product.

### b) 6-(4-Ethylphenoxy)hexanenitrile

Prepared as described for 6-(3-Ethylphenoxy)hexanenitrile, (Example 109b). Starting from 1-(5-bromopentoxy)-4-ethyl-benzene (2.71 g, 10.0 mmol) gave 1.24 g (57%) of the product. MS (ESI⁺): m/z 218 [M+H]⁺.

### c) 6-(4-Ethylphenoxy)hexanoic acid

Prepared as described for 6-(3-ethylphenoxy)hexanoic acid, (Example 109c). Starting from 6-(4-ethylphenoxy)hexanenitrile (1.24 g, 5.7 mmol) afforded 1.25 g (93%) of the title compound. MS (ESI⁺): m/z 237 [M+H]⁺.

### d) 1-Bromo-7-(4-ethylphenoxy)heptan-2-one

Prepared as described for 1-bromo-7-(3-ethylphenoxy)heptan-2-one, (Example 109d). Starting from 6-(4-ethylphenoxy)hexanoic acid (1.25 g) gave 512 mg of the title compound.

### e) 5-[5-(4-Ethylphenoxy)pentyl]-6H-1,3,4-thiadiazin-2-amine

Prepared as described for 5-[5-(3-ethylphenoxy)pentyl]-6H-1,3,4-thiadiazin-2-amine, (Example 109e). Starting from 1-bromo-7-(4′-ethylphenoxyl)-ketone-2-one (313 mg, 1.00 mmol) and thiosemicarbazide (100 mg, 1.10 mmol) gave 37 mg (12%) of the product. ¹H NMR (400 MHz, DMSO-d₆) δ 7.11-7.07 (m, 2H), 6.84-6.79 (m, 2H), 3.91 (t, J=6.5 Hz, 2H), 3.38 (s, 2H), 2.57-2.43 (m, 4H, partially obscured by the solvent signal), 1.76-1.67 (m, 2H), 1.67-1.57 (m, 2H), 1.49-1.39 (m, 2H), 1.13 (t, J=7.6 Hz, 3H). MS (ESI⁺): m/z 306 [M+H]⁺.

### Example 111

### 5-[2-(3-Phenylpropoxy)ethyl]-6H-1,3,4-thiadiazin-2-amine

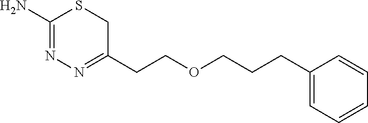 [SMILES: NC1=NN=C(CCOCCCc2ccccc2)CS1]

### a) 4-(3-Phenylpropoxy)butan-2-one

To a stirred solution of methyl vinyl ketone (581 μL, 7.13 mmol) and 3-phenyl-1-propanol (971 μL, 7.13 mmol) was added concentrated sulfuric acid (125 μL) and water (125 μL) at 0° C. After addition, the ice-water bath was removed and the reaction mixture was allowed to reach room temperature and was stirred overnight. The reaction mixture was diluted with ethyl acetate (30 mL) and washed with saturated aqueous NaHCO₃ (30 mL), brine (15 mL), dried over sodium sulfate, filtered and concentrated at reduced pressure. The residue was triturated with heptane and the combined heptane phases were concentrated. The crude material was purified by column chromatography (heptane/ethyl acetate 95:5 to 90:10) to yield 517 mg (35%) of the title compound. MS (ESI⁺): m/z 207 [M+H]⁺.

### b) 1-Bromo-4-(3-phenylpropoxy)butan-2-one

To a solution of 4-(3-phenylpropoxy)butan-2-one (0.517 g, 2.51 mmol) in dichloromethane (20 mL) and methanol (10 mL) was added tetra-n-butylammonium tribromide (1.33 g, 2.76 mmol). The flask was flushed with nitrogen and the reaction was stirred at room temperature overnight. To the reaction mixture was added 2 drops of water and the mixture concentrated at reduced pressure. The crude was purified by column chromatography (heptane/ethyl acetate 95:5 to 90:10) to give 156 mg (22%) of the product. MS (ESI⁺): m/z 285, 287 [M+H]⁺.

### c) 5-[2-(3-Phenylpropoxy)ethyl]-6H-1,3,4-thiadiazin-2-amine

To a solution of 1-bromo-4-(3-phenylpropoxy)butan-2-one (100 mg, 0.35 mmol) in ethanol (600 μL) was added thiosemicarbazide (32 mg, 0.35 mmol) at room temperature The vial was flushed with nitrogen and the reaction was left to stir overnight. The mixture was concentrated at reduced pressure and the crude material was purified by column chromatography (1 to 15% methanol in dichloromethane) to afford 5.2 mg (5%) of the title compound.

### Example 112

### 5-[2-(4-Methoxybutoxy)ethyl]-6H-1,3,4-thiadiazin-2-amine

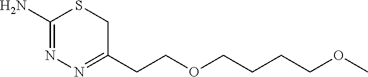 [SMILES: COCCCCOCCC1=NN=C(N)SC1]

### a) 4-(4-Methoxybutoxy)butan-2-one

To a stirred solution of methyl vinyl ketone (581 μL, 7.13 mmol) and 4-methoxybutanol (743 mg, 7.13 mmol) was added concentrated sulfuric acid (125 μL) dropwise and water (125 μL) at 0° C. After addition, the ice-water bath was removed and the reaction mixture was allowed to attain room temperature and was stirred for 4 h. The reaction mixture was diluted with ethyl acetate (40 mL) and washed with saturated aqueous NaHCO₃ (40 mL), brine (20 mL), dried over sodium sulfate, filtered and concentrated at reduced pressure. The crude product was purified by column chromatography (10 to 30% ethyl acetate in heptane) to give 587 mg (47%) of the title compound. MS (ESI⁺): m/z 175 [M+H]⁺.

### b) 1-Bromo-4-(4-methoxybutoxy)butan-2-one

To a solution of 4-(4-methoxybutoxy)butan-2-one (587 mg, 3.37 mmol) in dichloromethane (20 mL) and methanol (10 mL) was added tetra-n-butylammonium tribromide (1.79 g, 3.71 mmol). The flask was flushed with nitrogen and the reaction was stirred at room temperature overnight. To the reaction mixture was added a drop of water and the mixture was concentrated at reduced pressure. The crude was purified by column chromatography (0 to 32% ethyl acetate in heptane) to yield 196 mg (23%) of the product. ¹H NMR (400 MHz, CDCl3 ) δ 3.95 (s, 2H), 3.70 (t, J=6.1 Hz, 2H), 3.46-3.42 (m, 2H), 3.40-3.35 (m, 2H), 3.32 (s, 3H), 2.87 (t, J=6.1 Hz, 2H), 1.62-1.58 (m, 4H). MS (ESI⁺): m/z 253, 255 [M+H]⁺.

### c) 5-[2-(4-Methoxybutoxy)ethyl]-6H-1,3,4-thiadiazin-2-amine

To a solution of 1-bromo-4-(4-methoxybutoxy)butan-2-one (100 mg, 0.40 mmol) in ethanol was added thiosemicarbazide (36.0 mg, 0.40 mmol) at room temperature. The vial was flushed with nitrogen and the reaction was left to stir overnight. The solvent was evaporated and the residue was purified with column chromatography (0 to 20% methanol in ethyl acetate). The material was then recrystallized from methanol/ethyl acetate to afford 24 mg (25%) of the title compound. ¹H NMR (400 MHz, DMSO-d₆) δ 3.69 (s, 2H), 3.63 (t, J=6.2 Hz, 2H), 3.40-3.36 (m, 2H), 3.30-3.26 (m, 2H), 3.20 (s, 3H), 2.73 (t, J=6.2 Hz, 2H), 1.51-1.46 (m, 4H). MS (ESI⁺): m/z 286 [M+H]⁺.

### Example 113

### 5-[4-[3-(2,4-Difluorophenyl)propoxy]butyl]-6H-1,3,4-thiadiazin-2-amine

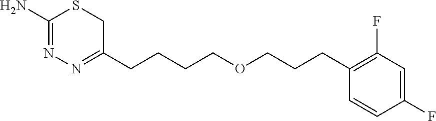 [SMILES: NC1=NN=C(CCCCOCCCc2ccc(F)cc2F)CS1]

### a) 3-(2,4-Difluorophenyl)propan-1-ol

The title compound was prepared according to a modified literature procedure. [Nuclear Medicine and Biology 2010, 37(5), 605-614]2-(2,4-Difluorophenyl)propanic acid (1.36 g, 7.32 mmol) was dissolved in 30 ml of dry tetrahydrofuran and was cooled to 0° C. Then lithium aluminium hydride (417 mg, 11.0 mmol) was added and the reaction was stirred at 0° C. for 1 h. The reaction was allowed to reach room temperature and was stirred overnight. The reaction was quenched with 2 ml of saturated aqueous NH₄Cl and the mixture concentrated to dryness. The residue was taken up in ethyl acetate and the organic phase was washed with water (2×25 mL) then brine (1×25 mL). The organic layer was then separated and dried over magnesium sulfate, filtered and concentrated to give 1224 mg (97%) of the product, which was used in the next step without further purification. b) 5-[3-(2,4-Difluorophenyl)propoxy]pentanoic acid

The title compound was prepared according to a modified literature procedure [J. Med. Chem. 2013, 56(10), 3852-3865]. Sodium hydride (569 mg, 14.2 mmol) was mixed with dimethylformamide (20 mL) and stirred at room temperature for 5 min. Then 3-(2,4-difluorophenyl)propan-1-ol (1.22 g, 7.11 mmol) was added and the temperature increase to 60° C. and stirred for 15 min. Then 5-chloropentanoic acid (0.83 mL, 7.11 mmol) was added at 60° C. and the reaction was stirred for another 3 h. The reaction was concentrated to dryness and the residue was suspended in diethyl ether and washed with water (3×30 mL). The aqueous solution was acidified with aqueous 2 M HCl to pH 3 and then extracted with ethyl acetate (4×30 mL). The combined organic phases were washed with water and a saturated brine solution. The organic phase was dried over sodium sulfate, filtered and concentrated to dryness. The crude was then purified by flash column chromatography (heptane/ethyl acetate 85:15) to give 386 mg (20%) of the product. MS (ESI⁺): m/z 273 [M+H]⁺.

### c) 1-Bromo-6-[3-(2,4-difluorophenyl)propoxy]hexan-2-one

5-[3-(2,4-Difluorophenyl)propoxy]pentanoic acid (665 mg, 2.57 mmol) was dissolved in toluene (25 mL) and oxalyl chloride (0.33 mL, 3.86 mmol) was added dropwise at 0° C. The reaction was stirred at 0° C. for 1 h. The solvent was removed carefully under vacuo to afford 231 mg of the crude product, which was used directly in the next step without further purification.

To a solution of 5-[3-(2,4-difluorophenyl)propoxy]pentanoyl chloride (406 mg, 1.40 mmol) in THF (10 mL) was added trimethylsilyldiazomethane (2 M in hexanes, 1.75 mL, 3.49 mmol) dropwise at 0° C. The reaction was allowed to stir at 0° C. for 1 h. The reaction was then allowed to reach room temperature and stirred for another 1 h. The solvent was removed by a flow of nitrogen and the crude intermediate was taken to the next step.

The crude intermediate was dissolved in DCM (8 mL) and cooled to 0° C. Then HBr (47% HBr/acetic acid)/EtOAc 1:1 (8.0 mL) was added dropwise and the reaction stirred for 1 h at 0° C. The mixture was diluted with water (30 mL) and the mixture extracted with ethyl acetate (3×30 mL). The combined organic layers were washed with water (20 mL) and brine (2×20 mL), dried over sodium sulfate, filtered and evaporated. The crude was taken to the next step without further purification.

### d) 5-[4-[3-(2,4-Difluorophenyl)propoxy]butyl]-6H-1,3,4-thiadiazin-2-amine

Thiosemicarbazide (66.3 mg, 0.730 mmol) and 1-bromo-6-[3-(2,4-difluorophenyl)propoxy]hexan-2-one (231 mg, 0.66 mmol) were mixed in ethanol (3 mL) and the reaction was stirred at room temperature overnight. The reaction was concentrated to dryness and the residue was taken up in ethyl acetate. The organic phase was washed with water and a saturated brine solution. The organic layer was dried over sodium sulfate, filtered and concentrated to dryness. The crude was then purified by flash column chromatography (100% ethyl acetate then 10% methanol in ethyl acetate) to give 21.8 mg (10%) of the product.

### Example 114

### 5-[4-(2-Phenylethoxy)butyl]-6H-1,3,4-thiadiazin-2-amine

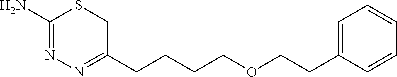 [SMILES: NC1=NN=C(CCCCOCCc2ccccc2)CS1]

### a) 6-(2-Phenylethoxy)hexan-2-one

Sodium hydride (500 mg, 12.5 mmol) was mixed with 20 mL of N,N-dimethylformamide and stirred for 5 min. Then 2-phenylethanol (1.2 mL, 10 mmol) was added and the reaction was stirred at room temperature for 30 min. The reaction was then heated to 60° C. and 6-iodohexan-2-one (1.45 mL, 10 mmol) was added. The reaction was stirred at 60° C. for another 3 h. The reaction was concentrated to dryness and the residue was suspended in water and extracted with ethyl acetate (4×30 mL). The combined organic phases were washed with water (2×30 mL) and saturated brine solution (1×20 mL). The organic layer was dried over sodium sulfate, filtered and concentrated to dryness. The crude was then purified by flash column chromatography (heptane/Ethyl acetate 85:15) to afford 84 mg (4%) of the product.

### b) 1-Bromo-6-(2-phenylethoxy)hexan-2-one

6-(2-Phenylethoxy)hexan-2-one (84 mg, 0.38 mmol) was dissolved in methanol (4 mL) and cooled to 0° C. Then bromine (0.02 mL, 0.40 mmol) was added dropwise at 0° C. during 1 h. The reaction was then allowed to reach room temperature and was stirred overnight. The reaction was concentrated to dryness and the residue was taken up in ethyl acetate (25 mL) and the organic phase was washed with water (2×10 mL) and saturated brine solution (1×10 mL). The organic layer was dried over magnesium sulfate, filtered and concentrated to dryness to give 112 mg of the crude product, which was used in the next step without further purification.

### c) 5-[4-(2-Phenylethoxy)butyl]-6H-1,3,4-thiadiazin-2-amine

Thiosemicarbazide (37.5 mg, 0.410 mmol) and 1-bromo-6-(2-phenylethoxy)hexan-2-one (112 mg, 0.370 mmol) were mixed in ethanol (3 mL) and the reaction was stirred at room temperature overnight. The reaction was concentrated to dryness and the residue was taken up in ethyl acetate (60 mL). The organic phase was washed with water then a saturated brine solution. The organic layer was dried over sodium sulfate, filtered and concentrated to dryness. The crude was then purified by flash column chromatography (100% ethyl acetate then 10% methanol in ethyl acetate) to afford 26 mg (24%) of the product.

### Example 115

### 4-(2-Amino-6H-1,3,4-thiadiazin-5-yl)butyl acetate

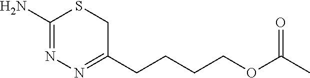

### a) Hex-5-ynyl acetate

The title compound was prepared according to a literature procedure. [Org. Lett. 2008, 10(17), 3793-3796] Hex-5-yn-1-ol (1.00 mL, 9.07 mmol, 1 equiv.) was dissolved in dichloromethane (5 mL) and the solution was put under an inert atmosphere. Pyridine (857 μL, 10.6 mmol, 1.2 equiv.) and acetyl acetate (1.00 mL, 10.6 mmol, 1.2 equiv.) were added and the reaction mixture was left to stir overnight. More dichloromethane was thereafter added and the organic phase was washed two times with water and dried over sodium sulfate. The solvent was removed at reduced pressure and the title compound was purified by column chromatography (heptane/ethyl acetate 4:1) and isolated in 960 mg (76%) yield.

### b) 6-Bromohex-5-ynyl acetate

The title compound was prepared according to a modified literature procedure. [Org. Lett. 2013, 78(18), 9190-9195] Hex-5-ynyl acetate (1.04 mL, 6.69 mmol, 1 equiv.) was dissolved in acetone (12 mL). N-Bromosuccinimide (1.31 g, 7.36 mmol, 1.1 equiv.) was added, followed by silver nitrate (114 mg, 0.670 mmol, 10 mol %) and the reaction mixture was stirred overnight at room temperature protected from light. The solid was thereafter filtered off and the solvent was removed at reduced pressure. The crude mixture was partially dissolved in heptane/ethyl acetate 4:1, the solid was filtered off and the solvent removed under reduced pressure. The title compound was purified by column chromatography (heptane/ethyl acetate, 4:1) and was isolated in 1.30 g (89%) yield.

### c) (6-Bromo-5-oxo-hexyl) acetate

The title compound was prepared according to a modified literature procedure. [Org. Lett. 2013, 78(18), 9190-9195] 6-Bromohex-5-ynyl acetate (635 mg, 2.90 mmol, 1 equiv.) was dissolved in 1,2-dichloroethane (25 mL) and the reaction was put under an inert atmosphere. 2-Dicyclohexylphosphino-2′,4′,6′-triisopropylbiphenyl gold(I) bis(trifluoromethanesulfonyl)imide (XPhosAuNTf₂, 99 mg, 0.10 mmol, 3.6 mol %) was added followed by water (0.16 mL, 8.7 mmol, 3 equiv.) and the reaction mixture was left to stir at room temperature overnight. The solvent was thereafter removed at reduced pressure and the title compound was purified by column chromatography (heptane/ethyl acetate 4:1) to afford 671 mg (98%) of the title compound. MS (ESI⁺): m/z 254, 256 [M+18]⁺.

### d) 4-(2-Amino-6H-1,3,4-thiadiazin-5-yl)butyl acetate

(6-Bromo-5-oxo-hexyl) acetate (200 mg, 0.840 mmol, 1 equiv.) was dissolved in ethanol (3 mL) containing HBr (48 wt. % in water, 105 μL, 0.930 mmol, 1.1 equiv.) and thiosemicarbazide (85 mg 0.93 mmol, 1.1 equiv.) was added. The reaction was stirred at room temperature overnight. After completion of the reaction the solvent was removed at reduced pressure and the products purified by column chromatography (ethyl acetate to ethyl acetate/methanol 9:1) The product was washed repeatedly with ethyl acetate and dried to give 64 mg (33%) of the title compound.

### Example 116

### Ethyl 3-[3-[4-(2-amino-6H-1,3,4-thiadiazin-5-yl)butoxymethyl]phenyl]propanoate

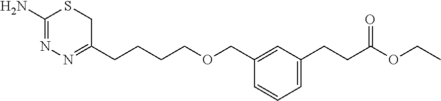 [SMILES: CCOC(=O)CCc1cccc(COCCCCC2=NN=C(N)SC2)c1]

### a) 6-[(3-Bromophenyl)methoxy]hexan-2-one

The title compound was prepared as described in Example 73b.

### b) Ethyl 3-[3-(5-oxohexoxymethyl)phenyl]propanoate

The title compound was prepared according to a modified literature procedure. [J. Org. Chem. 2009, 74(10), 3626-3631] To palladium(II) acetate (35 mg, 0.16 mmol, 10 mol %), 2-dicyclohexylphosphino-2′,6′-diisopropoxybiphenyl (RuPhos, 147 mg, 0.316 mmol, 20 mol %), potassium (3-ethoxy-3-oxopropyl)trifluoroborate (657 mg, 3.16 mmol, 2 equiv.) and K₂CO₃ (654 mg, 4.73 mmol, 3 equiv.) was added 6-[(3-bromophenyl)methoxy]hexan-2-one (450 mg, 1.58 mmol, 1 equiv.) in toluene (3.0 mL) followed by water (0.30 mL) under an inert atmosphere. The reaction mixture was heated to 80° C. and was stirred at that temperature overnight. More water and ethyl acetate were added and the two phases were filtered through a pad of Celite. The phases were separated and the product was extracted two additional times with ethyl acetate. The combined organic phases were washed with brine, dried over sodium sulfate and the solvent was removed under reduced pressure. Because an insufficient conversion of the aryl bromide was achieved, the reaction was repeated. The crude mixture was redissolved in toluene (3.0 mL) and was added to the reaction vessel containing palladium(II) acetate (35 mg, 0.16 mmol), 2-dicyclohexylphosphino-2′,6′-diisopropoxybiphenyl (RuPhos, 145 mg, 0.311 mmol), potassium (3-ethoxy-3-oxopropyl)trifluoroborate (108 mg, 0.519 mmol) and K₂CO₃ (127 mg, 0.919 mmol) under an inert atmosphere. Water (0.30 mL) was thereafter added, the reaction mixture was heated to 80° C. and was stirred at that temperature overnight. More water and ethyl acetate were added and the two phases were filtered through a pad of Celite. The phases were separated and the product was extracted two additional times with ethyl acetate. The combined organic phases were washed with brine, dried over sodium sulfate and the solvent was removed under reduced pressure. The title compound was purified by column chromatography (heptane/ethyl acetate 4:1) to give 153 mg (32%) of the title compound. MS (ESI⁺): m/z 307 [M+H]⁺.

### c) Ethyl 3-[3-[(6-bromo-5-oxo-hexoxy)methyl]phenyl]propanoate

Ethyl 3-[3-(5-oxohexoxymethyl)phenyl]propanoate (70 mg, 0.0.23 mmol, 1 equiv.) was dissolved in dichloromethane (3.0 mL) and methanol (1.5 mL). Tetra-n-butylammonium tribromide (121 mg, 1.1 equiv.) was thereafter added and the reaction was stirred at room temperature overnight. The reaction was quenched with 1 drop of water and the solvent was removed at reduced pressure. The title compound was purified by column chromatography (heptane/ethyl acetate 9:1) and was isolated as a 2:1 mixture of ethyl 3-[3-[(6-bromo-5-oxo-hexoxy)methyl]phenyl]propanoate (A) and methyl 3-[3-[(6-bromo-5-oxo-hexoxy)methyl]phenyl]propanoate (B) in 37 mg yield. Compound A: MS (ESI⁺): m/z 402, 404 [M+18]⁺; Compound B: MS (ESI⁺): m/z 388, 390 [M+18]⁺.

### d) Ethyl 3-[3-[4-(2-amino-6H-1,3,4-thiadiazin-5-yl)butoxymethyl]phenyl]propanoate

A 2:1 mixture of ethyl 3-[3-[(6-bromo-5-oxo-hexoxy)methyl]phenyl]propanoate and methyl 3-[3-[(6-bromo-5-oxo-hexoxy)methyl]phenyl]propanoate (25 mg, 0.060 mmol, 1 equiv.) was dissolved in ethanol (1.0 mL) containing HBr (48 wt. % in water, 7.3 μL, 0.060 mmol, 1 equiv.) and thiosemicarbazide (5.9 mg, 0.060 mmol, 1 equiv.) was thereafter added. The reaction was stirred at room temperature and was followed by LC/MS. After 1.5 h, the solvent was removed at reduced pressure. The title compound was purified by column chromatography (ethyl acetate to ethyl acetate/methanol 9:1) and was isolated as a 4:1 mixture of ethyl 3-[3-[4-(2-amino-6H-1,3,4-thiadiazin-5-yl)butoxymethyl]phenyl]propanoate (A) and methyl 3-[3-[4-(2-amino-6H-1,3,4-thiadiazin-5-yl)butoxymethyl]phenyl]propanoate (B) in 13 mg yield.

### Example 117

### 2-[4-(2-Amino-6H-1,3,4-thiadiazin-5-yl)butoxymethyl]benzonitrile

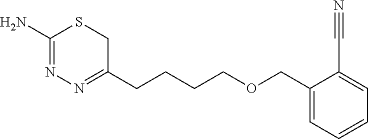 [SMILES: N#Cc1ccccc1COCCCCC1=NN=C(N)SC1]

### a) 2-(5-Oxohexoxymethyl)benzonitrile

Potassium hydroxide (125 mg, 2.23 mmol) was added to a solution of 2-(hydroxymethyl)benzonitrile (297 mg, 2.23 mmol) in toluene (1.0 mL). The mixture was stirred at room temperature for 30 min before 6-iodohexan-2-one (756 mg, 3.34 mmol) was added. The reaction was heated to 60° C. for 13 h. The residue was washed with a dichloromethane/methanol (14:1) mixture in order to remove solid. Then the solvent was removed under reduced pressure to give 244 mg (47%) of the title compound. MS (ESI⁺): m/z 232 [M+H]⁺.

### b) 2-[(6-Bromo-5-oxo-hexoxy)methyl]benzonitrile

To a solution of 2-(5-oxohexoxymethyl)benzonitrile (170 mg, 0.735 mmol) in dichloromethane (4.0 mL) and methanol (2.0 mL) was added tetra-n-butylammonium tribromide (390 mg, 0.806 mmol) and the reaction mixture was stirred overnight at room temperature. The mixture was extracted with ethyl acetate (3×30 mL) and washed with water (5×20 mL). The organic layers were combined, washed with brine, dried over magnesium sulfate and concentrated under reduced pressure. The product was purified by flash chromatography on silica gel (heptane/ethyl acetate 2:3) to give 110 mg (41%) of the title compound. MS (ESI⁺): m/z 327, 329 [M+H+OH]⁻.

### c) 2-[4-(2-Amino-6H-1,3,4-thiadiazin-5-yl)butoxymethyl]benzonitrile

Thiosemicarbazide (95.3 mg, 1.05 mmol) was added to a solution of 4-[(6-bromo-5-oxo-hexoxy)methyl]benzonitrile (295 mg, 0.950 mmol) and HBr (48% in water, 0.12 mL, 1.05 mmol) in ethanol (4.0 mL). The solution mixture was stirred for 30 min at room temperature. The solvent was removed under reduced pressure. The product was purified by flash chromatography on silica (dichloromethane/methanol 95:5) to give 32 mg (35%) of the title compound.

Analysis: ¹H-NMR Data

### 5-(2-Methoxy-ethyl)-6H-[1,3,4]thiadiazin-2-ylamine

¹H-NMR (400 MHz, DMSO-d₆, δ): 10.50-9.50 (broad, 2H), 3.61-3.57 (m, 4H), 3.26 (s, 3H), 2.72 (t, 2H,). Mass (APCI, +ve scan): 174 (100%; M+H)

### 5-m-tolyl-6H-[1,3,4]thiadiazin-2-yl amine hydrobromide

¹H-NMR (400 MHz, DMSO-d₆, δ): 13.50-13.0 (br, 1H), 10.0-9.40 (br, 2H), 7.71-7.67 (m, 2H), 7.45-7.38 (m, 2H), 4.26 (s, 2H), 2.38 (s, 3H). Mass (APCI, +ve scan): 206 (100%; M+H)

### 5-(3-chloro-phenyl)-6H-[1,3,4]thiadiazin-2-yl amine hydrobromide

¹H-NMR (400 MHz, DMSO-d₆, δ): 13.40 (bs, 1H), 10.11 (bs, 1H), 9.34 (bs, 1H), 7.93 (s, 1H), 7.86 (d, 1H), 7.65 (d, 1H, J=7.9 Hz), 7.60-7.56 (m, 1H), 4.29 (s, 2H). Mass (APCI, +ve scan): 226 (100%; M+H)

### 5-(3-methoxy-phenyl)-6H-[1,3,4]thiadiazin-2-ylamine hydrobromide

¹H-NMR (400 MHz, DMSO-d₆, δ): 10.2-9.5 (br, 2H), 7.47-7.42 (m, 3H), 7.17-7.12 (m, 1H), 4.27 (s, 2H), 3.81 (3H, s). Mass (APCI, +ve scan): 222 (100%; M+H)

### (2-Amino-6H-[1,3,4]thiadiazin-5-yl)-acetic acid benzyl ester

¹H NMR (400 MHz, CDCl3, δ): 10.60 (s, 1H), 7.35 (m, ˜5H, merged with solvent), 5.11 (s, 2H), 4.45 (s, 1H), 4.18 (broad s, 2H), 3.40 (s, 2H). Mass (APCI, +ve scan): 264.2 (100%, M+1)

### (2-amino-6H-[1,3,4]thiadiazin-5-yl)-acetic acid ethyl ester hydrochloride

¹H NMR (400 MHz, DMSO-d₆, δ): 13.2 (broad), 9.9 (broad), 4.91 (s, 2H), 3.75 (S, 2H), 2.40 (q, 2H), 1.05 (t, 3H). Mass (APCI, +ve scan): 202 (100%; M+H).

### 5-(2-isopropoxy-ethyl)-6H-[1,3,4]thiadiazin-2 ylamine hydrobromide

¹H NMR (400 MHz, DMSO-d₆, δ): 12.88 (bs, 1H), 9.69 (bs, 1H), 9.11 (bs, 1H), 3.73 (s, 2H), 3.63 (t, 2H), 3.58-3.50 (m, 1H), 2.71 (t, 2H), 1.06 (d, 6H). Mass (APCI, +ve scan): 202 (100%; M+H)

### 5-(2-butoxy-ethyl)-6H-[1,3,4]thiadiazin-2-ylamine hydrobromide

¹H NMR (400 MHz, DMSO-d₆, δ): 12.91 (bs, 1H), 9.34 (bs, 2H), 3.72 (s, 2H), 3.63 (t, 2H), 3.37 (t, 2H), 2.74 (t, 2H), 1.47-1.43 (m, 2H), 1.30-1.25 (m, 2H), 0.86 (t, 3H). Mass (APCI, +ve scan): 216 (100%; M+H)

### 5-(2-cyclohexyloxy-ethyl)-6H-[1,3,4]thiadiazin-2-ylamine. Hydrobromide

¹H NMR (400 MHz, DMSO-d₆, δ): 12.90 (bs, 1H), 9.59 (bs, 1H), 9.15 (bs, 1H), 3.72 (s, 2H), 3.66 (t, 2H), 3.28-3.25 (m, 1H), 2.71 (t, 2H), 1.80-1.78 (m, 2H), 1.64-1.62 (m, 2H), 1.23-1.19 (m, 6H). Mass (APCI, +ve scan): 242 (100%; M+H).

### 5-(2-benzyloxy-ethyl)-6H-[1,3,4]thiadiazin-2-ylamine. hydrobromide

¹H NMR (400 MHz, DMSO-d₆, δ): 12.92 (bs, 1H), 9.56 (broad, 2H), 7.36-7.27 (m, 5H), 3.74 (s, 2H), 3.72 (t, 2H, J=6.3 Hz), 2.80 (t, 2H, J=6.2 Hz). Mass (APCI, +ve scan): 250 (100%; M+H).

### (2-amino-6H-[1,3,4]thiadiazin-5-yl) acetic acid butyl ester

¹H NMR (400 MHz, CDCl3 , δ): 10.65 (broad s, 1H), 4.39 (s, 1H), 4.15 (t, 1H), 4.04 (t, 1H), 3.61 (s, 1H), 3.42 (s, 1H), 3.30 (s, 0.5H), 1.62 (m, 2H), 1.38 (m, 2H), 0.95 (t, 3H). Mass (APCI, +ve scan): 230.2 (100%, M+1).

### 5-(4-propyl-phenyl)-6H-[1,3,4]thiadiazin-2-ylamine. hydrobromide

¹H NMR (400 MHz, DMSO-d₆, δ): 13.28 (bs, 1H), 9.84-9.0 (broad, 2H), 7.81 (d, 2H, J=8.2 Hz), 7.36 (d, 2H, J=8.2 Hz), 4.26 (s, 2H), 2.62 (t, 2H, J=7.4 Hz), 1.65-1.56 (m, 2H), 0.89 (t, 3H, J=7.3 Hz). Mass (APCI, +ve scan): 234 (100%; M+H)

### 5-(4-cyclohexyl-phenyl)-6H-[1,3,4]thiadiazine-2-ylamine.hydrobromide

¹H NMR (400 MHz, DMSO-d₆, δ): 13.26 (bs, 1H), 9.92 (bs, 1H), 9.23 (bs, 1H), 7.81 (d, 2H, J=8.3 Hz), 7.39 (d, 2H, J=Hz), 4.25 (s, 2H), 2.60-2.54 (m, 1H), 1.80-1.69 (m, 5H), 1.47-1.23 (m, 5H).). Mass (APCI, +ve scan): 274 (100%; M+H).

### 6-Butoxy-5,6,7,8-tetrahydro-4aH-benzo[1,3,4]thiadiazin-3-ylamine

¹H NMR (400 MHz, D₂O, δ): 4.20 (t,1H); 3.90(m, 1H); 3.67(t, 2H), 2.85(t, 1H), 2.67(m, 2H); 2.34 (d, 1H); 1.92(q, 1H), 1.77(m, 1H), 1.59(m, 2H); 1.43(m, 2H); 0.98(t, 3H). Mass (APCI, +ve scan): m/z 242 (100%, M+H)

### 5-(4-phenyl-butyl)-6H-[1,3,4]thiadiazin-3-ylamine hydrobromide

¹H NMR (400 MHz, DMSO-d₆): 13.10 (broad, 1H), 9.53 (broad, 2H), 7.29 (m, 2H), 7.19-7.15 (m, 3H), 3.70 (s, 2H), 2.59 (t, 2H, J=6.4 Hz), 2.53 (t, 2H, J=6.3 Hz), 1.60-1.59 (m, 4H). Mass (APCI, +ve scan): 248 (100%; M+H).

### 5-(5-phenoxy-pentyl-6H-[1,3,4]thiadiazine-2-ylamine. hydrobromide

¹H NMR (400 MHz, DMSO-d6): 13.13 (broad, 1H), 9.61 (broad, 2H), 7.26 (t, 2H, J=7.6 Hz), 6.90 (d, 3H, J=8.3 Hz), 3.94 (t, 2H, J=6.2 Hz), 3.71 (s, 2H), 2.53 (t, 2H, J=7.0 Hz), 1.75-1.71 (m, 2H), 1.65-1.60 (m, 2H), 1.48-1.45 (m, 2H). Mass (APCI, +ve scan): 278 (100%; M+H).

### 10,10a-dihydro-9H-1-thia-3,4-diaza-phenanthren-2-ylamine hydrobromide

¹H NMR (400 MHz, DMSO-d₆): 8.01 (d, 1H, J=7.8 Hz), 7.50-7.46 (m, 1H), 7.38-7.33 (m, 2H), 4.37-4.32 (m, 1H), 2.96-2.83 (m, 2H), 2.50-2.45 (m, 1H), 1.88-1.81 (m, 1H). Mass (APCI, +ve scan): 218 (100%; M+H)

### (2-Amino-6H-[1,3,4]thiadiazin-5-yl)-acetic acid isopropyl ester

¹H NMR (400 MHz, CDCl₃ ): δ 10.70 (broad s, 1H), 5.16-4.98 (m, 1H), 4.45 (broad s, 1H), 4.35 (s, 1H), 3.59 (s, 0.57H isomer), 3.40 (s, 1H), 3.25 (s, 0.55H, isomer), 1.23 (d, 6H). Mass (APCI, +ve scan): m/z 216.0 (100%, M+H).

### 5-heptyl-4H-[1,3,4]thiadiazin-2-ylamine

¹H NMR (400 MHz, DMSO-d₆): 8.22 (bs, 1H), 6.17 (s, 1H), 4.67 (bs, 2H), 2.37 (t, 2H, J=7.4 Hz), 1.53 (t, 2H, J=7.0 Hz), 1.24 (m, 10 H), 0.85 (t, 3H, J=6.2 Hz). Mass (APCI, +ve scan): 214 (100%; M+H)

### 4-(2-amino-4H-[1,3,4]thiadiazin-5-yl)-benzoic acid ethyl ester

¹H NMR (400 MHz, DMSO-d₆): 8.65 (bs, 1H), 7.95-7.91 (m, 4H), 7.31 (s, 1H), 4.89 (bs, 1H), 4.30 (q, 2H), 1.32 (t, 3H, J=7.0 Hz). Mass (APCI, +ve scan): 264 (100%; M+H)

### 5-(4-phenyl-butyl)-6H-[1,3,4]thiadiazin-2-ylamine. hydrobromide

¹H NMR (400 MHz, DMSO-d₆): 13.80-13.0 (broad, 1H), 9.90-9.50 (broad, 2H), 7.50 (s, 2H), 7.20 (s, 1H), 4.21 (s, 2H), 2.33 (s, 6H). Mass (APCI, +ve scan): 220 (100%; M+H).

### 5-(3-methoxy-propyl)-6H-[1,3,4]thiadiazin-2-ylamine. hydrobromide

¹H NMR (400 MHz, DMSO-d₆): 12.90 (bs, 1H), 9.83 (bs, 1H), 9.07 (bs, 1H), 3.74 (s, 2H), 3.34 (t, 2H, J=6.3 Hz), 3.21 (s, 3H), 2.54 (t, 2H, J=7.3 Hz), 1.83-1.76 (m, 2H). Mass (APCI, +ve scan): 188 (100%; M+H)

### 5-(5-methoxy-pentyl)-6H-[1,3,4]thiadiazin-2-ylamine. hydrobromide

¹H NMR (400 MHz, DMSO-d₆): 6 12.89 (bs, 1H), 9.77 (bs, 1H), 9.04 (bs, 1H), 3.72 (s, 2H), 3.29 (t, 2H, J=6.4 Hz), 3.20 (s, 3H), 2.50 (t, 2H), 1.61-1.54 (m, 2H), 1.52-1.45 (m, 2H), 1.33-1.28 (m, 2H). Mass (APCI, +ve scan): 216 (100%; M+H).

### 3-(2-amino-6H-[1,3,4]thiadiazin-5-yl)-benzoic acid ethyl ester. hydrobromide

¹H NMR (400 MHz, DMSO-d₆): 13.28 (broad, 1H), 10.09 (broad, 1H), 9.36 (broad, 1H), 8.43 (t, 1H, J=1.4 Hz), 8.17-8.12 (m, 2H), 7.71 (t, 1H, J=7.8 Hz), 4.35 (q, 2H), 4.34 (s, 2H), 1.34 (t, 3H, J=7.0 Hz). Mass (APCI, +ve scan): 264 (100%; M+H)

### 5-[3-(butoxy-phenyl)-6H-[1,3,4]thiadiazin-2-ylamine

¹H NMR (400 MHz, DMSO-d₆): 7.43-7.42 (m, 2H), 7.33 (t, 1H, J=8.1 Hz), 6.97 (d, 1H, J=8.3 Hz), 4.01 (t, 2H, J=6.4 Hz), 3.64 (s, 2H), 1.73-1.67 (m, 2H), 1.48-1.42 (m, 2H), 0.94 (t, 3H, J=7.3 Hz). Mass (APCI, +ve scan): 264 (100%; M+H).

### 5-(4a, 8a-dihydro-naphthalen-1-yl)-6H-[1,3,4]thiadiazin-2-ylamine

¹H NMR (400 MHz, DMSO-d₆): 8.60 (bs, 1H), 8.44 (d, 1H, J=9.0 Hz), 7.93 (d, 1H, J=9.0 Hz), 7.88 (d, 1H, J=8.1 Hz), 7.63 (d, 1H, J=6.8 Hz), 7.52-7.47 (m, 3H), 6.86 (s, 1H), 4.88 (bs, 2H). Mass (APCI, +ve scan): 242 (100%; M+H)

### 5-naphthalen-2-yl-6H-[1,3,4]thiadiazin-2-ylamine. hydrobromide

¹H NMR (400 MHz, DMSO-d₆): 13.41 (broad, 1H), 10.06 (broad, 1H), 9.32 (broad, 1H), 8.50 (s, 1H), 8.06-7.99 (m, 4H), 7.67-7.62 (m, 2H), 4.44 (s, 2H). Mass (APCI, +ve scan): 242 (100%; M+H).

### 5-[2-(4-bromo-phenoxy)-ethyl]-6H-[1,3,4]thiadiazin-2-ylamine

¹H NMR (400 MHz, DMSO-d₆): 7.45 (d, 2H, J=8.9 Hz), 6.92 (d, 2H, J=9.0 Hz), 4.26 (t, 2H, J=6.0 Hz), 3.79 (s, 2H), 2.99 (t, 2H, J=5.9 Hz). Mass (APCI, +ve scan): 314.21 (100%; M+H)

### 5-(2-phenoxy-ethyl)-6H-[1,3,4]thiadiazin-2-ylamine. hydrobromide

¹H NMR (400 MHz, DMSO-d₆): 12.99 (bs, 1H), 9.51-9.30 (broad, 2H), 7.31-7.27 (m, 2H), 6.96-6.92 (m, 3H), 4.26 (t, 2H, J=6.0 Hz), 3.80 (s, 2H), 2.99 (t, 2H, J=6.0 Hz). Mass (APCI, +ve scan): 236 (100%; M+H).

### 5-[2-(2-methoxy-ethoxy)-ethyl]-6H-[1,3,4]thiadiazin-2-ylamine.hydrobromide

¹H NMR (400 MHz, DMSO-d₆): 12.85 (broad, 1H), 10.20-9.0 (broad, 2H), 3.74 (s, 2H), 3.68 (t, 2H, J=6.2 Hz), 3.52-3.50 (m, 2H), 3.43-3.40 (m, 2 Hz), 3.22 (s, 3H), 2.74 (t, 2H, J=6.0 Hz). Mass (APCI, +ve scan): 218 (100%; M+H).

### 5-(2-hexyloxy-ethyl)-6H-[1,3,4]thiadiazin-2-ylamine. hydrobromide

¹H NMR (400 MHz, DMSO-d₆): 12.88 (bs, 1H), 9.78 (bs, 1H), 9.08 (bs, 1H), 3.74 (s, 2H), 3.63 (t, 2H, J=6.1 Hz), 3.37 (t, 2H, J=6.5 Hz), 2.74 (t, 2H, J=6.0 Hz), 1.45-1.41 (m, 2H), 1.30-1.20 (m, 6H), 0.85 (t, 3H, J=6.1 Hz). Mass (APCI, +ve scan): 244 (100%; M+H).

### 5-[2-(biphenyl-4-ylmethoxy)-ethyl]-6H-[1,3,4]-thiadiazin-2-ylamine.hydrobromide

¹H NMR (400 MHz, DMSO-d₆): 12.92 (broad, 1H), 9.30-9.11 (broad, 2H), 7.67-7.34 (m, 4H), 7.46 (t, 2H, J=7.3 Hz), 7.41-7.36 (m, 3H), 4.53 (s, 2H), 3.76-3.73 (m, 4H), 2.82 (t, 2H, J=6.2 Hz). Mass (APCI, +ve scan): 326 (100%; M+H)

### 5-[2-(4-methyl-benzyloxy)-ethyl]-6H-[1,3,4]thiadiazin-2-ylamine.hydrobromide

¹H NMR (400 MHz, DMSO-d₆): 12.93 (broad, 1H), 9.60 (broad, 1H), 9.22 (broad, 1H), 3.74 (s, 2H), 3.68 (t, 2H, J=6.2 Hz), 2.78 (t, 2H, J=6.1 Hz), 2.28 (s, 3H). Mass (APCI, +ve scan): 264 (100%; M+H)

### 5-[2-(1-methyl-butoxy)-ethyl]-6H-[1,3,4]thiadiazin-2-ylamine. hydrobromide

¹H NMR (400 MHz, DMSO-d₆): 9.50-9.0 (broad, 2H), 3.73-3.67 (m, 3H), 3.60-3.56 (m, 1H), 3.40-3.36 (m, 1H), 2.70 (t, 2H, J=6.1 Hz), 1.42-1.38 (m, 1H), 1.30-1.23 (m, 3H), 1.04 (d, 3H, J=6.0 Hz), 0.85 (t, 3H, J=7.2 Hz). Mass (APCI, +ve scan): 230 (100%; M+H)

### 4-[2-(2-amino-6H-[1,3,4]thiadiazin-5-yl)-ethoxymethyl]-benzonitrile.hydrobromide

¹H NMR (400 MHz, DMSO-d₆): 9.80-9.10 (broad, 2H), 7.81 (d, 2H, J=8.0 Hz), 7.50 (d, 2H, J=8.1 Hz), 4.59 (s, 2H), 3.77-3.72 (m, 4H), 2.82 (t, 2H, J=6.0 Hz). Mass (APCI, +ve scan): 275 (100%; M+H)

### 5-[2-(4-propoxy-phenyl)-ethyl]-6H-[1,3,4]thiadiazin-2-ylamine. Hydrobromide

¹H NMR (400 MHz, DMSO-d₆): 9.80-8.80 (broad, 2H), 7.14 (d, 2H, J=8.5 Hz), 6.83 (d, 2H, J=8.5 Hz), 3.87 (t, 2H, J=6.5 Hz), 3.60 (s, 2H), 2.84-2.80 (m, 2H), 2.76-2.72 (m, 2H), 1.74-1.65 (m, 2H), 0.96 (t, 3H, J=7.4 Hz). Mass (APCI, +ve scan): 278 (100%; M+H).

### 5-[2-(4-methoxy-benzyloxy)-ethyl]-6H-[1,3,4]thiadiazin-2-ylamine. hydrobromide

¹H NMR (400 MHz, DMSO-d₆): 9.50-9.10 (broad, 1H), 7.23 (d, 2H, J=8.5 Hz), 6.89 (d, 2H, J=8.5 Hz), 4.40 (s, 2H), 4.37 (s, 3H), 3.69-3.65 (m, 4H), 2.77 (t, 2H, J=6.1 Hz). Mass (APCI, +ve scan): 280 (100%; M+H).

### 5-(2-amino-6H-[1,3,4]thiadiazin-5-yl)-pentanoic acid phenylamide.hydrobromide

¹H NMR (400 MHz, DMSO-d₆): 12.88 (bs, 1H), 9.88 (s, 1H), 9.75 (bs, 1H), 9.03 (bs, 1H), 7.58 (d, 2H, J=7.9 Hz), 7.28 (t, 2H, J=7.6 Hz), 7.01 (t, 2H, J=7.3 Hz), 3.73 (s, 2H), 2.60-2.50 (m, 2H), 2.36-2.32 (m, 2H), 1.65-1.58 (m, 4H). Mass (APCI, +ve scan): 291 (100%; M+H).

### 5-(6-phenyl-hexyl)-6H-[1,3,4]-thiadiazin-2-ylamine. hydrobromide

¹H NMR (400 MHz, DMSO-d₆): 12.87 (bs, 1H), 9.73 (bs, 1H), 9.06 (bs, 1H), 7.28-7.25 (m, 2H), 7.18-7.14 (m, 3H), 3.71 (s, 2H), 2.58-2.54 (m, 2H), 1.57-1.54 (m, 4H), 1.33-1.30 (m, 4H), 1.24 (t, 2H, J=3.6 Hz). Mass (APCI, +ve scan): 276 (100%; M+H)

### 5-(6-phenoxy-hexyl)-6H-[1,3,4]thiadiazin-2-ylamine. hydrobromide

¹H NMR (400 MHz, DMSO-d₆): 12.88 (bs, 1H), 9.58-9.0 (broad, 2H), 7.27 (t, 2H, J=7.8 Hz), 6.92-6.89 (m, 3H), 3.94 (t, 2H, J=6.4 Hz), 3.73 (s, 2H), 2.53-2.50 (m, 2H), 1.73-1.67 (m, 2H), 1.63-1.57 (m, 2H), 1.45-1.33 (m, 4H). Mass (APCI, +ve scan): 292 (100%; M+H)

### 5-(3-phenoxy-propyl)-6H-[1,3,4]thiadiazin-2-ylamine. hydrobromide

¹H NMR (400 MHz, DMSO-d₆): 10.50-9.0 (broad, 2H), 7.28 (t, 2H, J=7.8 Hz), 6.93-6.90 (m, 3H), 4.00 (t, 2H, J=6.2 Hz), 3.65 (s, 2H), 2.67 (t, 2H, J=7.3 Hz), 2.06-1.99 (m, 2H). Mass (APCI, +ve scan): 250 (100%; M+H).

### 5-phenoxymethyl-6H-[1,3,4]thiadiazin-2-ylamine. hydrobromide

¹H NMR (400 MHz, DMSO-d₆): 13.30-12.80 (broad, 1H), 10.00-9.30 (broad, 2H), 7.32 (t, 2H, J=7.6 Hz), 7.01-6.97 (m, 3H), 4.97 (s, 2H), 3.85 (s, 2 H). Mass (APCI, +ve scan): 222 (100%; M+H)

### 5-(2-p-tolyloxy-ethyl)-6H-[1,3,4]thiadiazin-2-ylamine. hydrobromide

¹H NMR (400 MHz, DMSO-d₆): 13.10-12.90 (broad, 1H), 9.80-9.60 (broad, 1H), 9.30-9.00 (broad, 1H), 7.08 (d, 2H, J=8.4 Hz), 6.82 (d, 2H, J=8.5 Hz), 4.22 (t, 2H, J=6.1 Hz), 3.79 (s, 2H), 2.97 (t, 2H, J=6.0 Hz), 2.22 (s, 3H). Mass (APCI, +ve scan): 250 (100%; M+H).

### 5-[2-(biphenyl-4-yloxy)-ethyl]-6H-[1,3,4]thiadiazin-2-ylamine. hydrobromide

¹H NMR (400 MHz, DMSO-d₆): 13.0-12.90 (broad, 1H), 9.90-9.0 (broad, 2H), 7.60 (d, 4H, J=8.3 Hz), 7.43 (t, 2H, J=7.5 Hz), 7.31 (t, 1H, J=7.3 Hz), 7.03 (d, 2H, J=8.6 Hz), 4.32 (t, 2H, J=6.0 Hz), 3.82 (s, 2H), 3.02 (t, 2H, J=5.9 Hz). Mass (APCI, +ve scan): 312 (100%; M+H)

### 5-(4-phenoxy-butyl)-6H-[1,3,4]thiadiazin-2-ylamine

¹H NMR (400 MHz, DMSO-d₆): 10.00-8.60 (broad, 1H), 7.27 (t, 2H, J=7.8 Hz), 6.92-6.90 (m, 3H), 3.97 (t, 2H, J=5.6 Hz), 3.64 (s, 2H), 2.57(t, 2H, J=6.6 Hz), 1.75-1.73 (m, 4H). Mass (APCI, +ve scan): 264 (100%; M+H).

### 5-phenoxymethyl-6H-[1,3,4]thiadiazin-2-ylamine

¹H NMR (400 MHz, DMSO-d₆): 10.50-9.0 (broad, 2H), 4.22 (s, 2H), 3.71 (s, 2H), 3.28 (s, 3H). Mass (APCI, +ve scan): 160 (100%; M+H)

### 6-(2-amino-6H-[1,3,4]thiadiazin-5-yl)-hexanoic acid ethyl ester.hydrobromide

¹H NMR (400 MHz, DMSO-d₆): 13.0-12.80 (broad, 1H), 9.90-9.70 (broad, 1H), 8.90-9.20 (broad, 1H), 4.03 (q, 2H), 3.72 (s, 2H), 2.55-2.47 (m, 2H), 2.27 (t, 2H, J=7.4 Hz), 1.60-1.50 (m, 4H), 1.33-1.27 (m, 2H), 1.16 (t, 3H, J=7.1 Hz). Mass (APCI, +ve scan): 258 (100%; M+H).

### 6-(2-amino-6H-[1,3,4]thiadiazin-5-yl)-hexanoic acid. trifluoroacetic acid

¹H NMR (400 MHz, DMSO-d₆): 13.20-12.80 (broad, 1H), 12.10-11.90 (broad, 1H), 9.70-9.20 (broad, 2H), 3.70 (s, 2H), 2.50-2.47 (m, 2H), 2.20 (t, 2H, J=7.3 Hz), 1.60-1.47 (m, 4H), 1.40-1.30 (m, 2H). Mass (APCI, +ve scan): 230 (100%; M+H).

### 5-(4-methoxy-butyl)-6H-[1,3,4]thiadiazin-2-ylamine.hydrobromide

¹H NMR (400 MHz, D₂O): 3.63 (s, 2H), 3.49 (t, 2H, J=6.3 Hz), 3.33 (s, 3H), 2.60 (t, 2H, J=6.9 Hz), 1.69-1.62 (m, 4H). Mass (APCI, +ve scan): 202 (100%; M+H).

### 5-(7-methoxy-heptyl)-6H-[1,3,4]thiadiazin-2-ylamine. hydrobromide

¹H NMR (400 MHz, DMSO-d₆): δ 13.20-12.40 (broad, 1H), 10.00-8.90 (broad, 2H), 3.73 (s, 2H), 3.28 (t, 2H, J=6.4 Hz), 3.20 (s, 3H), 2.50-2.48 (m, 2H), 1.60-1.55 (m, 2H), 1.50-1.44 (m, 2H), 1.35-1.20 (m, 6H). Mass (APCI, +ve scan): 244 (100%; M+H).

### 5-(7-Phenoxy-heptyl)-6H-[1,3,4]thiadiazin-2-ylamine.hydrobromide

1H NMR (400 MHz, DMSO-d₆): δ 10.00-8.50 (broad, 3H), 7.26 (t, 2H, J=7.8 Hz), 6.92-6.89 (m, 3H), 3.93 (t, 2H, J=6.4 Hz), 3.55 (s, 2H), 2.47 (t, 2H, J=7.5 Hz), 1.73-1.66 (m, 2H), 1.58-1.54 (m, 2H), 1.45-1.30 (m, 6H). Mass (APCI, +ve scan): 306 (100%; M+H)

### 5-(6-Methoxy-hexyl)-6H-[1,3,4]thiadiazin-2-ylamine.hydrobromide

1H NMR (400 MHz, DMSO-d₆): δ d 12.83 (bs, 1H), 9.38 (bs, 1H), 3.90 (s, 2H), 3.28 (t, 2H, J=6.5 Hz), 3.20 (s, 3H), 2.49-2.47 (m, 2H), 1.60-1.50 (m, 2H), 1.50-1.40 (m, 2H), 1.35-1.30 (m, 4H). Mass (APCI, +ve scan): 230 (100%; M+H).

### 5-[5-(Methyl-phenyl-amino)-pentyl]-6H-[1,3,4]thiadiazin-2-ylamine.hydrobromide

1H NMR (400 MHz, DMSO-d₆): δ 12.89 (bs, 1H), 9.78 (bs, 1H), 9.03 (bs, 1H), 7.42 (t, 2H, J=7.7 Hz), 7.30-7.27 (m, 2H), 7.20-7.18 (m, 1H), 3.71 (s, 2H), 3.42 (t, 2H, J=7.6 Hz), 3.04 (s, 3H), 2.46-2.40 (m, 2H), 1.54-1.48 (m, 2H), 1.41-1.37 (m, 2H), 1.29-1.27 (m, 2H). Mass (APCI, +ve scan): 291 (100%; M+H).

### 5-(5-Pyridin-2-yl-pentyl)-6H-[1,3,4]thiadiazin-2-ylamine

1H NMR (400 MHz, DMSO-d₆): δ 8.45 (d, 1H, J=4.2 Hz), 7.69-7.64 (m, 1H), 7.23 (d, 1H, J=7.7 Hz), 7.17 (t, 1H, J=5.3 Hz), 6.42 (broad, 2H), 3.09 (s, 2H), 2.71 (t, 2H, J=7.5 Hz), 2.39 (t, 2H, J=7.3 Hz), 1.72-1.64 (m, 2H), 1.62-1.54 (m, 2H), 1.36-1.23 (m, 2H). Mass (APCI, +ve scan): 263 (100%; M+H).

### 4-(5-methoxy-pentyl)-5-methyl-4H-[1,3,4]-thiadiazin-2-ylamine

¹H NMR (400 MHz, DMSO-d₆): δ 8.50 (bs, 1H), 6.18 (s, 1H), 5.20 (broad, 1H), 3.26 (t, 2H, J=6.5 Hz, in D₂O exchange), 3.20 (s, 3H), 2.71 (t, 2H, J=6.6 Hz), 2.06 (s, 3H), 1.51-1.32 (m, 6H). Mass (APCI, +ve scan): 230 (100%; M+H).

### ((E)-5-Hept-1-enyl)-6H-[1,3,4]thiadiazin-2-ylamine.hydrobromide

1H NMR (400 MHz, DMSO-d₆): δ 13.20 (broad, 1H), 9.70-9.40 (broad, 2H), 6.64-6.56 (m, 1H), 6.30 (d, 1H, J=16.9 Hz), 3.98 (s, 2H), 2.29-2.23 (q, 2H), 1.43 (t, 2H, J=7.1 Hz), 1.30-1.14 (m, 4H), 0.87 (t, 3H, J=6.5 Hz). Mass (APCI, +ve scan): 212 (100%; M+H).

### 5-[5-(4-Fluoro-phenoxy)-pentyl]-6H-[1,3,4]thiadiazin-2-ylamine hydrobromide

1H NMR (400 MHz, DMSO-d₆): δ 7.09 (t, 2H, J=8.8 Hz), 6.94-6.90 (m, 2H), 6.80-6.60 (broad, 2H), 3.92 (t, 2H, J=6.3 Hz), 3.11 (s, 2H), 2.42 (t, 2H, J=7.2 Hz), 1.73-1.68 (m, 2H), 1.63-1.57 (m, 2H), 1.47-1.43 (m, 2H). Mass (APCI, +ve scan): 296 (100%; M+H).

### 2-[5-(2-Amino-6H-[1,3,4]thiadiazin-5-yl)-pentyl]-isoindole-1,3-dione.hydrobromide

1H NMR (400 MHz, DMSO-d₆): δ 12.90 (broad, 1H), 9.50-9.20 (broad, 2H), 7.87-7.82 (m, 4H), 3.70 (s, 2H), 3.56 (t, 2H, J=7.0 Hz), 2.47 (t, 2H, J=7.3 Hz, in D20 exchange), 1.63-1.56 (m, 4H), 1.34-1.27 (m, 2H). Mass (APCI, +ve scan): 331 (100%; M+H).

### 5-(4-chloro-phenyl)-4-methyl-4H-[1,3,4]thiadiazin-2-ylamine

1H NMR (400 MHz, DMSO-d₆): δ 8.67 (s, 1H), 7.80 (d, 2H, J=8.4 Hz), 7.41 (d, 2H, J =8.8 Hz), 7.17 (s, 1H), 2.50 (s, 3H). Mass (APCI, +ve scan): 240 (100%; M+H).

### 5-(5-Methoxy-pentyl)-4-methyl-4H-[1,3,4]thiadiazin-2-ylamine

1H NMR (400 MHz, DMSO-d₆): δ 8.60-8.40 (broad, 1H), 6.22 (s, 1H), 5.8-5.0 (broad, 1H), 3.28 (t, 2H, J=6.4 Hz), 3.20 (s, 3H), 2.47 (s, 3H, in D20 exchange) 2.39 (t, 2H, J=7.1 Hz), 1.58-1.45 (m, 4H), 1.32-1.27 (m, 2H). Mass (APCI, +ve scan): 230 (100%; M+H).

### 5-(4-Benzofuran-2-yl-butyl)-6H-[1,3,4]thiadiazin-2-ylamine.hydrobromide

1H NMR (400 MHz, DMSO-d₆): δ 13.10-12.90 (broad, 1H), 9.80-9.30 (broad, 2H), 7.54-7.47 (m, 2H), 7.23-7.16 (m, 2H), 6.60 (s, 1H), 3.70 (s, 2H), 2.80 (t, 2H, J=6.8 Hz), 2.56 (t, 2H, J=7.3 Hz), 1.77-1.70 (m, 4H). Mass (APCI, +ve scan): 288 (100%; M+H).

### 5-(5-Benzyloxy-pentyl)-6H-[1,3,4]thiadiazin-2-ylamine.hydrobromide

1H NMR (400 MHz, DMSO-d₆): δ 13.20-12.80 (broad, 1H), 9.80-8.80 (broad, 2H), 7.36-7.27 (m, 5H), 4.43 (s, 2H), 3.71 (s, 2H), 3.41 (t, 2H, J=6.2 Hz), 2.53-2.50 (m, 2H), 1.61-1.54 (m, 4H), 1.39-1.23 (m, 2H). Mass (APCI, +ve scan): 292 (100%; M+H).

### 5-(4-(benzyloxy)butyl)-6H-1,3,4-thiadiazin-2-amine hydrobromide

1H NMR (400 MHz, DMSO-d₆): δ 7.31 (m, 5H), 4.42 (s, 2H), 3.63 (s, 2 H), 3.41 (t, 2H), 2.52 (m, 2H), 1.58 (m, 4H). Mass (ESI, +ve scan): 278 (100%, M+H).

### 5-(2-(2-Phenoxyethoxy) ethyl)-6H-1,3,4-thiadiazin-2-amine hydrochloride

1H NMR (400 MHz, DMSO-d₆): δ 13.13 (br s, 1H), 9.95 (br s, 1H), 9.20 (br s, 1H), 7.28 (t, 2H), 6.94-6.91 (m, 3H), 4.09-4.06 (m, 2H), 3.77-3.70 (m, 6H), 2.77 (t, 2H). Mass (APCI, +ve scan): m/z 280.1 (100%, (M-HCl)+H

### 5-(5-(Pyridin-2-yloxy) pentyl)-6H-1, 3, 4-thiadiazin-2-amine hydro chloride

1H NMR (400 MHz, CDCl3 ): δ 13.18 (br s, 1H), 9.93-9.28 (br s, 2H), 8.14-8.13 (m, 1H), 7.70-7.66 (m, 1H), 6.96-6.93 (m, 1H), 6.77 (d, 1H), 4.23 (t, 2H), 3.72 (s, 2H), 2.52 (m, 2H), 1.76-1.59 (m, 4H), 1.47-1.39 (m, 2H). Mass (APCI, +ve scan): m/z 279.1 100%, [(M-HCl)+H].

### 5-(4-(4-Ethylphenoxy)butyl)-6H-1,3,4-thiadiazin-2-amine hydrochloride

1H NMR (400 MHz, CD3OD): δ 7.07 (d, 2H), 6.80 (d, 2H), 3.98 (t, 2H), 3.67 (s, 2H), 2.67-2.63 (m, 2H), 2.58-2.53 (m, 2H), 1.85-1.83 (m, 4H), 1.18 (t, 3H). Mass (APCI, +ve scan): m/z 292.1 (100%, [(M-HCl)+H].

### 6-(2-Butoxyethyl)-4H-[1,3,4]thiadiazin-2-ylamine.hydrobromide

1H NMR (400 MHz, DMSO-d₆): δ 13.12 (bs, 1H), 9.94 (bs, 1H), 9.17 (bs, 1H), 7.65 (d, 1H, J=4.2 Hz), 4.14-4.09 (m, 1H), 3.48 (t, 2H, J=5.7 Hz), 3.36 (t, 2H, J=6.4 Hz), 2.17-2.08 (m, 1H), 1.99-1.90 (m, 1H), 1.51-1.44 (m, 2H), 1.36-1.26 (m, 2H), 0.87 (t, 3H, J=7.2 Hz). Mass (APCI, +ve scan): 216 (100%; M+H).

### 5-(4-Benzyloxybutyl)-6H-1,3,4-thiadiazin-2-amine

¹H NMR (400 MHz, DMSO-d₆) δ 7.37-7.25 (m, 5 H), 4.42 (s, 2 H), 3.63 (s, 2 H), 3.41 (t, J=6.1 Hz, 2 H), 2.52 (m, 2 H, partially obscured by the solvent peak), 1.68-1.53 (m, 4 H). MS (ESI⁺): m/z 278 [M+H]⁺.

### 5-[4-[(2,4-Difluorophenyl)methoxy]butyl]-6H-1,3,4-thiadiazin-2-amine

¹H NMR (400 MHz, CD₃OD) δ=7.49-7.41 (m, 1H), 6.99-6.91 (m, 2H), 4.52 (s, 2H), 3.66 (s, 2H), 3.55 (t, J=6.0 Hz, 2H), 2.59 (t, J=7.3 Hz, 2H), 1.80-1.63 (m, 4H); MS (ESI⁺): m/z 314 [M+H]⁺.

### 5-[4-[[4-(Trifluoromethyl)phenyl]methoxy]butyl]-6H-1,3,4-thiadiazin-2-amine

¹H NMR (400 MHz, CD₃OD) δ=7.64 (d, J=8.2 Hz, 2H), 7.53 (d, J=8.0 Hz, 2H), 4.59 (s, 2H), 3.67 (s, 2H), 3.57 (t, J=6.0 Hz, 2H), 2.62 (t, J=7.3 Hz, 2H), 1.84-1.66 (m, 4H); MS (ESI⁺): m/z 346 [M+H]⁺.

### 5-[4-[(3-Chlorophenyl)methoxy]butyl]-6H-1,3,4-thiadiazin-2-amine

¹H NMR (400 MHz, CD₃OD) δ=7.37-7.23 (m, 4H), 4.49 (s, 2H), 3.67 (s, 2H), 3.54 (t, J=6.0 Hz, 2H), 2.61 (t, J=7.3 Hz, 2H), 1.82-1.65 (m, 4H); MS (ESI⁺): m/z 312 [M+H]⁺.

### 5-[4-[(4-Fluoro-3-methyl-phenyl)methoxy]butyl]-6H-1,3,4-thiadiazin-2-amine

¹H NMR (400 MHz, CD₃OD) δ=7.20 (d, J=7.4 Hz, 1H), 7.18-7.12 (m, 1H), 6.98 (t, J=9.0 Hz, 1H), 4.43 (s, 2H), 3.66 (s, 2H), 3.51 (t, J=6.0 Hz, 2H), 2.59 (t, J=7.3 Hz, 2H), 2.26 (s, 3H), 1.81-1.63 (m, 4H); MS (ESI⁺): m/z 310 [M+H]⁺.

### 5-[4-[(3-Bromophenyl)methoxy]butyl]-6H-1,3,4-thiadiazin-2-amine

¹H NMR (400 MHz, CD₃OD) δ=7.20 (d, J=7.4 Hz, 1H), 7.18-7.12 (m, 1H), 6.98 (t, J=9.0 Hz, 1H), 4.43 (s, 2H), 3.66 (s, 2H), 3.51 (t, J=6.0 Hz, 2H), 2.59 (t, J=7.3 Hz, 2H), 2.26 (s, 3H), 1.81-1.63 (m, 4H); ¹³C NMR (101 MHz, CD₃OD) δ=166.4, 158.3, 142.7, 131.6, 131.5, 131.2, 127.4, 123.4, 72.9, 71.0, 36.8, 30.0, 24.8, 23.5; MS (ESI⁺): m/z 356, 358 [M+H]⁺.

### 5-[4-[(4-Bromophenyl)methoxy]butyl]-6H-1,3,4-thiadiazin-2-amine

¹H NMR (400 MHz, CD₃OD) δ=7.49 (dm, J=8.4 Hz, 2H), 7.26 (dm, J=8.4 Hz, 2H), 4.47 (s, 2H), 3.67 (s, 2H), 3.53 (t, J=6.0 Hz, 2H), 2.60 (t, J=7.3 Hz, 2H), 1.82-1.63 (m, 4H); MS (ESI⁺): m/z 356, 358 [M+H]⁻.

### 5-[4-[(2-Bromophenyl)methoxy]butyl]-6H-1,3,4-thiadiazin-2-amine

¹H NMR (400 MHz, DMSO-d₆) δ=7.61 (dd, J=8.0, 1.2 Hz, 1H), 7.47 (dd, J=7.6, 1.6 Hz, 1H), 7.40 (td, J=7.5, 1.2 Hz, 1H), 7.25 (tm, J=7.6 Hz, 1H), 4.49 (s, 2H), 3.72 (s, 2H), 3.52 (t, J=6.0 Hz, 2H), 2.54 (t, J=7.1 Hz, 2H), 1.73-1.55 (m, 4H). MS (ESI⁺): m/z 356, 358 [M+H]⁺.

### 5-[4-[(4-Chloro-2-fluoro-phenyl)methoxy]butyl]-6H-1,3,4-thiadiazin-2-amine

¹H NMR (400 MHz, DMSO-d₆) δ=7.49-7.40 (m, 2H), 7.30 (dd, J=8.2, 2.0 Hz, 1H), 4.48 (s, 2H), 3.70 (s, 2H), 3.47 (t, J=6.0 Hz, 2H), 2.51 (2H, peak obscured by the solvent peak), 1.69-1.52 (m, 4H); MS (ESI⁺): m/z 330 [M+H]⁺.

### 5-[4-[(3,5-Dichlorophenyl)methoxy]butyl]-6H-1,3,4-thiadiazin-2-amine

¹H NMR (400 MHz, DMSO-d₆) δ=7.52 (t, J=2.0 Hz, 1H), 7.36 (dt, J=2.0, 0.5 Hz, 2H), 4.47 (d, J=0.5 Hz, 2H), 3.50-3.41 (m, 4H), 2.49 (2H, peak obscured by the solvent peak), 1.70-1.53 (m, 4H). MS (ESI⁺): m/z 346 [M+H]⁺.

### 5-[4-[(3-Ethylphenyl)methoxy]butyl]-6H-1,3,4-thiadiazin-2-amine

¹H NMR (400 MHz, CD₃OD) δ=7.24 (t, J=7.5 Hz, 1H), 7.19-7.16 (m, 1H), 7.15-7.11 (m, 2H), 4.47 (s, 2H), 3.66 (s, 2H), 3.53 (t, J=6.1 Hz, 2H), 2.68-2.56 (m, 4H), 1.83-1.62 (m, 4H), 1.22 (t, J=7.6 Hz, 3H); MS (ESI⁺): m/z 306 [M+H]⁺.

### 5-[4-[(3-Vinylphenyl)methoxy]butyl]-6H-1,3,4-thiadiazin-2-amine

¹H NMR (400 MHz, DMSO-d₆) δ=7.39 (dm, J=9.0 Hz, 2H), 7.32 (t, J=7.5 Hz, 1H), 7.23 (dm, J=7.6 Hz, 1H), 6.74 (dd, J=17.7 Hz, 10.9, 1H), 5.82 (dd, J=17.7, 0.9 Hz, 1H), 5.26 (dd, J=10.9, 0.8 Hz, 1H), 4.45 (s, 2H), 3.45 (t, J=6.0 Hz, 2H), 3.11 (s, 2H), 2.42 (t, J=7.1 Hz, 2H), 1.68-1.49 (m, 4H); MS (ESI⁻): m/z 304 [M+H]⁺.

5-[4-[[3-[(E)-Prop-1-enyl]phenyl]methoxy]butyl]-6H-1,3,4-thiadiazin-2-amine/5-[4-[(3-allylphenyl)methoxy]butyl]-6H-1,3,4-thiadiazin-2-amine

¹H NMR (400 MHz, DMSO-d₆) δ=7.36-7.07 (m, A:4H, B:4H), 6.41 (dm, J=15.8 Hz, A:1H), 6.29 (dq, J=15.8, 6.4 Hz, A:1H), 5.94 (ddt, J=16.9, 10.0, 6.8 Hz, B:1H), 5.08 (dm, J=16.9 Hz, B:1H), 5.04 (dm, J=10.0 Hz, B:1H), 4.42 (s, A:2H, B:2H), 3.46-3.41 (m, A:2H, B:2H), 3.36 (dm, J=6.8 Hz, B:2H), 3.11 (s, A:2H, B:2H), 2.42 (t, J=7.0 Hz, A:2H, B:2H), 1.84 (dd, J=6.4, 1.5 Hz, A:3H), 1.69-1.50 (m, A:4H, B:4H); MS (ESI⁺): m/z 318 [M+H]⁺.

### 5-[4-[(3-Cyclopropylphenyl)methoxy]butyl]-6H-1,3,4-thiadiazin-2-amine

¹H NMR (400 MHz, CD₃OD) δ=7.20 (t, J=7.6 Hz, 1H), 7.09 (dm, J=7.6 Hz, 1H), 7.05-7.03 (m, 1H), 6.99 (dm, J=7.7 Hz, 1H), 4.45 (s, 2H), 3.65 (s, 2H), 3.52 (t, J=6.1 Hz, 2H), 2.59 (t, J=7.3 Hz, 2H), 1.90 (tt, J=8.4, 5.1 Hz, 1H), 1.81-1.62 (m, 4H), 0.98-0.92 (m, 2H), 0.69-0.64 (m, 2H). ¹³C NMR (101 MHz, CD₃OD) δ=166.4, 158.4, 145.5, 139.6, 129.3, 126.1, 125.98, 125.95, 74.0, 70.7, 36.8, 30.0, 24.8, 23.5, 16.1, 9.6; MS (ESI⁺): m/z 318 [M+H]⁺.

### 5-[4-[(3-Prop-1-ynylphenyl)methoxy]butyl]-6H-1,3,4-thiadiazin-2-amine

¹H NMR (400 MHz, DMSO-d₆) δ=7.39-7.21 (m, 4H), 4.42 (s, 2H), 3.44 (t, J=6.1 Hz, 2H), 3.10 (s, 2H), 2.42 (t, J=7.1 Hz, 2H), 2.03 (s, 2H), 1.71-1.49 (m, 4H); MS (ESI⁺): m/z 316 [M+H]⁺.

### 5-[4-[(4-Fluorophenyl)methoxy]butyl]-6H-1,3,4-thiadiazin-2-amine hydrobromide

¹H NMR (400 MHz, DMSO-d₆) δ 7.32-7.38 (m, 2H), 7.20-7.13 (m, 2H), 4.43 (s, 2H), 3.72 (s, 2H), 3.44 (t, J=6.1 Hz, 2H), 2.53 (t, J=6.9 Hz, 2H, partially obscured by the solvent peak), 1.69-1.53 (m, 4H). MS (ESI⁺): m/z 296 [M+H]⁺.

### 4-[4-(2-Amino-6H-1,3,4-thiadiazin-5-yl)butoxymethyl]benzonitrile hydrobromide

¹H NMR (400 MHz, DMSO-d₆) δ 7.84-7.80 (m, 2H), 7.53-7.49 (m, 2H), 4.55 (s, 2H), 3.73 (s, 2H), 3.48 (t, J=6.0 Hz, 2H), 2.53 (t, J=7.1 Hz, 2H), 1.70-1.55 (m, 4H). MS (ESI⁺): m/z 303 [M+H]⁺.

### 3-[4-(2-Amino-6H-1,3,4-thiadiazin-5-yl)butoxymethyl]benzonitrile hydrobromide

¹H NMR (400 MHz, DMSO-d₆) δ 7.78-7.74 (m, 2H), 7.69-7.64 (m, 1H), 7.60-7.55 (m, 1H), 4.51 (s, 2H), 3.74 (s, 2H), 3.47 (t, J=6.0 Hz, 2H), 2.53 (t, J=7.2 Hz, 2H), 1.70-1.55 (m, 4H). MS (ESI⁺): m/z 303 [M+H]⁺.

### 5-[6-(4-Bromo-2,6-difluoro-phenyl)hexyl]-6H-1,3,4-thiadiazin-2-amine hydrobromide

¹H NMR (400 MHz, DMSO-d₆) δ 7.54-7.48 (m, 2H), 4.46 (s, 2H), 3.70 (s, 2H), 3.43 (t, J=6.0 Hz, 2H), 2.50 (t, 2H, partially obscured by the solvent peak), 1.65-1.47 (m, 4H). MS (ESI⁺): m/z 392, 394 [M+H]⁺.

### 5-[4-[(2-Chloro-6-fluoro-phenyl)methoxy]butyl]-6H-1,3,4-thiadiazin-2-amine

¹H NMR (400 MHz, CD₃OD) δ 7.35 (td, J=8.2, 6.0 Hz, 1H), 7.27 (dt, J=8.1, 1.0 Hz, 1H), 7.13-7.07 (m, 1H), 4.66 (d, J=2.2 Hz, 2H), 3.57 (t, J=5.9 Hz, 2H), 3.25 (s, 2H), 2.53 (t, J=7.3 Hz, 2H), 1.76-1.61 (m, 4H). MS (ESI⁺): m/z 330 [M+H]⁺.

### 5-[4-[(2,5-Difluorophenyl)methoxy]butyl]-6H-1,3,4-thiadiazin-2-amine

¹H NMR (400 MHz, CD₃OD) δ 7.21-7.14 (m, 1H), 7.13-6.99 (m, 2H), 4.54 (s, 2H), 3.58 (t, J=6.0 Hz, 2H), 3.48 (s, 2H), 2.59 (t, J=7.2 Hz, 2H), 1.82-1.65 (m, 4H). MS (ESI⁺): m/z 314 [M+H]⁺.

### 5-[4-[(4-Chloro-3-fluoro-phenyl)methoxy]butyl]-6H-1,3,4-thiadiazin-2-amine

¹H NMR (400 MHz, CD₃OD) δ 7.47-7.39 (m, 1H), 7.24-7.21 (m, 1H), 7.16-7.12 (m, 1H), 4.49 (s, 2H), 3.66 (s, 2H), 3.55 (t, J=6.0 Hz, 2H), 2.61 (t, J=7.3 Hz, 2H), 1.82-1.65 (m, 4H). MS (ESI⁺): m/z 330 [M+H]⁺.

### 5-[4-[(3,5-Difluorophenyl)methoxy]butyl]-6H-1,3,4-thiadiazin-2-amine

¹H NMR (400 MHz, CD₃OD) δ 6.97-6.91 (m, 2H), 6.87-6.80 (m, 1H), 4.51 (s, 2H), 3.68 (s, 2H), 3.55 (t, J=6.1 Hz, 2H), 2.62 (t, J=7.3 Hz, 2H), 1.83-1.75 (m, 2H), 1.75-1.66 (m, 2H). MS (ESI⁺): m/z 314 [M+H]⁺.

### 5-[4-[[3-(Trifluoromethyl)phenyl]methoxy]butyl]-6H-1,3,4-thiadiazin-2-amine

¹H NMR (400 MHz, CD₃OD) δ 7.68-7.50 (m, 4H), 4.58 (s, 2H), 3.67 (s, 2H), 3.57 (t, J=6.0 Hz, 2H), 2.62 (t, J=7.3 Hz, 2H), 1.84-1.66 (m, 4H). MS (ESI⁺): m/z 346 [M+H]⁺.

### 5-[4-[(4-Chlorophenyl)methoxy]butyl]-6H-1,3,4-thiadiazin-2-amine

¹H NMR (400 MHz, CD₃OD) δ 7.34-7.32 (m, 4H), 4.48 (s, 2H), 3.53 (t, J=6.0 Hz, 2H), 3.28 (d, J=3.5 Hz, 2H), 2.55 (t, J=7.3 Hz, 2H), 1.79-1.63 (m, 4H). MS (ESI⁺): m/z 312 [M+H]⁺.

### 5-[4-(m-Tolylmethoxy)butyl]-6H-1,3,4-thiadiazin-2-amine

¹H NMR (400 MHz, CD₃OD) δ 7.21 (t, J=7.5 Hz, 1H), 7.17-7.13 (m, 1H), 7.13-7.08 (m, 2H), 4.46 (s, 2H), 3.52 (t, J=6.1 Hz, 2H), 2.59 (t, J=7.3 Hz, 2H), 2.33 (s, 3H), 1.81-1.72 (m, 2H), 1.72-1.63 (m, 2H). MS (ESI⁻): m/z 292 [M+H]⁺.

### 5-[4-[(2,3,4-Trifluorophenyl)methoxy]butyl]-6H-1,3,4-thiadiazin-2-amine

¹H NMR (400 MHz, DMSO-d₆) δ=7.36-7.25 (m, 2H), 4.51 (s, 2H), 3.66 (s, 2H), 3.46 (t, J=6.0, 2H), 2.53-2.48 (2 H, partially obscured by the solvent peak), 1.67-1.52 (m, 4H); MS (ESI⁺): m/z 332 [M+H]⁺.

### 5-[4-[(2,3-Difluorophenyl)methoxy]butyl]-6H-1,3,4-thiadiazin-2-amine

¹H NMR (400 MHz, DMSO-d₆) δ=7.42-7.33 (m, 1H), 7.28-7.17 (m, 2H), 4.54 (s, 2H), 3.48 (t, J=6.0, 2H), 3.39 (s, 2H), 2.46 (t, J=7.0, 2H), 1.67-1.51 (m, 4H); MS (ESI⁺): m/z 314 [M+H]⁺.

### 5-[4-[(3-Bromo-5-fluoro-phenyl)methoxy]butyl]-6H-1,3,4-thiadiazin-2-amine

¹H NMR (400 MHz, DMSO-d₆) δ=7.45 (app. dt, J=8.5, 2.1, 1H), 7.37 (br. s, 1H), 7.18 (dm, J=9.5, 1H), 4.48 (s, 2H), 3.61 (s, 2H), 3.46 (t, J=6.0, 2H), 2.54-2.48 (2H, partially obscured by the solvent peak), 1.70-1.53 (m, 4H); MS (ESI⁺): m/z 374, 376 [M+H]⁺.

### 5-[4-[(2,4,6-Trifluorophenyl)methoxy]butyl]-6H-1,3,4-thiadiazin-2-amine

¹H NMR (400 MHz, DMSO-d₆) δ=7.27-7.16 (m, 2H), 4.45 (s, 2H), 3.71 (s, 2H), 3.43 (t, J=6.0, 2H), 2.53-2.45 (2H partially obscured by the solvent peak), 1.64-1.48 (m, 4H). MS (ESI⁺): m/z 332 [M+H]⁺.

### 5-[4-[(2,6-Difluorophenyl)methoxy]butyl]-6H-1,3,4-thiadiazin-2-amine

¹H NMR (400 MHz, DMSO-d₆) δ=7.50-7.42 (m, 1H), 7.16-7.07 (m, 2H), 4.49 (s, 2H), 3.71 (s, 2H), 3.44 (t, J=6.0, 2H), 2.54-2.44 (2H, partially obscured by the solvent peak), 1.65-1.48 (m, 4H); MS (ESI⁺): m/z 314 [M+H]⁺.

### 5-[4-[(2,6-Difluoro-4-methoxy-phenyl)methoxy]butyl]-6H-1,3,4-thiadiazin-2-amine

¹H NMR (400 MHz, DMSO-d₆) δ=6.75 (dm, J=9.8, 2H), 4.40 (s, 2H), 3.78 (s, 3H), 3.70 (s, 2H), 3.41 (t, J=6.0, 2H), 2.52-2.46 (2H, partially obscured by the solvent peak), 1.63-1.49 (m, 4H); MS (ESI⁻): m/z 344 [M+H]⁺.

### 5-[4-[(4-Bromo-3-fluoro-phenyl)methoxy]butyl]-6H-1,3,4-thiadiazin-2-amine

¹H NMR (400 MHz, DMSO-d₆) δ=7.68 (app. t, J=7.8, 1H), 7.30 (dd, J=9.8, 1.9, 1H), 7.13 (dd, J=8.2, 1.4, 1H), 4.45 (s, 2H), 3.74 (s, 2H), 3.46 (t, J=6.0, 2H), 2.57-2.46 (2H, partially obscured by the solvent peak), 1.69-1.54 (m, 4H); MS (ESI⁺): m/z 374, 376 [M+H]⁺.

### 5-[4-(1,3-Benzothiazol-2-ylmethoxy)butyl]-6H-1,3,4-thiadiazin-2-amine

¹H NMR (400 MHz, DMSO-d₆) δ=8.10 (ddd, J=7.9, 1.3, 0.6, 1H), 7.97 (ddd, J=8.1, 1.2, 0.7, 1H), 7.54-7.41 (m, 2H), 4.90 (s, 2H), 3.74 (s, 2H), 3.63 (t, J=6.0, 2H), 2.57 (t, J=7.0, 2H), 1.75-1.60 (m, 4H); MS (ESI⁺): m/z 335 [M+H]⁺.

### 5-[4-[(2-Nitrophenyl)methoxy]butyl]-6H-1,3,4-thiadiazin-2-amine

¹H NMR (400 MHz, DMSO-d₆) δ=8.05-8.00 (m, 1H), 7.78-7.70 (m, 2H), 7.60-7.54 (m, 1H), 4.78 (s, 2H), 3.73 (s, 2H), 3.50 (t, J=6.0, 2H), 2.53 (t, J=7.1, 2H, partially obscured by the solvent peak), 1.70-1.53 (m, 4H); MS (ESI⁺): m/z 323 [M+H]⁺.

### 5-[4-(Tetrahydrofuran-2-ylmethoxy)butyl]-6H-1,3,4-thiadiazin-2-amine

¹H NMR (400 MHz, DMSO-d₆) δ=3.90 (m, 1H), 3.37 (s, 2H), 3.72-3.68 (m, 1H), 3.64-3.57 (m, 1H), 3.41 (t, J=6.2, 2H), 3.32 (d, 2H), 2.53 (t, J=7.1, 2H, partially obscured by the solvent peak), 1.91-1.72 (m, 2H), 1.65-1.57 (m, 2H), 1.56-1.46 (m, 4H); MS (ESI⁺): m/z 272 [M+H]⁺.

### 5-[4-[(2-Methylcyclopropyl)methoxy]butyl]-6H-1,3,4-thiadiazin-2-amine

¹H NMR (major isomer, 400 MHz, DMSO-d₆) δ=3.73 (s, 2H), 3.41-3.31 (m, 2H), 3.26-3.11 (m, 2H), 2.53 (t, J=7.0, 2H, partially obscured by the solvent peak), 1.68-1.58 (m, 2H), 1.56-1.48 (m, 2H), 0.99 (d, J=5.9, 3H), 0.71-0.53 (m, 2H); 0.33-0.26 (m, 1H), 0.23-0.16 (m, 1H); MS (ESI⁺): m/z 256 [M+H]⁺.

### 5-[4-[(2,4-Dimethylphenyl)methoxy]butyl]-6H-1,3,4-thiadiazin-2-amine

¹H NMR (400 MHz, DMSO-d₆) δ=7.14 (d, J=7.6, 1H), 6.99-6.93 (m, 2H), 4.39 (s, 2H), 3.72 (s, 2H), 3.43 (t, J=6.0, 2H), 2.54-2.51 (m, 2H, partially obscured by the solvent peak), 2.24 (s, 3H), 2.23 (s, 3H), 1.68-1.51 (m, 4H); MS (ESI⁺): m/z 306 [M+H]⁺.

### 5-[4-[(2-Chlorophenyl)methoxy]butyl]-6H-1,3,4-thiadiazin-2-amine

¹H NMR (400 MHz, DMSO-d₆) δ=7.50-7.43 (m, 2H), 7.38-7.30 (m, 2H), 4.53 (s, 2H), 3.73 (s, 2H), 3.52 (t, J=6.0, 2H), 2.54 (t, J=7.1, 2H, partially obscured by the solvent peak), 1.71-1.56 (m, 4H); MS (ESI⁺): m/z 312 [M+H]⁺.

### 5-[4-[(5-Methyl-2-thienyl)methoxy]butyl]-6H-1,3,4-thiadiazin-2-amine

¹H NMR (400 MHz, DMSO-d₆) δ 6.81 (d, J=3.3 Hz, 1H), 6.66-6.64 (m, 1H), 4.51 (s, 2H), 3.41 (t, J=6.1 Hz, 2H), 3.13 (s, 2H), 2.43-2.37 (m, 5H), 1.64-1.46 (m, 4H). MS (ESI⁺): m/z 298 [M+H]⁺.

### 5-[4-[(4-Chlorophenyl)methylsulfanyl]butyl]-6H-1,3,4-thiadiazin-2-amine

¹H NMR (400 MHz, CD₃OD) δ 7.31-7.29 (m, 4H), 3.70 (s, 2H), 3.65 (s, 2H), 2.55 (t, J=7.3 Hz, 2H), 2.46 (t, J=7.1 Hz, 1H), 1.77-1.67 (m, 2H), 1.66-1.56 (m, 1H). MS (ESI⁺): m/z 328 [M+H]⁺.

### 5-[5-(3-Ethylphenoxy)pentyl]-6H-1,3,4-thiadiazin-2-amine

¹H NMR (400 MHz, DMSO-d₆) δ 7.19-7.12 (m, 1H), 6.78-6.67 (m, 3H), 3.93 (t, J=6.5 Hz, 2H), 3.14 (s, 2H), 2.55 (q, J=7.6 Hz, 2H), 2.46-2.41 (m, 2H), 1.77-1.67 (m, 2H), 1.67-1.57 (m, 2H), 1.50-1.37 (m, 2H), 1.16 (t, J=7.6 Hz, 3H). MS (ESI⁺): m/z 306 [M+H]⁺.

### 5-[5-(4-Ethylphenoxy)pentyl]-6H-1,3,4-thiadiazin-2-amine

¹H NMR (400 MHz, DMSO-d₆) δ 7.11-7.07 (m, 2H), 6.84-6.79 (m, 2H), 3.91 (t, J=6.5 Hz, 2H), 3.38 (s, 2H), 2.57-2.43 (m, 4H, partially obscured by the solvent signal), 1.76-1.67 (m, 2H), 1.67-1.57 (m, 2H), 1.49-1.39 (m, 2H), 1.13 (t, J=7.6 Hz, 3H). MS (ESI⁺): m/z 306 [M+H]⁺.

### 5-[2-(3-Phenylpropoxy)ethyl]-6H-1,3,4-thiadiazin-2-amine

¹H NMR (400 MHz, DMSO-d₆) δ 7.30-7.24 (m, 2H), 7.20-7.14 (m, 3H), 3.68-3.61 (m, 4H), 3.39 (t, J=6.4 Hz, 2H), 2.74 (t, J=6.3 Hz, 2H), 2.62-2.56 (m, 2H), 1.82-1.73 (m, 2H). MS (ESI⁺): m/z 278 [M+H]⁺.

### 5-[2-(4-Methoxybutoxy)ethyl]-6H-1,3,4-thiadiazin-2-amine

¹H NMR (400 MHz, DMSO-d₆) δ 3.69 (s, 2H), 3.63 (t, J=6.2 Hz, 2H), 3.40-3.36 (m, 2H), 3.30-3.26 (m, 2H), 3.20 (s, 3H), 2.73 (t, J=6.2 Hz, 2H), 1.51-1.46 (m, 4H). MS (ESI⁻): m/z 286 [M+H]⁺.

### 5-[4-[3-(2,4-Difluorophenyl)propoxy]butyl]-6H-1,3,4-thiadiazin-2-amine

¹H NMR (400 MHz, CD₃OD) δ 7.29-7.22 (m, 1H), 6.90-6.83 (m, 2H), 3.69 (s, 2H), 3.48-3.41 (m, 4H), 2.69 (t, J=7.6 Hz, 2H), 2.61 (t, J=7.3 Hz, 2H), 1.88-1.80 (m, 2H), 1.78-1.71 (m, 2H), 1.69-1.60 (m, 2H). MS (ESI⁺): m/z 342 [M+H]⁺.

### 5-[4-(2-Phenylethoxy)butyl]-6H-1,3,4-thiadiazin-2-amine

¹H NMR (400 MHz, CD₃OD) δ 7.29-7.20 (m, 4H), 7.20-7.14 (m, 1H), 3.65 (t, J=6.8 Hz, 2H), 3.59 (s, 2H), 3.48 (t, J=6.0 Hz, 2H), 2.85 (t, J=6.8 Hz, 2H), 2.54 (t, J=7.3 Hz, 2H), 1.73-1.57 (m, 4H). MS (ESI⁺): m/z 292 [M+H]⁺.

### 4-(2-Amino-6H-1,3,4-thiadiazin-5-yl)butyl acetate

¹H NMR (400 MHz, CD₃OD) δ=4.10 (t, J=6.1, 2H), 3.70 (s, 2H), 2.62 (t, J=7.1, 2H), 2.03 (s, 3H), 1.81-1.67 (m, 4H); MS (ESI⁺): m/z 230 [M+H]⁺.

### Ethyl 3-[3-[4-(2-amino-6H-1,3,4-thiadiazin-5-yl)butoxymethyl]phenyl]propanoate

¹H NMR (400 MHz, DMSO-d₆) δ=7.28-7.23 (m, A:1H, B:1H), 7.17-7.11 (m, A:3H, B:3H), 4.41 (s, A:2H, B:2H), 4.03 (q, J=7.1, A:2H), 3.65 (s, A:2H, B:2H), 3.57 (s, B:3H), 3.43 (t, J=6.0, A:2H, B:2H), 2.84 (t, J=7.5, A:2H, B:2H), 2.64-2.57 (m, A:2H, B:2H), 2.51 (t, J=7.3, A:2H, B:2H, partially obscured by the solvent peak), 1.70-1.51 (m, A:4H, B:4H), 1.14 (t, J=7.1, A:3H); MS (ESI⁺): m/z 378 [M+H]⁺(A), MS (ESI⁺): m/z 364 [M+H]⁺(B).

### 2-[4-(2-Amino-6H-1,3,4-thiadiazin-5-yl)butoxymethyl]benzonitrile

¹H NMR (400 MHz, DMSO-d₆) δ=7.85 (dd, J=7.7, 0.9, 1H), 7.71 (td, J=7.6, 1.3, 1H), 7.62-7.59 (m, 1H), 7.51 (td, J=7.6, 1.2, 1H), 4.61 (s, 2H), 3.53-3.49 (m, 4H), 2.49 (partially obscured by the solvent peak, 2H), 1.68-1.58 (m, 4H). MS (ESI⁺): m/z 303 [M+H]⁺.

### Example 118

### Minimum Inhibitory Concentration

Between 1 and 5 mgs of compound were accurately weighed out into a sterile Eppendorf tube. The compound was dissolved in DMSO to give a solution containing 5 mg/mL. Tubes were stored at −20° C. until required.

On the day of testing thawed solutions were vortex mixed to ensure homogeneity. 30 μL of solution was removed and added to 570 μL of sterile water in a separate sterile Eppendorf. The thoroughly mixed solution was used to prepare a series of doubling dilutions in water, in a deep well plate. Thirteen replicate plates were prepared using a Minitrak by aspirating 20 μL from each well into eleven clear polystyrene 96 well plates.

Spores of Aspergillus spp. (Aspergillus fumigatus [two strains], Aspergillus terreus [two strains], Aspergillus niger and Aspergillus flavus) were harvested from cultures grown on Sabarauds agar for 5 days, and resuspended in PBS/Tween 80 to approx 1×10⁷ cfu/mL. Each organism suspension was diluted in YAG medium (1% glucose, 1% ammonium chloride and 0.5% yeast extract) to 0.5-2×10⁴cfu/mL. 80 μL of an organism suspension was added to each well of the plate containing drug dilutions.

This typically produced MIC plates with a drug range 50-0.05 mg/L and organism inocula of 1-2×10⁴ cfu/mL for Aspergillus spp. All plates were incubated for 24 hrs at 35° C. Growth was assessed by monitoring the optical density at 485 nm for each well. The MIC of a compound is the lowest drug concentration that inhibits growth of an organism by >70% compared with a drug free control. MICs are recorded as mg/L. In cases where the MIC of an organism is >=0.05 mg/L the MIC is repeated using a concentration range of 0.5-0.0005 mg/L.

Assays were also performed in RPMI medium (Roswell Park Memorial Media 1640). To perform MIC tests in this medium, dilutions of compounds are prepared in microtitre plates as described above. Fungal strains to be tested are grown and harvested in an identical manner to that described above, and each organism suspension was diluted in RPMI medium, typically containing 2% glucose and 0.135 M MOPS buffer (pH 7.0) to 0.5-2×10⁴cfu/mL, rather than in YAG medium. 80 μL of an organism suspension was added to each well of the plate containing drug dilutions.

This produced MIC plates with a drug range 50-0.05 mg/L and organism inocula of 1-2×10⁴ cfu/mL. All plates were incubated for 24-48 hrs at 35° C. Growth was assessed by monitoring the optical density at 485 nm for each well. The MIC of a compound is the lowest drug concentration that inhibits growth of an organism by >80% compared with a drug free control. The following organisms were tested: C. albicans, C. parapsilosis, C. tropicalis, C. glabrata, C. krusei, A. niger, A. fumigatus, A. terreus, A. flavus, A. terreus 49, and A. fumigatus 210.

Results are shown in Table 1. In this table, the MIC results have been banded into grades as follows:

YAG media: 1: ≥5 mg/L; 2: 1-5 mg/L; 3: 0.2-1 mg/L; 4: <0.2 mg/L

RPMI media: 1: ≥20 mg/L; 2: 5-20 mg/L; 3: 1-5 mg/L; 4: <1 mg/L

As can be seen, MIC values indicated the potential for broad spectrum activity with MIC values appearing more potent in YAG media.

TABLE 1

| | | | | | | A. FUMIGATUS | | | |
|---|---|---|---|---|---|---|---|---|---|
| | A. NIGER | A. FUMIGATUS | A. TERREUS | A. FLAVUS | A. TERREUS 49 | 210 | C. ALBICANS | C. PARAPSILOSIS | C. TROPICALIS |
| Structure Name | YAG media | | | | | | RPMI media | | |
| 5-[4-(4-ethylphenoxy)butyl]-6~{H}-1,3,4-thiadiazin-2- | 3 | 3 | 4 | 4 | 4 | 3 | 4 | 4 | 4 |
| amine | | | | | | | | | |
| 5-[5-(2-pyridyloxy)pentyl]-6~{H}-1,3,4-thiadiazin-2- | 4 | 4 | 4 | 4 | 4 | 4 | 4 | 4 | 4 |
| amine | | | | | | | | | |
| 5-[2-(2-phenoxyethoxy)ethyl]-6~{H}-1,3,4-thiadiazin-2- | 4 | 4 | 4 | 4 | 4 | 4 | 3 | 4 | 3 |
| amine | | | | | | | | | |
| 5-[4-[[2-(2-phenylethyl)phenyl]methoxy]butyl]-6~{H}- | 1 | 1 | 2 | 2 | 1 | 1 | 2 | 2 | 2 |
| 1,3,4-thiadiazin-2-amine | | | | | | | | | |
| 5-[4-[(3,5-dichlorophenyl)methoxy]butyl]-6~{H}-1,3,4- | 3 | 3 | 3 | 4 | 3 | 2 | 2 | 3 | 2 |
| thiadiazin-2-amine | | | | | | | | | |
| 5-[4-[(4-chloro-2-fluoro-phenyl)methoxy]butyl]-6~{H}- | 3 | 4 | 4 | 4 | 4 | 4 | 4 | 4 | 4 |
| 1,3,4-thiadiazin-2-amine | | | | | | | | | |
| 5-[4-[(2-cyclopropylphenyl)methoxy]butyl]-6~{H}-1,3,4- | 2 | 2 | 2 | 3 | 2 | 2 | 3 | 3 | 3 |
| thiadiazin-2-amine | | | | | | | | | |
| 5-[4-[[2-(trifluoromethyl)phenyl]methoxy]butyl]-6~{H}- | 3 | 3 | 2 | 2 | 2 | 2 | 2 | 1 | 2 |
| 1,3,4-thiadiazin-2-amine | | | | | | | | | |
| 5-[4-[(2,5-difluorophenyl)methoxy]butyl]-6~{H}-1,3,4- | 4 | 4 | 4 | 4 | 4 | 4 | 3 | 3 | 3 |
| thiadiazin-2-amine | | | | | | | | | |
| 5-[4-[(2-chloro-6-fluoro-phenyl)methoxy]butyl]-6~{H}- | 3 | 3 | 3 | 3 | 3 | 3 | 2 | 2 | 2 |
| 1,3,4-thiadiazin-2-amine | | | | | | | | | |
| 5-[4-[(3-butylphenyl)methoxy]butyl]-6~{H}-1,3,4- | 3 | 1 | 2 | 3 | 2 | 1 | 2 | 2 | 2 |
| thiadiazin-2-amine | | | | | | | | | |
| 5-[4-[(4-bromophenyl)methoxy]butyl]-6~{H}-1,3,4- | 4 | 4 | 4 | 4 | 4 | 4 | 4 | 4 | 4 |
| thiadiazin-2-amine | | | | | | | | | |
| 5-[4-[(4-chlorophenyl)methylsulfanyl]butyl]-6~{H}- | 4 | 3 | 4 | 4 | 4 | 3 | 3 | 3 | 3 |
| 1,3,4-thiadiazin-2-amine | | | | | | | | | |
| 5-[4-(2-phenylethoxy)butyl]-6~{H}-1,3,4-thiadiazin-2- | 3 | 3 | 3 | 4 | 3 | 3 | 3 | 3 | 3 |
| amine | | | | | | | | | |
| 4-(2-amino-6~{H}-1,3,4-thiadiazin-5-yl)butyl acetate | 3 | 4 | 4 | 4 | 4 | 4 | 3 | 4 | 3 |
| 5-[4-[(3-ethylphenyl)methoxy]butyl]-6~{H}-1,3,4- | 4 | 4 | 4 | 4 | 4 | 3 | 4 | 4 | 4 |
| thiadiazin-2-amine | | | | | | | | | |
| 5-[4-[[3-(2-phenylethyl)phenyl]methoxy]butyl]-6~{H}- | 2 | 2 | 2 | 2 | 2 | 1 | 2 | 2 | 2 |
| 1,3,4-thiadiazin-2-amine | | | | | | | | | |
| 5-[4-[3-(2,4-difluorophenyl)propoxy]butyl]-6~{H}-1,3,4- | 3 | 3 | 3 | 4 | 3 | 3 | 4 | 4 | 3 |
| thiadiazin-2-amine | | | | | | | | | |
| 5-[4-[(3-phenylphenyl)methoxy]butyl]-6~{H}-1,3,4- | 2 | 1 | 2 | 2 | 2 | 1 | 2 | 2 | 2 |
| thiadiazin-2-amine | | | | | | | | | |
| 5-[4-[[3-(trifluoromethyl)phenyl]methoxy]butyl]-6~{H}- | 3 | 3 | 2 | 3 | 2 | 3 | 3 | 2 | 2 |
| 1,3,4-thiadiazin-2-amine | | | | | | | | | |
| 5-[4-[(4-chloro-3-fluoro-phenyl)methoxy]butyl]-6~{H}- | 4 | 4 | 4 | 4 | 4 | 4 | 4 | 4 | 4 |
| 1,3,4-thiadiazin-2-amine | | | | | | | | | |
| 5-[4-[(3,5-difluorophenyl)methoxy]butyl]-6~{H}-1,3,4- | 4 | 4 | 4 | 4 | 4 | 4 | 4 | 4 | 4 |
| thiadiazin-2-amine | | | | | | | | | |
| 5-[4-[(3-phenoxyphenyl)methoxy]butyl]-6~{H}-1,3,4- | 2 | 1 | 1 | 2 | 1 | 1 | 3 | 3 | 3 |
| thiadiazin-2-amine | | | | | | | | | |
| 5-[4-[(2,4-dichlorophenyl)methoxy]butyl]-6~{H}-1,3,4- | 3 | 2 | 2 | 2 | 2 | 2 | 2 | 2 | 2 |
| thiadiazin-2-amine | | | | | | | | | |
| 5-[4-[(3-bromophenyl)methoxy]butyl]-6~{H}-1,3,4- | 4 | 4 | 4 | 4 | 4 | 4 | 4 | 4 | 4 |
| thiadiazin-2-amine | | | | | | | | | |
| 5-[4-[(3-cyclopropylphenyl)methoxy]butyl]-6~{H}-1,3,4- | 3 | 3 | 3 | 3 | 3 | 3 | 4 | 4 | 4 |
| thiadiazin-2-amine | | | | | | | | | |
| 5-[4-[(4-fluoro-3-methyl-phenyl)methoxy]butyl]-6~{H}- | 4 | 4 | 4 | 4 | 4 | 4 | 4 | 4 | 4 |
| 1,3,4-thiadiazin-2-amine | | | | | | | | | |
| 5-[4-[(4-fluorophenyl)methoxy]butyl]-6~{H}-1,3,4- | 2 | 2 | 2 | 2 | 2 | 2 | 2 | 2 | 2 |
| thiadiazin-2-amine | | | | | | | | | |
| 5-[4-[(4-methoxyphenyl)methoxy]butyl]-6~{H}-1,3,4- | 2 | 2 | 2 | 2 | 2 | 2 | 2 | 2 | 2 |
| thiadiazin-2-amine | | | | | | | | | |
| 5-[4-[(2,4-difluorophenyl)methoxy]butyl]-6~{H}-1,3,4- | 4 | 4 | 4 | 4 | 4 | 4 | 4 | 4 | 4 |
| thiadiazin-2-amine | | | | | | | | | |
| 5-[4-[(3-chlorophenyl)methoxy]butyl]-6~{H}-1,3,4- | 4 | 4 | 4 | 4 | 4 | 4 | 4 | 4 | 4 |
| thiadiazin-2-amine | | | | | | | | | |
| 5-[4-(m-tolylmethoxy)butyl]-6~{H}-1,3,4-thiadiazin-2- | 3 | 3 | 3 | 3 | 3 | 3 | 3 | 3 | 3 |
| amine | | | | | | | | | |
| 5-[4-[(4-butylphenyl)methoxy]butyl]-6~{H}-1,3,4- | 3 | 1 | 2 | 3 | 2 | 1 | 2 | 3 | 2 |
| thiadiazin-2-amine | | | | | | | | | |
| 5-[4-[[4-(trifluoromethyl)phenyl]methoxy]butyl]-6~{H}- | 4 | 4 | 4 | 4 | 4 | 4 | 4 | 4 | 4 |
| 1,3,4-thiadiazin-2-amine | | | | | | | | | |
| 5-[4-[(4-chlorophenyl)methoxy]butyl]-6~{H}-1,3,4- | 4 | 4 | 4 | 4 | 4 | 4 | 4 | 4 | 3 |
| thiadiazin-2-amine | | | | | | | | | |
| 5-[5-(4-butylphenoxy)pentyl]-6~{H}-1,3,4-thiadiazin-2- | 3 | 1 | 2 | 2 | 2 | 2 | 2 | 3 | 2 |
| amine | | | | | | | | | |
| 5-[2-[(4-butylphenyl)methoxy]ethyl]-6~{H}-1,3,4- | 3 | 1 | 3 | 3 | 2 | 1 | 2 | 2 | 2 |
| thiadiazin-2-amine | | | | | | | | | |
| 5-[2-[3-(4-methoxyphenyl)propoxy]ethyl]-6~{H}-1,3,4- | 2 | 2 | 2 | 2 | 2 | 2 | 2 | 3 | 2 |
| thiadiazin-2-amine | | | | | | | | | |
| 5-[5-(4-ethylphenoxy)pentyl]-6~{H}-1,3,4-thiadiazin-2- | 4 | 3 | 4 | 4 | 4 | 3 | 4 | 4 | 4 |
| amine | | | | | | | | | |
| 5-[2-(4-methoxybutoxy)ethyl]-6~{H}-1,3,4-thiadiazin-2- | 3 | 3 | 3 | 3 | 3 | 3 | 3 | 3 | 2 |
| amine | | | | | | | | | |
| 5-[2-(3-phenylpropoxy)ethyl]-6~{H}-1,3,4-thiadiazin-2- | 4 | 4 | 4 | 4 | 4 | 4 | 4 | 4 | 3 |
| amine | | | | | | | | | |
| butyl 4-(2-amino-6~{H}-1,3,4-thiadiazin-5-yl)benzoate | 3 | 3 | 2 | 3 | 2 | 3 | 1 | 1 | 1 |
| 5-[5-(3-ethylphenoxy)pentyl]-6~{H}-1,3,4-thiadiazin-2- | 4 | 4 | 4 | 4 | 4 | 4 | 4 | 4 | 4 |
| amine | | | | | | | | | |
| 5-(4-butoxyphenyl)-4-methyl-1,3,4-thiadiazin-2-amine | 4 | 4 | 3 | 4 | 3 | 4 | 1 | 3 | 3 |
| 5-(4-benzyloxybutyl)-6~{H}-1,3,4-thiadiazin-2-amine | 4 | 4 | 4 | 4 | 4 | 4 | 4 | 4 | 4 |
| 5-(4-methoxyphenyl)-4-methyl-1,3,4-thiadiazin-2-amine | 4 | 4 | 4 | 4 | 4 | 4 | 4 | 4 | 3 |
| 5-(benzofuran-2-yl)-4-methyl-1,3,4-thiadiazin-2-amine | 2 | 2 | 3 | 2 | 3 | 3 | 1 | 1 | 1 |
| 4-(2-amino-4-methyl-1,3,4-thiadiazin-5-yl)benzonitrile | 4 | 3 | 4 | 4 | 4 | 4 | 3 | 3 | 2 |
| ethyl 4-(2-amino-4-methyl-1,3,4-thiadiazin-5-yl)benzoate | 3 | 4 | 4 | 4 | 3 | 4 | 3 | 4 | 3 |
| 5-(3,4-dichlorophenyl)-4-methyl-1,3,4-thiadiazin-2-amine | 2 | 2 | 2 | 1 | 3 | 2 | 1 | 1 | 1 |
| 4-methyl-5-(p-tolyl)-1,3,4-thiadiazin-2-amine | 3 | 4 | 4 | 3 | 4 | 4 | 2 | 3 | 1 |
| 5-[2-(4-pentoxyphenyl)ethyl]-6~{H}-1,3,4-thiadiazin-2- | 3 | 1 | 2 | 3 | 3 | 1 | 1 | 3 | 3 |
| amine | | | | | | | | | |
| 5-(5-benzyloxypentyl)-6~{H}-1,3,4-thiadiazin-2-amine | 3 | 4 | 3 | 4 | 4 | 4 | 3 | 4 | 3 |
| 5-[4-(benzofuran-2-yl)butyl]-6~{H}-1,3,4-thiadiazin-2- | 3 | 3 | 4 | 4 | 3 | 3 | 3 | 4 | 3 |
| amine | | | | | | | | | |
| 5-(5-methoxypentyl)-4-methyl-3~{H}-1,3,4-thiadiazin-2- | 3 | 3 | 3 | 4 | 4 | 4 | 4 | 4 | 3 |
| imine | | | | | | | | | |
| 5-(4-chlorophenyl)-4-methyl-1,3,4-thiadiazin-2-amine | 4 | 4 | 4 | 4 | 4 | 4 | 3 | 4 | 2 |
| 2-[5-(2-amino-6~{H}-1,3,4-thiadiazin-5- | 4 | 4 | 4 | 4 | 4 | 4 | 1 | 3 | 1 |
| yl)pentyl]isoindoline-1,3-dione | | | | | | | | | |
| 5-[5-(4-fluorophenoxy)pentyl]-6~{H}-1,3,4-thiadiazin-2- | 4 | 4 | 4 | 4 | 4 | 4 | 4 | 4 | 4 |
| amine | | | | | | | | | |
| 5-[(~{E})-hept-1-enyl]-6~{H}-1,3,4-thiadiazin-2-amine | 2 | 2 | 2 | 2 | 2 | 2 | 2 | 2 | 1 |
| 4-(5-methoxypentyl)-5-methyl-1,3,4-thiadiazin-2-amine | 1 | 1 | 2 | 2 | 2 | 2 | 1 | 2 | 1 |
| 5-[5-(2-pyridyl)pentyl]-6~{H}-1,3,4-thiadiazin-2-amine | 4 | 3 | 3 | 3 | 4 | 4 | 2 | 3 | 2 |
| 5-[5-(~{N}-methylanilino)pentyl]-6~{H}-1,3,4- | 3 | 2 | 3 | 3 | 3 | 2 | 1 | 2 | 1 |
| thiadiazin-2-amine | | | | | | | | | |
| 5-(6-methoxyhexyl)-6~{H}-1,3,4-thiadiazin-2-amine | 4 | 4 | 4 | 4 | 4 | 4 | 4 | 4 | 3 |
| 6-(2-butoxyethyl)-4~{H}-1,3,4-thiadiazin-2-amine | 1 | 2 | 2 | 2 | 2 | 2 | 1 | 1 | 1 |
| 5-(7-phenoxyheptyl)-6~{H}-1,3,4-thiadiazin-2-amine | 4 | 4 | 4 | 4 | 4 | 3 | 3 | 4 | 3 |
| 5-(7-methoxyheptyl)-6~{H}-1,3,4-thiadiazin-2-amine | 4 | 4 | 4 | 4 | 4 | 4 | 4 | 4 | 4 |
| 5-(4-methoxybutyl)-6~{H}-1,3,4-thiadiazin-2-amine | 3 | 3 | 3 | 3 | 3 | 4 | 3 | 4 | 3 |
| 6-(2-amino-6~{H}-1,3,4-thiadiazin-5-yl)hexanoic acid | 2 | 2 | 2 | 2 | 2 | 2 | 1 | 1 | 1 |
| ethyl 6-(2-amino-6~{H}-1,3,4-thiadiazin-5-yl)hexanoate | 4 | 3 | 4 | 4 | 4 | 4 | 4 | 4 | 3 |
| 5-(methoxymethyl)-6~{H}-1,3,4-thiadiazin-2-amine | 2 | 2 | 2 | 2 | 2 | 2 | 2 | 2 | 2 |
| 5-(4-phenoxybutyl)-6~{H}-1,3,4-thiadiazin-2-amine | 4 | 3 | 4 | 4 | 4 | 3 | 4 | 4 | 3 |
| 5-[2-(4-phenylphenoxy)ethyl]-6~{H}-1,3,4-thiadiazin-2- | 2 | 1 | 2 | 2 | 2 | 1 | 1 | 1 | 1 |
| amine | | | | | | | | | |
| 5-[2-(4-methylphenoxy)ethyl]-6~{H}-1,3,4-thiadiazin-2- | 3 | 3 | 3 | 3 | 3 | 4 | 1 | 4 | 2 |
| amine | | | | | | | | | |
| 5-(phenoxymethyl)-6~{H}-1,3,4-thiadiazin-2-amine | 3 | 4 | 3 | 4 | 3 | 4 | 2 | 4 | 2 |
| 5-(3-phenoxypropyl)-6~{H}-1,3,4-thiadiazin-2-amine | 4 | 4 | 4 | 4 | 3 | 4 | 2 | 4 | 3 |
| 5-(6-phenoxyhexyl)-6~{H}-1,3,4-thiadiazin-2-amine | 4 | 4 | 4 | 4 | 4 | 4 | 3 | 4 | 4 |
| 5-(6-phenylhexyl)-6~{H}-1,3,4-thiadiazin-2-amine | 4 | 4 | 4 | 4 | 3 | 4 | 2 | 4 | 3 |
| 5-(2-amino-6~{H}-1,3,4-thiadiazin-5-yl)-~{N}-phenyl- | 2 | 3 | 2 | 3 | 2 | 3 | 1 | 2 | 1 |
| pentanamide | | | | | | | | | |
| 5-[2-[(4-methoxyphenyl)methoxy]ethyl]-6~{H}-1,3,4- | 3 | 3 | 3 | 3 | 3 | 3 | 2 | 3 | 2 |
| thiadiazin-2-amine | | | | | | | | | |
| 5-[2-(4-propoxyphenyl)ethyl]-6~{H}-1,3,4-thiadiazin-2- | 4 | 4 | 4 | 3 | 4 | 4 | 4 | 4 | 3 |
| amine | | | | | | | | | |
| 4-[2-(2-amino-6~{H}-1,3,4-thiadiazin-5- | 4 | 4 | 4 | 3 | 4 | 4 | 2 | 2 | 1 |
| yl)ethoxymethyl]benzonitrile | | | | | | | | | |
| 5-[2-(1-methylbutoxy)ethyl]-6~{H}-1,3,4-thiadiazin-2- | 4 | 2 | 3 | 2 | 2 | 3 | 2 | 3 | 1 |
| amine | | | | | | | | | |
| 5-[2-(p-tolylmethoxy)ethyl]-6~{H}-1,3,4-thiadiazin-2- | 4 | 4 | 4 | 3 | 4 | 4 | 3 | 3 | 2 |
| amine | | | | | | | | | |
| 5-[2-[(4-phenylphenyl)methoxy]ethyl]-6~{H}-1,3,4- | 3 | 1 | 3 | 3 | 3 | 1 | 1 | 2 | 2 |
| thiadiazin-2-amine | | | | | | | | | |
| 5-(2-hexoxyethyl)-6~{H}-1,3,4-thiadiazin-2-amine | 4 | 4 | 4 | 4 | 4 | 4 | 4 | 4 | 3 |
| 5-[2-(2-methoxyethoxy)ethyl]-6~{H}-1,3,4-thiadiazin-2- | 3 | 2 | 3 | 2 | 3 | 2 | 2 | 2 | 1 |
| amine | | | | | | | | | |
| 5-(2-phenoxyethyl)-6~{H}-1,3,4-thiadiazin-2-amine | 3 | 4 | 4 | 3 | 3 | 4 | 3 | 4 | 2 |
| 5-[2-(4-bromophenoxy)ethyl]-6~{H}-1,3,4-thiadiazin-2- | 4 | 4 | 3 | 4 | 1 | 4 | 1 | 3 | 2 |
| amine | | | | | | | | | |
| 5-(2-naphthyl)-6~{H}-1,3,4-thiadiazin-2-amine | 1 | 2 | 1 | 1 | 1 | 1 | 1 | 1 | 1 |
| 5-(1-naphthyl)-4~{H}-1,3,4-thiadiazin-2-amine | 1 | 1 | 1 | 1 | 1 | 1 | 1 | 2 | 1 |
| 5-(4-butoxyphenyl)-6~{H}-1,3,4-thiadiazin-2-amine | 3 | 3 | 3 | 3 | 1 | 3 | 1 | 1 | 1 |
| 5-(3-butoxyphenyl)-6~{H}-1,3,4-thiadiazin-2-amine | 1 | 1 | 1 | 1 | 1 | 1 | 1 | 1 | 1 |
| 5-(3,4-dichlorophenyl)-6~{H}-1,3,4-thiadiazin-2-amine | 2 | 1 | 1 | 1 | 1 | 1 | 1 | 1 | 1 |
| ethyl 3-(2-amino-6~{H}-1,3,4-thiadiazin-5-yl)benzoate | 1 | 1 | 1 | 1 | 1 | 1 | 1 | 1 | 1 |
| 5-(5-methoxypentyl)-6~{H}-1,3,4-thiadiazin-2-amine | 4 | 4 | 4 | 4 | 4 | 4 | 4 | 4 | 3 |
| 5-(3-methoxypropyl)-6~{H}-1,3,4-thiadiazin-2-amine | 4 | 4 | 3 | 4 | 4 | 4 | 3 | 3 | 2 |
| 5-(3,5-dimethylphenyl)-6~{H}-1,3,4-thiadiazin-2-amine | 2 | 1 | 1 | 1 | 1 | 2 | 1 | 1 | 1 |
| ethyl 4-(2-amino-4~{H}-1,3,4-thiadiazin-5-yl)benzoate | 4 | 4 | 4 | 4 | 3 | 4 | 3 | 4 | 2 |
| 5-heptyl-4~{H}-1,3,4-thiadiazin-2-amine | 4 | 4 | 4 | 4 | 3 | 4 | 3 | 3 | 3 |
| isopropyl 2-(2-amino-6~{H}-1,3,4-thiadiazin-5-yl)acetate | | | | | | | 1 | 2 | 1 |
| 5,6-dihydro-4~{a}~{H}-benzo[h][4,1,2]benzothiadiazin- | 1 | 2 | 1 | 1 | 2 | 1 | 1 | 1 | 1 |
| 3-amine | | | | | | | | | |
| 5,6,7,8-tetrahydro-1~{H}-4,1,2-benzothiadiazin-3-amine | 2 | 2 | 2 | 1 | 2 | 3 | 1 | 1 | 1 |
| 5-(5-phenoxypentyl)-6~{H}-1,3,4-thiadiazin-2-amine | 4 | 4 | 4 | 4 | 4 | 4 | 4 | 4 | 4 |
| 5-(4-phenylbutyl)-6~{H}-1,3,4-thiadiazin-2-amine | 4 | 4 | 4 | 4 | 4 | 4 | 2 | 4 | 2 |
| 6-butoxy-5,6,7,8-tetrahydro-4~{a}~{H}-4,1,2- | 2 | 1 | 2 | 2 | 2 | 3 | 1 | 1 | 1 |
| benzothiadiazin-3-amine | | | | | | | | | |
| 5-(4-cyclohexylphenyl)-6~{H}-1,3,4-thiadiazin-2-amine | 1 | 1 | 1 | 1 | 1 | 1 | 1 | 1 | 1 |
| 5-(4-propylphenyl)-6~{H}-1,3,4-thiadiazin-2-amine | 2 | 3 | 3 | 2 | 3 | 3 | 1 | 1 | 1 |
| 5-(4-isopropylphenyl)-6~{H}-1,3,4-thiadiazin-2-amine | 1 | 1 | 2 | 1 | 2 | 1 | 1 | 1 | 1 |
| butyl 2-(2-amino-6~{H}-1,3,4-thiadiazin-5-yl)acetate | 3 | 2 | 2 | 2 | 3 | 3 | 1 | 3 | 1 |
| 5-(2-benzyloxyethyl)-6~{H}-1,3,4-thiadiazin-2-amine | 3 | 3 | 3 | 3 | 3 | 2 | 1 | 3 | 1 |
| 5-[2-(cyclohexoxy)ethyl]-6~{H}-1,3,4-thiadiazin-2-amine | 2 | 2 | 2 | 2 | 2 | 1 | 1 | 2 | 1 |
| 5-(2-butoxyethyl)-6~{H}-1,3,4-thiadiazin-2-amine | 4 | 4 | 4 | 3 | 4 | 4 | 1 | 4 | 2 |
| 5-(2-isopropoxyethyl)-6~{H}-1,3,4-thiadiazin-2-amine | 1 | 4 | 2 | 1 | 2 | 1 | 1 | 3 | 1 |
| 5-(2-pyridyl)-6~{H}-1,3,4-thiadiazin-2-amine | 2 | 1 | 1 | 1 | 1 | 1 | 1 | 1 | 1 |
| 4-(2-amino-6~{H}-1,3,4-thiadiazin-5-yl)benzonitrile | 2 | 1 | 2 | 2 | 2 | 2 | 1 | 1 | 1 |
| 5-(4-methoxyphenyl)-6~{H}-1,3,4-thiadiazin-2-amine | 2 | 2 | 3 | 2 | 3 | 2 | 1 | 1 | 1 |
| (2-amino-6~{H}-1,3,4-thiadiazin-5-yl)methyl propanoate | 3 | 3 | 3 | 3 | 3 | 3 | 1 | 3 | 2 |
| methyl 2-(2-amino-6~{H}-1,3,4-thiadiazin-5-yl)acetate | 2 | 2 | 2 | 2 | 2 | 2 | 1 | 2 | 1 |
| benzyl 2-(2-amino-6~{H}-1,3,4-thiadiazin-5-yl)acetate | 2 | 1 | 2 | 1 | 2 | 1 | 1 | 2 | 1 |
| 5-(3-methoxyphenyl)-6~{H}-1,3,4-thiadiazin-2-amine | 1 | 1 | 1 | 1 | 1 | 1 | 1 | 1 | 1 |
| 5-(4-chlorophenyl)-6~{H}-1,3,4-thiadiazin-2-amine | 2 | 3 | 3 | 3 | 3 | 2 | 1 | 1 | 1 |
| 5-(3-chlorophenyl)-6~{H}-1,3,4-thiadiazin-2-amine | 1 | 1 | 1 | 1 | 1 | 1 | 1 | 1 | 1 |
| 5-(p-tolyl)-6~{H}-1,3,4-thiadiazin-2-amine | 3 | 3 | 2 | 3 | 3 | 3 | 1 | 2 | 1 |
| 5-(m-tolyl)-6~{H}-1,3,4-thiadiazin-2-amine | 2 | 1 | 2 | 1 | 2 | 1 | 1 | 1 | 1 |
| 5-(2-methoxyethyl)-6~{H}-1,3,4-thiadiazin-2-amine | 3 | 3 | 3 | 3 | 3 | 2 | 1 | 2 | 1 |
| 5-phenyl-6~{H}-1,3,4-thiadiazin-2-amine | 3 | 3 | 3 | 3 | 3 | 3 | 1 | 1 | 1 |
| ethyl 2-(2-amino-6~{H}-1,3,4-thiadiazin-5-yl)acetate | 3 | 2 | 3 | 2 | 3 | 2 | 3 | 3 | 2 |

| | C. GLABRATA | C. KRUSEI | A. NIGER | A. FUMIGATUS | A. TERREUS | A. FLAVUS | A. TERREUS 49 | A. FUMIGATUS 210 |
|---|---|---|---|---|---|---|---|---|
| Structure Name | RPMI media | | | | | | | |
| 5-[4-(4-ethylphenoxy)butyl]-6~{H}-1,3,4-thiadiazin-2- | 2 | 4 | 2 | 2 | 3 | 2 | 2 | 2 |
| amine | | | | | | | | |
| 5-[5-(2-pyridyloxy)pentyl]-6~{H}-1,3,4-thiadiazin-2- | 3 | 4 | 4 | 4 | 4 | 4 | 4 | 3 |
| amine | | | | | | | | |
| 5-[2-(2-phenoxyethoxy)ethyl]-6~{H}-1,3,4-thiadiazin-2- | 2 | 4 | 3 | 3 | 4 | 3 | 4 | 3 |
| amine | | | | | | | | |
| 5-[4-[[2-(2-phenylethyl)phenyl]methoxy]butyl]-6~{H}- | 1 | 2 | 1 | 1 | 2 | 1 | 2 | 1 |
| 1,3,4-thiadiazin-2-amine | | | | | | | | |
| 5-[4-[(3,5-dichlorophenyl)methoxy]butyl]-6~{H}-1,3,4- | 2 | 2 | 2 | 2 | 2 | 2 | 2 | 1 |
| thiadiazin-2-amine | | | | | | | | |
| 5-[4-[(4-chloro-2-fluoro-phenyl)methoxy]butyl]-6~{H}- | 3 | 4 | 3 | 2 | 3 | 4 | 3 | 2 |
| 1,3,4-thiadiazin-2-amine | | | | | | | | |
| 5-[4-[(2-cyclopropylphenyl)methoxy]butyl]-6~{H}-1,3,4- | 2 | 2 | 1 | 2 | 2 | 1 | 2 | 1 |
| thiadiazin-2-amine | | | | | | | | |
| 5-[4-[[2-(trifluoromethyl)phenyl]methoxy]butyl]-6~{H}- | 1 | 2 | 1 | 1 | 1 | 1 | 1 | 1 |
| 1,3,4-thiadiazin-2-amine | | | | | | | | |
| 5-[4-[(2,5-difluorophenyl)methoxy]butyl]-6~{H}-1,3,4- | 2 | 3 | 2 | 2 | 2 | 2 | 2 | 2 |
| thiadiazin-2-amine | | | | | | | | |
| 5-[4-[(2-chloro-6-fluoro-phenyl)methoxy]butyl]-6~{H}- | 2 | 3 | 2 | 2 | 2 | 2 | 2 | 1 |
| 1,3,4-thiadiazin-2-amine | | | | | | | | |
| 5-[4-[(3-butylphenyl)methoxy]butyl]-6~{H}-1,3,4- | 1 | 2 | 2 | 2 | 2 | 2 | 2 | 1 |
| thiadiazin-2-amine | | | | | | | | |
| 5-[4-[(4-bromophenyl)methoxy]butyl]-6~{H}-1,3,4- | 4 | 4 | 3 | 2 | 4 | 4 | 3 | 2 |
| thiadiazin-2-amine | | | | | | | | |
| 5-[4-[(4-chlorophenyl)methylsulfanyl]butyl]-6~{H}- | 2 | 3 | 3 | 2 | 2 | 3 | 2 | 2 |
| 1,3,4-thiadiazin-2-amine | | | | | | | | |
| 5-[4-(2-phenylethoxy)butyl]-6~{H}-1,3,4-thiadiazin-2- | 2 | 3 | 2 | 2 | 2 | 3 | 2 | 1 |
| amine | | | | | | | | |
| 4-(2-amino-6~{H}-1,3,4-thiadiazin-5-yl)butyl acetate | 2 | 4 | 2 | 3 | 3 | 3 | 3 | 3 |
| 5-[4-[(3-ethylphenyl)methoxy]butyl]-6~{H}-1,3,4- | 2 | 4 | 2 | 2 | 2 | 3 | 2 | 2 |
| thiadiazin-2-amine | | | | | | | | |
| 5-[4-[[3-(2-phenylethyl)phenyl]methoxy]butyl]-6~{H}- | 1 | 2 | 1 | 1 | 1 | 2 | 1 | 1 |
| 1,3,4-thiadiazin-2-amine | | | | | | | | |
| 5-[4-[3-(2,4-difluorophenyl)propoxy]butyl]-6~{H}-1,3,4- | 2 | 4 | 2 | 2 | 2 | 3 | 2 | 1 |
| thiadiazin-2-amine | | | | | | | | |
| 5-[4-[(3-phenylphenyl)methoxy]butyl]-6~{H}-1,3,4- | 1 | 2 | 1 | 1 | 2 | 2 | 1 | 1 |
| thiadiazin-2-amine | | | | | | | | |
| 5-[4-[[3-(trifluoromethyl)phenyl]methoxy]butyl]-6~{H}- | 1 | 2 | 2 | 2 | 2 | 2 | 2 | 1 |
| 1,3,4-thiadiazin-2-amine | | | | | | | | |
| 5-[4-[(4-chloro-3-fluoro-phenyl)methoxy]butyl]-6~{H}- | 4 | 4 | 4 | 4 | 3 | 4 | 3 | 2 |
| 1,3,4-thiadiazin-2-amine | | | | | | | | |
| 5-[4-[(3,5-difluorophenyl)methoxy]butyl]-6~{H}-1,3,4- | 3 | 4 | 3 | 4 | 3 | 3 | 3 | 2 |
| thiadiazin-2-amine | | | | | | | | |
| 5-[4-[(3-phenoxyphenyl)methoxy]butyl]-6~{H}-1,3,4- | 1 | 3 | 1 | 1 | 1 | 1 | 1 | 1 |
| thiadiazin-2-amine | | | | | | | | |
| 5-[4-[(2,4-dichlorophenyl)methoxy]butyl]-6~{H}-1,3,4- | 1 | 2 | 1 | 1 | 2 | 2 | 2 | 1 |
| thiadiazin-2-amine | | | | | | | | |
| 5-[4-[(3-bromophenyl)methoxy]butyl]-6~{H}-1,3,4- | 3 | 4 | 3 | 4 | 3 | 4 | 3 | 2 |
| thiadiazin-2-amine | | | | | | | | |
| 5-[4-[(3-cyclopropylphenyl)methoxy]butyl]-6~{H}-1,3,4- | 2 | 4 | 2 | 3 | 2 | 2 | 2 | 2 |
| thiadiazin-2-amine | | | | | | | | |
| 5-[4-[(4-fluoro-3-methyl-phenyl)methoxy]butyl]-6~{H}- | 3 | 4 | 3 | 4 | 3 | 4 | 3 | 2 |
| 1,3,4-thiadiazin-2-amine | | | | | | | | |
| 5-[4-[(4-fluorophenyl)methoxy]butyl]-6~{H}-1,3,4- | 2 | 2 | 1 | 1 | 1 | 1 | 1 | 1 |
| thiadiazin-2-amine | | | | | | | | |
| 5-[4-[(4-methoxyphenyl)methoxy]butyl]-6~{H}-1,3,4- | 2 | 2 | 1 | 1 | 1 | 1 | 1 | 1 |
| thiadiazin-2-amine | | | | | | | | |
| 5-[4-[(2,4-difluorophenyl)methoxy]butyl]-6~{H}-1,3,4- | 4 | 4 | 4 | 4 | 4 | 4 | 4 | 3 |
| thiadiazin-2-amine | | | | | | | | |
| 5-[4-[(3-chlorophenyl)methoxy]butyl]-6~{H}-1,3,4- | 3 | 4 | 3 | 4 | 3 | 4 | 3 | 2 |
| thiadiazin-2-amine | | | | | | | | |
| 5-[4-(m-tolylmethoxy)butyl]-6~{H}-1,3,4-thiadiazin-2- | 2 | 3 | 2 | 3 | 2 | 3 | 2 | 2 |
| amine | | | | | | | | |
| 5-[4-[(4-butylphenyl)methoxy]butyl]-6~{H}-1,3,4- | 2 | 3 | 2 | 1 | 1 | 2 | 1 | 1 |
| thiadiazin-2-amine | | | | | | | | |
| 5-[4-[[4-(trifluoromethyl)phenyl]methoxy]butyl]-6~{H}- | 2 | 4 | 3 | 2 | 3 | 3 | 2 | 2 |
| 1,3,4-thiadiazin-2-amine | | | | | | | | |
| 5-[4-[(4-chlorophenyl)methoxy]butyl]-6~{H}-1,3,4- | 3 | 4 | 3 | 3 | 3 | 4 | 3 | 2 |
| thiadiazin-2-amine | | | | | | | | |
| 5-[5-(4-butylphenoxy)pentyl]-6~{H}-1,3,4-thiadiazin-2- | 2 | 3 | 1 | 1 | 2 | 1 | 2 | 1 |
| amine | | | | | | | | |
| 5-[2-[(4-butylphenyl)methoxy]ethyl]-6~{H}-1,3,4- | 2 | 3 | 2 | 1 | 2 | 3 | 2 | 2 |
| thiadiazin-2-amine | | | | | | | | |
| 5-[2-[3-(4-methoxyphenyl)propoxy]ethyl]-6~{H}-1,3,4- | 1 | 3 | 1 | 1 | 1 | 1 | 1 | 1 |
| thiadiazin-2-amine | | | | | | | | |
| 5-[5-(4-ethylphenoxy)pentyl]-6~{H}-1,3,4-thiadiazin-2- | 2 | 4 | 3 | 2 | 2 | 3 | 2 | 2 |
| amine | | | | | | | | |
| 5-[2-(4-methoxybutoxy)ethyl]-6~{H}-1,3,4-thiadiazin-2- | 2 | 4 | 2 | 2 | 2 | 2 | 2 | 2 |
| amine | | | | | | | | |
| 5-[2-(3-phenylpropoxy)ethyl]-6~{H}-1,3,4-thiadiazin-2- | 2 | 4 | 3 | 4 | 3 | 3 | 3 | 3 |
| amine | | | | | | | | |
| butyl 4-(2-amino-6~{H}-1,3,4-thiadiazin-5-yl)benzoate | 1 | 1 | 1 | 1 | 1 | 1 | 1 | 1 |
| 5-[5-(3-ethylphenoxy)pentyl]-6~{H}-1,3,4-thiadiazin-2- | 3 | 4 | 3 | 2 | 3 | 3 | 3 | 2 |
| amine | | | | | | | | |
| 5-(4-butoxyphenyl)-4-methyl-1,3,4-thiadiazin-2-amine | 2 | 2 | 3 | 4 | 3 | 4 | 3 | 4 |
| 5-(4-benzyloxybutyl)-6~{H}-1,3,4-thiadiazin-2-amine | 3 | 4 | 3 | 4 | 4 | 4 | 3 | 3 |
| 5-(4-methoxyphenyl)-4-methyl-1,3,4-thiadiazin-2-amine | 1 | 4 | 3 | 4 | 4 | 3 | 4 | 4 |
| 5-(benzofuran-2-yl)-4-methyl-1,3,4-thiadiazin-2-amine | 1 | 2 | 2 | 2 | 2 | 1 | 2 | 2 |
| 4-(2-amino-4-methyl-1,3,4-thiadiazin-5-yl)benzonitrile | 1 | 4 | 3 | 3 | 4 | 3 | 4 | 3 |
| ethyl 4-(2-amino-4-methyl-1,3,4-thiadiazin-5-yl)benzoate | 2 | 3 | 3 | 4 | 4 | 4 | 4 | 4 |
| 5-(3,4-dichlorophenyl)-4-methyl-1,3,4-thiadiazin-2-amine | 1 | 1 | 1 | 2 | 2 | 1 | 2 | 1 |
| 4-methyl-5-(p-tolyl)-1,3,4-thiadiazin-2-amine | 1 | 3 | 3 | 1 | 4 | 3 | 4 | 1 |
| 5-[2-(4-pentoxyphenyl)ethyl]-6~{H}-1,3,4-thiadiazin-2- | 1 | 3 | 3 | 2 | 3 | 4 | 3 | 2 |
| amine | | | | | | | | |
| 5-(5-benzyloxypentyl)-6~{H}-1,3,4-thiadiazin-2-amine | 2 | 4 | 3 | 3 | 3 | 3 | 3 | 3 |
| 5-[4-(benzofuran-2-yl)butyl]-6~{H}-1,3,4-thiadiazin-2- | 2 | 3 | 3 | 3 | 3 | 3 | 3 | 3 |
| amine | | | | | | | | |
| 5-(5-methoxypentyl)-4-methyl-3~{H}-1,3,4-thiadiazin-2- | 1 | 4 | 3 | 2 | 3 | 3 | 3 | 3 |
| imine | | | | | | | | |
| 5-(4-chlorophenyl)-4-methyl-1,3,4-thiadiazin-2-amine | 1 | 4 | 4 | 4 | 3 | 4 | 3 | 4 |
| 2-[5-(2-amino-6~{H}-1,3,4-thiadiazin-5- | 1 | 4 | 3 | 2 | 2 | 2 | 2 | 1 |
| yl)pentyl]isoindoline-1,3-dione | | | | | | | | |
| 5-[5-(4-fluorophenoxy)pentyl]-6~{H}-1,3,4-thiadiazin-2- | 2 | 4 | 4 | 4 | 4 | 4 | 4 | 3 |
| amine | | | | | | | | |
| 5-[(~{E})-hept-1-enyl]-6~{H}-1,3,4-thiadiazin-2-amine | 1 | 2 | 1 | 2 | 1 | 2 | 1 | 1 |
| 4-(5-methoxypentyl)-5-methyl-1,3,4-thiadiazin-2-amine | 1 | 3 | 1 | 1 | 1 | 1 | 1 | 1 |
| 5-[5-(2-pyridyl)pentyl]-6~{H}-1,3,4-thiadiazin-2-amine | 1 | 4 | 3 | 3 | 2 | 2 | 2 | 2 |
| 5-[5-(~{N}-methylanilino)pentyl]-6~{H}-1,3,4- | 1 | 2 | 1 | 1 | 1 | 1 | 1 | 1 |
| thiadiazin-2-amine | | | | | | | | |
| 5-(6-methoxyhexyl)-6~{H}-1,3,4-thiadiazin-2-amine | 2 | 4 | 4 | 4 | 3 | 3 | 3 | 3 |
| 6-(2-butoxyethyl)-4~{H}-1,3,4-thiadiazin-2-amine | 1 | 1 | 2 | 1 | 2 | 2 | 3 | 1 |
| 5-(7-phenoxyheptyl)-6~{H}-1,3,4-thiadiazin-2-amine | 2 | 3 | 4 | 3 | 2 | 3 | 3 | 3 |
| 5-(7-methoxyheptyl)-6~{H}-1,3,4-thiadiazin-2-amine | 2 | 4 | 4 | 4 | 3 | 3 | 3 | 4 |
| 5-(4-methoxybutyl)-6~{H}-1,3,4-thiadiazin-2-amine | 1 | 4 | 2 | 3 | 2 | 2 | 2 | 2 |
| 6-(2-amino-6~{H}-1,3,4-thiadiazin-5-yl)hexanoic acid | 1 | 2 | 1 | 1 | 1 | 1 | 1 | 1 |
| ethyl 6-(2-amino-6~{H}-1,3,4-thiadiazin-5-yl)hexanoate | 1 | 4 | 3 | 4 | 3 | 3 | 3 | 4 |
| 5-(methoxymethyl)-6~{H}-1,3,4-thiadiazin-2-amine | 1 | 4 | 1 | 1 | 2 | 2 | 1 | 1 |
| 5-(4-phenoxybutyl)-6~{H}-1,3,4-thiadiazin-2-amine | 2 | 4 | 3 | 3 | 3 | 3 | 3 | 3 |
| 5-[2-(4-phenylphenoxy)ethyl]-6~{H}-1,3,4-thiadiazin-2- | 1 | 1 | 1 | 1 | 1 | 1 | 1 | 1 |
| amine | | | | | | | | |
| 5-[2-(4-methylphenoxy)ethyl]-6~{H}-1,3,4-thiadiazin-2- | 1 | 3 | 1 | 1 | 1 | 1 | 1 | 1 |
| amine | | | | | | | | |
| 5-(phenoxymethyl)-6~{H}-1,3,4-thiadiazin-2-amine | 1 | 4 | 2 | 3 | 2 | 2 | 2 | 2 |
| 5-(3-phenoxypropyl)-6~{H}-1,3,4-thiadiazin-2-amine | 3 | 4 | 2 | 3 | 2 | 3 | 2 | 2 |
| 5-(6-phenoxyhexyl)-6~{H}-1,3,4-thiadiazin-2-amine | 2 | 3 | 4 | 2 | 3 | 3 | 3 | 2 |
| 5-(6-phenylhexyl)-6~{H}-1,3,4-thiadiazin-2-amine | 2 | 3 | 4 | 2 | 2 | 3 | 2 | 2 |
| 5-(2-amino-6~{H}-1,3,4-thiadiazin-5-yl)-~{N}-phenyl- | 1 | 2 | 1 | 1 | 1 | 1 | 1 | 1 |
| pentanamide | | | | | | | | |
| 5-[2-[(4-methoxyphenyl)methoxy]ethyl]-6~{H}-1,3,4- | 1 | 3 | 1 | 2 | 1 | 1 | 1 | 1 |
| thiadiazin-2-amine | | | | | | | | |
| 5-[2-(4-propoxyphenyl)ethyl]-6~{H}-1,3,4-thiadiazin-2- | 2 | 3 | 3 | 3 | 3 | 3 | 3 | 3 |
| amine | | | | | | | | |
| 4-[2-(2-amino-6~{H}-1,3,4-thiadiazin-5- | 1 | 3 | 2 | 2 | 2 | 2 | 2 | 1 |
| yl)ethoxymethyl]benzonitrile | | | | | | | | |
| 5-[2-(1-methylbutoxy)ethyl]-6~{H}-1,3,4-thiadiazin-2- | 1 | 3 | 1 | 1 | 2 | 1 | 1 | 1 |
| amine | | | | | | | | |
| 5-[2-(p-tolylmethoxy)ethyl]-6~{H}-1,3,4-thiadiazin-2- | 1 | 3 | 3 | 3 | 3 | 3 | 3 | 3 |
| amine | | | | | | | | |
| 5-[2-[(4-phenylphenyl)methoxy]ethyl]-6~{H}-1,3,4- | 1 | 2 | 2 | 1 | 3 | 2 | 2 | 1 |
| thiadiazin-2-amine | | | | | | | | |
| 5-(2-hexoxyethyl)-6~{H}-1,3,4-thiadiazin-2-amine | 2 | 4 | 4 | 4 | 4 | 4 | 4 | 4 |
| 5-[2-(2-methoxyethoxy)ethyl]-6~{H}-1,3,4-thiadiazin-2- | 1 | 3 | 2 | 1 | 2 | 1 | 2 | 1 |
| amine | | | | | | | | |
| 5-(2-phenoxyethyl)-6~{H}-1,3,4-thiadiazin-2-amine | 1 | 4 | 1 | 1 | 2 | 2 | 1 | 1 |
| 5-[2-(4-bromophenoxy)ethyl]-6~{H}-1,3,4-thiadiazin-2- | 1 | 2 | 2 | 2 | 2 | 3 | 2 | 2 |
| amine | | | | | | | | |
| 5-(2-naphthyl)-6~{H}-1,3,4-thiadiazin-2-amine | 1 | 1 | 1 | 1 | 1 | 1 | 1 | 1 |
| 5-(1-naphthyl)-4~{H}-1,3,4-thiadiazin-2-amine | 1 | 2 | 1 | 2 | 1 | 1 | 1 | 1 |
| 5-(4-butoxyphenyl)-6~{H}-1,3,4-thiadiazin-2-amine | 1 | 1 | 1 | 1 | 1 | 1 | 1 | 1 |
| 5-(3-butoxyphenyl)-6~{H}-1,3,4-thiadiazin-2-amine | 1 | 1 | 1 | 1 | 1 | 1 | 1 | 1 |
| 5-(3,4-dichlorophenyl)-6~{H}-1,3,4-thiadiazin-2-amine | 1 | 1 | 1 | 1 | 1 | 1 | 1 | 1 |
| ethyl 3-(2-amino-6~{H}-1,3,4-thiadiazin-5-yl)benzoate | 1 | 1 | 1 | 1 | 1 | 1 | 1 | 1 |
| 5-(5-methoxypentyl)-6~{H}-1,3,4-thiadiazin-2-amine | 1 | 4 | 4 | 4 | 4 | 4 | 4 | 4 |
| 5-(3-methoxypropyl)-6~{H}-1,3,4-thiadiazin-2-amine | 1 | 4 | 3 | 3 | 2 | 3 | 2 | 2 |
| 5-(3,5-dimethylphenyl)-6~{H}-1,3,4-thiadiazin-2-amine | 1 | 1 | 1 | 1 | 1 | 1 | 1 | 1 |
| ethyl 4-(2-amino-4~{H}-1,3,4-thiadiazin-5-yl)benzoate | 1 | 3 | 3 | 4 | 3 | 4 | 3 | 3 |
| 5-heptyl-4~{H}-1,3,4-thiadiazin-2-amine | 3 | 3 | 2 | 2 | 2 | 3 | 2 | 2 |
| isopropyl 2-(2-amino-6~{H}-1,3,4-thiadiazin-5-yl)acetate | 1 | 2 | 1 | 1 | 1 | 1 | 1 | 1 |
| 5,6-dihydro-4~{a}~{H}-benzo[h][4,1,2]benzothiadiazin- | 1 | 1 | 1 | 1 | 1 | 1 | 1 | 1 |
| 3-amine | | | | | | | | |
| 5,6,7,8-tetrahydro-1~{H}-4,1,2-benzothiadiazin-3-amine | 2 | 2 | 1 | 1 | 1 | 1 | 1 | 1 |
| 5-(5-phenoxypentyl)-6~{H}-1,3,4-thiadiazin-2-amine | 2 | 4 | 4 | 4 | 4 | 4 | 4 | 4 |
| 5-(4-phenylbutyl)-6~{H}-1,3,4-thiadiazin-2-amine | 2 | 3 | 3 | 3 | 2 | 4 | 3 | 2 |
| 6-butoxy-5,6,7,8-tetrahydro-4~{a}~{H}-4,1,2- | 1 | 2 | 1 | 1 | 1 | 1 | 1 | 1 |
| benzothiadiazin-3-amine | | | | | | | | |
| 5-(4-cyclohexylphenyl)-6~{H}-1,3,4-thiadiazin-2-amine | 1 | 1 | 1 | 1 | 1 | 1 | 1 | 1 |
| 5-(4-propylphenyl)-6~{H}-1,3,4-thiadiazin-2-amine | 1 | 1 | 1 | 1 | 1 | 1 | 1 | 1 |
| 5-(4-isopropylphenyl)-6~{H}-1,3,4-thiadiazin-2-amine | 1 | 1 | 1 | 1 | 1 | 1 | 1 | 1 |
| butyl 2-(2-amino-6~{H}-1,3,4-thiadiazin-5-yl)acetate | 1 | 4 | 2 | 1 | 2 | 2 | 2 | 2 |
| 5-(2-benzyloxyethyl)-6~{H}-1,3,4-thiadiazin-2-amine | 1 | 2 | 1 | 1 | 2 | 2 | 2 | 1 |
| 5-[2-(cyclohexoxy)ethyl]-6~{H}-1,3,4-thiadiazin-2-amine | 1 | 2 | 1 | 1 | 2 | 2 | 1 | 1 |
| 5-(2-butoxyethyl)-6~{H}-1,3,4-thiadiazin-2-amine | 1 | 4 | 2 | 4 | 3 | 3 | 3 | 3 |
| 5-(2-isopropoxyethyl)-6~{H}-1,3,4-thiadiazin-2-amine | 1 | 3 | 1 | 1 | 1 | 1 | 2 | 1 |
| 5-(2-pyridyl)-6~{H}-1,3,4-thiadiazin-2-amine | 1 | 1 | 1 | 1 | 1 | 1 | 1 | 1 |
| 4-(2-amino-6~{H}-1,3,4-thiadiazin-5-yl)benzonitrile | 1 | 1 | 1 | 1 | 1 | 1 | 1 | 1 |
| 5-(4-methoxyphenyl)-6~{H}-1,3,4-thiadiazin-2-amine | 1 | 1 | 1 | 1 | 1 | 1 | 1 | 1 |
| (2-amino-6~{H}-1,3,4-thiadiazin-5-yl)methyl propanoate | 1 | 3 | 1 | 2 | 2 | 2 | 2 | 1 |
| methyl 2-(2-amino-6~{H}-1,3,4-thiadiazin-5-yl)acetate | 1 | 3 | 1 | 1 | 1 | 1 | 1 | 1 |
| benzyl 2-(2-amino-6~{H}-1,3,4-thiadiazin-5-yl)acetate | 1 | 2 | 1 | 1 | 1 | 1 | 1 | 1 |
| 5-(3-methoxyphenyl)-6~{H}-1,3,4-thiadiazin-2-amine | 1 | 1 | 1 | 1 | 1 | 1 | 1 | 1 |
| 5-(4-chlorophenyl)-6~{H}-1,3,4-thiadiazin-2-amine | 1 | 1 | 2 | 1 | 1 | 1 | 1 | 1 |
| 5-(3-chlorophenyl)-6~{H}-1,3,4-thiadiazin-2-amine | 1 | 1 | 1 | 1 | 1 | 1 | 1 | 1 |
| 5-(p-tolyl)-6~{H}-1,3,4-thiadiazin-2-amine | 1 | 2 | 2 | 2 | 2 | 1 | 1 | 2 |
| 5-(m-tolyl)-6~{H}-1,3,4-thiadiazin-2-amine | 1 | 1 | 1 | 1 | 1 | 1 | 1 | 1 |
| 5-(2-methoxyethyl)-6~{H}-1,3,4-thiadiazin-2-amine | 1 | 4 | 3 | 2 | 3 | 3 | 2 | 2 |
| 5-phenyl-6~{H}-1,3,4-thiadiazin-2-amine | 1 | 2 | 1 | 1 | 2 | 1 | 1 | 1 |
| ethyl 2-(2-amino-6~{H}-1,3,4-thiadiazin-5-yl)acetate | 1 | 3 | 2 | 2 | 2 | 2 | 2 | 2 |

1. A compound which is a diazine of formula (I) or a tautomer thereof, or a pharmaceutically acceptable salt thereof, for use as an antifungal agent: 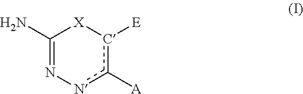
wherein:
X represents O or S;
the moiety 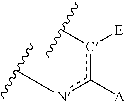
represents —N(D)-C(A)═C(E)- or —N═C(A)-C(R¹)(E)-;
D represents H or C₁-C₆ alkyl, wherein the alkyl group of D is unsubstituted or is substituted with 1, 2 or 3 substituents selected from halogen, OH, and C₁-C₂ alkoxy; and wherein the alkyl group of D is uninterrupted or is interrupted by —O—, —C(O)—, —OC(O)— or —C(O)O—;
R¹ is H or C₁-C₂ alkyl;
one group selected from A and E represents a group Q1, and the other group selected from A and E represents a group Q2;
Q1 represents
(i) H or C₁-C₈ alkyl, wherein the alkyl group of Q1 is unsubstituted or is substituted with 1, 2 or 3 substituents selected from halogen, OH, and C₁-C₂ alkoxy; and wherein the alkyl group of Q1 is uninterrupted or is interrupted by —O—, —C(O)—, —OC(O)— or —C(O)O—;
or
(ii) an alkylene group which is bonded to an atom of group Q2 to form a C₅-C₆ carbocyclyl or 5- to 6-membered heterocyclyl moiety, wherein the carbocylyl or heterocyclyl moiety is saturated or partially unsaturated; and wherein the carbocyclyl or heterocyclyl moiety is unsubstituted or is substituted with 1, 2 or 3 substituents selected from halogen, C₁-C₄ alkoxy, unsubstituted C₁-C₄ alkyl and C₁-C₄ alkyl substituted with 1, 2 or 3 substituents independently selected from halogen and —OH;
Q2 represents a group -L-T or -T, wherein
L is selected from C₁-C₁₂ alkylene and C₂-C₁₂ alkenylene, wherein the alkylene or alkenylene group of L is unsubstituted or is substituted with 1, 2 or 3 groups selected from halogen, C₁-C₄ alkoxy and —OH; and
wherein the alkylene or alkenylene group of L may optionally terminate in and/or be interrupted by a heteromoiety selected from —O—, —S—, —C(O)—, —OC(O)—, —C(O)O—, —NR²—, —NR²C(O)—, and —C(O)NR²—; and
when Q2 is -L-T, then T is H, aryl, heteroaryl, cycloalkyl or heterocyclyl, and when Q2 is -T, then T is aryl, heteroaryl, cycloalkyl or heterocyclyl, wherein the aryl, heteroaryl, cycloalkyl or heterocyclyl group of T is unsubstituted or is substituted by 1, 2 or 3 groups V;
each group V is independently selected from C₁-C₆ alkoxy, unsubstituted C₁-C₁₀ alkyl, C₁-C₁₀ alkyl which is substituted with 1, 2 or 3 groups selected from halogen and C₁-C₃ alkoxy, C₂-C₆ alkenyl, C₂-C₆ alkynyl, C₃-C₆ cycloalkyl, halogen, aryl, (C₁-C₆ alkyl)-aryl, aryloxy, aryloxy-(C₁-C₆ alkyl), —CN, NO₂, —(C₁-C₆ alkyl)-C(O)O(C₁-C₆ alkyl) and —C(O)O(C₁-C₆ alkyl); and
R² is H or C₁-C₂ alkyl.

2. A compound for use according to claim 1 wherein
X represents O or S;
the moiety 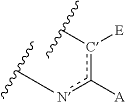
represents —N(D)-C(A)═C(E)- or —N═C(A)-C(R¹)(E)-;
D represents H or C₁-C₆ alkyl, wherein the alkyl group of D is unsubstituted or is substituted with 1, 2 or 3 substituents selected from halogen, OH, and C₁-C₂ alkoxy; and wherein the alkyl group of D is uninterrupted or is interrupted by —O—, —C(O)—, —OC(O)— or —C(O)O—;
R¹ is H or C₁-C₂ alkyl;
E represents
H or C₁-C₈ alkyl, wherein the alkyl group of E is unsubstituted or is substituted with 1, 2 or 3 substituents selected from halogen, OH, and C₁-C₂ alkoxy; and wherein the alkyl group of E is uninterrupted or is interrupted by —O—, —C(O)—, —OC(O)— or —C(O)O—;
or
(ii) an alkylene group which is bonded to an atom of group A to form a C₅-C₆ carbocyclyl or 5- to 6-membered heterocyclyl moiety, wherein the carbocylyl or heterocyclyl moiety is saturated or partially unsaturated; and
wherein the carbocyclyl or heterocyclyl moiety is unsubstituted or is substituted with 1, 2 or 3 substituents selected from halogen, C₁-C₄ alkoxy, unsubstituted C₁-C₄ alkyl and C₁-C₄ alkyl substituted with 1, 2 or 3 substituents independently selected from halogen and —OH;
A represents a group -L-T or -T, wherein
L is selected from C₁-C₁₂ alkylene and C₂-C₁₂ alkenylene, wherein the alkylene or alkenylene group of L is unsubstituted or is substituted with 1, 2 or 3 groups selected from halogen, C₁-C₄ alkoxy and —OH; and wherein the alkylene or alkenylene group of L may optionally terminate in and/or be interrupted by a heteromoiety selected from —O—, —S—, —C(O)—, —OC(O)—, —C(O)O—, —NR²—, —NR²C(O)—, and —C(O)NR²—; and
when A is -L-T, then T is H, aryl, heteroaryl, cycloalkyl or heterocyclyl, and when A is -T, then T is aryl, heteroaryl, cycloalkyl or heterocyclyl, wherein the aryl, heteroaryl, cycloalkyl or heterocyclyl group of T is unsubstituted or is substituted by 1, 2 or 3 groups V;
each group V is independently selected from C₁-C₆ alkoxy, unsubstituted alkyl, C₁-C₁₀ alkyl which is substituted with 1, 2 or 3 groups selected from halogen and C₁-C₃ alkoxy, C₂-C₆ alkenyl, C₂-C₆ alkynyl C₃-C₆ cycloalkyl, halogen, aryl, (C₁-C₆ alkyl)-aryl, aryloxy, aryloxy-(C₁-C₆ alkyl), —CN, NO₂, —(C₁-C₆ alkyl)-C(O)O(C₁-C₆ alkyl), and —C(O)O(C₁-C₆ alkyl); and
R² is H or C₁-C₂ alkyl.

3. A compound for use according to claim 1 or 2 wherein X is S.

4. A compound for use according to any one of the preceding claims wherein:
(i) the compound is of Formula (Ia) 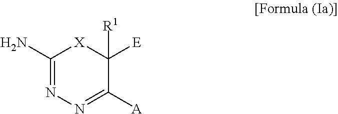
and
R¹ is H or C₁-C₂ alkyl; or
(ii) the compound is of Formula (Ib) 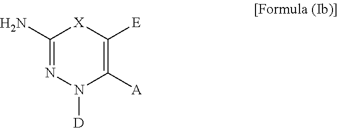
and
D is H or C₁-C₆ alkyl; wherein the alkyl group is unsubstituted or is substituted with 1 or 2 substituents selected from halogen, OH and C₁-C₂ alkoxy.

5. A compound for use according to any one of the preceding claims wherein:
(i) E is H or C₁-C₆ alkyl which is unsubstituted or is substituted with 1 substituent selected from halogen and C₁-C₂ alkoxy; and wherein the alkyl group of E is uninterrupted or is interrupted by —O—; or
(ii) E is a C₁-C₃ alkylene group which is bonded to an atom of group A to form a C₅-C₆ carbocyclyl moiety, which is unsubstituted or is substituted by 1 or 2 substituents selected from halogen, C₁-C₄ alkoxy and unsubstituted C₁-C₄ alkyl.

6. A compound for use according to any one of the preceding claims wherein:
A is -L-T; and
L is selected from C₁-C₁₀ alkylene and C₂-C₁₀ alkenylene, wherein L is unsubstituted or is substituted by 1 or 2 groups selected from halogen and C₁-C₄ alkoxy; and wherein L is uninterrupted or is interrupted by a heteromoiety selected from —O—, —S—, —C(O)O—, —NR²—, and —C(O)NR²—; and wherein L may optionally terminate in a heteromoiety selected from —O—, —S—, —C(O)O—, —NR²—, and —C(O)NR²—

7. A compound for use according to any one of the preceding claims wherein:
A is L-T, and T is H or aryl or heteroaryl, or A is -T and T is aryl or heteroaryl
when T is aryl or heteroaryl then the aryl or heteroaryl group is unsubstituted or is substituted by 1 or 2 groups V; and
each V is independently selected from C₁-C₄ alkoxy, unsubstituted C₁-C₄ alkyl, C₁-C₄ alkyl which is substituted with 1, 2 or 3 halogen atoms, C₂-C₄ alkenyl, C₂-C₄ alkynyl, C₃-C₆ cycloalkyl, halogen, aryl, (C₁-C₄ alkyl)-aryl, aryloxy, aryloxy-(C₁-C₄ alkyl), —CN, NO₂, —(C₁-C₄ alkyl)-C(O)O(C₁-C₄ alkyl), and —C(O)O(C₁-C₄ alkyl).

8. A compound for use according to any one of the preceding claims wherein the compound is of Formula (Ia): 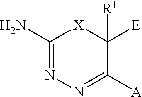
wherein:
X represents S;
R¹ represents H or C₁-C₂ alkyl;
E represents either:
(i) H or C₁-C₆ alkyl which is unsubstituted or is substituted with 1 substituent selected from halogen, and C₁-C₂ alkoxy; and wherein the alkyl group of E is uninterrupted or is interrupted by —O—; or
(ii) a C₁-C₆ alkylene group which is bonded to an atom of group A to form a C₅-C₆ carbocyclyl moiety which is unsubstituted or is substituted by 1 or 2 substituents selected from halogen, C₁-C₄ alkoxy and unsubstituted C₁-C₄ alkyl;
A represents the group -L-T or -T;
L is selected from C₁-C₁₀ alkylene and C₂-C₁₀ alkenylene, wherein the alkylene or alkenylene group is unsubstituted or is substituted by 1 or 2 groups selected from halogen, and C₁-C₄ alkoxy, and wherein the alkylene or alkenylene group is uninterrupted or is interrupted by a heteromoiety selected from —O—, —S—, —C(O)O—, —NR²—, and —C(O)NR²— and wherein the alkylene or alkenylene group optionally terminates in a heteromoiety selected from —O—, —S—, —C(O)—, —OC(O)—, —C(O)O—, —NR²—, —NR²C(O)—, and —C(O)NR²—, wherein R² represents H or methyl;
when A is -L-T, then T is H or represents a 5- to 10-membered heteroaryl group or a 6- to 10-membered aryl group, and when A is -T, then T represents a 5- to 10-membered heteroaryl group or a 6- to 10-membered aryl group; wherein the aryl or heteroaryl group of T is unsubstituted or is substituted by 1 or 2 groups V;
each group V is independently selected from C₁-C₄ alkoxy, unsubstituted C₁-C₄ alkyl, C₁-C₄ alkyl which is substituted with 1, 2 or 3 halogen atoms, C₂-C₄ alkenyl, C₂-C₄ alkynyl, C₃-C₆ cycloalkyl, halogen, aryl, (C₁-C₄ alkyl)-aryl, aryloxy, aryloxy-(C₁-C₄ alkyl), —CN, NO₂, —(C₁-C₄ alkyl)-C(O)O(C₁-C₄ alkyl), and —C(O)O(C₁-C₄ alkyl).

9. A compound for use according to any one of the preceding claims wherein:
the compound is of Formula (Ia): 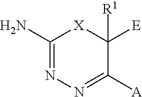
wherein
X represents S;
R¹ represents H or methyl
E represents either:
(i) H or C₄-C₆ alkyl which is unsubstituted and which is uninterrupted or is interrupted by —O—; or
(ii) E is a C₁-C₄ alkylene group which is bonded to an atom of group A to form a C₆ carbocyclylene group which is unsubstituted or is substituted by 1 substitutent selected from C₁-C₄ alkoxy
A represents the group -L-T or -T;
L is selected from C₁-C₁₀ alkylene and C₂-C₁₀ alkenylene, wherein the alkylene or alkenylene group is unsubstituted or is substituted by 1 group selected from halogen and C₁-C₄ alkoxy, and wherein the alkylene or alkenylene group is uninterrupted or is interrupted by a heteromoiety selected from —O—, —S—, and —C(O)O—, and wherein the alkylene or alkenylene group optionally terminates in a heteromoiety selected from —O—, and —C(O)O—
When A is L-T, then T is H or represents phenyl, napthyl, benzofuranyl, pyridyl, isoindoline-1,3-dione, benzothiazole, tetrahydrofuran, thienyl or cyclohexyl, and when A is T, then T represents phenyl, napthyl, benzofuranyl, pyridyl, isoindoline-1,3-dione, benzothiazole, tetrahydrofuran, thienyl or cyclohexyl;
wherein T is unsubstituted or is substituted by 1 V group;
Each V group is independently selected from C₁-C₄ alkoxy, unsubstituted C₁-C₄ alkyl, C₁-C₄ alkyl which is substituted with 1, 2 or 3 halogen atoms, C₂-C₄ alkenyl, C₂-C₄ alkynyl, C₃-C₆ cycloalkyl, halogen, aryl, (C₁-C₄ alkyl)-aryl, aryloxy, aryloxy-(C₁-C₄ alkyl), —CN, NO₂, —(C₁-C₄ alkyl)-C(O)O(C₁-C₄ alkyl), and —C(O)O(C₁-C₄ alkyl).

10. A compound for use according to any one of the preceding claims wherein:
the compound is of Formula (Ia): 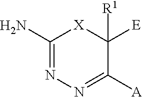
wherein:
X represents S;
R¹ represents H
E represents H or C₄-C₆ alkyl which is unsubstituted and which is uninterrupted or is interrupted by —O—;
A represents a group -L-T or -T;
L is selected from C₁-C₁₀ alkylene and C₂-C₁₀ alkenylene, wherein the alkylene or alkenylene group is unsubstituted, and wherein the alkylene or alkenylene group is uninterrupted or is interrupted by a heteromoiety selected from —O— and —C(O)O—, and wherein the alkylene or alkenylene group optionally terminates in —O—;
When A is L-T, then T is H or represents phenyl, and when A is T, then T is phenyl; wherein the phenyl group is unsubstituted or is substituted by 1 group V,
Each V is independently unsubstituted C₁-C₃ alkyl, halogen, —C(O)O(C₁-C₃ alkyl), (C₁-C₃ alkyl)-aryl and aryloxy.

11. A compound for use according claim 1 wherein the compound is selected from:
5-(5-pentoxypentyl)-6˜{H}-1,3,4-thiadiazin-2-amine; 5-[3-(2-phenylethoxy)propyl]-6˜{H}-1,3,4-thiadiazin-2-amine; 5-[4-(4-ethylphenoxy)butyl]-6˜{H}-1,3,4-thiadiazin-2-amine; 5-[5-(2-pyridyloxy)pentyl]-6˜{H}-1,3,4-thiadiazin-2-amine; 5-[2-(2-phenoxyethoxy)ethyl]-6˜{H}-1,3,4-thiadiazin-2-amine; 5-[4-[[2-(2-phenylethyl)phenyl]methoxy]butyl]-6˜{H}-1,3,4-thiadiazin-2-amine; 5-[4-[(3,5-dichlorophenyl)methoxy]butyl]-6˜{H}-1,3,4-thiadiazin-2-amine; 5-[4-[(4-chloro-2-fluoro-phenyl)methoxy]butyl]-6˜{H}-1,3,4-thiadiazin-2-amine; 5-[4-[(2-cyclopropylphenyl)methoxy]butyl]-6˜{H}-1,3,4-thiadiazin-2-amine; 5-[4-[[2-(trifluoromethyl)phenyl]methoxy]butyl]-6˜{H}-1,3,4-thiadiazin-2-amine; 5-[4-[(2,5-difluorophenyl)methoxy]butyl]-6˜{H}-1,3,4-thiadiazin-2-amine; 5-[4-[(2-chloro-6-fluoro-phenyl)methoxy]butyl]-6˜{H}-1,3,4-thiadiazin-2-amine; 5-[4-[(3-butylphenyl)methoxy]butyl]-6˜{H}-1,3,4-thiadiazin-2-amine; 5-[4-[(4-bromophenyl)methoxy]butyl]-6˜{H}-1,3,4-thiadiazin-2-amine; 5-[4-[(4-chlorophenyl)methylsulfanyl]butyl]-6˜{H}-1,3,4-thiadiazin-2-amine; 5-[4-(2-phenylethoxy)butyl]-6˜{H}-1,3,4-thiadiazin-2-amine; 4-(2-amino-6˜{H}-1,3,4-thiadiazin-5-yl)butyl acetate; 5-[4-[(3-ethylphenyl)methoxy]butyl]-6˜{H}-1,3,4-thiadiazin-2-amine; 5-[4-[[3-(2-phenylethyl)phenyl]methoxy]butyl]-6˜{H}-1,3,4-thiadiazin-2-amine; 5-[4-[3-(2,4-difluorophenyl)propoxy]butyl]-6˜{H}-1,3,4-thiadiazin-2-amine; 5-[4-[(3-phenylphenyl)methoxy]butyl]-6˜{H}-1,3,4-thiadiazin-2-amine; 5-[4-[[3-(trifluoromethyl)phenyl]methoxy]butyl]-6˜{H}-1,3,4-thiadiazin-2-amine; 5-[4-[(4-chloro-3-fluoro-phenyl)methoxy]butyl]-6˜{H}-1,3,4-thiadiazin-2-amine; 5-[4-[(3,5-difluorophenyl)methoxy]butyl]-6˜{H}-1,3,4-thiadiazin-2-amine; 5-[4-[(3-phenoxyphenyl)methoxy]butyl]-6˜{H}-1,3,4-thiadiazin-2-amine; 5-[4-[(2,4-dichlorophenyl)methoxy]butyl]-6˜{H}-1,3,4-thiadiazin-2-amine; 5-[4-[(3-bromophenyl)methoxy]butyl]-6˜{H}-1,3,4-thiadiazin-2-amine; 5-[4-[(3-cyclopropylphenyl)methoxy]butyl]-6˜{H}-1,3,4-thiadiazin-2-amine; 5-[4-[(4-fluoro-3-methyl-phenyl)methoxy]butyl]-6˜{H}-1,3,4-thiadiazin-2-amine; 5-[4-[(4-fluorophenyl)methoxy]butyl]-6˜{H}-1,3,4-thiadiazin-2-amine; 5-[4-[(4-methoxyphenyl)methoxy]butyl]-6˜{H}-1,3,4-thiadiazin-2-amine; 5-[4-[(2,4-difluorophenyl)methoxy]butyl]-6˜{H}-1,3,4-thiadiazin-2-amine; 5-[4-[(3-chlorophenyl)methoxy]butyl]-6˜{H}-1,3,4-thiadiazin-2-amine; 5-[4-(m-tolylmethoxy)butyl]-6˜{H}-1,3,4-thiadiazin-2-amine; 5-[4-[(4-butylphenyl)methoxy]butyl]-6˜{H}-1,3,4-thiadiazin-2-amine; 5-[4-[[4-(trifluoromethyl)phenyl]methoxy]butyl]-6˜{H}-1,3,4-thiadiazin-2-amine; 5-[4-[(4-chlorophenyl)methoxy]butyl]-6˜{H}-1,3,4-thiadiazin-2-amine; 5-[5-(4-butylphenoxy)pentyl]-6˜{H}-1,3,4-thiadiazin-2-amine; 5-[2-[(4-butylphenyl)methoxy]ethyl]-6˜{H}-1,3,4-thiadiazin-2-amine; 5-[2-[3-(4-methoxyphenyl)propoxy]ethyl]-6˜{H}-1,3,4-thiadiazin-2-amine; 5-[5-(4-ethylphenoxy)pentyl]-6˜{H}-1,3,4-thiadiazin-2-amine; 5-[2-(4-methoxybutoxy)ethyl]-6˜{H}-1,3,4-thiadiazin-2-amine; 5-[2-(3-phenylpropoxy)ethyl]-6˜{H}-1,3,4-thiadiazin-2-amine; butyl 4-(2-amino-6˜{H}-1,3,4-thiadiazin-5-yl)benzoate; 5-[5-(3-ethylphenoxy)pentyl]-6˜{H}-1,3,4-thiadiazin-2-amine; 5-(4-butoxyphenyl)-4-methyl-1,3,4-thiadiazin-2-amine; 5-(4-benzyloxybutyl)-6˜{H}-1,3,4-thiadiazin-2-amine; 5-(4-methoxyphenyl)-4-methyl-1,3,4-thiadiazin-2-amine; 5-(benzofuran-2-yl)-4-methyl-1,3,4-thiadiazin-2-amine; 4-(2-amino-4-methyl-1,3,4-thiadiazin-5-yl)benzonitrile; ethyl 4-(2-amino-4-methyl-1,3,4-thiadiazin-5-yl)benzoate; 5-(3,4-dichlorophenyl)-4-methyl-1,3,4-thiadiazin-2-amine; 4-methyl-5-(p-tolyl)-1,3,4-thiadiazin-2-amine; 5-[2-(4-pentoxyphenyl)ethyl]-6˜{H}-1,3,4-thiadiazin-2-amine; 5-(5-benzyloxypentyl)-6˜{H}-1,3,4-thiadiazin-2-amine; 5-[4-(benzofuran-2-yl)butyl]-6˜{H}-1,3,4-thiadiazin-2-amine; 5-(5-methoxypentyl)-4-methyl-3˜{H}-1,3,4-thiadiazin-2-imine; 5-(4-chlorophenyl)-4-methyl-1,3,4-thiadiazin-2-amine; 2-[5-(2-amino-6˜{H}-1,3,4-thiadiazin-5-yl)pentyl]isoindoline-1,3-dione; 5-[5-(4-fluorophenoxy)pentyl]-6˜{H}-1,3,4-thiadiazin-2-amine; 5-[(˜{E})-hept-1-enyl]-6˜{H}-1,3,4-thiadiazin-2-amine; 4-(5-methoxypentyl)-5-methyl-1,3,4-thiadiazin-2-amine; 5-[5-(2-pyridyl)pentyl]-6˜{H}-1,3,4-thiadiazin-2-amine; 5-[5-(˜{N}-methylanilino)pentyl]-6˜{H}-1,3,4-thiadiazin-2-amine; 5-(6-methoxyhexyl)-6˜{H}-1,3,4-thiadiazin-2-amine; 5-(7-phenoxyheptyl)-6˜{H}-1,3,4-thiadiazin-2-amine; 5-(7-methoxyheptyl)-6˜{H}-1,3,4-thiadiazin-2-amine; 5-(4-methoxybutyl)-6˜{H}-1,3,4-thiadiazin-2-amine; 6-(2-amino-6˜{H}-1,3,4-thiadiazin-5-yl)hexanoic acid; ethyl 6-(2-amino-6˜{H}-1,3,4-thiadiazin-5-yl)hexanoate; 5-(methoxymethyl)-6˜{H}-1,3,4-thiadiazin-2-amine; 5-(4-phenoxybutyl)-6˜{H}-1,3,4-thiadiazin-2-amine; 5-[2-(4-phenylphenoxy)ethyl]-6˜{H}-1,3,4-thiadiazin-2-amine; 5-[2-(4-methylphenoxy)ethyl]-6˜{H}-1,3,4-thiadiazin-2-amine; 5-(phenoxymethyl)-6˜{H}-1,3,4-thiadiazin-2-amine; 5-(3-phenoxypropyl)-6˜{H}-1,3,4-thiadiazin-2-amine; 5-(6-phenoxyhexyl)-6˜{H}-1,3,4-thiadiazin-2-amine; 5-(6-phenylhexyl)-6˜{H}-1,3,4-thiadiazin-2-amine; 5-(2-amino-6˜{H}-1,3,4-thiadiazin-5-yl)-˜{N}-phenyl-pentanamide; 5-[2-[(4-methoxyphenyl)methoxy]ethyl]-6˜{H}-1,3,4-thiadiazin-2-amine; 5-[2-(4-propoxyphenyl)ethyl]-6˜{H}-1,3,4-thiadiazin-2-amine; 4-[2-(2-amino-6˜{H}-1,3,4-thiadiazin-5-yl)ethoxymethyl]benzonitrile; 5-[2-(1-methylbutoxy)ethyl]-6˜{H}-1,3,4-thiadiazin-2-amine; 5-[2-(p-tolylmethoxy)ethyl]-6˜{H}-1,3,4-thiadiazin-2-amine; 5-[2-[(4-phenylphenyl)methoxy]ethyl]-6˜{H}-1,3,4-thiadiazin-2-amine; 5-(2-hexoxyethyl)-6˜{H}-1,3,4-thiadiazin-2-amine; 5-[2-(2-methoxyethoxy)ethyl]-6˜{H}-1,3,4-thiadiazin-2-amine; 5-(2-phenoxyethyl)-6˜{H}-1,3,4-thiadiazin-2-amine; 5-[2-(4-bromophenoxy)ethyl]-6˜{H}-1,3,4-thiadiazin-2-amine; 5-(2-naphthyl)-6˜{H}-1,3,4-thiadiazin-2-amine; 5-(1-naphthyl)-4˜{H}-1,3,4-thiadiazin-2-amine; 5-(4-butoxyphenyl)-6˜{H}-1,3,4-thiadiazin-2-amine; 5-(3-butoxyphenyl)-6˜{H}-1,3,4-thiadiazin-2-amine; 5-(3,4-dichlorophenyl)-6˜{H}-1,3,4-thiadiazin-2-amine; ethyl 3-(2-amino-6˜{H}-1,3,4-thiadiazin-5-yl)benzoate; 5-(5-methoxypentyl)-6˜{H}-1,3,4-thiadiazin-2-amine; 5-(3-methoxypropyl)-6˜{H}-1,3,4-thiadiazin-2-amine; 5-(3,5-dimethylphenyl)-6˜{H}-1,3,4-thiadiazin-2-amine; ethyl 4-(2-amino-4˜{H}-1,3,4-thiadiazin-5-yl)benzoate; 5-heptyl-4˜{H}-1,3,4-thiadiazin-2-amine; isopropyl 2-(2-amino-6˜{H}-1,3,4-thiadiazin-5-yl)acetate; 5,6-dihydro-4˜{a}˜{H}-benzo[h][4,1,2]benzothiadiazin-3-amine; 5,6,7,8-tetrahydro-1˜{H}-4,1,2-benzothiadiazin-3-amine; 5-(5-phenoxypentyl)-6˜{H}-1,3,4-thiadiazin-2-amine; 5-(4-phenylbutyl)-6˜{H}-1,3,4-thiadiazin-2-amine; 6-butoxy-5,6,7,8-tetrahydro-4˜{a}˜{H}-4,1,2-benzothiadiazin-3-amine; 5-(4-cyclohexylphenyl)-6˜{H}-1,3,4-thiadiazin-2-amine; 5-(4-propylphenyl)-6˜{H}-1,3,4-thiadiazin-2-amine; 5-(4-isopropylphenyl)-6˜{H}-1,3,4-thiadiazin-2-amine; butyl 2-(2-amino-6˜{H}-1,3,4-thiadiazin-5-yl)acetate; 5-(2-benzyloxyethyl)-6˜{H}-1,3,4-thiadiazin-2-amine; 5-[2-(cyclohexoxy)ethyl]-6˜{H}-1,3,4-thiadiazin-2-amine; 5-(2-butoxyethyl)-6˜{H}-1,3,4-thiadiazin-2-amine; 5-(2-isopropoxyethyl)-6˜{H}-1,3,4-thiadiazin-2-amine; 5-(2-pyridyl)-6˜{H}-1,3,4-thiadiazin-2-amine; 4-(2-amino-6˜{H}-1,3,4-thiadiazin-5-yl)benzonitrile; 5-(4-methoxyphenyl)-6˜{H}-1,3,4-thiadiazin-2-amine; (2-amino-6˜{H}-1,3,4-thiadiazin-5-yl)methyl propanoate; methyl 2-(2-amino-6˜{H}-1,3,4-thiadiazin-5-yl)acetate; benzyl 2-(2-amino-6˜{H}-1,3,4-thiadiazin-5-yl)acetate; 5-(3-methoxyphenyl)-6˜{H}-1,3,4-thiadiazin-2-amine; 5-(4-chlorophenyl)-6˜{H}-1,3,4-thiadiazin-2-amine; 5-(3-chlorophenyl)-6˜{H}-1,3,4-thiadiazin-2-amine; 5-(p-tolyl)-6˜{H}-1,3,4-thiadiazin-2-amine; 5-(m-tolyl)-6˜{H}-1,3,4-thiadiazin-2-amine; 5-(2-methoxyethyl)-6˜{H}-1,3,4-thiadiazin-2-amine; 5-phenyl-6˜{H}-1,3,4-thiadiazin-2-amine; ethyl 2-(2-amino-6˜{H}-1,3,4-thiadiazin-5-yl)acetate; 6-(2-butoxyethyl)-4˜{H}-1,3,4-thiadiazin-2-amine; 5-[4-[(2-Bromophenyl)methoxy]butyl]-6H-1,3,4-thiadiazin-2-amine; 5-[4-[(3-Vinylphenyl)methoxy]butyl]-6H-1,3,4-thiadiazin-2-amine; 5-[4-[[3-[(E)-Prop-1-enyl]phenyl]methoxy]butyl]-6H-1,3,4-thiadiazin-2-amine; 5-[4-[(3-allylphenyl)methoxy]butyl]-6H-1,3,4-thiadiazin-2-amine; 5-[4-[(3-Prop-1-ynylphenyl)methoxy]butyl]-6H-1,3,4-thiadiazin-2-amine; 4-[4-(2-Amino-6H-1,3,4-thiadiazin-5-yl)butoxymethyl]benzonitrile; 3-[4-(2-Amino-6H-1,3,4-thiadiazin-5-yl)butoxymethyl]benzonitrile; 5-[6-(4-Bromo-2,6-difluoro-phenyl)hexyl]-6H-1,3,4-thiadiazin-2-amine; 5-[4-[(2,3,4-Trifluorophenyl)methoxy]butyl]-6H-1,3,4-thiadiazin-2-amine; 5-[4-[(2,3-Difluorophenyl)methoxy]butyl]-6H-1,3,4-thiadiazin-2-amine; 5-[4-[(3-Bromo-5-fluoro-phenyl)methoxy]butyl]-6H-1,3,4-thiadiazin-2-amine; 5-[4-[(2,4,6-Trifluorophenyl)methoxy]butyl]-6H-1,3,4-thiadiazin-2-amine; 5-[4-[(2,6-Difluorophenyl)methoxy]butyl]-6H-1,3,4-thiadiazin-2-amine; 5-[4-[(2,6-Difluoro-4-methoxy-phenyl)methoxy]butyl]-6H-1,3,4-thiadiazin-2-amine; 5-[4-[(4-Bromo-3-fluoro-phenyl)methoxy]butyl]-6H-1,3,4-thiadiazin-2-amine; 5-[4-(1,3-Benzothiazol-2-ylmethoxy)butyl]-6H-1,3,4-thiadiazin-2-amine; 5-[4-[(2-Nitrophenyl)methoxy]butyl]-6H-1,3,4-thiadiazin-2-amine; 5-[4-(Tetrahydrofuran-2-ylmethoxy)butyl]-6H-1,3,4-thiadiazin-2-amine; 5-[4-[(2-Methylcyclopropyl)methoxy]butyl]-6H-1,3,4-thiadiazin-2-amine; 5-[4-[(2,4-Dimethylphenyl)methoxy]butyl]-6H-1,3,4-thiadiazin-2-amine; 5-[4-[(2-Chlorophenyl)methoxy]butyl]-6H-1,3,4-thiadiazin-2-amine; 5-[4-[(5-Methyl-2-thienyl)methoxy]butyl]-6H-1,3,4-thiadiazin-2-amine; Ethyl 3-[3-[4-(2-amino-6H-1,3,4-thiadiazin-5-yl)butoxymethyl]phenyl]propanoate; 2-[4-(2-Amino-6H-1,3,4-thiadiazin-5-yl)butoxymethyl]benzonitrile;
and tautomers thereof and salts thereof.

12. A compound for use according to claim 1 wherein the compound is selected from:
5-[4-(4-ethylphenoxy)butyl]-6˜{H}-1,3,4-thiadiazin-2-amine; 5-[5-(2-pyridyloxy)pentyl]-6˜{H}-1,3,4-thiadiazin-2-amine; 5-[2-(2-phenoxyethoxy)ethyl]-6˜{H}-1,3,4-thiadiazin-2-amine; 5-[4-[(3-bromophenyl)methoxy]butyl]-6˜{H}-1,3,4-thiadiazin-2-amine; 5-[4-[(2,4-difluorophenyl)methoxy]butyl]-6˜{H}-1,3,4-thiadiazin-2-amine; 5-(4-benzyloxybutyl)-6˜{H}-1,3,4-thiadiazin-2-amine; 5-(5-benzyloxypentyl)-6˜{H}-1,3,4-thiadiazin-2-amine; 5-(5-methoxypentyl)-4-methyl-3˜{H}-1,3,4-thiadiazin-2-imine; 5-[5-(4-fluorophenoxy)pentyl]-6˜{H}-1,3,4-thiadiazin-2-amine; 5-(2-hexoxyethyl)-6˜{H}-1,3,4-thiadiazin-2-amine; 5-(5-methoxypentyl)-6˜{H}-1,3,4-thiadiazin-2-amine; ethyl 4-(2-amino-4˜{H}-1,3,4-thiadiazin-5-yl)benzoate; 5-(5-phenoxypentyl)-6˜{H}-1,3,4-thiadiazin-2-amine and 5-(2-butoxyethyl)-6˜{H}-1,3,4-thiadiazin-2-amine,
and tautomers thereof and salts thereof

13. A compound which is a diazine of formula (I) or a tautomer thereof, or a pharmaceutically acceptable salt thereof 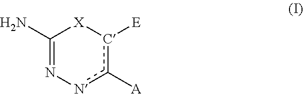
wherein the moiety 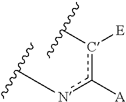
represents -N(D)-C(A)=C(E)- or -N=C(A)-C(R¹)(E)- as defined in claim 1, and wherein X, D, E, R¹, R², L, T and V are as defined in any one of claims 2 to 10, wherein A is -L-T, wherein
i) L is a linear alkylene or alkenylene moiety comprising from 5 to 12 carbon atoms; wherein L is unsubstituted or is substituted with 1, 2 or 3 groups selected from halogen, C₁-C₄ alkoxy and —OH; and wherein L may optionally terminate in and/or be interrupted by a heteromoiety selected from —O—, —S—, —C(O)—, —OC(O)—, —C(O)O—, —NR²—, —NR²C(O)—, and —C(O)NR²—;
and/or
ii) T is aryl, heteroaryl, cycloalkyl or heterocyclyl; wherein T is unsubstituted or is substituted by 1, 2 or 3 groups V;
wherein the compound is not: 2-(2-(2-amino-2H-1,3,4-thiadiazin-5-yl)ethyl)isoindoline-1,3-dione; 2-(1-(2-amino-2H-1,3,4-thiadiazin-5-yl)ethyl)isoindoline-1,3-dione; 5-benzyl-6H-1,3,4-thiadiazin-2-amine; 2-amino-5-benzyl-1,3,4-thiadiazine; 5-(3-nitrobenzyl)-6H-1,3,4-thiadiazin-2-amine; 5-(3-methylpentan-3-yl)-6H-1,3,4-thiadiazin-2-amine; or 5-[2-(5-nitro-2-furanyl)ethenyl]-6H-1,3,4-thiadiazin-2-amine.

14. A compound according to claim 13 wherein L is a C₅-C₁₂ alkylene moiety, wherein L is unsubstituted and wherein L terminates in a heteromoiety —O— and/or is interrupted by a heteromoiety —O—.

15. A composition comprising a compound according to claim 13 or claim 14 together with one or more pharmaceutically acceptable carriers and/or excipients and/or diluents.

16. A product comprising a compound as defined in any one of claims 1 to 14 and further comprising a second antifungal agent.

17. A compound, composition or product according to any one of claims 13 to 16 for use in a method of treatment of the human or animal body by therapy.

18. Use of a compound as defined in any one of claims 1 to 14, a composition as defined in claim 15 or a product as defined in claim 16 in the manufacture of a medicament for use in the prevention or treatment of fungal infection.

19. A method for the treatment or prevention of fungal infection, the method comprising administering to a subject in need of such treatment an effective amount of a compound as defined in any one of claims 1 to 14, a composition as defined in claim 15 or a product as defined in claim 16.

20. A compound for use according to any one of claims 1 to 12, a use according to claim 18 or a method according to claim 19 wherein the fungus to be treated is selected from Candida, Aspergillus, Cryptococcus, Histoplasma, Pneumocystis, Stachybotrys, Trichophyton, Absidia, Rhizopus, Fusarium, and Scedosporium.
